(19) United States
(12) Patent Application Publication
(10) Pub. No.: US 2009/0298894 A1
(43) Pub. Date: Dec. 3, 2009

(54) AMINO ACID COMPOUNDS

(75) Inventors: Yutaka Ohmori, Tokyo (JP); Takayuki Serizawa, Tokyo (JP); Kazuteru Sugie, Tokyo (JP); Kousuke Tanaka, Tokyo (JP); Akiko Matsumoto, Tokyo (JP)

Correspondence Address: BIRCH STEWART KOLASCH & BIRCH, PO BOX 747, FALLS CHURCH, VA, 22040-0747, US

(73) Assignee: Asahi Kasei Pharma Corporation, Tokyo (JP)

(21) Appl. No.: 12/426,508

(22) Filed: Apr. 20, 2009

Related U.S. Application Data
(60) Provisional application No. 61/046,490, filed on Apr. 21, 2008.

(51) Int. Cl. A61K 31/4245; C07D 271/06; A61P 37/06

(52) U.S. Cl. 514/364; 548/131

(57) ABSTRACT

[Problem] To provide novel compounds that are S1P1 receptor agonists and exhibit an immunosuppressive activities by inducing lymphocyte sequestration in secondary lymphoid tissues. In addition, to provide a pharmaceutical agent which comprises the compounds as an effective component, in particular to provide a therapeutic and/or prophylactic agent for an autoimmune disease and the like.

[Solving Means] Amino acid compounds that are represented by the following Formula (1) are provided

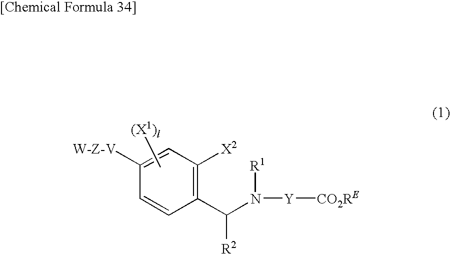

### TECHNICAL FIELD

The present invention is related to novel amine compounds that are S1P1/Edg1 receptor agonists and can produce lymphocyte sequestration in secondary lymphoid tissues, and thus are useful as an effective component for pharmaceuticals having an immunosuppressive activities, and an intermediate for preparing the compounds.

### BACKGROUND ART

Conventionally, for treating rheumatoid arthritis and other autoimmune diseases, anti-inflammatory pharmaceuticals such as steroids, have been used against inflammatory reactions that are caused by abnormal immunoreactions. However, this is basically a symptomatic treatment and is not a treatment which eradicates the basic cause of the disease. Meanwhile, development of a method for inhibiting immunoresponses is very important for inhibiting a rejection reaction after organ implant or cell implant or for curative and prophylactic treatment against various autoimmune diseases. In fact, immunosuppressive agents have been shown to be useful in a wide variety of autoimmune or chronic inflammatory diseases, including systemic lupus erythematosus, chronic rheumatoid arthritis, type I diabetes mellitus, inflammatory bowel disease, biliary cirrhosis, uveitis, multiple sclerosis and other disorders such as Crohn's disease, ulcerative colitis, bullous pemphigoid, sarcoidosis, psoriasis, autoimmune myositis, Wegener's granulomatosis, ichthyosis, Graves opthalmopathy, atopic dermatitis and asthma.

Although the underlying pathogenesis of each of these autoimmune diseases may be quite different, they have in common the occurrence of a variety of autoantibodies and/or self-reactive lymphocytes. Such self-reactivity may be due, in part, to a loss of the homeostatic controls under which the normal immune system operates. Similarly, following a bone-marrow or an organ transplantation, the host lymphocytes recognize the foreign tissue antigens and begin to produce both cellular and humoral responses including antibodies, cytokines and cytotoxic lymphocytes which lead to graft rejection.

As a result of an autoimmune or a rejection process, tissue destruction is caused by inflammatory cells and/or the mediators they release. Anti-inflammatory agents such as NSAIDs act principally by blocking the activity or secretion of these mediators but do nothing to modify the immunologic basis of the disease.

Cyclosporin A and tacrolimus are drugs used to prevent rejection of transplanted organs. Cyclosporin A and tacrolimus can inhibit in vivo immunoreactions which are activated to reject foreign proteins of a graft. Although Cyclosporin A and tacrolimus are effective in delaying or suppressing transplant rejection, they are known to cause several undesirable side effects including nephrotoxicity, neurotoxicity, and gastrointestinal discomfort. At present stage, an immunosuppressive agent having no such side effects is not developed yet. Under the circumstances, various studies have been carried out to develop a compound which has an excellent immunosuppressive activity and low toxicity.

The immunosuppressive compound FTY720 is a lymphocyte sequestration agent currently in clinical trials.

Agonistic activity of FTY720 on sphingosine 1-phosphate receptors induces the sequestration of lymphocytes (T-cells and B-cells) in lymph nodes and Peyer's patches without lymphodepletion. Thus, an agonist of sphingosine 1-phosphate receptors can function as an immunoregulatory agent which can induce reduction in lymphocytes which is based on redistribution from circulation to secondary lymph tissues without inducing systemic immunosuppression. Such immunosuppression is desirable to prevent rejection after organ transplantation and in the treatment of autoimmune diseases.

However, since a side effect of FTY720 has also been reported that bradycardia is found after administration (Non-Patent Document 1), sufficient caution is required for the use. As such, a pharmaceutical agent which has high efficacy and high safety is needed.

Although sphingosine 1-phosphate has been regarded as an intermediate metabolite in sphingosine metabolism, nowadays it is known to have an activity of promoting cell proliferation and an activity of controlling cell mobility. Thus, it is now clear that it is indeed a lipid mediator which has various physiological activities such as apoptosis activity, control of cell morphology, vascular contraction, etc. Activity of sphingosine 1-phosphate is based on signalling via plural G-protein coupled receptors, which are present on surface of cell membrane. At present, five subtype sphingosine 1-phosphate receptors have been identified (S1P1, S1P2, S1P3, S1P4, and S1P5, also known as endothelial differentiation genes Edg1, Edg5, Edg3, Edg6, Edg8). In addition, they have widespread cellular and tissue distribution and are well conserved in human and rodent species. It has been disclosed that ligand-induced activation of S1P1 and S1P3 can promote angiogenesis, chemotaxis, and adherence junction assembly, whereas agonistic activity of S1P2 promotes neurite retraction and also inhibits chemotaxis of cells. S1P4 is localized to hematopoietic cells and tissues, whereas S1P5 is primarily expressed as a neuronal receptor with some expression in lymphoid tissue.

Administration of sphingosine 1-phosphate to animals induces systemic sequestration of peripheral blood lymphocytes into secondary lymphoid organs, thus resulting in therapeutically useful immunosuppression. However, sphingosine 1-phosphate also has cardiovascular and bronchoconstrictor effects that limit its utility as a therapeutic agent. Intravenous administration of sphingosine 1-phosphate decreases the heart rate in rats (Non-Patent Document 2). The undesirable effects of sphingosine 1-phosphate are associated with its non-selective agonistic activity for all S1P receptors.

Under the circumstances, development of compounds that are selective for the subtypes of S1P receptor is needed.

Meanwhile, as a compound which has a similar function as the compounds of the present invention, the compounds disclosed in the Patent Document 1 to 3 have been known. However, they are all different from the compounds of the present invention in terms of characteristics of a chemical structure.

### PRIOR ART LITERATURES

### [Patent Documents]

[Patent Document 1] International Publication No. WO 03/105771 pamphlet

[Patent Document 2] International Publication No. WO 05/058848 pamphlet

[Patent Document 3] International Publication No. WO 02/044780 pamphlet

### [Non-Patent Documents]

[Non-Patent Document 1] J. Am. Soc. Nephrol., 13, 1073 (2002) [Non-Patent Document 2] Jpn. J. Pharmacol., 82, 338 (2000)

### SUMMARY OF THE INVENTION

### Problems to be Solved by the Invention

Object of the present invention is to provide novel compounds which can inhibit an immunoresponse with little side effect. More specifically, the present invention is related to novel compounds that are S1P1 receptor agonists, having immunosuppressive activities by producing lymphocyte sequestration in secondary lymphoid tissues. In addition, another object of the present invention is to provide pharmaceuticals which contain the compounds as an effective component. More specifically, according to the present invention, a prophylactic and/or therapeutic agent for eradicating basic causes of an autoimmune disease, etc. is provided.

### Means for Solving the Problems

In order to solve the problems described above, inventors of the present invention tried to find out compounds which have a selective and high agonistic activity for S1P1/Edg1 receptor, in particular compounds which have higher agonistic activity for the S1P1/Edg1 receptor over S1P3/Edg3 receptor. As a result of searching for an agonist that is selective for the S1P1 receptor, it was found that the novel amine compounds, represented by each of the following formulae, have a selective agonist activity for the S1P1 receptor, and they are also useful as an immunosuppressive agent. The present invention is completed based on such findings.

Specifically, the present invention is related to the followings.

Compounds represented by Formula 1, a possible stereoisomer, a racemate, a pharmaceutically acceptable salt, a hydrate, a solvate or a prodrug thereof:

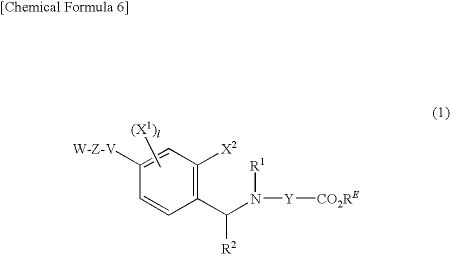

[in the Formula (1), W represents a monovalent group derived from a compound selected from benzene, thiophene, furan and pyridine by the removal of one hydrogen atom and the W may be substituted with one or two X^{W}, wherein X^{W} indicates a C1-C4 alkyl group which may be substituted with 1 to 9 fluorine atoms, a C1-C4 alkoxy group which may be substituted with 1 to 9 fluorine atoms, a halogen atom, a cyano group, a C1-C4 alkylthio group which may be substituted with 1 to 9 fluorine atoms, a C1-C4 alkylsulfinyl group which may be substituted with 1 to 9 fluorine atoms, a C1-C4 alkylsulfonyl group which may be substituted with 1 to 9 fluorine atoms, a C1-C4 acylamide group which may be substituted with 1 to 7 fluorine atoms, a C1-C4 alkylcarbamoyl group which may be substituted with 1 to 9 fluorine atoms, a C1-C4 alkylsulfonamide group which may be substituted with 1 to 9 fluorine atoms, a C1-C4 alkylsulfamoyl group which may be substituted with 1 to 9 fluorine atoms, a C1-C4 acyl group which may be substituted with 1 to 7 fluorine atoms, or a C1-C4 alkyl group which is substituted with one C1-C4 alkoxy group which may be substituted with 1 to 9 fluorine atoms or with one —OH group, and when it is substituted with two X^{W}, they can be the same or different from each other;

Z represents a divalent group derived from benzene by the removal of two hydrogen atoms, which binds to W— and —V— at para position and may be substituted with 1 to 4 X^{Z}, X^{Z} indicates a C1-C4 alkyl group which may be substituted with 1 to 9 fluorine atoms, a C1-C4 alkoxy group which may be substituted with 1 to 9 fluorine atoms, a halogen atom or a cyano group, and when it is substituted with two or more X^{Z}, they can be the same or different from each other;

V represents a divalent group derived from [1,2,4]-oxadiazole by the removal of two hydrogen atoms or —(CR^{V1}R^{V2})ₙ—(CR^{V3}R^{V4})ₖ—O—;

R^{V1}, R^{V2}, R^{V3}, and R^{V4} can be the same or different from each other, and each independently represent a hydrogen atom, a halogen atom, or a C1-C4 alkyl group which may be substituted with 1 to 5 halogen atoms;

n indicates an integer of 0 to 2, and when n is 0, —(CR^{V1}R^{V2})ₙ— means a single bond;

k indicates an integer of 0 or 1 and when k is 0, —(CR^{V3}R^{V4})ₖ— means a single bond;

X¹ indicates a C1-C4 alkyl group which may be substituted with 1 to 9 fluorine atoms, a C1-C4 alkoxy group which may be substituted with 1 to 9 fluorine atoms, or a halogen atom,

l indicates an integer of 0 to 3;

when l is 2 or 3, X¹ can be the same or different from each other;

R¹ indicates a hydrogen atom or a C1-C4 alkyl group which may be substituted with 1 to 5 halogen atoms, or is linked to X² via a C1 alkylene to form a 5-membered ring, wherein the C1 alkylene may be substituted with one or two C1-C4 alkyl groups (they may be also substituted with 1 to 5 halogen atoms);

R² indicates a hydrogen atom or a C1-C4 alkyl group which may be substituted with 1 to 5 halogen atoms, or is linked to X² via a C2 alkylene to form a 5-membered ring, wherein the C2 alkylene may be substituted with one or two C1-C4 alkyl groups (they may be also substituted with 1 to 5 halogen atoms), or is linked to X² via a C3 alkylene to form a 6-membered ring, wherein the C3 alkylene may be substituted with one or two C1-C4 alkyl groups (they may be also substituted with 1 to 5 halogen atoms);

any one of R¹ and R² is linked to X² to form a ring;

X² indicates a single bond;

Y indicates a cyclobutylene group and may be substituted with 1 to 4 X^{Y}, and it binds to —CO₂R^{E} and —NR¹— at position 1 and position 3 of the cyclobutylene group, respectively;

X^{Y} represents —OH, a halogen atom or a C1-C4 alkyl group;

the C1-C4 alkyl group described above may be substituted with 1 to 5 halogen atoms;

R^{E} indicates a hydrogen atom, a C1-C4 alkyl group, —(CH₂)ₘN(R^{E1})(R^{E2}) or —C(R^{E3})₂OC(O)A^{E}R^{E4};

m indicates an integer of 2 or 3;

R^{E1} and R^{E2} can be the same or different from each other and each independently represent a methyl group, an ethyl group, or a propyl group, or a nitrogen-containing saturated cycloalkyl group in which R^{E1} and R^{E2} are linked to each other to form a 3- to 6-membered ring together with a nitrogen atom, or form a morpholine group together with a nitrogen atom;

R^{E3} indicates a hydrogen atom, a methyl group, an ethyl group, or a propyl group;

R^{E4} indicates a C1-C4 alkyl group, C3-C6 cycloalkyl group, or a phenyl group, and;

A^{E} indicates a single bond or an oxygen atom.].

### [A1-2]

W represents a monovalent group derived from a compound selected from benzene, thiophene, and pyridine by the removal of one hydrogen atom and,

the W may be substituted with one or two X^{W}, and the X^{W} indicates a C1-C4 alkyl group which may be substituted with 1 to 9 fluorine atoms, a C1-C4 alkoxy group which may be substituted with 1 to 9 fluorine atoms, a halogen atom, a cyano group, or a C1-C4 alkylthio group which may be substituted with 1 to 9 fluorine atoms, and when it is substituted with two X^{W}, they can be the same or different from each other;

Z represents a divalent group derived from benzene by the removal of two hydrogen atoms, and it binds to W— and —V— at para position and may be substituted with 1 to 4 X^{Z}, wherein X^{Z} indicates a C1-C4 alkyl group which may be substituted with 1 to 9-fluorine atoms, a C1-C4 alkoxy group which may be substituted with 1 to 9 fluorine atoms, a halogen atom or a cyano group, and when it is substituted with two or more X^{Z}, they can be the same or different from each other;

-Z-V— is represented by the following Formula (2) (in the Formula (2), Z is as defined above),

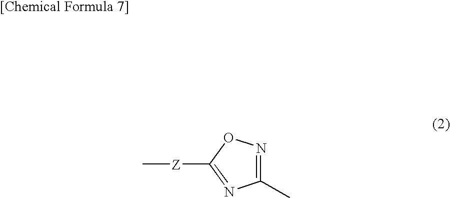

X¹ indicates a C1-C4 alkyl group which may be substituted with 1 to 9 fluorine atoms, a C1-C4 alkoxy group which may be substituted with 1 to 9 fluorine atoms, or a halogen atom,

l indicates an integer of 0 to 3;

when l is 2 or 3, X¹ can be the same or different from each other;

R¹ indicates a hydrogen atom, or is linked to X² via C1 alkylene to form a 5-membered ring, wherein the C1 alkylene may be substituted with one or two C1-C4 alkyl groups;

R² indicates a hydrogen atom, or is linked to X² via C2 alkylene to form a 5-membered ring, wherein the C2 alkylene may be substituted with one or two C1-C4 alkyl groups, or is linked to X² via C3 alkylene to form a 6-membered ring, wherein the C3 alkylene may be substituted with one or two C1-C4 alkyl groups;

any one of R¹ and R² is linked to X² to form a ring;

X² indicates a single bond;

Y indicates an unsubstituted cyclobutylene group and it binds to —CO₂R^{E} and —NR¹— at position 1 and position 3 of the cyclobutylene group, respectively;

R^{E} indicates a hydrogen atom or a C1-C4 alkyl group.

The compounds according to [A1] or [A1-2], a possible stereoisomer, a racemate, a pharmaceutically acceptable salt, a hydrate, a solvate or a prodrug thereof, wherein R¹ is linked to X² via a C1 alkylene which may be substituted with one or two C1-C4 alkyl group to form a 5-membered ring.

### [A2-2]

The compounds according to [A1] or [A1-2], a possible stereoisomer, a racemate, a pharmaceutically acceptable salt, a hydrate, a solvate or a prodrug thereof, wherein R¹ is linked to X² via a C1 alkylene to form a 5-membered ring.

### [A3]

The compounds according to [A1] or [A1-2], a possible stereoisomer, a racemate, a pharmaceutically acceptable salt, a hydrate, a solvate or a prodrug thereof, wherein R² is linked to X² via a C2 alkylene which may be substituted with one or two C1-C4 alkyl group to form a 5-membered ring.

### [A3-2]

The compounds according to [A1] or [A1-2], a possible stereoisomer, a racemate, a pharmaceutically acceptable salt, a hydrate, a solvate or a prodrug thereof, wherein R² is linked to X² via a C2 alkylene to form a 5-membered ring.

### [A4]

The compounds according to [A1] or [A1-2], a possible stereoisomer, a racemate, a pharmaceutically acceptable salt, a hydrate, a solvate or a prodrug thereof, wherein R² is linked to X² via a C3 alkylene which may be substituted with one or two C1-C4 alkyl group to form a 6-membered ring.

### [A4-2]

The compounds according to [A1] or [A1-2], a possible stereoisomer, a racemate, a pharmaceutically acceptable salt, a hydrate, a solvate or a prodrug thereof, wherein R² is linked to X² via a C3 alkylene to form a 6-membered ring.

### [A5]

The compounds according to any one of [A1] to [A4-2], a possible stereoisomer, a racemate, a pharmaceutically acceptable salt, a hydrate, a solvate or a prodrug thereof, wherein Y is an unsubstituted cyclobutylene group.

In addition, when the item numbers are referred to such as [A1] to [A4] to include a range and there is an additional item having branch number such as [A1-2], etc., an item having branch number such as [A1-2], etc. is also referred to, and it has the same meaning for the following descriptions.

### [A6]

The compounds according to any one of [A1] to [A5], a possible stereoisomer, a racemate, a pharmaceutically acceptable salt, a hydrate, a solvate or a prodrug thereof, wherein -Z-V— is represented by Formula (2) (in the Formula (2), Z is as defined above).

### [A7]

The compounds according to any one of [A1] to [A5], a possible stereoisomer, a racemate, a pharmaceutically acceptable salt, a hydrate, a solvate or a prodrug thereof, wherein -Z-V— is -Z-CR^{V1}R^{V2}—O— (in the formula, Z, R^{V1}, and R^{V2} are as defined above).

### [A8]

The compounds according to any one of [A1] to [A5], a possible stereoisomer, a racemate, a pharmaceutically acceptable salt, a hydrate, a solvate or a prodrug thereof, wherein -Z-V— is -Z-(CR^{V1}R^{V2})—(CR^{V3}R^{V4})—O— (in the formula, Z, R^{V1}, R^{V2}, R^{V3}, and R^{V4} are as defined above).

### [A8-2]

The compounds according to any one of [A1] to [A5], a possible stereoisomer, a racemate, a pharmaceutically acceptable salt, a hydrate, a solvate or a prodrug thereof, wherein -Z-V— is -Z-(CR^{V1}R^{V2})₂—CR^{V3}R^{V4}—O— (in the formula Z, R^{V1}, R^{V2}, R^{V3}, and R^{V4} are as defined above).

### [A9]

The compounds according to any one of [A1] to [A8-2], a possible stereoisomer, a racemate, a pharmaceutically acceptable salt, a hydrate, a solvate or a prodrug thereof, wherein bonding between Y and —NR¹— and bonding between Y and —CO₂R^{E} are in trans configuration.

### [A10]

The compounds according to any one of [A1] to [A9], a possible stereoisomer, a racemate, a pharmaceutically acceptable salt, a hydrate, a solvate or a prodrug thereof, wherein X^{W} is a C1-C4 alkyl group which may be substituted with 1 to 9-fluorine atoms, a C1-C4 alkoxy group which may be substituted with 1 to 9 fluorine atoms, a halogen atom, or a C1-C4 alkylthio group which may be substituted with 1 to 9 fluorine atoms.

### [A11]

The compounds according to any one of [A1] to [A10], a possible stereoisomer, a racemate, a pharmaceutically acceptable salt, a hydrate, a solvate or a prodrug thereof, wherein W is substituted with one or two X^{W}, and at least one X^{W} is a C1-C4 alkylthio group which may be substituted with 1 to 9 fluorine atoms, a C1-C4 acyl group which may be substituted with 1 to 7 fluorine atoms, or a C1-C4 alkyl group which is substituted with one C1-C4 alkoxy group which may be substituted with 1 to 9 fluorine atoms or with one —OH group, and when W is substituted with two X^{W}, they can be the same or different from each other.

### [A12]

The compounds according to any one of [A1] to [A11], a possible stereoisomer, a racemate, a pharmaceutically acceptable salt, a hydrate, a solvate or a prodrug thereof, wherein Z may be substituted with one to three X^{Z}, and X^{Z} is a C1-C4 alkyl group which may be substituted with 1 to 9 fluorine atoms, a C1-C4 alkoxy group which may be substituted with 1 to fluorine atoms, or a fluorine atom, and when Z is substituted with two or more X^{Z}, they can be the same or different from each other.

### [A13]

The compounds according to any one of [A1] to [A12], a possible stereoisomer, a racemate, a pharmaceutically acceptable salt, a hydrate, a solvate or a prodrug thereof, wherein Z is substituted with one to three X^{Z}, and X^{Z} is a methyl group or a fluorine atom, and when Z is substituted two or more X^{Z}, they can be the same or different from each other.

### [A14]

The compounds according to any one of [A1] to [A13], a possible stereoisomer, a racemate, a pharmaceutically acceptable salt, a hydrate, a solvate or a prodrug thereof, wherein Z is substituted with two X^{Z}, and X^{Z} is a methyl group or a fluorine atom, and two X^{Z} can be the same or different from each other.

### [A15]

The compounds according to any one of [A1] to [A14], a possible stereoisomer, a racemate, a pharmaceutically acceptable salt, a hydrate, a solvate or a prodrug thereof, wherein W-Z-V— is represented by Formula (3) (in the Formula (3), W and V are as defined above).

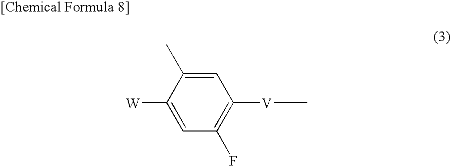

### [A16]

The compounds according to any one of [A1] to [A14], a possible stereoisomer, a racemate, a pharmaceutically acceptable salt, a hydrate, a solvate or a prodrug thereof, wherein W-Z-V— is represented by Formula (4) (in the Formula (4), W and V are as defined above).

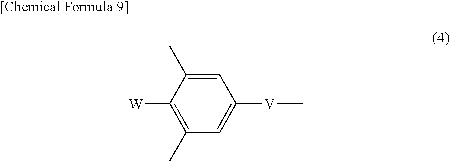

### [A17]

The compounds according to any one of [A1] to [A14], a possible stereoisomer, a racemate, a pharmaceutically acceptable salt, a hydrate, a solvate or a prodrug thereof, wherein W-Z-V— is represented by Formula (5) (in the Formula (5), W and V are as defined above).

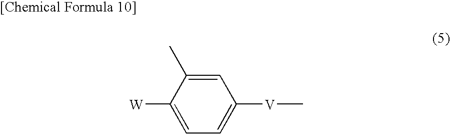

### [A18]

The compounds according to any one of [A1] to [A17], a possible stereoisomer, a racemate, a pharmaceutically acceptable salt, a hydrate, a solvate or a prodrug thereof, wherein X¹ is a trifluoromethyl group, a methyl group, an ethyl group, a fluorine atom, or a chlorine atom, and when two or more X¹ are present, they can be the same or different from each other.

### [A19]

The compounds according to any one of [A1] to [A18], a possible stereoisomer, a racemate, a pharmaceutically acceptable salt, a hydrate, a solvate or a prodrug thereof, wherein 1 is 1 and X¹ is a methyl group, a fluorine atom, or a chlorine atom.

### [A20]

The compounds according to any one of [A1] to [A19], a possible stereoisomer, a racemate, a pharmaceutically acceptable salt, a hydrate, a solvate or a prodrug thereof, wherein W is a monovalent group derived from benzene by the removal of one hydrogen atom.

### [A20-2]

The compounds according to any one of [A1] to [A20], a possible stereoisomer, a racemate, a pharmaceutically acceptable salt, a hydrate, a solvate or a prodrug thereof, wherein W is substituted with one X^{W}.

### [A20-3]

The compounds according to any one of [A1] to [A20], a possible stereoisomer, a racemate, a pharmaceutically acceptable salt, a hydrate, a solvate or a prodrug thereof, wherein W is substituted with two X^{W}.

### [A20-4]

The compounds according to any one of [A1] to [A20-3], a possible stereoisomer, a racemate, a pharmaceutically acceptable salt, a hydrate, a solvate or a prodrug thereof, wherein the X^{W} is a halogen atom or a C1-C4 alkyl group which may be substituted with 1 to 9 fluorine atoms.

### [A20-5]

The compounds according to any one of [A1] to [A20-3], a possible stereoisomer, a racemate, a pharmaceutically acceptable salt, a hydrate, a solvate or a prodrug thereof, wherein the X^{W} is a halogen atom.

### [A20-6]

The compounds according to any one of [A1] to [A20-5], a possible stereoisomer, a racemate, a pharmaceutically acceptable salt, a hydrate, a solvate or a prodrug thereof, wherein the W is substituted with two X^{W} and the two X^{W}, which can be the same or different from each other, are any one of a fluorine atom and a trifluoromethyl group.

### [A21]

The compounds according to any one of [A1] to [A19], a possible stereoisomer, a racemate, a pharmaceutically acceptable salt, a hydrate, a solvate or a prodrug thereof, wherein W is a monovalent group derived from thiophene by the removal of one hydrogen atom.

### [A21-2]

The compounds according to any one of [A1] to [A19], a possible stereoisomer, a racemate, a pharmaceutically acceptable salt, a hydrate, a solvate or a prodrug thereof, wherein W is a monovalent group derived from furan by the removal of one hydrogen atom.

### [A22]

The compounds according to any one of [A1] to [A19], a possible stereoisomer, a racemate, a pharmaceutically acceptable salt, a hydrate, a solvate or a prodrug thereof, wherein W is a monovalent group derived from pyridine by the removal of one hydrogen atom.

### [A22-2]

The compounds as described in any one of [A1] to [A22], a possible stereoisomer, a racemate, a pharmaceutically acceptable salt, a hydrate, a solvate or a prodrug thereof,

wherein R² is linked to X² via C2 alkylene to form a 5-membered ring,

l indicates 0,

R¹ is a hydrogen atom,

W is abenzene ring which may be substituted at meta position relative to the bonding with Z with one X^{W} selected from a group consisting of a trifluoromethyl group, a fluorine atom, and a chlorine atom,

Z represents a benzene ring substituted at ortho position relative to the bonding with W with one X^{Z} selected from a group consisting of a methyl group, a trifluoromethyl group, a f fluorine atom, a chlorine atom, and a cyano group,

-Z-V— is represented by the above Formula (2) (in the Formula (2), Z is as defined above),

Y represents an unsubstituted cyclobutylene group,

bonding between Y and —NR¹— and bonding between Y and —CO₂R^{E} are in trans configuration; and

R^{E} indicates a hydrogen atom.

### [A22-3]

The compounds as described in [A22-2], a possible stereoisomer, a racemate, a pharmaceutically acceptable salt, a hydrate, a solvate or a prodrug thereof,

wherein W is a benzene ring which may be substituted at meta position relative to the bonding with Z with one X^{W} selected from a group consisting of a trifluoromethyl group and a fluorine atom, and

Z represents a benzene ring substituted at ortho position relative to the bonding with W with one X^{Z} selected from a group consisting of a methyl group, a trifluoromethyl group and a fluorine atom.

### [A22-4]

The compounds as described in any one of [A1] to [A22-3], a possible stereoisomer, a racemate, a pharmaceutically acceptable salt, a hydrate, a solvate or a prodrug thereof, wherein W is a benzene ring which is substituted with a cyano group.

### [A22-5]

The compounds as described in any one of [A1] to [A22-4], a possible stereoisomer, a racemate, a pharmaceutically acceptable salt, a hydrate, a solvate or a prodrug thereof, wherein W is a benzene ring substituted with two X^{W} and the two X^{W} can be the same or different from each other and at least one of them is a cyano group and the other is a trifluoromethyl group, a fluorine atom, a chlorine atom, or a cyano group.

### [A22-6]

The compounds as described in any one of [A1] to [A22-5], a possible stereoisomer, a racemate, a pharmaceutically acceptable salt, a hydrate, a solvate or a prodrug thereof, wherein, for a case in which W is a benzene ring substituted with two X^{W}, the W is a benzene ring substituted with additional X^{W} selected from a group consisting of a C1-C4 alkyl group which may be substituted with 1 to 9 fluorine atoms, a C1-C4 alkoxy group which may be substituted with 1 to 9 fluorine atoms, a halogen atom, and a cyano group (two X^{W} can be the same or different from each other).

### [A23]

A pharmaceutical agent which comprises as an effective component the compounds according to any one of [A1] to [A22-6], a possible stereoisomer, a racemate, a pharmaceutically acceptable salt, a hydrate, a solvate or a prodrug thereof.

### [A24]

A S1P1/Edg1 receptor agonist which comprises as an effective component the compounds according to any one of [A1] to [A22-6], a possible stereoisomer, a racemate, a pharmaceutically acceptable salt, a hydrate, a solvate or a prodrug thereof.

### [A25]

The pharmaceutical agent according to [A23] which is used for prophylaxis and/or treatment of an autoimmune disease of mammals.

### [A26]

A method for the prophylaxis and/or treatment of an autoimmune disease of a mammal comprising administering to the mammal including human an effective amount of the compounds described in any one of [A1] to [A22-6], a possible stereoisomer, a racemate, a pharmaceutically acceptable salt, a hydrate, a solvate or a prodrug thereof.

### EFFECT OF THE INVENTION

The compounds of the present invention have a strong immunosuppressive activity when they are administered in a free or a salt form to a human or an animal. Thus, they are useful in a chemotherapy for treating a wide variety of autoimmune diseases or chronic inflammatory diseases including systemic lupus erythematosus, chronic rheumatoid arthritis, type-I diabetes, inflammatory bowel disease, biliary cirrhosis, uveitis, multiple sclerosis and other disorders, cancer, lymphoma, or leukemia, for example.

### BEST MODES FOR CARRYING OUT THE INVENTION

Herein below, the present invention will be explained in greater detail.

According to the present specification, a carbon atom is sometimes expressed simply as “C”, a hydrogen atom is sometimes expressed simply as “H”, an oxygen atom is sometimes expressed simply as “O”, a sulfur atom is sometimes expressed simply as “S”, and a nitrogen atom is sometimes expressed simply as “N”. In addition, a carbonyl group is sometimes expressed simply as “—CO—”, a carboxyl group is sometimes expressed simply as “—CO₂—”, a sulfinyl group is sometimes expressed simply as “SO”, a sulfonyl group is sometimes expressed simply as “SO₂”, an ether bond is sometimes expressed simply as “—O—”, and a thioether bond is sometimes expressed simply as “—S—” (in this case “—” represents a bond).

In the present specification, the C1-C4 alkyl group indicates a linear or branched alkyl group having 1 to 4 carbon atoms, and examples thereof include a methyl group, an ethyl group, a propyl group, a butyl group, or isomers thereof [normal (n), iso (iso), secondary (sec), tertiary (t) and the like] and the like. Regarding the C1-C4 alkoxy group, the C1-C4 alkylthio group and the like, it is the same for the alkyl moiety.

In the present specification, examples of the C1-C4 alkoxy group include a methoxy group, an ethoxy group, a propoxy group, a butoxy group and the like, or isomers thereof.

In the present specification, examples of the C1-C4 alkylthio group include a methylthio group, an ethylthio group, a propylthio group, a butylthio group and the like, or isomers thereof.

In the present specification, examples of the C3-C6 cycloalkyl group include a cyclopropyl group, a cyclobutyl group, a cyclopentyl group, or a cyclohexyl group.

In the present specification, examples of the C1-C4 alkylsulfinyl group include a methylsulfinyl group, an ethylsulfinyl group, a propylsulfinyl group, a butylsulfinyl group, or isomers thereof.

In the present specification, examples of the C1-C4 alkylsulfonyl group include a methylsulfonyl group, an ethylsulfonyl group, a propylsulfonyl group, a butylsulfonyl group, or isomers thereof.

In the present specification, examples of the C1-C4 acylamide group include a formamide group, an acetamide group, a propionamide group, a butylamide group, or isomers thereof.

In the present specification, examples of the C1-C4 alkylcarbamoyl group include a methylcarbamoyl group, an ethylcarbamoyl group, a propylcarbamoyl group, a butylcarbamoyl group, or isomers thereof.

In the present specification, examples of the C1-C4 alkylsulfonamide group include a methylsulfonamide group, an ethylsulfonamide group, a propylsulfonamide group, a butylsulfonamide group, or isomers thereof.

In the present specification, examples of the C1-C4 alkylsulfamoyl group include a methylsulfamoyl group, an ethylsulfamoyl group, a propylsulfamoyl group, a butylsulfamoyl group, or isomers thereof.

In the present specification, examples of the C1-C4 acyl group include a formyl group, an acetyl group, a propionyl group, a butyryl group, or isomers thereof.

In the present specification, examples of the halogen atom include a fluorine atom, a chlorine atom, a bromine atom or an iodine atom.

According to the present invention, the isomers include the followings, unless specifically described otherwise. For example, with respect to an alkyl group, an alkenyl group, an alkynyl group, an alkoxy group, an alkylene group, an alkenylene group, and an alkynylene group, a linear and a branched forms are all included. In addition, an isomer based on a double bond, a ring, or a fused ring (E or Z isomer, or a cis or a trans isomer), an isomer based on an asymmetric carbon (R- or S-isomer, an isomer based on α- or β-configuration, an enantiomer, or a diastereomer and the like), an optical isomer having optical activity (D- or L-form, or d- or l-form), an isomer based on a difference in polarity under chromatographic separation (highly polar form, or weakly polar form), an equilibrium compound, a rotationary isomer, a tautomer, or a mixture comprising them in any ratio, or a racemic mixture are all within the scope of the present invention.

Specific examples of an isomer that is based on a ring structure include a cis form in which two substituents are bonded in the same direction compared to the plane that is formed by the ring structure. Such bonding relationship is sometimes referred to as cis configuration. As an example, when —CO₂R^{E} and —NR¹— are bonded to a cyclobutylene group at position 1 and position 3, respectively, the chemical structure of a cis form is the same as Formula (I-1) described below. In addition, there is also a trans form in which two substituents are bonded in the opposite direction compared to the plane that is formed by the ring structure. Such bonding relationship is sometimes referred to as trans configuration. As an example, when —CO₂R^{E} and —NR¹— are bonded to a cyclobutylene group at position 1 and position 3, respectively, the chemical structure of a trans form is the same as Formula (I-2) described below.

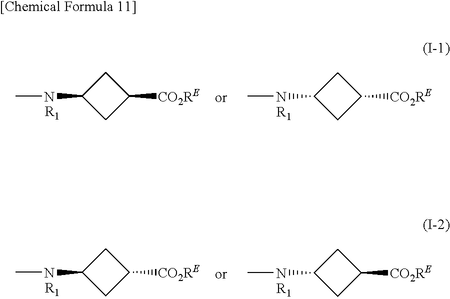

For clear understanding by a skilled person in the art, symbols described in the present specification are the same as described below, unless specifically described otherwise.

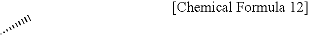

indicates a bond which is directed in back of the paper plane (i.e., α-configuration),

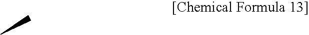

indicates a bond which is directed in front of the paper plane (i.e., β-configuration),

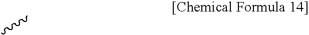

indicates a bond which is any one of α-configuration and β-configuration, or a mixture thereof, and

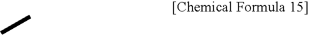

indicates a mixture of α-configuration and β-configuration.

As for a salt of the compounds of the present invention, a pharmaceutically acceptable salt is preferable. When a proton-donating substituent such as a carboxyl group, a phenolic hydroxyl group, or a tetrazole group and the like is comprised in the compound, any number of bases can be added to form a salt depending on the number of an acidic group included in the compound. For example, a base salt with a metal such as sodium and the like, an inorganic base such as ammonia and the like, an organic base such as triethylamine and the like can be mentioned. In addition, when a substituted or unsubstituted amino group is comprised in the compound, or a basic cyclic structure such as a pyridine ring, a quinoline ring and the like is comprised in the compound, any number of acids can be added to form a salt depending on the number of a basic substituent included in the compound. For example, an acid salt with an inorganic acid such as hydrochloric acid, sulfuric acid and the like, an organic acid such as acetic acid, citric acid and the like can be mentioned.

Herein below, the compounds having the Formula (1) will be explained in greater detail.

W represents a monovalent group derived from a compound selected from benzene, thiophene, furan and pyridine by the removal of one hydrogen atom. Preferably, W is a monovalent group derived from a compound selected from benzene, thiophene, and furan by the removal of one hydrogen atom. More preferably, W is a monovalent group derived from a compound selected from benzene and thiophene by the removal of one hydrogen atom. Still more preferably, W is a monovalent group derived from benzene by the removal of one hydrogen atom. Alternatively, W is still more preferably a monovalent group derived from thiophene by the removal of one hydrogen atom. Alternatively, there is also an embodiment in which W is still more preferably a monovalent group derived from furan by the removal of one hydrogen atom. Alternatively, there is also an embodiment in which W is still more preferably a monovalent group derived from pyridine by the removal of one hydrogen atom.

In addition to the above, there is other embodiment in which W is preferably a monovalent group derived from a compound selected from a group consisting of benzene, thiophene and pyridine by the removal of one hydrogen atom.

The W may be substituted with one or two X^{W}, and X^{W} indicates a C1-C4 alkyl group which may be substituted with 1 to 9 fluorine atoms, a C1-C4 alkoxy group which may be substituted with 1 to 9 fluorine atoms, a halogen atom, a cyano group, a C1-C4 alkylthio group which may be substituted with 1 to 9 fluorine atoms, a C1-C4 alkylsulfinyl group which may be substituted with 1 to 9 fluorine atoms, a C1-C4 alkylsulfonyl group which may be substituted with 1 to 9 fluorine atoms, a C1-C4 acylamide group which may be substituted with 1 to 7 fluorine atoms, a C1-C4 alkylcarbamoyl group which may be substituted with 1 to 9 fluorine atoms, a C1-C4 alkylsulfonamide group which may be substituted with 1 to 9 fluorine atoms, a C1-C4 alkylsulfamoyl group which may be substituted with 1 to 9 fluorine atoms, a C1-C4 acyl group which may be substituted with 1 to 7 fluorine atoms, or a C1-C4 alkyl group which is substituted with one C1-C4 alkoxy group which may be substituted with 1 to 9 fluorine atoms or with one —OH group, and when it is substituted with two X^{W}, they can be the same or different from each other. Preferred examples of X^{W} include a C1-C4 alkyl group which may be substituted with 1 to 9-fluorine atoms, a C1-C4 alkoxy group which may be substituted with 1 to 9 fluorine atoms, a halogen atom, and a cyano group. More preferred examples include a methyl group, an ethyl group, a trifluoromethyl group, a pentafluoroethyl group, a methoxy group, an ethoxy group, a trifluoromethoxy group, a fluorine atom, a chlorine atom, and a cyano group. Still more preferred examples include a methyl group, an ethyl group, a trifluoromethyl group, a methoxy group, a trifluoromethoxy group, a fluorine atom, and a cyano group. Even still more preferred examples include a methyl group, a trifluoromethyl group, and a fluorine atom. Further, there is also an embodiment in which a cyano group is even still more preferred. Further, there is also an embodiment in which a C1-C4 alkylthio group which may be substituted with 1 to 9 fluorine atoms is preferred, a methylthio group or an ethylthio group is more preferred and a methylthio group is still more preferred.

In addition, there are additional embodiments in which X^{W} is preferably a C1-C4 alkyl group which may be substituted with 1 to 9 fluorine atoms, a C1-C4 alkoxy group which may be substituted with 1 to 9 fluorine atoms, a halogen atom, or C1-C4 alkylthio group which may be substituted with 1 to 9 fluorine atoms, more preferably a methyl group, an ethyl group, a trifluoromethyl group, a pentafluoroethyl group, a methoxy group, an ethoxy group, a trifluoromethoxy group, a fluorine atom, a chlorine atom, a methylthio group, or an ethylthio group, still more preferably a methyl group, an ethyl group, a trifluoromethyl group, a methoxy group, a trifluoromethoxy group, a fluorine atom, or a methylthio group, and even still more preferably a methyl group, a trifluoromethyl group, a fluorine atom, or a methylthio group.

Alternatively, X^{W} is preferably a C1-C4 alkylsulfinyl group which may be substituted with 1 to 9 fluorine atoms, a C1-C4 alkylsulfonyl group which may be substituted with 1 to 9-fluorine atoms, a C1-C4 acylamide group which may be substituted with 1 to 7 fluorine atoms, a C1-C4 alkylcarbamoyl group which may be substituted with 1 to 9 fluorine atoms, a C1-C4 alkylsulfonamide group which may be substituted with 1 to 9 fluorine atoms, a C1-C4 alkylsulfamoyl group which may be substituted with 1 to 9 fluorine atoms, a C1-C4 acyl group which may be substituted with 1 to 7 fluorine atoms, or a C1-C4 alkyl group which is substituted with one C1-C4 alkoxy group which may be substituted with 1 to 9 fluorine atoms or with one —OH group, more preferably a methylsulfonyl group, a trifluoromethylsulfonyl group, an acetylamide group, a methylcarbamoyl group, a methylsulfonamide group, a methylaminosulfonyl group, an acetyl group, a methoxymethyl group, or a hydroxymethyl group, still more preferably a methylsulfonyl group, an acetylamide group, a methylsulfonyl group, an acetyl group or a methoxymethyl group, and even still more preferably a methylsulfonyl group, an acetyl group or a methoxymethyl group, and most preferably a methylsulfonyl group or a methoxymethyl group.

Further, when W is substituted with one or two X^{W}, there is an embodiment in which at least one X^{W} is preferably a C1-C4 alkylthio group which may be substituted with 1 to 9 fluorine atoms, a C1-C4 acyl group which may be substituted with 1 to 7 fluorine atoms, or a C1-C4 alkyl group which is substituted with one C1-C4 alkoxy group which may be substituted with 1 to fluorine atoms or with one —OH group, more preferably a methylthio group, an ethylthio group, an acetyl group, a trifluoroacetyl group, a methoxymethyl group, or a hydroxymethyl group, still more preferably a methylthio group, an acetyl group, a trifluoroacetyl group, a methoxymethyl group, or a hydroxymethyl group, and even still more preferably a methylthio group, an acetyl group, a methoxymethyl group, or a hydroxymethyl group.

In addition, there is other embodiment in which W is preferably unsubstituted.

In addition, there is other preferred embodiment, wherein W may be substituted with one or two X^{W}, and when it is substituted with two X^{W}, the two X^{W} can be the same or different from each other, and X^{W} indicates a C1-C4 alkyl group which may be substituted with 1 to 9 fluorine atoms, a C1-C4 alkoxy group which may be substituted with 1 to 9 fluorine atoms, a halogen atom, a cyano group or a C1-C4 alkylthio group which may be substituted with 1 to 9 fluorine atoms.

When W is a benzene ring substituted with two X^{W}, the W may be substituted with additional X^{W} which is selected from a group consisting of a C1-C4 alkyl group which may be substituted with 1 to 9 fluorine atoms, a C1-C4 alkoxy group which may be substituted with 1 to 9 fluorine atoms, a halogen atom and a cyano group, and the X^{W} can be the same or different from each other, (thus, W may be substituted with a total of three X^{W}), and in such case, three X^{W} are preferably a methyl group, a trifluoromethyl group, a methoxy group, a fluorine atom, a chlorine atom or a cyano group, more preferably a trifluoromethyl group, a fluorine atom, a chlorine atom or a cyano group, still more preferably a fluorine atom, a chlorine atom or a cyano group, even still more preferably a fluorine atom, or a cyano group, and most preferably a cyano group. In addition, there is other embodiment in which a fluorine atom is more preferable.

In addition, there is other embodiment in which a trifluoromethyl group, a fluorine atom and a chlorine atom are more preferable, a trifluoromethyl group and a chlorine atom are still more preferable, and a trifluoromethyl group is most preferable. In addition, there is other embodiment in which a chlorine atom is most preferable.

In addition, it is preferable that at least one X^{W} is at ortho position relative to the bonding of W to Z, and it is more preferable that at least one X^{W} is at ortho position relative to the bonding of W to Z and two X^{W} are at meta position relative to the bonding of W to Z. In addition, there is other embodiment in which two X^{W} are preferably at ortho position relative to the bonding of W to Z and one X^{W} is at meta position relative to the bonding of W to Z.

Z represents a divalent group derived from benzene by the removal of two hydrogen atoms, which binds to W— and —V— at para position and may be substituted with 1 to 4X^{Z}, wherein X^{Z} indicates a C1-C4 alkyl group which may be substituted with 1 to 9 fluorine atoms, a C1-C4 alkoxy group which may be substituted with 1 to 9 fluorine atoms, a halogen atom or a cyano group, and when it is substituted with two or more X^{Z}, they can be the same or different from each other. With respect to X^{Z} for Z, it is preferably a C1-C4 alkyl group which may be substituted with 1 to 9 fluorine atoms or a halogen atom, more preferably a C1-C2 alkyl group which may be substituted with possible number of a fluorine atom or a fluorine atom, still more preferably a methyl group, an ethyl group, a trifluoromethyl group, a pentafluoroethyl group, or a fluorine atom, even still more preferably a methyl group, a trifluoromethyl group, or a fluorine atom, most preferably a methyl group or a fluorine atom, and most preferably a methyl group.

When Z is substituted with one or two X^{Z}, X^{Z} is preferably a methyl group, an ethyl group, a trifluoromethyl group, or a fluorine atom, more preferably a methyl group or a fluorine atom, and still more preferably a methyl group. In addition, there is other embodiment in which a methyl group or a trifluoromethyl group is still more preferred.

When Z is substituted with one X^{Z}, X^{Z} is preferably a methyl group, an ethyl group, a trifluoromethyl group, or a fluorine atom, more preferably a methyl group, or a trifluoromethyl group and still more preferably a methyl group. In addition, there is other embodiment in which a trifluoromethyl group is still more preferred.

When Z is substituted with two X^{Z}, X^{Z} is preferably a methyl group, an ethyl group, a trifluoromethyl group, or a fluorine atom, more preferably a methyl group, a trifluoromethyl group or a fluorine atom, still more preferably a methyl group or a fluorine atom, and most preferably a methyl group. In addition, there is other embodiment in which a trifluoromethyl group is most preferred.

When Z is substituted with two X^{Z}, combination of X^{Z} is preferably (a methyl group, a fluorine atom), (a methyl group, a methyl group) or (a trifluoromethyl group, a fluorine atom), more preferably (a methyl group, a fluorine atom) or (a methyl group, a methyl group), and still more preferably (a methyl group, a methyl group). In addition, there is other embodiment in which (a methyl group, a fluorine atom) is still more preferred.

Regarding combination of position of X^{Z} when Z is substituted with two X^{Z}, two X^{Z} are preferably in an ortho position or a para position in case the combination of two X^{Z} is (a methyl group, a fluorine atom) or (a trifluoromethyl group, a fluorine atom). In this case, a methyl group or a trifluoromethyl group is preferably in an ortho position relative to W.

Further, regarding combination of position of X^{Z} when Z is substituted with two X^{Z}, two X^{Z} are preferably all in an ortho position relative to W in case the combination of X^{Z} is (a methyl group, a methyl group).

Further, more specifically, there is other embodiment in which Z is preferably substituted with three X^{Z}. Still further, there is other embodiment in which Z is substituted with one to three X^{Z}.

More specific examples of W-Z-V— include the following Formulae (3) to (5).

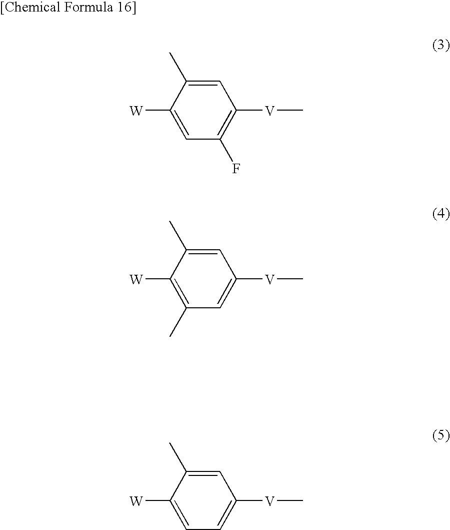

V represents a divalent group derived from [1,2,4]-oxadiazole by the removal of two hydrogen atoms or —(CR^{V1}R^{V2})ₙ—(CR^{V3}R^{V4})ₖ—O—. More preferably, V is a divalent group derived from [1,2,4]-oxadiazole by the removal of two hydrogen atoms. There is other embodiment in which V is preferably —(CR^{V1}R^{V2})ₙ—(CR^{V3}R^{V4})ₖ—O—.

When V is a divalent group derived from [1,2,4]-oxadiazole by the removal of two hydrogen atoms, preferred example of binding position of V to W-Z- and —Ar— is described below (binding position for W-Z-, and binding position for —Ar—).

When V is a divalent group derived from [1,2,4]-oxadiazole by the removal of two hydrogen atoms, (5, 3) is preferable. In addition, there is other embodiment in which (3, 5) is preferable. (5, 3) is sometimes described as “the binding position for V to W-Z- and —Ar— is position 5 and position 3 of V, respectively.” In addition, (5, 3) is sometimes expressed as the following Formula (2).

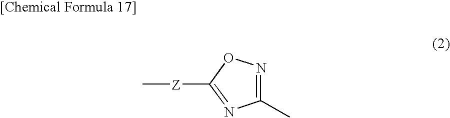

When V is —(CR^{V1}R^{V2})ₙ—(CR^{V3}R^{V4})ₖ—R^{V1}, R^{V2}, R^{V3}, and R^{V4} can be the same or different from each other, and each independently represent a hydrogen atom, a halogen atom, or a C1-C4 alkyl which may be substituted with 1 to 5 halogen atoms;

n is an integer of 0 to 2, and when n is 0, —(CR^{V1}R^{V2})ₙ— means a single bond;

k indicates an integer of 0 or 1 and when k is 0, —(CR^{V3}R^{V4})ₖ— means a single bond.

R^{V1}, R^{V2}, R^{V3}, and R^{V4} are preferably a hydrogen atom, a fluorine atom, a methyl group, or an ethyl group. More preferably, they are a hydrogen atom or a methyl group and still more preferably a hydrogen atom. In addition, there is other embodiment in which a fluorine atom is more preferred.

Preferably, n is 2. In this case, k is preferably 0 and there is other embodiment in which k is preferably 1. In addition, there is other embodiment in which one of n and k is preferably and the other is 1. In addition, there is other embodiment in which both n and k are preferably 1.

X¹ represents a C1-C4 alkyl group which may be substituted with 1 to 9 fluorine atoms, a C1-C4 alkoxy group which may be substituted with 1 to 9 fluorine atoms, or a halogen atom. X¹ is preferably a methyl group, a trifluoromethyl group, an ethyl group, a methoxy group, a trifluoromethoxy group, a fluorine atom, or a chlorine atom. More preferably it is a methyl group, a trifluoromethyl group, an ethyl group, a fluorine atom, or a chlorine atom. Still more preferably, it is a methyl group, a fluorine atom, or a chlorine atom. Even still more preferably, it is a methyl group, a trifluoromethyl group, or a fluorine atom. Most preferably it is a methyl group or a fluorine atom. Still most preferably it is a methyl group. In addition, there is other embodiment in which a fluorine atom is most preferable. In addition, there is other embodiment in which a chlorine atom is most preferable.

l represents an integer of 0 to 3. Preferably, l is 0 or 1, and more preferably it is 0. In addition, there is other embodiment in which 1 is preferably 1.

R¹ represents a hydrogen atom or a C1-C4 alkyl group, or R¹ is linked to X² via a C1 alkylene to form a 5-membered ring, wherein the C1 alkylene may be substituted with one or two C1-C4 alkyl groups.

R² indicates a hydrogen atom or a C1-C4 alkyl group, or is linked to X² via a C2 alkylene to form a 5-membered ring, wherein the C2 alkylene may be substituted with one or two C1-C4 alkyl, or is linked to X² via a C3 alkylene to form a 6-membered ring, wherein the C3 alkylene may be substituted with one or two C1-C4 alkyl groups.

Any one of R¹ and R² is linked to X² to form a ring.

When R¹ or R² is linked to X² to form a ring, it is preferable that R¹ is linked to X² via a C1 alkylene to form a 5-membered ring. In addition, there is other preferred embodiment in which R² is linked to X² via a C2 alkylene to form a 5-membered ring. In addition, there is other preferred embodiment in which R² is linked to X² via a C3 alkylene to form a 6-membered ring.

X² represents a single bond. That is, X² is linked to any one of R¹ and R² to form a ring.

The phrase “R¹ is linked to X² via C1 alkylene to form a 5-membered ring, wherein the C1 alkylene may be substituted with one or two C1-C4 alkyl groups” means that, the framework structure of the Formula (1), i.e., the portion shown as the following Formula (6) in the Formula (1), is described as the following Formula (7) (in the Formula (7), X³¹ and X³² indicate a hydrogen atom or a C1-C4 alkyl group, and V, X¹ and l are as defined above).

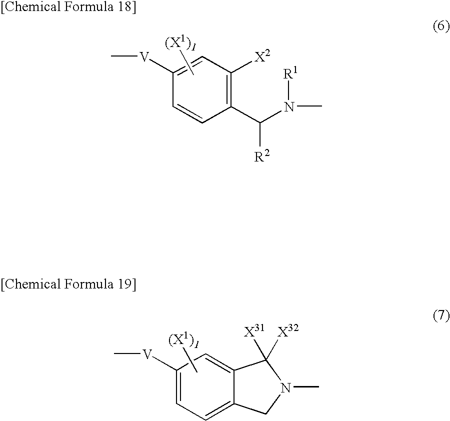

That is, for the above case, the Formula (1) is overall expressed with the following Formula (7-2) (in the Formula (7-2), W, Z, V, X¹, l, R², Y, R^{E}, X³¹ and X³² are as defined above).

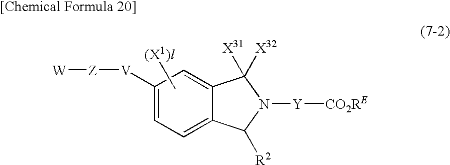

It is preferable that X³¹ and X³² each independently represent a hydrogen atom, a methyl group, or an ethyl group. More preferably, they are hydrogen atom or a methyl group, and still more preferably a hydrogen atom.

It is preferable that both of X³¹ and X³² are a hydrogen atom, one of them is a methyl group, both of them are a methyl group, one of them is an ethyl group or both of them are an ethyl group. More preferably, both of them are a hydrogen atom, one of them is a methyl group or both of them are a methyl group. Still more preferably, both of them are a hydrogen atom, or one of them is a methyl group. Most preferably, both of them are a hydrogen atom.

The phrase “R² is linked to X² via C2 alkylene to form a 5-membered ring, wherein the C2 alkylene may be substituted with one or two C1-C4 alkyl groups” means that, the framework structure of the Formula (1), i.e., the portion shown as the following Formula (6) in the Formula (1), is described as the following Formula (8) (in the Formula (8), X³¹, X³², X³³, and X³⁴ indicate a hydrogen atom or a C1-C4 alkyl group, and V, X¹, l, and R¹ are as defined above).

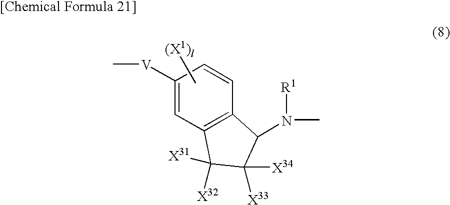

That is, for the above case, the Formula (1) is overall expressed with the following Formula (8-2) (in the Formula (8-2), W, Z, V, X¹, l, R¹, Y, R^{E}, X³¹, X³², X³³ and X³⁴ are as defined above).

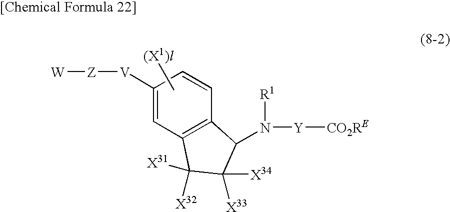

It is preferable that X³¹, X³², X³³, and X³⁴ are all a hydrogen atom, one or two of them are a C1-C4 alkyl group, for example one of them is a methyl group, two of them are a methyl group, one of them is an ethyl group or two of them are an ethyl group. More preferably, all of them are a hydrogen atom, one of them is a methyl group, or two of them are a methyl group. Still more preferably, all of them are a hydrogen atom, or one of them is a methyl group. Most preferably, all of them are a hydrogen atom.

The phrase “R² is linked to X² via C3 alkylene to form a 6-membered ring, wherein the C3 alkylene may be substituted with one or two C1-C4 alkyl groups” means that, the framework structure of the Formula (1), i.e., the portion shown as the following Formula (6) in the Formula (1), is described as the following Formula (9) (in the Formula (9), X³¹, X³², X³³, X³⁴, X³⁵, and X³⁶ indicate a hydrogen atom or a C1-C4 alkyl group, and V, X¹, l, and R¹ are as defined above)

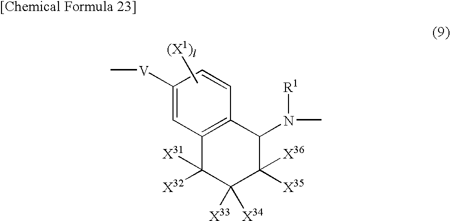

That is, for the above case, the Formula (1) is overall expressed with the following Formula (9-2) (in the Formula (9-2), W, Z, V, X¹, l, R¹, Y, R^{E}, X³¹, X³², X³³, X³⁴, X³⁵, and X³⁶ are as defined above).

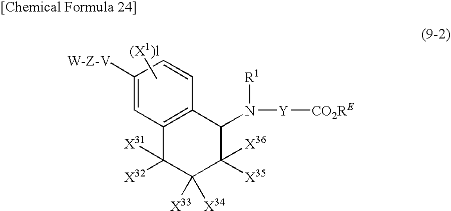

It is preferable that X³¹, X³², X³³, X³⁴, X³⁵, and X³⁶ are all a hydrogen atom, one or two of them are a C1-C4 alkyl group, for example one of them is a methyl group, two of them are a methyl group, one of them is an ethyl group or two of them are an ethyl group. More preferably, all of them are a hydrogen atom, one of them is a methyl group, or two of them are a methyl group. Still more preferably, all of them are a hydrogen atom, or one of them is a methyl group. Most preferably, all of them are a hydrogen atom.

With respect to a C1 alkylene which may be substituted with one or two C1-C4 alkyl group for the case in which R¹ is linked to X² via the C1 alkylene to form a 5-membered ring, wherein the C1 alkylene may be substituted with one or two C1-C4 alkyl groups, it is preferably unsubstituted, substituted with one methyl group, substituted with two methyl groups, substituted with one ethyl group, or substituted with two ethyl groups. More preferably, it is unsubstituted, substituted with one methyl group or substituted with two methyl groups. Still more preferably, it is unsubstituted, or substituted with one methyl group. Most preferably, it is unsubstituted. There is other embodiment in which it is most preferably substituted with one methyl group. In addition, there is other embodiment in which it is most preferably substituted with two methyl groups.

With respect to a C2 alkylene which may be substituted with one or two C1-C4 alkyl group for the case in which R² is linked to X² via the C2 alkylene to form a 5-membered ring, wherein the C2 alkylene may be substituted with one or two C1-C4 alkyl groups, it is preferably unsubstituted, substituted with one methyl group, substituted with two methyl groups, substituted with one ethyl group, or substituted with two ethyl groups. More preferably, it is unsubstituted, substituted with one methyl group or substituted with two methyl groups. Still more preferably, it is unsubstituted, or substituted with one methyl group. Most preferably, it is unsubstituted. There is other embodiment in which it is most preferably substituted with one methyl group. In addition, there is other embodiment in which it is most preferably substituted with two methyl groups.

With respect to a C3 alkylene which may be substituted with one or two C1-C4 alkyl groups for the case in which R² is linked to X² via the C3 alkylene to form a 6-membered ring, wherein the C3 alkylene may be substituted with one or two C1-C4 alkyl groups, it is preferably unsubstituted, substituted with one methyl group, substituted with two methyl groups, substituted with one ethyl group, or substituted with two ethyl groups. More preferably, it is unsubstituted, substituted with one methyl group or substituted with two methyl groups. Still more preferably, it is unsubstituted, or substituted with one methyl group. Most preferably, it is unsubstituted. There is other embodiment in which it is most preferably substituted with one methyl group. In addition, there is other embodiment in which it is most preferably substituted with two methyl groups.

With respect to R¹ for the case in which R¹ indicates a hydrogen atom or a C1-C4 alkyl, it is preferably a hydrogen atom, a methyl group, or an ethyl group. More preferably it is a hydrogen atom or a methyl group. Still more preferably, it is a hydrogen atom. In addition, there is other embodiment in which a methyl group is still more preferable.

With respect to R² for the case in which R² indicates a hydrogen atom or a C1-C4 alkyl group, it is preferably a hydrogen atom, a methyl group, or an ethyl group. More preferably it is a hydrogen atom or a methyl group. Still more preferably, it is a hydrogen atom. In addition, there is other embodiment in which a methyl group is still more preferable.

Y indicates a cyclobutylene group and may be substituted with 1 to 4 X^{Y}, and it binds to —CO₂R^{E} and —NR¹— at position 1 and position 3 of the cyclobutylene group, respectively;

X^{Y} indicates —OH, a halogen atom, or a C1-C4 alkyl group which may be substituted with 1 to 5 halogen atoms;

With respect to X^{Y} for Y, it is preferably a methyl group, an ethyl group, or a fluorine atom. More preferably, it is a methyl group or a fluorine atom. Still more preferably, it is a methyl group. In addition, there is other embodiment in which a fluorine atom is preferable. With respect to the number of X^{Y}, it is preferably 1 or 2. More preferably it is 1. In addition, there is other embodiment in which it is preferably 4.

In addition, there is other embodiment in which Y is preferably unsubstituted.

Further, regarding the bonding relationship between Y and —NR¹— or Y and —CO₂R^{E}, a cis or a trans configuration can be exemplified. Trans configuration is preferable. In addition, there is other embodiment in which cis configuration is preferable.

R^{E} indicates a hydrogen atom, a C1-C4 alkyl group, (CH₂)ₘN(R^{E1})(R^{E2}) or —C(R^{E3})₂OC(O)A^{E}R^{E4};

m indicates an integer of 2 or 3;

R^{E1} and R^{E2} can be the same or different from each other and each independently represent a methyl group, an ethyl group, or a propyl group, or a nitrogen-containing saturated cycloalkyl group in which R^{E1} and R^{E2} are linked to each other to form a 3- to 6-membered ring together with a nitrogen atom, or form a morpholine group together with a nitrogen atom;

R^{E3} indicates a hydrogen atom, a methyl group, an ethyl group, or a propyl group;

R^{E4} indicates a C1-C4 alkyl group, C3-C6 cycloalkyl group, or a phenyl group, and;

A^{E} indicates a single bond or an oxygen atom.

R^{E} is preferably a hydrogen atom or a C1-C4 alkyl group. More preferably, it is a hydrogen atom, a methyl group, or an ethyl group. Still more preferably, it is a hydrogen atom or an ethyl group. Most preferably, it is a hydrogen atom. In addition, there is other embodiment in which an ethyl group is most preferable.

Combination of the substituents for the compounds represented by the Formula (1) is not specifically limited, and preferred examples thereof include the following:

<A1> A compound in which W is monovalent group derived from benzene by the removal of one hydrogen atom;
<A2> A compound in which W is monovalent group derived from thiophene by the removal of one hydrogen atom;
<A3> A compound in which W is monovalent group derived from furan by the removal of one hydrogen atom;
<A4> A compound in which W is monovalent group derived from pyridine by the removal of one hydrogen atom;
<B1> A compound in which X^{W} is a methyl group;
<B2> A compound in which X^{W} is an ethyl group;
<B3> A compound in which X^{W} is a trifluoromethyl group;
<B4> A compound in which X^{W} is a pentafluoroethyl group;
<B5> A compound in which X^{W} is a methoxy group;
<B6> A compound in which X^{W} is an ethoxy group;
<B7> A compound in which X^{W} is a trifluoromethoxy group;
<B8> A compound in which X^{W} is a fluorine atom;
<B9> A compound in which X^{W} is a chlorine atom;
<B10> A compound in which X^{W} is a cyano group;
<B11> A compound in which X^{W} is a methylthio group;
<B12> A compound in which X^{W} is an ethylthio group;
<B13> A compound in which X^{W} is a methylsulfonyl group;
<B14> A compound in which X^{W} is a trifluoromethylsulfonyl group;
<B15> A compound in which X^{W} is an acetylamide group;
<B16> A compound in which X^{W} is a methylcarbamoyl group;
<B17> A compound in which X^{W} is a methylsulfonamide group;
<B18> A compound in which X^{W} is a methylaminosulfonyl group;
<B19> A compound in which X^{W} is an acetyl group;
<B20> A compound in which X^{W} is a methoxymethyl group;
<B21> A compound in which X^{W} is a hydroxymethyl group;
<B22> A compound in which W is unsubstituted;
<B23> A compound in which W is monovalent group derived from unsubstituted benzene by the removal of one hydrogen atom;
<B24> A compound in which W is monovalent group derived from unsubstituted thiophene by the removal of one hydrogen atom;
<B25> A compound in which W is monovalent group derived from unsubstituted pyridine by the removal of one hydrogen atom;
<B26> A compound in which W is substituted with one X^{W};
<B27> A compound in which W is monovalent group derived from benzene substituted with one X^{W} by the removal of one hydrogen atom;
<B28> A compound in which W is monovalent group derived from thiophene substituted with one X^{W} by the removal of one hydrogen atom;
<B29> A compound in which W is substituted with one methyl group;
<B30> A compound in which W is substituted with one trifluoromethyl group;
<B31> A compound in which W is substituted with one methoxy group;
<B32> A compound in which W is substituted with one fluorine atom;
<B33> A compound in which W is substituted with one chlorine atom;
<B34> A compound in which W is monovalent group derived from benzene substituted with one methyl group by the removal of one hydrogen atom;
<B35> A compound in which W is monovalent group derived from benzene substituted with one trifluoromethyl group by the removal of one hydrogen atom;
<B36> A compound in which W is monovalent group derived from benzene substituted with one fluorine atom by the removal of one hydrogen atom;
<B37> A compound in which W is substituted with two X^{W};
<B38> A compound in which W is monovalent group derived from benzene substituted with two X^{W} by the removal of one hydrogen atom;
<B39> A compound in which W is monovalent group derived from thiophene substituted with two X^{W} by the removal of one hydrogen atom;
<B40> A compound in which W is monovalent group derived from benzene substituted with two methyl groups by the removal of one hydrogen atom;
<B41> A compound in which W is monovalent group derived from benzene substituted with two trifluoromethyl groups by the removal of one hydrogen atom;
<B42> A compound in which W is monovalent group derived from benzene substituted with two fluorine atoms by the removal of one hydrogen atom;
<B43> A compound in which W is monovalent group derived from benzene substituted with two chlorine atoms by the removal of one hydrogen atom;
<B44> A compound in which W is monovalent group derived from benzene substituted with one trifluoromethyl group and one fluorine atom by the removal of one hydrogen atom;
<B45> A compound in which W is monovalent group derived from benzene substituted with one trifluoromethyl group and one chlorine atom by the removal of one hydrogen atom;
<B46> A compound in which W is monovalent group derived from thiophene substituted with two trifluoromethyl groups by the removal of one hydrogen atom;
<B47> A compound in which W is monovalent group derived from thiophene substituted with two fluorine atoms by the removal of one hydrogen atom;
<B48> A compound in which W is monovalent group derived from thiophene substituted with two chlorine atoms by the removal of one hydrogen atom;
<B49> A compound in which W is monovalent group derived from thiophene substituted with one trifluoromethyl group and one fluorine atom by the removal of one hydrogen atom;
<B50> A compound in which W is monovalent group derived from thiophene substituted with one trifluoromethyl group and one chlorine atom by the removal of one hydrogen atom;
<C1> A compound corresponding to <B1> among any of the above <A1> to <A4>;
<C2> A compound corresponding to <B2> among any of the above <A1> to <A4>;
<C3> A compound corresponding to <B3> among any of the above <A1> to <A4>;
<C4> A compound corresponding to <B4> among any of the above <A1> to <A4>;
<C5> A compound corresponding to <B5> among any of the above <A1> to <A4>;
<C6> A compound corresponding to <B6> among any of the above <A1> to <A4>;
<C7> A compound corresponding to <B7> among any of the above <A1> to <A4>;
<C8> A compound corresponding to <B8> among any of the above <A1> to <A4>;
<C9> A compound corresponding to <B9> among any of the above <A1> to <A4>;
<C10> A compound corresponding to <B10> among any of the above <A1> to <A4>;
<C11> A compound corresponding to <B11> among any of the above <A1> to <A4>;
<C12> A compound corresponding to <B12> among any of the above <A1> to <A4>;
<C13> A compound corresponding to <B13> among any of the above <A1> to <A4>;
<C14> A compound corresponding to <B14> among any of the above <A1> to <A4>;
<C15> A compound corresponding to <B15> among any of the above <A1> to <A4>;
<C16> A compound corresponding to <B16> among any of the above <A1> to <A4>;
<C17> A compound corresponding to <B17> among any of the above <A1> to <A4>;
<C18> A compound corresponding to <B18> among any of the above <A1> to <A4>;
<C19> A compound corresponding to <B19> among any of the above <A1> to <A4>;
<C20> A compound corresponding to <B20> among any of the above <A1> to <A4>;
<C21> A compound corresponding to <B21> among any of the above <A1> to <A4>;
<C22> A compound corresponding to <B22> among any of the above <A1> to <A4>;
<C23> A compound corresponding to <B23> among any of the above <A1> to <A4>;
<C24> A compound corresponding to <B24> among any of the above <A1> to <A4>;
<C25> A compound corresponding to <B25> among any of the above <A1> to <A4>;
<C26> A compound corresponding to <B26> among any of the above <A1> to <A4>;
<C27> A compound corresponding to <B27> among any of the above <A1> to <A4>;
<C28> A compound corresponding to <B28> among any of the above <A1> to <A4>;
<C29> A compound corresponding to <B29> among any of the above <A1> to <A4>;
<C30> A compound corresponding to <B30> among any of the above <A1> to <A4>;
<C31> A compound corresponding to <B31> among any of the above <A1> to <A4>;
<C32> A compound corresponding to <B32> among any of the above <A1> to <A4>;
<C33> A compound corresponding to <B33> among any of the above <A1> to <A4>;
<C34> A compound corresponding to <B34> among any of the above <A1> to <A4>;
<C35> A compound corresponding to <B35> among any of the above <A1> to <A4>;
<C36> A compound corresponding to <B36> among any of the above <A1> to <A4>;
<C37> A compound corresponding to <B37> among any of the above <A1> to <A4>;
<C38> A compound corresponding to <B38> among any of the above <A1> to <A4>;
<C39> A compound corresponding to <B39> among any of the above <A1> to <A4>;
<C40> A compound corresponding to <B40> among any of the above <A1> to <A4>;
<C41> A compound corresponding to <B41> among any of the above <A1> to <A4>;
<C42> A compound corresponding to <B42> among any of the above <A1> to <A4>;
<C43> A compound corresponding to <B43> among any of the above <A1> to <A4>;
<C44> A compound corresponding to <B44> among any of the above <A1> to <A4>;
<C45> A compound corresponding to <B45> among any of the above <A1> to <A4>;
<C46> A compound corresponding to <B46> among any of the above <A1> to <A4>;
<C47> A compound corresponding to <B47> among any of the above <A1> to <A4>;
<C48> A compound corresponding to <B48> among any of the above <A1> to <A4>;
<C49> A compound corresponding to <B49> among any of the above <A1> to <A4>;
<C50> A compound corresponding to <B50> among any of the above <A1> to <A4>;
<D1> A compound in which at least one X^{Z} is a C1-C2 alkyl group which may be substituted with 1 to 5 fluorine atoms;
<D2> A compound in which X^{Z} is a methyl group;
<D3> A compound in which X^{Z} is an ethyl group;
<D4> A compound in which X^{Z} is a trifluoromethyl group;
<D5> A compound in which X^{Z} is a pentafluoroethyl group;
<D6> A compound in which X^{Z} is a fluorine atom;
<E1> A compound corresponding to <D1> among any of the above <A1> to <C50>;
<E2> A compound corresponding to <D2> among any of the above <A1> to <C50>;
<E3> A compound corresponding to <D3> among any of the above <A1> to <C50>;
<E4> A compound corresponding to <D4> among any of the above <A1> to <C50>;
<E5> A compound corresponding to <D5> among any of the above <A1> to <C50>;
<E6> A compound corresponding to <D6> among any of the above <A1> to <C50>;
<F1> A compound in which Z is substituted with one X^{Z};
<F2> A compound in which Z is substituted with two X^{Z};
<F3> A compound in which Z is substituted with three X^{Z};
<F4> A compound in which Z is substituted with four X^{Z};
<G1> A compound corresponding to <F1> among any of the above <A1> to <E6>;
<G2> A compound corresponding to <F2> among any of the above <A1> to <E6>;
<G3> A compound corresponding to <F3> among any of the above <A1> to <E6>;
<G4> A compound corresponding to <F4> among any of the above <A1> to <E6>;
<H1> A compound in which each of X^{Z} is a methyl group and a fluorine atom, respectively, for the case, wherein Z is substituted with two X^{Z};
<H2> A compound in which both X^{Z} is a methyl group, for the case, wherein Z is substituted with two X^{Z};
<H3> A compound in which each of X^{Z} is a trifluoromethyl group and a fluorine atom, respectively, for the case, wherein Z is substituted with two X^{Z};
<I1> A compound corresponding to <H1> among any of the above <F2>, <G2>;
<I2> A compound corresponding to <H2> among any of the above <F2>, <G2>;
<I3> A compound corresponding to <H3> among any of the above <F2>, <G2>;
<J1> A compound in which X^{Z} is at ortho position relative to W when Z is substituted with one X^{Z};
<J2> A compound in which X^{Z} is at meta position relative to W when Z is substituted with one X^{Z};
<J3> A compound in which both X^{Z} are at ortho position relative to W when Z is substituted with two X^{Z};
<J4> A compound in which one X^{Z} is at ortho position and the other X^{Z} is at meta position relative to W and the two X^{Z} are at para position relative to each other when Z is substituted with two X^{Z};
<J5> A compound in which one X^{Z} is at ortho position and the other X^{Z} is at meta position relative to W and the two X^{Z} are at ortho position relative to each other when Z is substituted with two X^{Z};
<K1> A compound corresponding to <J1> among any of the above <F1>, <G1>;
<K2> A compound corresponding to <J2> among any of the above <F1>, <G1>;
<K3> A compound corresponding to <J3> among any of the above <H1> to <13>;
<K4> A compound corresponding to <J4> among any of the above <H1> to <13>;
<K5> A compound corresponding to <J5> among any of the above <H1> to <13>;
<L1> A compound in which V is a divalent group derived from [1,2,4]-oxadiazole by the removal of two hydrogen atoms;
<L2> A compound in which V is a divalent group derived from [1, 2, 4]-oxadiazole by the removal of two hydrogen atoms and the position at which V is bonded to W-Z- is position 5 of the V;
<L3> A compound in which V is a divalent group derived from [1, 2, 4]-oxadiazole by the removal of two hydrogen atoms and the position at which V is bonded to W-Z- is position 3 of the V;
<L4> A compound in which V is —(CR^{V1}R^{V2})₂—(CR^{V3}R^{V4})—O—;
<L5> A compound in which V is —(CR^{V1}R^{V2})—(CR^{V3}R^{V4})—O—;
<L6> A compound in which V is —(CR^{V1}R^{V2})₂—O—;
<L7> A compound in which V is —CH₂CH₂—O—;
<L8> A compound in which V is —CH₂—O—;
<M1> A compound corresponding to <L1> among any of the above <A1> to <K5>;
<M2> A compound corresponding to <L2> among any of the above <A1> to <K5>;
<M3> A compound corresponding to <L3> among any of the above <A1> to <K5>;
<M4> A compound corresponding to <L4> among any of the above <A1> to <K5>;
<M5> A compound corresponding to <L5> among any of the above <A1> to <K5>;
<M6> A compound corresponding to <L6> among any of the above <A1> to <K5>;
<M7> A compound corresponding to <L7> among any of the above <A1> to <K5>;
<M8> A compound corresponding to <L8> among any of the above <A1> to <K5>;
<N1> A compound in which 1 is 0;
<N2> A compound in which 1 is 1;
<N3> A compound in which at least one X¹ is a methyl group when is an integer of 1 to 3;
<N4> A compound in which at least one X¹ is a ethyl group when is an integer of 1 to 3;
<N5> A compound in which at least one X¹ is a trifluoromethyl group when 1 is an integer of 1 to 3;
<N6> A compound in which at least one X¹ is a methoxy group when is an integer of 1 to 3;
<N7> A compound in which at least one X¹ is a fluorine atom when is an integer of 1 to 3;
<N8> A compound in which at least one X¹ is a chlorine atom when is an integer of 1 to 3;
<O1> A compound corresponding to <N1> among any of the above <A1> to <M8>;
<O2> A compound corresponding to <N2> among any of the above <A1> to <M8>;
<O3> A compound corresponding to <N3> among any of the above <A1> to <M8>;
<O4> A compound corresponding to <N4> among any of the above <A1> to <M8>;
<O5> A compound corresponding to <N5> among any of the above <A1> to <M8>;
<O6> A compound corresponding to <N6> among any of the above <A1> to <M8>;
<O7> A compound corresponding to <N7> among any of the above <A1> to <M8>;
<O8> A compound corresponding to <N8> among any of the above <A1> to <M8>;
<P1> A compound in which R¹ is a hydrogen atom;
<P2> A compound in which R¹ is a methyl group;
<P3> A compound in which R¹ is an ethyl group;
<P4> A compound in which R¹ is linked to X² via a C1 alkylene to form a 5-membered ring;
<P5> A compound in which R¹ is linked to X² via an unsubstituted C1 alkylene to form a 5-membered ring;
<P6> A compound in which R¹ is linked to X² via a C1 alkylene substituted with a methyl group to form a 5-membered ring;
<P7> A compound in which R¹ is linked to X² via a C1 alkylene substituted with two methyl groups to form a 5-membered ring;
<P8> A compound in which R¹ is linked to X² via a C1 alkylene substituted with an ethyl group to form a 5-membered ring;
<P9> A compound in which R¹ is linked to X² via a C1 alkylene substituted with two ethyl groups to form a 5-membered ring;
<Q1> A compound corresponding to <P1> among any of the above <A1> to <O8>;
<Q2> A compound corresponding to <P2> among any of the above <A1> to <O8>;
<Q3> A compound corresponding to <P3> among any of the above <A1> to <O8>;
<Q4> A compound corresponding to <P4> among any of the above <A1> to <O8>;
<Q5> A compound corresponding to <P5> among any of the above <A1> to <O8>;
<Q6> A compound corresponding to <P6> among any of the above <A1> to <O8>;
<Q7> A compound corresponding to <P7> among any of the above <A1> to <O8>;
<Q8> A compound corresponding to <P8> among any of the above <A1> to <O8>;
<Q9> A compound corresponding to <P9> among any of the above <A1> to <O8>;
<R1> A compound in which R² is a hydrogen atom;
<R2> A compound in which R² is methyl group;
<R3> A compound in which R² is an ethyl group;
<R4> A compound in which R² is linked to X² via a C2 alkylene to form a 5-membered ring;
<R5> A compound in which R² is linked to X² via an unsubstituted C2 alkylene to form a 5-membered ring;
<R6> A compound in which R² is linked to X² via a C2 alkylene substituted with a methyl group to form a 5-membered ring;
<R7> A compound in which R² is linked to X² via a C2 alkylene substituted with two methyl groups to form a 5-membered ring;
<R8> A compound in which R² is linked to X² via a C2 alkylene substituted with an ethyl group to form a 5-membered ring;
<R9> A compound in which R² is linked to X² via a C2 alkylene substituted with two ethyl groups to form a 5-membered ring;
<R10> A compound in which R² is linked to X² via a C3 alkylene to form a 6-membered ring;
<R11> A compound in which R² is linked to X² via an unsubstituted C3 alkylene to form a 6-membered ring;
<R12> A compound in which R² is linked to X² via a C3 alkylene substituted with a methyl group to form a 6-membered ring;
<R13> A compound in which R² is linked to X² via a C3 alkylene substituted with two methyl groups to form a 6-membered ring;
<R14> A compound in which R² is linked to X² via a C3 alkylene substituted with an ethyl group to form a 6-membered ring;
<R15> A compound in which R² is linked to X² via a C3 alkylene substituted with two ethyl groups to form a 6-membered ring;
<S1> A compound corresponding to <R1> among any of the above <A1> to <O8>;
<S2> A compound corresponding to <R2> among any of the above <A1> to <O8>;
<S3> A compound corresponding to <R3> among any of the above <A1> to <O8>;
<S4> A compound corresponding to <R4> among any of the above <A1> to <O8>;
<S5> A compound corresponding to <R5> among any of the above <A1> to <O8>;
<S6> A compound corresponding to <R6> among any of the above <A1> to <O8>;
<S7> A compound corresponding to <R7> among any of the above <A1> to <O8>;
<S8> A compound corresponding to <R8> among any of the above <A1> to <O8>;
<S9> A compound corresponding to <R9> among any of the above <A1> to <O8>;
<S10> A compound corresponding to <R10> among any of the above <A1> to <O8>;
<S11> A compound corresponding to <R11> among any of the above <A1> to <O8>;
<S12> A compound corresponding to <R12> among any of the above <A1> to <O8>;
<S13> A compound corresponding to <R13> among any of the above <A1> to <O8>;
<S14> A compound corresponding to <R14> among any of the above <A1> to <O8>;
<S15> A compound corresponding to <R15> among any of the above <A1> to <O8>;
<T1> A compound in which X^{Y} is a methyl group;
<T2> A compound in which Y is unsubstituted;
<U1> A compound corresponding to <T1> among any of the above <A1> to <S15>;
<U2> A compound corresponding to <T2> among any of the above <A1> to <S15>;
<V1> A compound in which bonding between Y and —NR¹— and bonding between Y and —CO₂R^{E} are in cis configuration;
<V2> A compound in which bonding between Y and —NR¹— and bonding between Y and —CO₂R^{E} are in trans configuration;
<W1> A compound corresponding to <V1> among any of the above <A1> to <U2>;
<W2> A compound corresponding to <V2> among any of the above <A1> to <U2>;
<X1> A compound in which R^{E} is a hydrogen atom;
<X2> A compound in which R^{E} is a methyl group;
<X3> A compound in which R^{E} is an ethyl group;
<Y1> A compound corresponding to <X1> among any of the above <A1> to <W2>;
<Y2> A compound corresponding to <X2> among any of the above <A1> to <W2>;
<Y3> A compound corresponding to <X3> among any of the above <A1> to <W2>.

Specifically, preferred examples of the compounds of the present invention include the following compounds, but the scope of the present invention is not limited thereto:

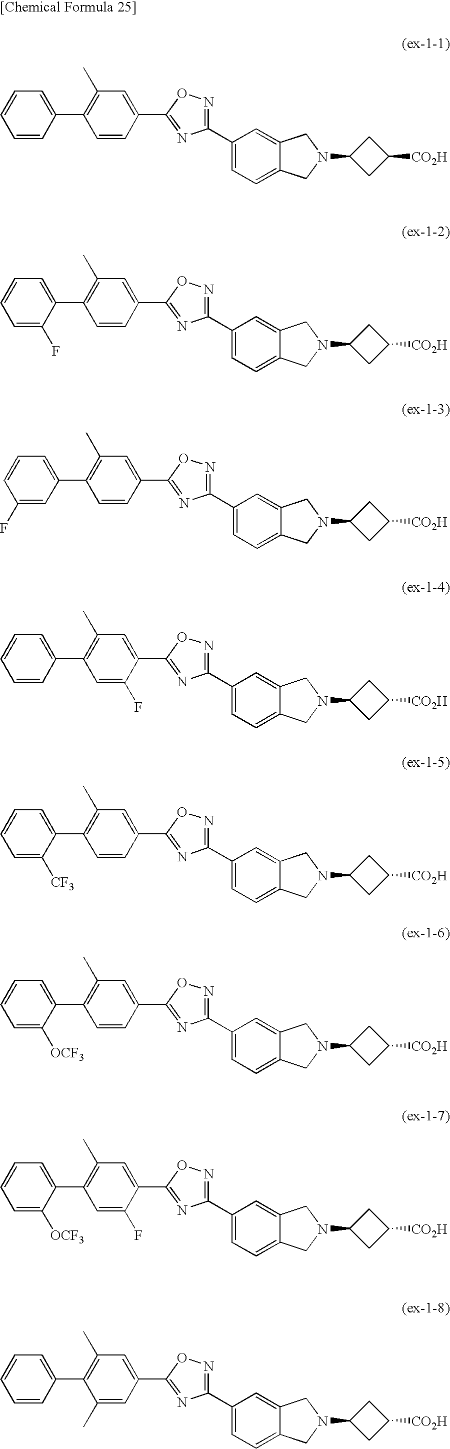

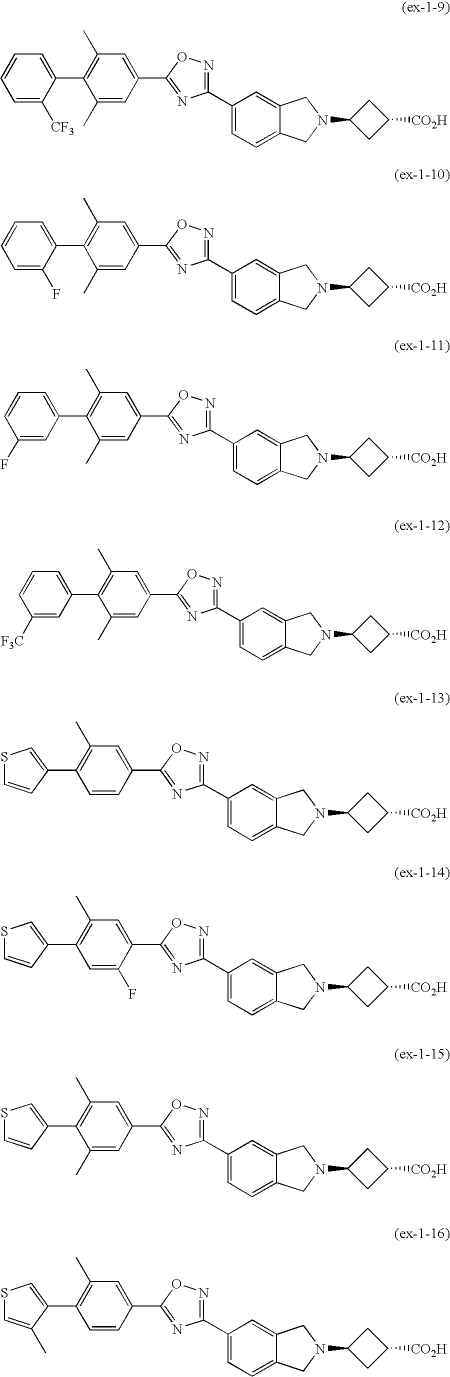

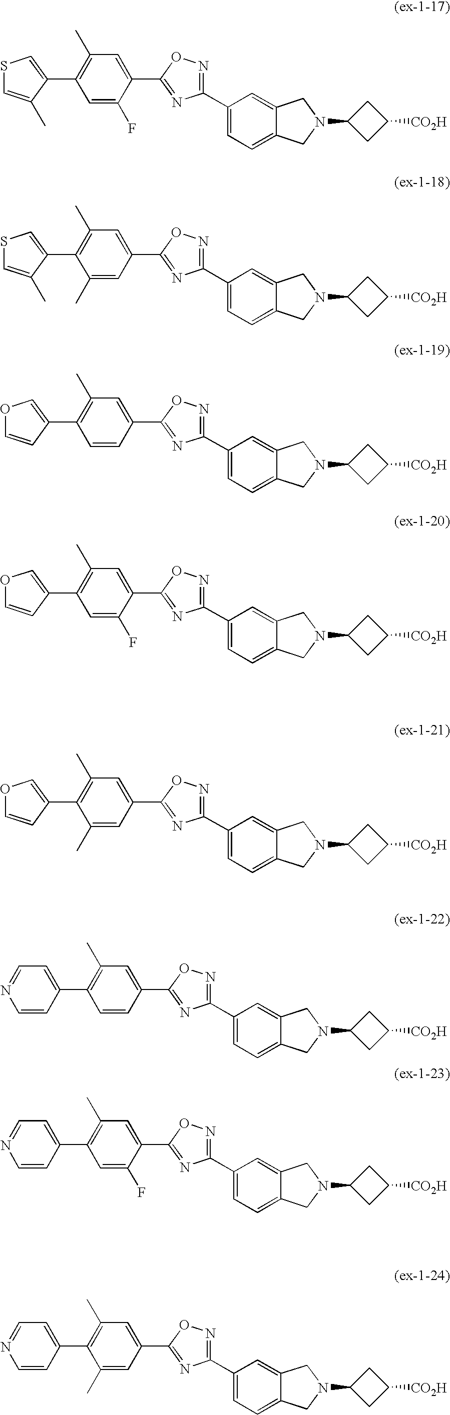

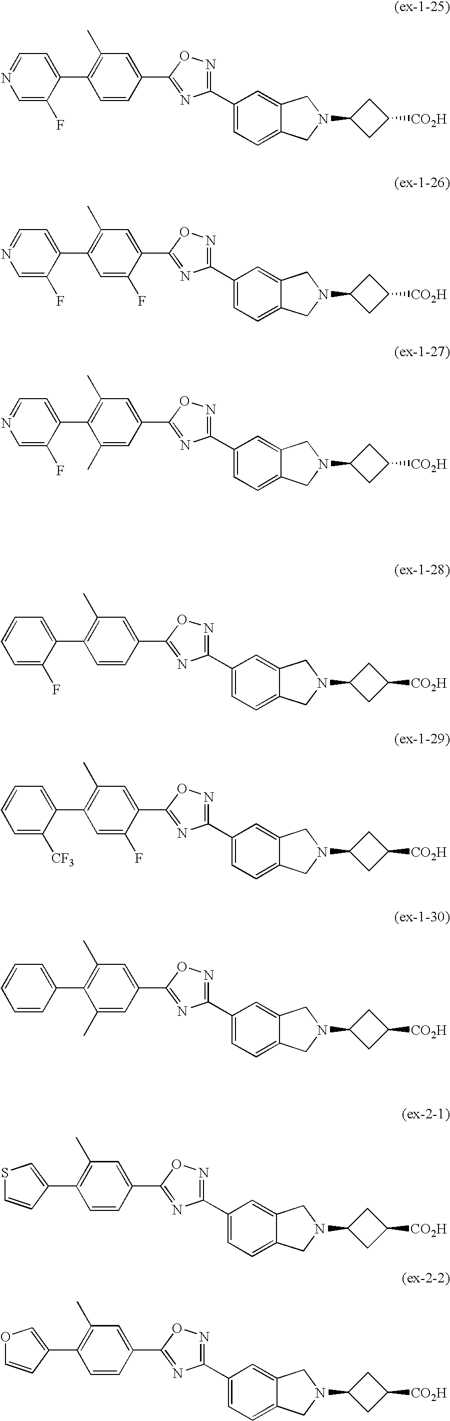

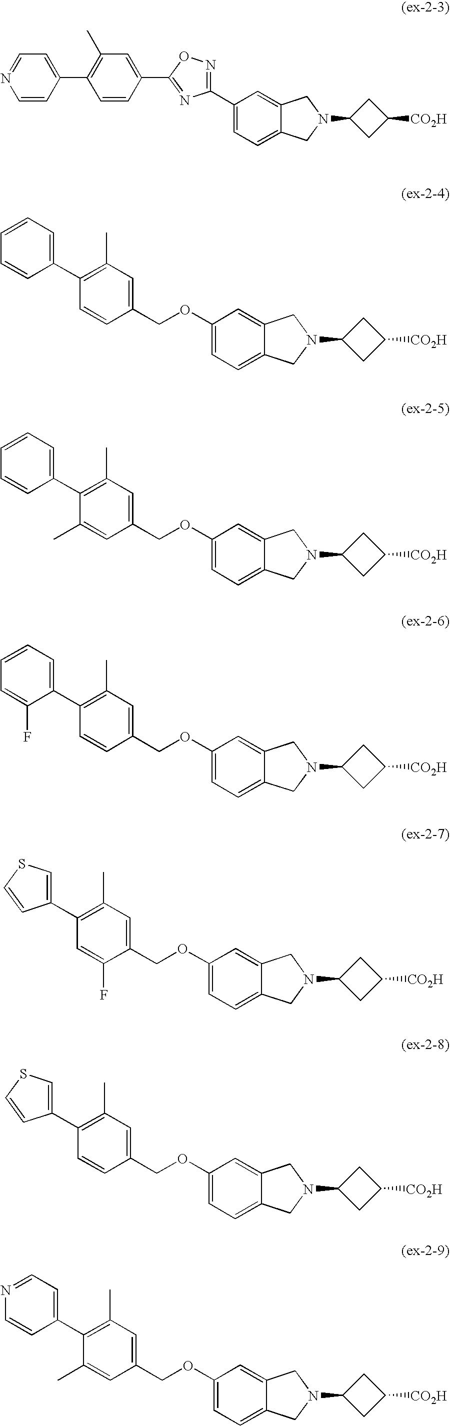

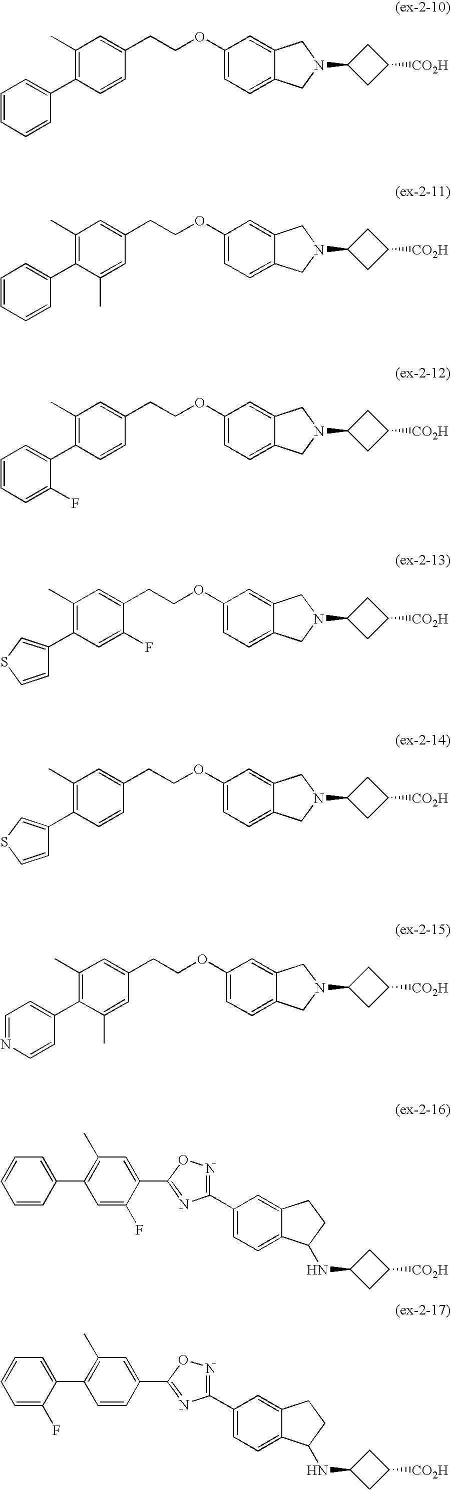

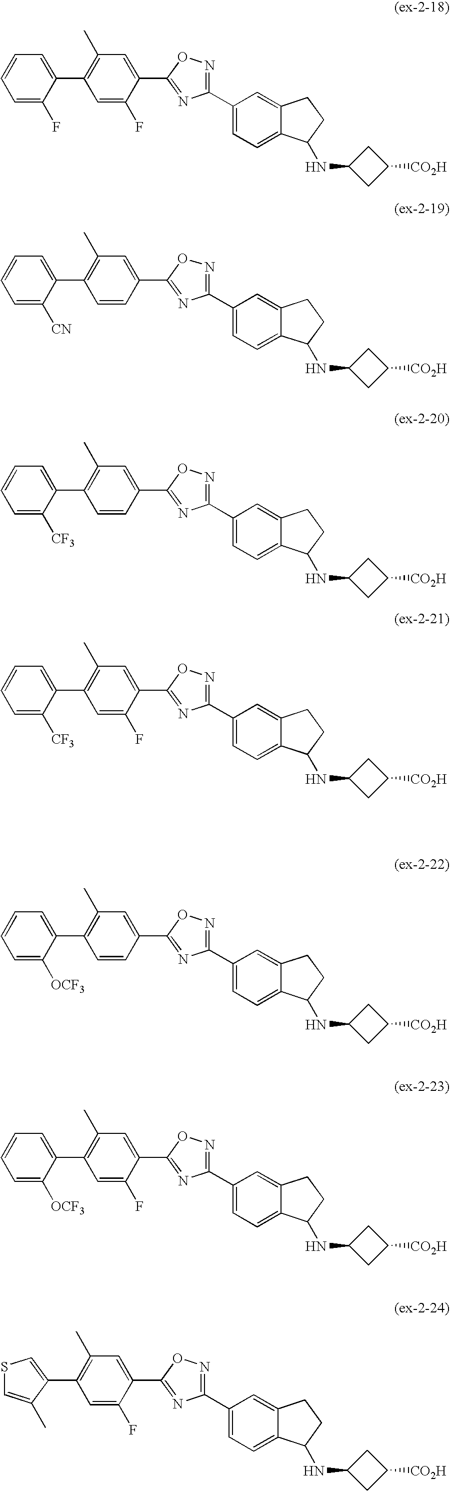

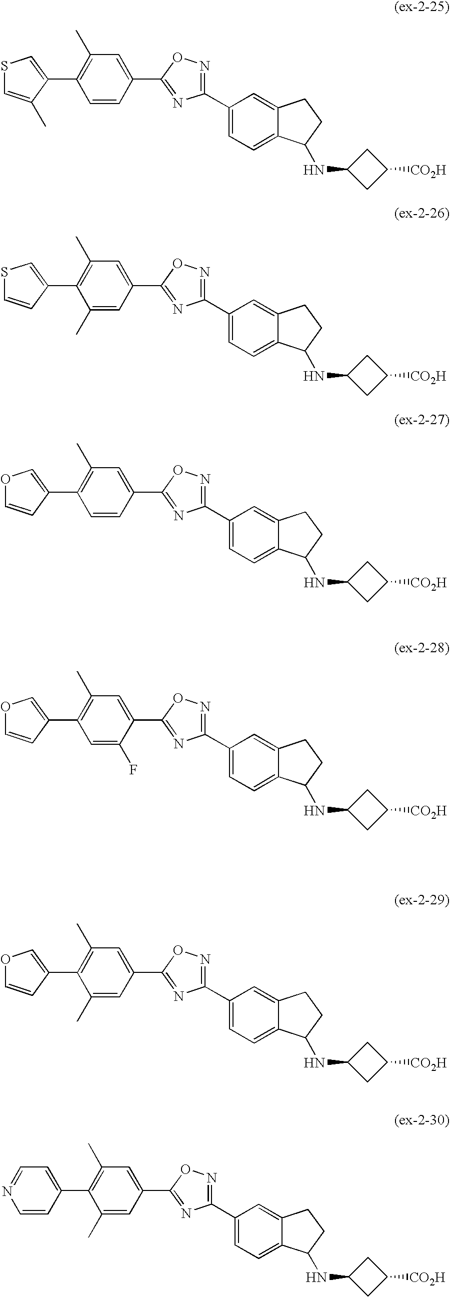

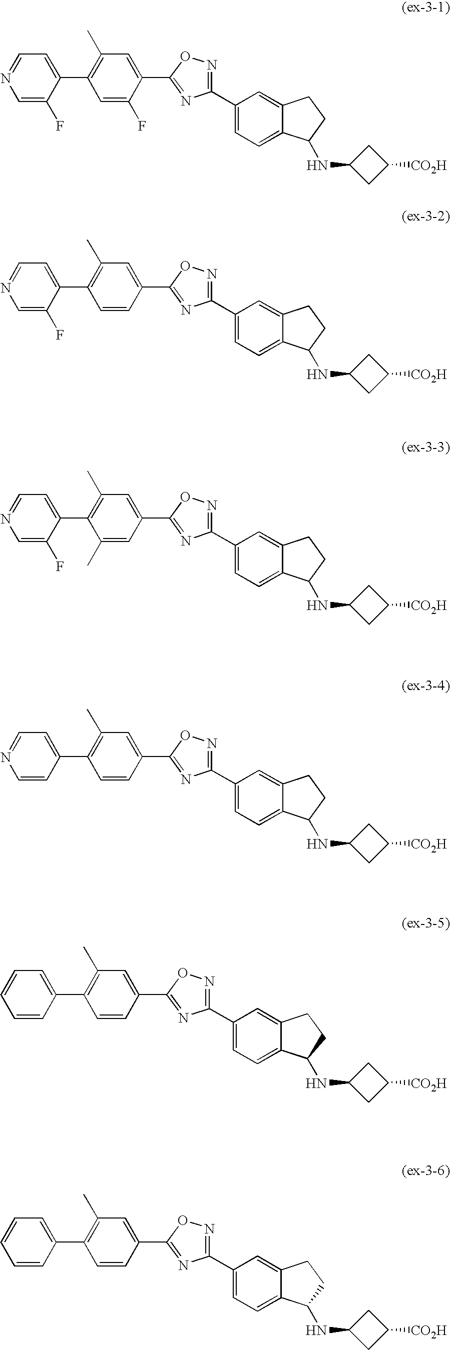

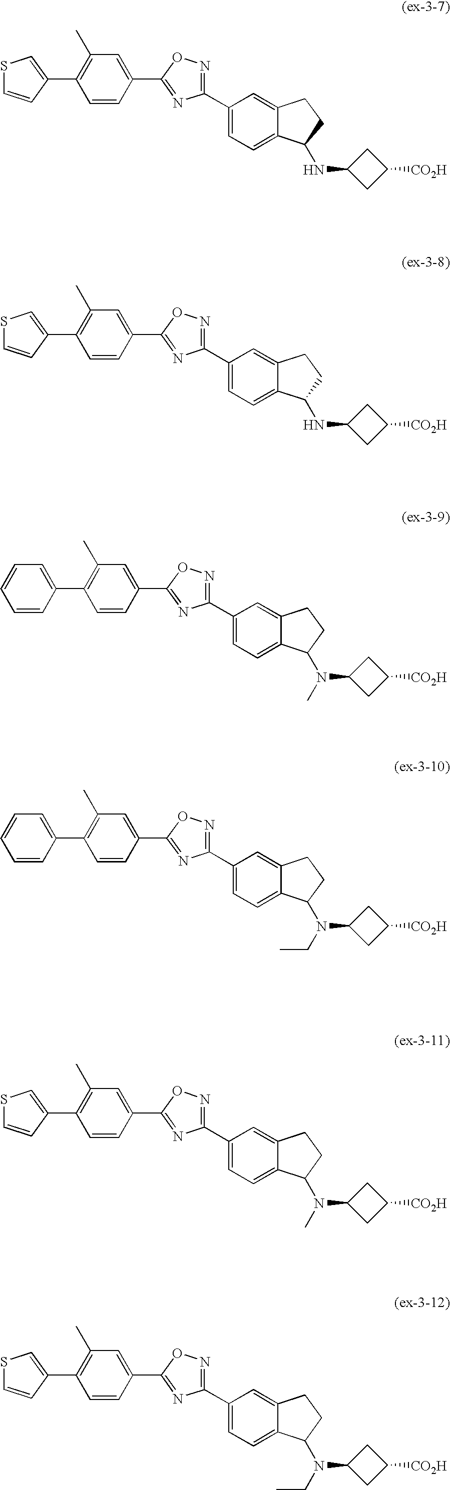

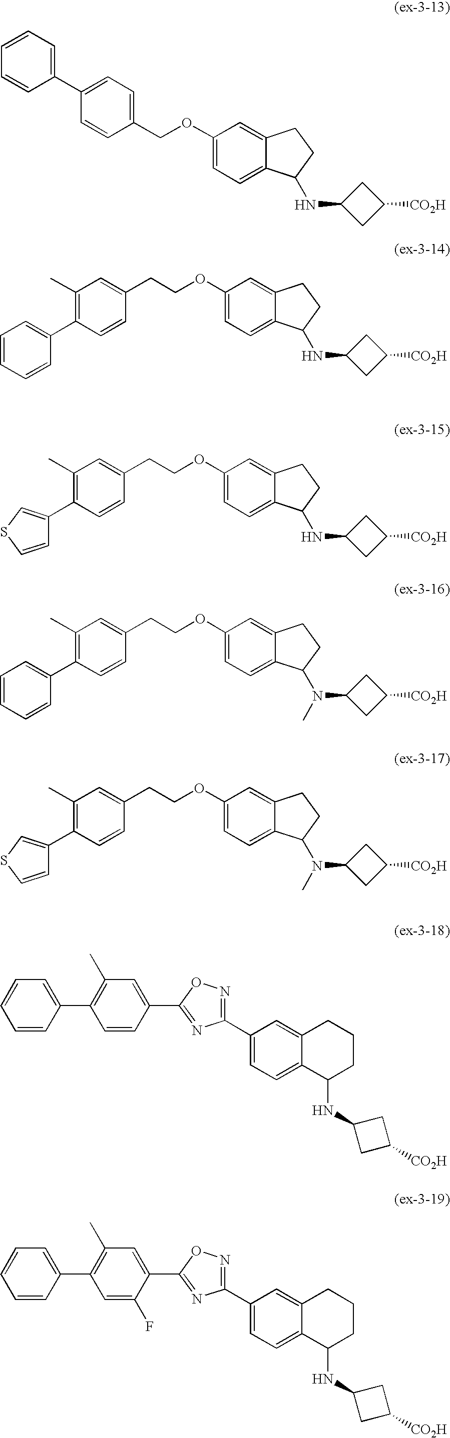

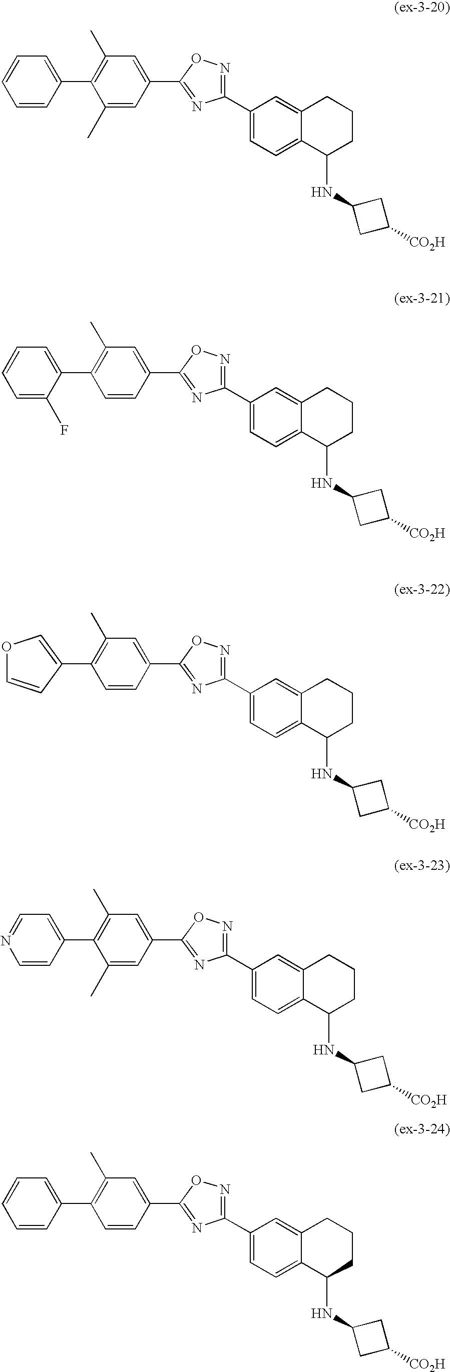

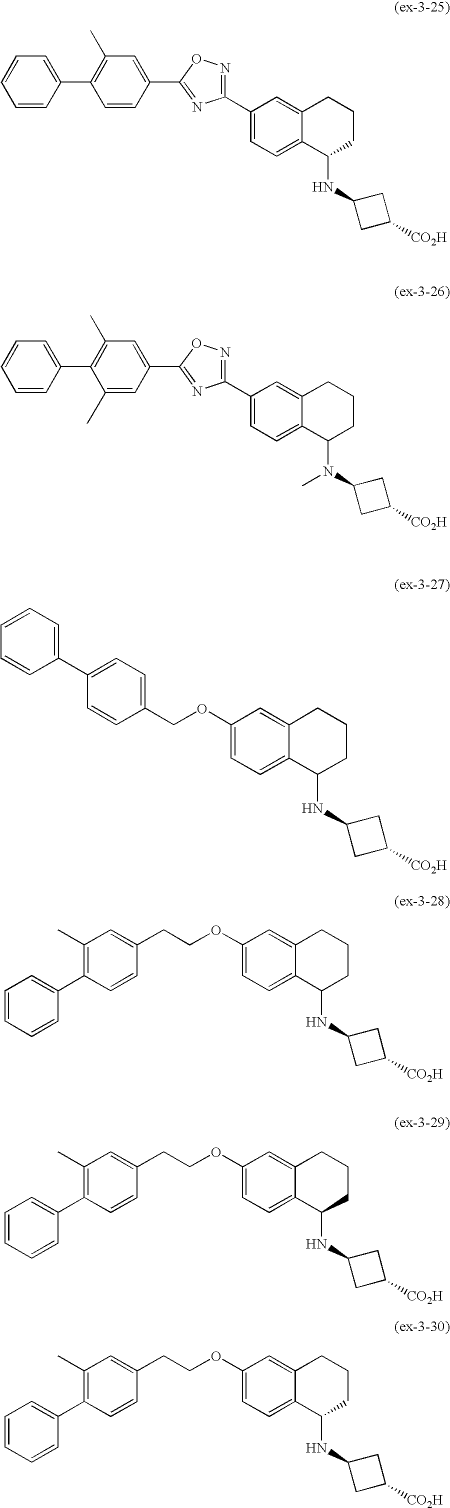

Further, a possible stereoisomer, a racemate, a pharmaceutically acceptable salt, a hydrate, a solvate or a prodrug of the compounds is also within the scope of the present invention.

The compounds represented by the Formula (1) can be prepared according to the method described below, for example. However, a method for the production of the compounds of the present invention is not limited to the following method.

For each of the chemical reactions, reaction time is not specifically limited. Instead, since the progress of the reaction can be easily followed according to a known analytical means, the reaction can be terminated when yield for a desired product reaches maximum value.

The compounds represented by the Formula (1) can be prepared according to a reverse synthetic pathway of the following reaction route, for example (i.e., reaction process for preparing method A; herein below, it is sometimes described as “route A”).

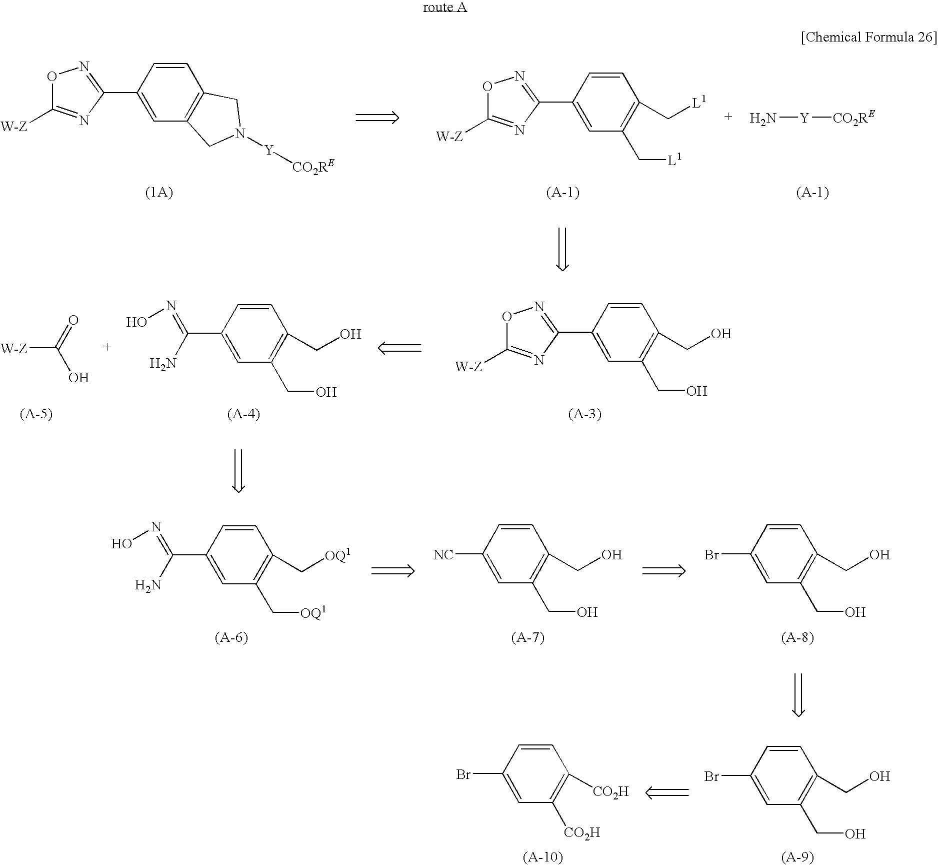

(Regarding a reaction process for preparing method A, the compound represented by Formula (1A) corresponds to a compound represented by the Formula (1) in which -Z-V— is represented by the Formula (2) and R¹ is linked to X² via a C1 alkylene to form a 5-membered ring. W, Z, Y and R^{E} are as defined above, L¹ is a leaving group, Q¹ is a protecting group for protecting a hydroxy group. Examples of Q¹ include a siliylether protecting group such as tert-butyldimethylsilyl group, etc. In addition, at least one of these groups can be used as protected).

The compounds represented by Formula (1A) can be prepared by alkylation reaction between compounds represented by Formula (A-1) and compounds represented by Formula (A-2). For such alkylation reaction, a base can be added, if necessary.

L¹ as a leaving group include a halogen atom or an acyloxy group and the like. Preferred examples of halogen atom include a chlorine atom, a bromine atom or an iodine atom. Preferred examples of acyloxy group include an alkylsulfonyloxy group which may be halogenated, an arylsulfonyloxy group which may be substituted or an alkyloxy sulfonyloxy group, and the like. Preferred examples of alkylsulfonyloxy group which may be halogenated include a methane sulfonyloxy group, a trifluoromethane sulfonyloxy group and the like. Preferred examples of arylsulfonyloxy group which may be substituted include a benzene sulfonyloxy group, a para-toluene sulfonyloxy group and the like. Preferred examples of alkyloxysulfonyloxy group include a methoxysulfonyloxy group, an ethoxysulfonyloxy group and the like.

For the alkylation reaction, the compounds represented by the Formula (A-1) are usually used in a molar amount of at least 0.9 to no more than 10 times, preferably at least 0.5 to no more than 3 times the molar amount of the compounds represented by the Formula (A-2). Examples of inert solvent that can be used for the present invention include halogenated hydrocarbons such as dichloromethane, chloroform and the like, ethers such as tetrahydrofuran, dioxane, diethyl ether and the like, dimethylsulfoxide, N,N-dimethyl formamide, acetonitrile. These can be used alone or as a mixture. Examples of bases that can be used for the present invention include alkali metal compounds such as sodium hydrocarbonate, sodium hydroxide, sodium hydride, potassium carbonate, sodium carbonate, potassium hydroxide, sodium methylate and the like, or organic tertiary amines such as pyridine, trimethylamine, triethylamine, N,N-diisopropylethylamine, N-methylmorpholine and the like. These are usually used in a molar amount of at least 1 to no more than 20 times, preferably at least 1 to no more than 10 times the molar amount of the compounds represented by the Formula (A-1). Reaction temperature is preferably −30° C. or more, and more preferably 0° C. or more. Further, it is preferably 150° C. or less, and more preferably 120° C. or less.

Reaction time may vary depending on starting compounds, a base, a solvent, reaction temperature and the like. In general, it is in the range of 30 minutes to 72 hours, and preferably in the range of 1 to 48 hours.

If at least one protecting group is present in the compounds represented by the Formula (1A), the compounds represented by the Formula (1) in which R¹ is linked to X² via a C1 alkylene to form a 5-membered ring can be prepared by deprotecting all the protecting groups simultaneously or one by one. Deprotection can be carried out according to any known method, for example, according to a method described in Protective Groups in Organic Synthesis, John Wiley and Sons (1999). For a case in which no protecting group is present in the compounds represented by the Formula (1A), a skilled person in the art would easily understand that the compounds represented by the Formula (1A) correspond to the compounds represented by the Formula (1) in which R¹ is linked to X² via a C1 alkylene to form a 5-membered ring.

Compounds represented by the Formula (A-2) for the reaction process for the preparing method A can be obtained as a commercially available product as described in Table 1 or can be prepared according to the method described in Reference examples 1 to 6, for example.

Unless specifically described otherwise, in each table, “No.” indicates compound number, “structure” indicates a chemical structure and “suppl.” indicates a supplier name. Further, abbreviations included in “suppl.” column are as follows. “AMRI”; product of AMRI INC., “TCI”; product of Tokyo Chemical Industry, Co., Ltd., “Ald”; product of Aldrich Company, “Wako”; product of Wako Pure Chemical Industries, Ltd., “Fro”; product of Frontier INC., “Butt”; product of Butt Park Ltd., “Acr”; product of Acros Chemicals, “Tyg”; product of Tyger Co., “Lan”; product of Lancaster Company.

TABLE 1

| No. | structure | suppl. |
|---|---|---|
| A-2-A | 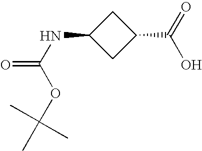 | AMRI |
| A-2-B | 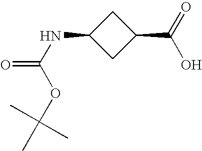 | AMRI |

Compounds represented by the Formula (A-1) can be prepared from the compounds represented by the Formula (A-3).

For the compounds represented by the Formula (A-1) in which L¹ is an acyloxy group, for example, the compounds represented by the Formula (A-3) can be reacted in an inert solvent with a corresponding acyl halide in the presence of a base to give the compounds represented by the Formula (A-1). Examples of such acyl halide include para-toluene sulfonyl chloride, methanesulfonyl chloride, and the like.

Examples of base that can be used for the acylation include triethylamine, diisoropylethylamine, pyridine and the like.

Type of a solvent used for the acylation is not specifically limited if it is inert to the acylation. Examples thereof include a saturated hydrocarbon solvent, a halogenated hydrocarbon solvent, an ether solvent, an aromatic hydrocarbon solvent and the like. These can be used alone or as a mixture comprising them in any mixing ratio. Examples of saturated hydrocarbon solvent include pentane, hexane, heptane and cyclohexane, and examples of halogenated hydrocarbon solvent include dichloromethane, chloroform, and 1,2-dichloroethane. Examples of ether solvent include tetrahydrofuran, diethyl ether, and 1,4-dioxane, and examples of aromatic hydrocarbon solvent include toluene, xylene and the like. Preferred examples include dichloromethane, chloroform, diethyl ether, tetrahydrofuran, toluene and the like.

For the acylation reaction, the acyl halide is preferably used in a molar amount of at least 0.5 times, and more preferably at least 1 times, to the molar amount of the compounds represented by the Formula (A-3). Further, it is preferably used in a molar amount of 10 times or less, and more preferably 2 times or less, compared to the molar amount of the compounds of the Formula (A-3).

For the acylation reaction, the base is preferably used in a molar amount of at least 1 times, and more preferably at least 2 times, compared to the molar amount of the acyl halide.

Reaction temperature may vary depending on starting compounds, a solvent, and the like. In general, the reaction is preferably carried out in the temperature range of −30° C. to room temperature.

Reaction time may vary depending on starting compounds, a solvent, reaction temperature and the like. In general, it is in the range of one minute to 12 hours.

For the compounds represented by the Formula (A-1) in which L¹ is a bromine atom, the compounds represented by the Formula (A-3) can be reacted in an inert solvent with carbon tetrabromide in the presence of triphenyl phosphine to give the compounds represented by the Formula (A-1).

Type of a solvent used for the halogenation is not specifically limited if it is inert to the halogenation. Examples thereof include a saturated hydrocarbon solvent, a halogenated hydrocarbon solvent, an ether solvent, an aromatic hydrocarbon solvent and the like. These can be used alone or as a mixture comprising them in any mixing ratio. Examples of saturated hydrocarbon solvent include pentane, hexane, heptane and cyclohexane, and examples of halogenated hydrocarbon solvent include dichloromethane, chloroform, and 1,2-dichloroethane. Examples of ether solvent include tetrahydrofuran, diethyl ether, and 1,4-dioxane, and examples of aromatic hydrocarbon solvent include toluene, xylene and the like. Preferred examples include dichloromethane, chloroform, diethyl ether, tetrahydrofuran, toluene and the like.

For the halogenation reaction, carbon tetrachloride is preferably used in a molar amount of at least 0.5 times, and more preferably at least 1 times, compared to the molar amount of the compounds represented by the Formula (A-3). Further, it is preferably used in a molar amount of 10 times or less, and more preferably 5 times or less, compared to the molar amount of the compounds of the Formula (A-3).

For the halogenation reaction, triphenyl phosphine is preferably used in a molar amount of at least 1 times, and also 5 times or less, compared to the molar amount of carbon tetrabromide.

Reaction temperature may vary depending on starting compounds, a solvent, and the like. In general, the reaction is preferably carried out in the temperature range of −30° C. to 50° C.

Reaction time may vary depending on starting compounds, a solvent, reaction temperature and the like. In general, it is in the range of one minute to 12 hours.

The compound represented by the Formula (A-3) can be prepared by subjecting the compounds represented by the Formula (A-4) and the compounds represented by the Formula (A-5) to a condensation reaction in the presence of a dehydration condensation agent.

The condensation reaction can be carried out, if necessary, in the presence of 1 to 1.5 equivalents of 1-hydroxybenzotriazole (HOBT) and/or a catalytic amount to 5 equivalents of base compared to the compounds represented by Formula (A-4). Examples of a dehydration condensation agent include dicyclohexylcarbodiimide (DCC) or 1-ethyl-3-(3-dimethylaminopropyl)carbodiimide hydrochloric acid salt (WSC.HCl) and the like. Among these, WSC is preferred.

An inert solvent that can be used for the condensation reaction is not specifically limited if it is inert to the reaction, and examples thereof include a nitrile solvent, an amide solvent, a halogenated hydrocarbon solvent, an ether solvent and the like. These can be used as a mixture comprising them in any mixing ratio. Preferred examples of a nitrile solvent include acetonitrile and the like. Preferred examples of an amide solvent include N,N-dimethylformamide and the like. Preferred examples of an ether solvent include tetrahydrofuran and the like.

As for a base, a strong base such as hydrides of an alkali metal or an alkali earth metal, amides of an alkali metal or an alkali earth metal, lower alkoxides of an alkali metal or an alkali earth metal and the like, an inorganic base such as hydroxides of an alkali metal or an alkali earth metal, carbonates of an alkali metal or an alkali earth metal, hydrocarbonates of an alkali metal or an alkali earth metal and the like, an organic amine, or an organic base such as basic heterocyclic compound and the like can be mentioned. Examples of hydrides of an alkali metal or an alkali earth metal include lithium hydride, sodium hydride, calcium hydride, potassium hydride and the like. Examples of amides of an alkali metal or an alkali earth metal include lithium amide, sodium amide, lithium diisopropylamide, lithium dicyclohexylamide, lithium hexamethyldisilazide, sodium hexamethyldisilazide, potassium hexamethyldisilazide and the like. Examples of lower alkoxides of an alkali metal or an alkali earth metal include sodium methoxide, sodium ethoxide, or potassium tert-butoxide and the like. Examples of hydroxides of an alkali metal or an alkali earth metal include sodium hydroxide, potassium hydroxide, lithium hydroxide, barium hydroxide and the like. Examples of carbonates of an alkali metal or an alkali earth metal include sodium carbonate, potassium carbonate, cesium carbonate and the like. Examples of hydrocarbonates of an alkali metal or an alkali earth metal include sodium hydrocarbonate, potassium hydrocarbonate, and the like. Examples of organic amines include triethylamine, diisopropylethylamine, N-methylmorpholilne, 4-dimethylaminopyridine, DBU (1,8-diazabicyclo[5.4.0]undec-7-ene), DBN (1,5-diazabicyclo[4.3.0]non-5-ene) and the like. Examples of organic bases such as basic heterocyclic compound include pyridine, imidazole, 2,6-lutidine and the like. Among the bases described above, triethylamine, diisopropylethylamine, 4-dimethylaminopyridine and the like are preferred.

Reaction temperature may vary depending on starting compounds, a solvent, and the like. In general, the reaction is preferably carried out in the temperature range of 0° C. to 150° C. Preferably, it is in the temperature range of room temperature to 120° C.

Reaction time may vary depending on starting compounds, a base, a solvent, reaction temperature and the like. In general, it is in the range of one hour to 24 hours.

The compounds represented by the Formula (A-4) can be prepared by deprotection reaction of the compounds represented by the Formula (A-6).

Deprotection can be carried out according to any known method, for example, according to a method described in Protective Groups in Organic Synthesis, John Wiley and Sons (1999).

The compounds represented by the Formula (A-6) can be prepared by reacting the compounds represented by the Formula (A-7) with hydroxylamine hydrochloric acid salt in the presence of a base.

Examples of base which can be used for the reaction include an inorganic base such as sodium hydrocarbonate, sodium carbonate, potassium carbonate and the like and an organic base such as triethylamine, diisopropylethylamine, pyridine and the like.

An organic solvent that can be used for the reaction is not specifically limited if it is inert to the reaction. Examples thereof include an alcohol solvent such as methanol, ethanol and the like, an ether solvent such as diethyl ether, tetrahydrofuran, 1,4-dioxane and the like, an amide solvent such as N,N-dimethyl formamide and the like and a mixture solvent comprising them in any mixing ratio.

Reaction temperature may vary depending on starting compounds, a solvent, and the like. In general, the reaction is preferably carried out in the temperature range of room temperature to 150° C. Preferably, it is in the temperature range of room temperature to 120° C. Reaction time may vary depending on starting compounds, a solvent, reaction temperature and the like. In general, it is in the range of 30 minutes to 72 hours, preferably in the range of 1 to 48 hours.

The compounds represented by the Formula (A-7) can be prepared by cynataion of the compounds represented by the Formula (A-8).

As for a source for cyanide required for the reaction, zinc cyanide, copper cyanide, potassium cyanide, sodium cyanide, and the like can be mentioned.

For carrying out the reaction, diethyl zinc, copper sulfate and the like can be also used, if necessary.

An organic solvent that can be used for the reaction is not specifically limited if it is inert to the reaction. Examples thereof include an amide solvent such as N,N-dimethyl formamide, N-methylpyrrolidone and the like, an ether solvent such as 1,4-dioxane and the like, pyridine, quinoline, and a mixture solvent comprising them in any mixing ratio.

When zinc cyanide is used as a source of cyanide, a palladium catalyst is used together. Examples thereof include tetrakis(triphenylphosphine)palladium, tetrakis(methyldiphenylphosphine)palladium, dichlorobis(triphenylphosphine)palladium, dichlorobis(tri o-tolylphosphine)palladium, dichlorobis(tricyclohexylphosphine)palladium, dichlorobis(triethylphosphine)palladium, palladium acetate, palladium chloride, chlorobis(acetonitrile)palladium, tris(dibenzylideneacetone)dipalladium, chlorobis(diphenylphosphinoferrocene)palladium and the like. In addition, a catalyst produced by using palladium acetate, tris(dibenzylideneacetone)dipalladium, and the like and any ligand can be also used. With respect to valency of palladium, it can be either 0 or +2. Examples of a ligand for palladium include a phosphine type ligand such as trifurylphosphine, tri(o-tolyl)phosphine, tri(cyclohexyl)phosphine, tri(t-butyl)phosphine, dicyclohexylphenylphosphine, 1,1′-bis(di-t-butylphosphino)ferrocene, 2-dicyclohexylphosphino-2′-dimethylamino-1,1′-biphenyl, 2-(di-t-butylphosphino) biphenyl and the like or a non-phosphine type ligand such as imidazol-2-ylidene carbene and the like.

For the reaction in which zinc cyanide is used as a source of cyanide, amount of the palladium catalyst is preferably 0.01 to 20 mol %, and more preferably 0.1 to 10 mol % compared to reacting materials.

For the reaction in which zinc cyanide is used as a source of cyanide, the reaction temperature may vary depending on starting compounds, a catalyst, a base, a solvent, and the like. In general, the reaction is preferably carried out in the temperature range of 0° C. to 150° C. Preferably, it is in the temperature range of room temperature to 120° C. Reaction time may vary depending on starting compounds, a catalyst, a base, a solvent, reaction temperature and the like. In general, it is in the range of 30 minutes to 72 hours, preferably in the range of 1 to 48 hours.

For the reaction in which copper cyanide is used as a source of cyanide, the reaction temperature may vary depending on starting compounds, a solvent, and the like. In general, the reaction is preferably carried out in the temperature range of 100° C. to 300° C. Preferably, it is in the temperature range of 150° C. to 250° C. Reaction time may vary depending on starting compounds, a solvent, reaction temperature and the like. In general, it is in the range of 30 minutes to 72 hours, preferably in the range of 1 to 48 hours.

The compounds represented by the Formula (A-8) can be prepared by protection reaction of the compounds represented by the Formula (A-9).

Protection can be carried out according to any known method, for example, according to a method described in Protective Groups in Organic Synthesis, John Wiley and Sons (1999).

The compounds represented by the Formula (A-9) can be prepared by reduction of the compounds represented by the Formula (A-10).

As for a reducing agent which can be used for the reaction, lithium aluminum hydride, sodium borohydride, a borane complex and the like can be mentioned. Preferred examples of a metal hydride complex include lithium aluminum hydride and the like. Preferred examples of a borane complex include a borane-dimethylsulfide complex and the like.

Type of a solvent used for the reduction of the compounds represented by the Formula (A-10) is not specifically limited if it is inert to the reduction. Examples thereof include a saturated hydrocarbon solvent, a halogenated hydrocarbon solvent, an ether solvent, an aromatic hydrocarbon solvent and the like. These can be used alone or as a mixture solvent comprising them in any mixing ratio. Examples of saturated hydrocarbon solvent include pentane, hexane, heptane, cyclohexane and the like, and examples of halogenated hydrocarbon solvent include dichloromethane, chloroform, 1,2-dichloroethane and the like. Examples of ether solvent include tetrahydrofuran, diethylether, 1,4-dioxane and the like, and examples of aromatic hydrocarbon solvent include toluene, xylene and the like. Preferred examples include diethyl ether, tetrahydrofuran, toluene and a mixture solvent comprising them in any mixing ratio, and the like.

The reducing agent is preferably used in a molar amount of at least 0.1 times, more preferably at least 1 times the molar amount of the compounds represented by the Formula (A-10). In addition, preferably it is used in a molar amount of no more than 100 times, more preferably no more than 10 times the molar amount of the compounds represented by the Formula (A-10).

Reaction temperature may vary depending on starting compounds, a reducing agent, a solvent, and the like. In general, the reaction is preferably carried out at the temperature of −100° C. or more. Preferably, it is carried out at the temperature of 100° C. or less.

Reaction time may vary depending on starting compounds, a reducing agent, a solvent, reaction temperature and the like. In general, it is in the range of 5 minutes to 12 hours.

With respect to the compounds represented by the Formula (A-10), for example, 4-bromophthalic acid can be obtained as a commercially available product (manufactured by Tokyo Chemical Industry Co., Ltd.).

The compounds represented by the Formula (A-5) can be prepared according to a reverse synthetic pathway of the following reaction route, for example (i.e., reaction process for preparing method B; herein below, it is sometimes described as “route B”).

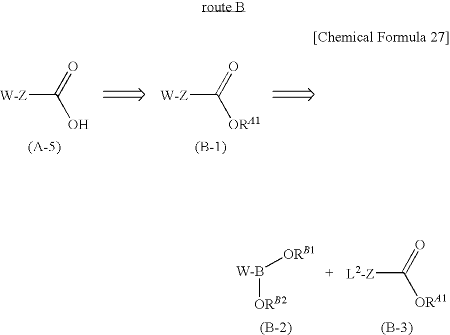

(Regarding a reaction process for preparing method B, W and Z are as defined above. R^{A1} is a hydrogen atom or a substituted alkyl group, and for R^{A1} as an alkyl group, a protecting group such as a benzyl group, a methyl group, an ethyl group and the like can be mentioned. R^{B1}, R^{B2} can be the same or different from each other, a hydrogen atom or a C1-4 alkyl group or R^{B1} and R^{B2} may together to form a 1,1,2,2-tetramethylethylene group. L² is a leaving group, and preferred examples of L² include a chlorine atom, a bromine atom, an iodine atom, a trifluoromethanesulfonyloxy group and the like. Further, one or more of these groups can be protected).

For a case in which R^{A1} is a hydrogen atom, a skilled person in the art would easily understand that the compounds represented by the Formula (A-5) correspond to the compounds represented by the Formula (B-1). Further, a skilled person in the art would easily understand that the carboxylic acid compounds represented by the Formula (A-5) or the Formula (B-1) can be produced according to the method described in the present scheme.

Deprotection of the compounds represented by the Formula (B-1) to obtain the compounds represented by the Formula (A-5) can be carried out according to any known method, for example, according to a method described in Protective Groups in Organic Synthesis, John Wiley and Sons (1999).

The compounds represented by the Formula (B-1) can be prepared by Suzuki reaction of the compounds represented by the Formula (B-2) and the compounds represented by the Formula (B-3) in the presence of a palladium catalyst. Examples of palladium catalyst which can be used for the Suzuki reaction include tetrakis(triphenylphosphine)palladium, tetrakis(methyldiphenylphosphine)palladium, dichlorobis(triphenylphosphine)palladium, dichlorobis(tri-o-tolylphosphine)palladium, dichlorobis(tricyclohexylphosphine)palladium, dichlorobis(triethylphosphine)palladium, palladium acetate, palladium chloride, chlorobis(acetonitrile)palladium, tris(dibenzylideneacetone)dipalladium, chlorobis(diphenylphosphinoferrocene)palladium and the like. In addition, a catalyst produced by using palladium acetate, tris(dibenzylideneacetone)dipalladium, and the like and any ligand can be also used. With respect to valency of palladium, it can be either 0 or +2. Examples of a ligand for palladium include a phosphine type ligand such as trifurylphosphine, tri(o-tolyl)phosphine, tri(cyclohexyl)phosphine, tri(t-butyl)phosphine, dicyclohexylphenylphosphine, 1,1′-bis(di-t-butylphosphino)ferrocene, 2-dicyclohexylphosphino-2′-dimethylamino-1,1′-biphenyl, 2-(di-t-butylphosphino)biphenyl and the like, or a non-phosphine type ligand such as imidazol-2-ylidene carbene and the like.

Amount of the palladium catalyst used for the Suzuki reaction is preferably 0.01 to 20 mol %, and more preferably 0.1 to 10 mol %. A base which can be used for the Suzuki reaction include, for example, sodium carbonate, potassium carbonate, cesium carbonate, cesium fluoride, potassium fluoride, potassium phosphate, potassium acetate, triethylamine, potassium hydroxide, sodium hydroxide, sodium methoxide, lithium methoxide and the like.

Type of a solvent used for the Suzuki reaction is not specifically limited if it is inert to the reaction. Examples thereof include a hydrocarbon solvent such as toluene, xylene, hexane and the like, a halogenated hydrocarbon solvent such as dichloromethane, chloroform and the like, a sulfoxide solvent such as dimethylsulfoxide and the like, an amide solvent such as dimethyl formamide and the like, an ether solvent such as tetrahydrofuran, dioxane, diglyme and the like, an alcohol solvent such as methanol, ethanol and the like, a nitrile solvent such as acetonitrile and the like, a ketone solvent such as acetone, cyclohexanone and the like, an ester solvent such as ethyl acetate and the like, and a heterocyclic solvent such as pyridine and the like. These can be used as a mixture comprising them in any mixing ratio. In addition, with respect to a solvent system, any one of two-phase system comprising water and an organic solvent, water-comprising organic solvent, or homogeneous organic solvent system can be used.

Reaction temperature may vary depending on starting compounds, a catalyst, a base, a solvent, and the like. In general, the reaction is preferably carried out in the temperature range of 0° C. to 150° C. Preferably, it is in the temperature range of room temperature to 120° C. Reaction time may vary depending on starting compounds, a catalyst, a base, a solvent, reaction temperature and the like. In general, it is in the range of 30 minutes to 72 hours, preferably in the range of 1 to 48 hours.

With respect to the reaction process for the preparing method B, the compounds represented by the Formula (B-2) can be obtained as a commercially available product as described in Table 2, for example.

TABLE 2

| No. | structure | suppl. |
|---|---|---|
| B-2-A | 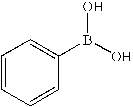 | TCI |
| B-2-B | 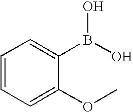 | Ald |
| B-2-C | 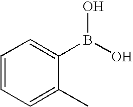 | Ald |
| B-2-D | 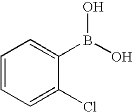 | Ald |
| B-2-E | 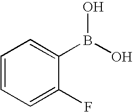 | Ald |
| B-2-F | 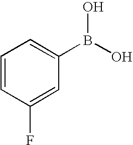 | Wako |
| B-2-G | 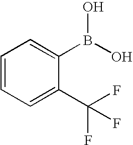 | Wako |
| B-2-H | 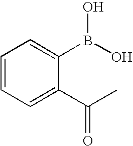 | Ald |
| B-2-I | 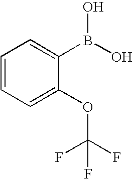 | Wako |
| B-2-J | 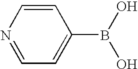 | Wako |
| B-2-K | 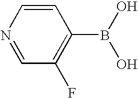 | Fro |
| B-2-L | 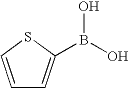 | Ald |
| B-2-M | 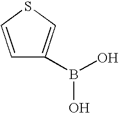 | Ald |
| B-2-N | 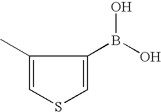 | Ald |
| B-2-O | 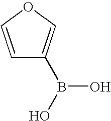 | Ald |

With respect to the reaction process for the preparing method B, the compounds represented by the Formula (B-3) can be obtained as a commercially available product as described in Table 3, for example.

TABLE 3

| No. | structure | suppl. |
|---|---|---|
| B-3-A | 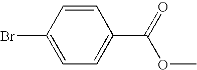 | TCI |
| B-3-B | 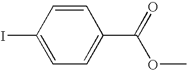 | TCI |
| B-3-C | 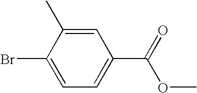 | TCI |
| B-3-D | 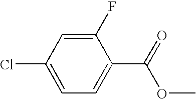 | Wako |
| B-3-E | 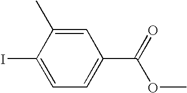 | TCI |
| B-3-F | 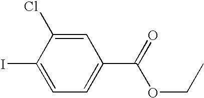 | Butt |

The compounds represented by the Formula (1) can be prepared according to a reverse synthetic pathway of the following reaction route, for example (i.e., reaction process for preparing method C; herein below, it is sometimes described as “route C”).

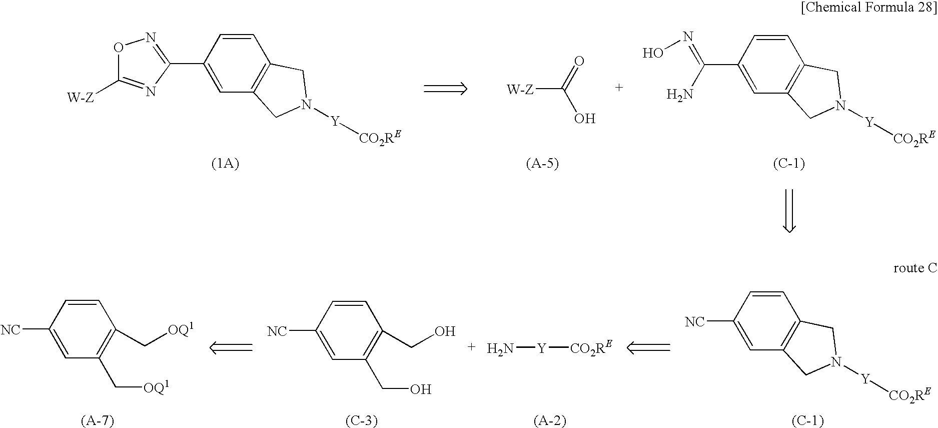

(Regarding a reaction process for preparing method C, the compound represented by Formula (1A) corresponds to a compound represented by the Formula (1) in which -Z-V— is represented by the Formula (2) and R¹ is linked to X² via a C1 alkylene to form a 5-membered ring. W, Z, Y, R^{E}, L¹ and Q¹ are as defined above. In addition, one or more of these groups can be protected).

With respect to a method of producing the compounds represented by the Formula (1A) from the compounds represented by the Formula (A-5) and the compounds represented by the Formula (C-1), the method which is the same as the method of producing the compounds represented by the Formula (A-3) from the compounds represented by the Formula (A-4) and the compounds represented by the Formula (A-5) can be exemplified.

With respect to a method of producing the compounds represented by the Formula (C-1) from the compounds represented by the Formula (C-2), the method which is the same as the method of producing the compounds represented by the Formula (A-6) from the compounds represented by the Formula (A-7) can be exemplified.

With respect to a method of producing the compounds represented by the Formula (C-2) from the compounds represented by the Formula (C-3) and the compounds represented by the Formula (A-2), the method which is the same as the method of producing the compounds represented by the Formula (1A) from the compounds represented by the Formula (A-1) and the compounds represented by the Formula (A-2) can be exemplified.

With respect to a method of producing the compounds represented by the Formula (C-3) from the compounds represented by the Formula (A-7), the method which is the same as the method of producing the compounds represented by the Formula (A-1) from the compounds represented by the Formula (A-3) or the method of producing the compounds represented by the Formula (A-4) from the compounds represented by the Formula (A-6) can be exemplified.

The compounds represented by the Formula (1) can be prepared according to a reverse synthetic pathway of the following reaction route, for example (i.e., reaction process for preparing method D; herein below, it is sometimes described as “route D”).

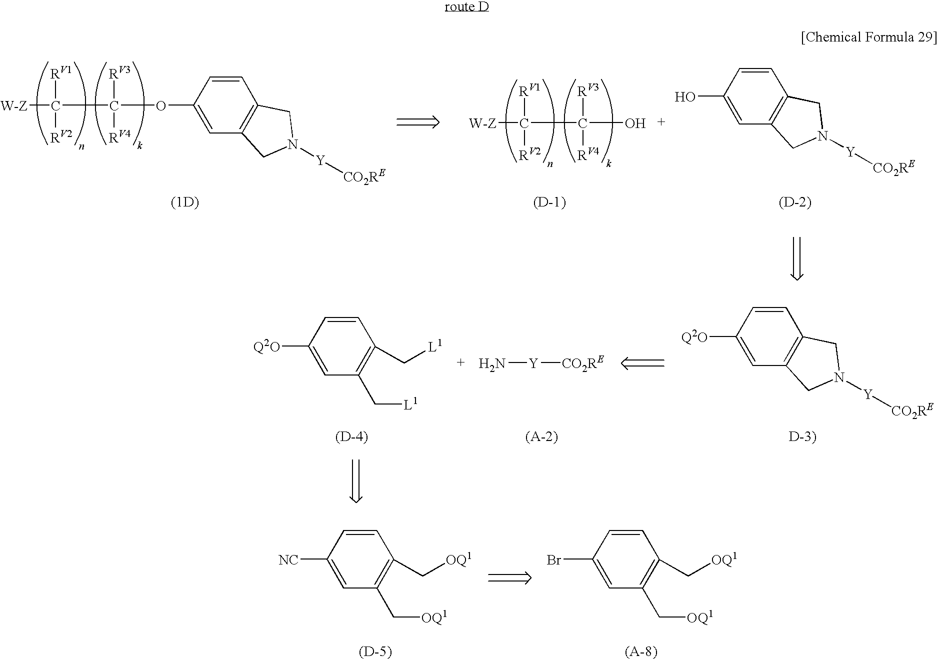

(Regarding a reaction process for preparing method D, the compound represented by Formula (1D) corresponds to a compound represented by the Formula (1) in which -Z-V— is -Z-(CR^{V1}R^{V2})ₙ—(CR^{V3}R^{V4})ₖ—O— and R¹ is linked to X² via a C1 alkylene to form a 5-membered ring. W, Z, R^{V1}, R^{V2}, R^{V3}, R^{V4}, n, k, Y, R^{E}, L¹, and Q¹ are as defined above. Q² is a protecting group for protecting a phenolic hydroxy group. Examples of Q² include an alkyl protecting group such a methyl group, a benzyl group, etc. In addition, one or more of these groups can be protected).

The compounds represented by the Formula (1D) can be prepared by Mitsunobu reaction between the compounds represented by the Formula (D-1) and the compounds represented by the Formula (D-2).

The azo compounds that can be used for the Mitsunobu reaction include ethyl azodicarboxylate, diisopropyl azodicarboxylate, N,N,N′,N′-tetramethyl azodicarboxamide, N,N,N′,N′-tetraisopropyl azodicarboxamide and the like.

The azo compound is used for the Mitsunobu reaction preferably in a molar amount of at least 0.5 times, more preferably at least 1 times the molar amount of the compounds represented by the Formula (D-1). In addition, it is used preferably in a molar amount of no more than 20 times, more preferably no more than 10 times the molar amount of the compounds represented by the Formula (D-1).

Examples of a phosphine reagent used for the Mitsunobu reaction include triphenylphosphine, tri n-butylphosphine and the like.

The phosphine reagent is used for the Mitsunobu reaction preferably in a molar amount of at least 0.5 times, more preferably at least 1 times the molar amount of the compounds represented by the Formula (D-1). In addition, it is used preferably in a molar amount of no more than 20 times, more preferably no more than 10 times the molar amount of the compounds represented by the Formula (D-1).

Type of a solvent used for the Mitsunobu reaction is not specifically limited if it is inert to the reduction. Examples thereof include a saturated hydrocarbon solvent, a halogenated hydrocarbon solvent, an ether solvent, an aromatic hydrocarbon solvent and the like. These can be used alone or as a mixture solvent comprising them in any mixing ratio. Examples of saturated hydrocarbon solvent include pentane, hexane, heptane and cyclohexane, and examples of halogenated hydrocarbon solvent include dichloromethane, chloroform, 1,2-dichloroethane and the like. Examples of ether solvent include tetrahydrofuran, diethyl ether, and 1,4-dioxane, and examples of aromatic hydrocarbon solvent include toluene, xylene and the like. Preferred examples include hexane, dichoromethane, chloroform, tetrahydrofuran, diethyl ether, toluene and a mixture solvent comprising them in any mixing ratio, and the like.

Reaction temperature for the Mitsunobu reaction is preferably −50° C. or more. More preferably, it is −30° C. or more. In addition, it is the same or less than the boiling point of a solvent used for the reaction. More preferably, it is the same or less than 30° C.

Reaction time for the Mitsunobu reaction may vary depending on starting compounds, a base, a solvent, reaction temperature and the like. In general, it is in the range of 5 minutes to hours.

The compounds represented by the Formula (D-2) can be prepared by deprotection reaction of the compounds represented by the Formula (D-3).

Deprotection can be carried out according to any known method, for example, according to a method described in Protective Groups in Organic Synthesis, John Wiley and Sons (1999).

With respect to a method of producing the compounds represented by the Formula (D-3) from the compounds represented by the Formula (D-4) and the compounds represented by the Formula (A-2), the method which is the same as the method of producing the compounds represented by the Formula (1A) from the compounds represented by the Formula (A-1) and the compounds represented by the Formula (A-2) can be exemplified.

With respect to a method of producing the compounds represented by the Formula (D-4) from the compounds represented by the Formula (D-5), the method which is the same as the method of producing the compounds represented by the Formula (C-3) from the compounds represented by the Formula (A-7) can be exemplified.

The compounds represented by the Formula (D-5) can be prepared by reaction of the compounds represented by the Formula (A-8) with an alcohol such as methanol, benzyl alcohol and the like.

An organic solvent that can be used for the reaction is not specifically limited if it is inert to the reaction. Examples thereof include a hydrocarbon solvent such as toluene, xylene and the like, an amide solvent such as dimethyl formamide and the like, and an ether solvent such as tetrahydrofuran, dioxane, diglyme and the like. In addition, two or more kinds of organic solvent can be used as a mixture.

Examples of a catalyst which can be used for the reaction include tetrakis(triphenylphosphine)palladium, tetrakis(methyldiphenylphosphine)palladium, dichlorobis(triphenylphosphine)palladium, dichlorobis(tri-o-tolylphosphine)palladium, dichlorobis(tricyclohexylphosphine)palladium, dichlorobis(triethylphosphine)palladium, palladium acetate, palladium chloride, chlorobis(acetonitrile)palladium, tris(dibenzylideneacetone)dipalladium, or chlorobis(diphenylphosphinoferrocene)palladium and the like. In addition, a catalyst produced by using palladium acetate, tris(dibenzylideneacetone)dipalladium, and the like and any ligand can be also used. With respect to valency of palladium, it can be either 0 or +2. Examples of a ligand for palladium include a phosphine type ligand such as trifurylphosphine, tri(o-tolyl)phosphine, tri(cyclohexyl)phosphine, tri(t-butyl)phosphine, dicyclohexylphenylphosphine, 1,1′-bis(di-t-butylphosphino)ferrocene, 2-dicyclohexylphosphino-2′-dimethylamino-1,1′-biphenyl, 2-(di-t-butylphosphino)biphenyl and the like, or a non-phosphine type ligand such as imidazol-2-ylidene carbene and the like.

Amount of the palladium catalyst used for the reaction is preferably 0.01 to 20 mol %, and more preferably 0.1 to 10 mol % compared to reacting materials.

Reaction temperature for the reaction may vary depending on starting compounds, a catalyst, a solvent, and the like. In general, the reaction is preferably carried out in the temperature range of 0° C. to 150° C. More preferably, it is in the temperature range of room temperature to 120° C. Reaction time may vary depending on starting compounds, a catalyst, a solvent, reaction temperature and the like. In general, it is in the range of 30 minutes to 72 hours, preferably in the range of 1 to 48 hours.

With respect to the reaction process for the preparing method D, the compounds represented by the Formula (D-1) can be obtained as a commercially available product as described in Table 4, for example.

TABLE 4

| No. | structure | suppl. |
|---|---|---|
| D-1-A | 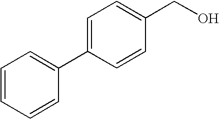 | TCI |
| D-1-B | 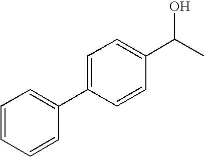 | Wako |
| D-1-C | 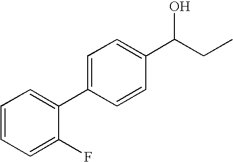 | Acr |
| D-1-D | 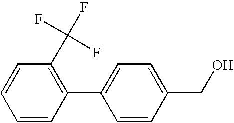 | Tyg |
| D-1-E | 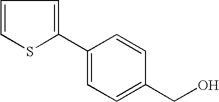 | Acr |

The compounds represented by the Formula (D-1) can be prepared according to a reverse synthetic pathway of the following reaction route, for example (i.e., reaction process for preparing method E; herein below, it is sometimes described as “route E”).

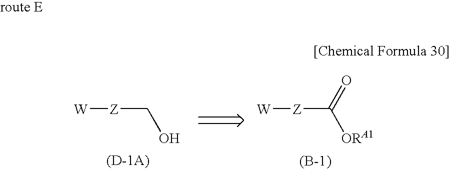

(Regarding a reaction process for preparing method E, the compound represented by Formula (D-1A) corresponds to a compound represented by the Formula (D-1) in which n is 1 and both R^{V1} and R^{V2} are a hydrogen atom. W, Z and R^{A1} are as defined above. In addition, one or more of these groups can be protected).

As for a reducing agent which can be used for the reaction, lithium aluminum hydride, sodium borohydride, a borane complex and the like can be mentioned. A metal hydride complex is preferred, and preferred examples thereof include lithium aluminum hydride and the like. Preferred examples of a borane complex include a borane-dimethylsulfide complex and the like.

Type of a solvent used for the reduction of the compounds represented by the Formula (B-1) is not specifically limited if it is inert to the reduction. Examples thereof include a saturated hydrocarbon solvent, a halogenated hydrocarbon solvent, an ether solvent, an aromatic hydrocarbon solvent and the like. These can be used alone or as a mixture comprising them in any mixing ratio. Examples of saturated hydrocarbon solvent include pentane, hexane, heptane and cyclohexane, and examples of halogenated hydrocarbon solvent include dichloromethane, chloroform, 1,2-dichloroethane and the like. Examples of ether solvent include tetrahydrofuran, diethyl ether, and 1,4-dioxane, and examples of aromatic hydrocarbon solvent include toluene, xylene and the like. Preferred examples include diethyl ether, tetrahydrofuran, toluene and a mixture solvent comprising them in any mixing ratio, and the like.

The reducing agent is used in a molar amount of at least 0.1 times, preferably at least 1 times the molar amount of the compounds represented by the Formula (B-1). In addition, it is used in a molar amount of no more than 100 times, preferably no more than 10 times the molar amount of the compounds represented by the Formula (B-1).

Reaction temperature may vary depending on starting compounds, a reducing agent, a solvent, and the like. In general, the reaction is preferably carried out at the temperature of −100° C. or more. In addition, the reaction is preferably carried out at the temperature of 100° C. or less.

Reaction time may vary depending on starting compounds, a reducing agent, a solvent, reaction temperature and the like. In general, it is in the range of 5 minutes to 12 hours.

The compounds represented by the Formula (D-1) can be prepared according to a reverse synthetic pathway of the following reaction route, for example (i.e., reaction process for Synthetic method F; herein below, it is sometimes described as “route F”).

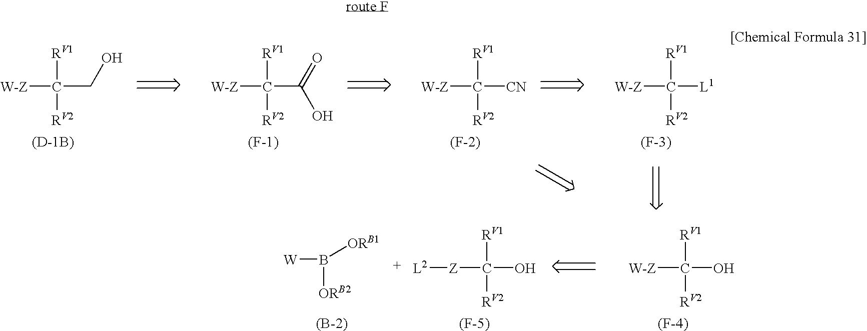

(Regarding a reaction process for preparing method F, the compound represented by Formula (D-1B) corresponds to a compound represented by the Formula (D-1) in which n is 1, k is 1 and both R^{V3} and R^{V4} are a hydrogen atom. W, Z, R^{V1}, R^{V2}, L¹, L², R^{B1} and R^{B2} are as defined above. In addition, one or more of these groups can be protected).

With respect to a method of producing the compounds represented by the Formula (D-1B) from the compounds represented by the Formula (F-1), the method which is the same as the method of producing the compounds represented by the Formula (D-1A) from the compounds represented by the Formula (B-1) can be exemplified.

The compounds represented by the Formula (F-1) can be prepared by hydrolysis of the compounds represented by the Formula (F-2).

Examples of a base used for the hydrolysis reaction include a metal hydroxide such as lithium hydroxide, sodium hydroxide, potassium hydroxide and the like. Further, examples of a solvent used for the hydrolysis reaction include water, water-comprising organic solvent system and an organic solvent system.

Type of an organic solvent used for the solvent system is not specifically limited if it is inert to the hydrolysis reaction. Examples thereof include an alcohol solvent such as methanol, ethanol, 2-propanol and the like, an ether solvent such as tetrahydrofuran, 1,4-dioxane and the like, and a mixture solvent comprising them in any mixing ratio. The base is preferably used for the reaction in a molar amount of at least times, more preferably at least 1 times the molar amount of the compounds represented by the Formula (N2-34). In addition, it is preferably used in a molar amount of no more than 50 times, more preferably no more than 10 times the molar amount of the compounds represented by the Formula (N2-34). Reaction temperature may vary depending on starting compounds, a base, a solvent, and the like. For example, it can be in the range of 0° C. to reflux temperature of a solvent.

The compounds represented by the Formula (F-2) can be prepared by cyanation of the compounds represented by the Formula (F-3).

Examples of a source for cyanide used for the reaction include potassium cyanide, sodium cyanide, benzylcyanide trimethyl ammonium and the like.

An organic solvent that can be used for the reaction is not specifically limited if it is inert to the reaction. Examples thereof include an amide solvent such as N,N-dimethyl formamide and the like, a halogenated hydrocarbon solvent such as dichloromethane and the like, an aromatic hydrocarbon solvent such as benzene, toluene, xylene and the like, acetonitrile, dimethyl sulfoxide, and a mixture solvent comprising them in any mixing ratio.

If necessary, sodium iodide, crown ether and the like can be also present for the reaction.

The source material for cyanide is used in a molar amount of at least 0.9 times but no more than 10 times, preferably at least 0.5 times but no more than 3 times the molar amount of the reacting materials.

Reaction temperature for the reaction may vary depending on starting compounds, a catalyst, a solvent, and the like. In general, the reaction is preferably carried out in the temperature range of 0° C. to 150° C. More preferably, it is in the temperature range of room temperature to 120° C. Reaction time may vary depending on starting compounds, a catalyst, a solvent, reaction temperature and the like. In general, it is in the range of 30 minutes to 72 hours, preferably in the range of 1 to 48 hours.

With respect to a method of producing the compounds represented by the Formula (F-3) from the compounds represented by the Formula (F-4), the method which is the same as the method of producing the compounds represented by the Formula (A-1) from the compounds represented by the Formula (A-3) can be exemplified.

As an alternative method, the compounds represented by the Formula (F-2) can be prepared from the compounds represented by the Formula (F-4) by cyanation. The reagents used for the reaction include sodium cyanide and the like as a source for cyanide, a phosphine compound such as triphenylphosphine and the like, carbon tetrachloride and the like. The organic solvent is not specifically limited if it is inert to the reaction. Examples thereof include dimethylsulfoxide and the like. Reaction temperature for the reaction may vary depending on starting compounds, a catalyst, a solvent, and the like. In general, the reaction is preferably carried out in the temperature range of room temperature to 200° C. Reaction time may vary depending on starting compounds, a catalyst, a solvent, reaction temperature and the like. In general, it is in the range of 30 minutes to 72 hours, preferably in the range of 1 to 48 hours.

With respect to a method of producing the compounds represented by the Formula (F-4) from the compounds represented by the Formula (B-2) and the compounds represented by the Formula (F-5), the method which is the same as the method of producing the compounds represented by the Formula (B-1) from the compounds represented by the Formula (B-2) and the compounds represented by the Formula (B-3) can be exemplified.

With respect to the reaction process for the preparing method F, the compounds represented by the Formula (F-4) can be obtained as a commercially available product as described in Table 4, for example.

With respect to the reaction process for the preparing method F, the compounds represented by the Formula (F-5) can be obtained as a commercially available product as described in Table 5, for example.

TABLE 5

| No. | structure | suppl. |
|---|---|---|
| F-5-A | 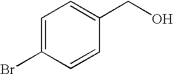 | Ald |
| F-5-B | 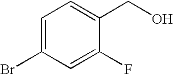 | Lan |
| F-5-C | 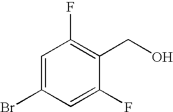 | Ald |
| F-5-D | 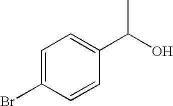 | TCI |
| F-5-E | 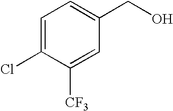 | Wako |
| F-5-F | 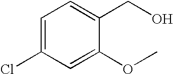 | Ald |

The compounds represented by the Formula (1) can be prepared according to a reverse synthetic pathway of the following reaction route, for example (i.e., reaction process for preparing method G; herein below, it is sometimes described as “route G”).

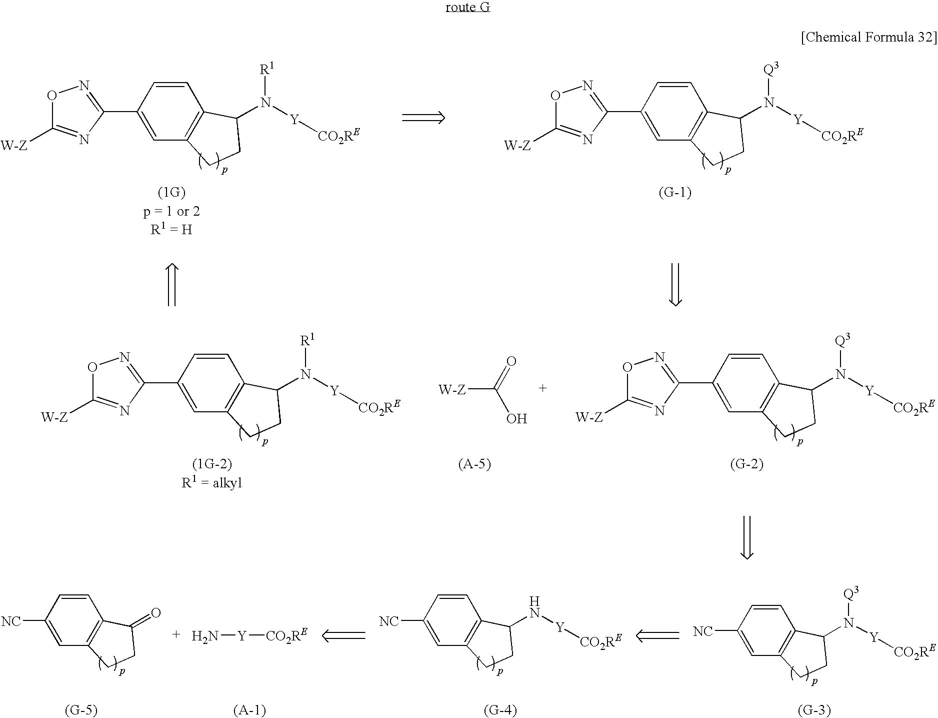

(Regarding a reaction process for preparing method G, the compounds represented by Formula (1G) or Formula (1G-2) correspond to a compound represented by the Formula (1) in which -Z-V— is represented by the Formula (2) and R2 is linked to X² via a C2 or C3 alkylene to form a 5- or 6-membered ring. W, Z, R1, Y and R^{E} are as defined above. Q3 is a protecting group for protecting an amino group. Preferred examples of Q³ include a carbamate protecting group, and Boc (tert-butyloxycarbonyl) or Cbz (benzyloxycarbonyl) can be also mentioned, but not limited thereto. In addition, one or more of these groups can be protected).

The compounds represented by the Formula (1G-2) can be prepared based on an alkylation reaction between the compounds represented by the Formula (1G) and a known alkylating agent R¹L¹ (wherein, R¹ represents a C1-4 alkyl group which may be substituted with one to five halogen atoms, and L¹ is as defined above.)

With respect to the reaction, the method which is the same as the method of producing the compounds represented by the Formula (1A) from the compounds represented by the Formula (A-1) and the compounds represented by the Formula (A-2) can be exemplified.

Compounds represented by the Formula (1G) can be prepared by deprotection reaction of the compounds represented by the Formula (G-1).

Deprotection can be carried out according to any known method, for example, according to a method described in Protective Groups in Organic Synthesis, John Wiley and Sons (1999).

The compounds represented by the Formula (G-1) can be prepared by condensation reaction of the compounds represented by the Formula (G-2) and the compounds represented by the Formula (A-5) in the presence of a dehydration condensation agent.

With respect to the reaction, the method which is the same as the method of producing the compounds represented by the Formula (A-3) from the compounds represented by the Formula (A-4) and the compounds represented by the Formula (A-5) can be exemplified.

With respect to a method of producing the compounds represented by the Formula (G-2) from the compounds represented by the Formula (G-3), the method which is the same as the method of producing the compounds represented by the Formula (A-6) from the compounds represented by the Formula (A-7) can be exemplified.

Compounds represented by the Formula (G-3) can be prepared by protection reaction of the compounds represented by the Formula (G-4).

Protection can be carried out according to any known method, for example, according to a method described in Protective Groups in Organic Synthesis, John Wiley and Sons (1999).

The compounds represented by the Formula (G-4) can be prepared by reductive amination of the compounds represented by the Formula (G-5) and the compounds represented by the Formula (A-2).

Preparation of the Compounds Represented by the Formula (G-4) based on the reductive amination can be carried out according to a known reductive amination methods described in literature (for example, New Experimental Chemistry Series, 4^{th} ed., Vol. 20, Chapter 6, Maruzen, and Robert, M. B. et. al., Tetrahedron Letters, 39, 3451 (1998)).

As for a reducing agent which can be used for the reductive amination, examples include, hydrogen, lithium aluminumhydride, sodium borohydride, sodium cyanoborohydride, borohydride triacetate, borane, formic acid-triethylamine complex and the like, but not limited thereto. Preferred examples are hydrogen, sodium borohydride, sodium cyanoborohydride, borohydride triacetate, borane, or formic acid-triethylamine complex.

Type of a solvent used for the reaction is not specifically limited if it is inert to the reduction. Examples thereof include an alcohol solvent, a saturated hydrocarbon solvent, a halogenated hydrocarbon solvent, an ether solvent, an aromatic hydrocarbon solvent, N,N-dimethyl formamide, dimethylsulfoxide and the like. These can be used alone or as a mixture solvent comprising them in any mixing ratio. Examples of an alcohol solvent include methanol, ethanol, 2-propanol and the like. Examples of saturated hydrocarbon solvent include pentane, hexane, heptane and cyclohexane, and examples of halogenated hydrocarbon solvent include dichloromethane, chloroform, 1,2-dichloroethane and the like. Examples of ether solvent include tetrahydrofuran, diethylether, 1,4-dioxane and the like, and examples of aromatic hydrocarbon solvent include toluene, xylene and the like. Preferred examples include 2-propanol, dichloromethane, tetrahydrofuran, toluene, N,N-dimethyl formamide and the like.

The reducing agent is preferably used in a molar amount of at least 0.1 times, more preferably at least 1 times the molar amount of the compounds represented by the Formula (G-5). In addition, it is preferably used in a molar amount of no more than 100 times, more preferably no more than 10 times the molar amount of the compounds represented by the Formula (G-5). Reaction temperature is not specifically limited. However, the reaction is preferably carried out at the temperature of −20° C. or more. More preferably, it is carried out at the temperature of 0° C. or more.

Reaction time may vary depending on starting compounds, a solvent, reaction temperature and the like. In general, it is in the range of 30 minutes to 72 hours, preferably in the range of 1 to 48 hours.

The compounds represented by the Formula (G-5) for the reaction process for the preparing method G can be prepared, for example, according to the method described in Reference example 7 or Reference example 12.

The compounds represented by the Formula (1) can be prepared according to a reverse synthetic pathway of the following reaction route, for example (i.e., reaction process for preparing method H; herein below, it is sometimes described as “route H”).

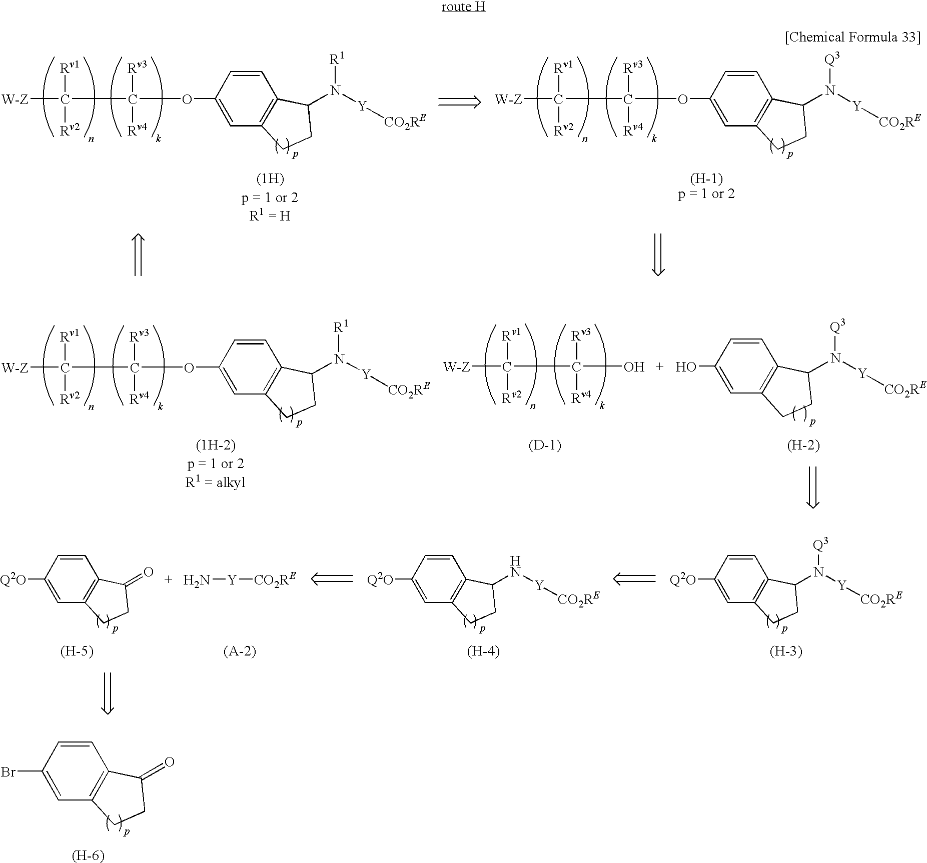

(Regarding a reaction process for preparing method H, the compounds represented by Formula (1H) or Formula (1H-2) correspond to a compound represented by the Formula (1) in which -Z-V— is -Z-(CR^{V1}R^{V2})ₙ—(CR^{V3}R^{V4})ₖ—O— and R² is linked to X² via a or C3 alkylene to form a 5- or 6-membered ring. W, Z, R¹, R^{V1}, R^{V2}, R^{V3}, R^{V4}, n, k, Y, R^{E}, Q², and Q³ are as defined above. In addition, one or more of these groups can be protected).

The compounds represented by the Formula (1H-2) can be prepared based on an alkylation reaction between the compounds represented by the Formula (1H) and a known alkylating agent R¹L¹ (wherein, R¹ represents a C1-4 alkyl group which may be substituted with one to five halogen atoms, and L¹ is as defined above)

With respect to the reaction, the method which is the same as the method of producing the compounds represented by the Formula (1A) from the compounds represented by the Formula (A-1) and the compounds represented by the Formula (A-2) can be exemplified.

With respect to a method of producing the compounds represented by the Formula (1H) from the compounds represented by the Formula (H-1), the method which is the same as the method of producing the compounds represented by the Formula (1G) from the compounds represented by the Formula (G-1) can be exemplified.

With respect to a method of producing the compounds represented by the Formula (H-1) from the compounds represented by the Formula (H-2) and the compounds represented by the Formula (D-1), the method which is the same as the method of producing the compounds represented by the Formula (1D) from the compounds represented by the Formula (D-1) and the compounds represented by the Formula (D-2) can be exemplified.

With respect to a method of producing the compounds represented by the Formula (H-2) from the compounds represented by the Formula (H-3), the method which is the same as the method of producing the compounds represented by the Formula (D-2) from the compounds represented by the Formula (D-3) can be exemplified.

With respect to a method of producing the compounds represented by the Formula (H-3) from the compounds represented by the Formula (H-4), the method which is the same as the method of producing the compounds represented by the Formula (G-3) from the compounds represented by the Formula (G-4) can be exemplified.

With respect to a method of producing the compounds represented by the Formula (H-4) from the compounds represented by the Formula (H-5) and the compounds represented by the Formula (A-2), the method which is the same as the method of producing the compounds represented by the Formula (G-4) from the compounds represented by the Formula (G-5) and the compounds represented by the Formula (A-2) can be exemplified.

With respect to a method of producing the compounds represented by the Formula (H-5) from the compounds represented by the Formula (H-6), the method which is the same as the method of producing the compounds represented by the Formula (D-5) from the compounds represented by the Formula (A-8) can be exemplified.

In addition, for obtaining the compounds represented by the Formula (H-5), 5-hydroxy-1-indanone (manufactured by Tokyo Chemical Industry Co., Ltd.) or 6-hydroxy-1-tetralone (manufactured by Aldrich Company) as a commercially available product can be used after appropriate protection, for example.

Protection can be carried out according to any known method, for example, according to a method described in Protective Groups in Organic Synthesis, John Wiley and Sons (1999).

The compounds represented by the Formula (H-6) can be obtained as a commercially available product, for example, 5-bromo-1-indanone (Tokyo Chemical Industry Co., Ltd.) and 6-bromo-1-tetralone (manufactured by J&W Pharmlab).

The method of producing the compounds of the present invention is not limited those described herein. For example, the compounds of the present invention can be produced by modifying and converting the substituents of a precursor compound via a single reaction or multiple reactions in combination disclosed in general chemical literatures, etc.

With respect to the compounds of the present invention, exemplary method for producing a compound comprising an asymmetric carbon atom includes, in addition to the above described method based on asymmetric reduction, a method which uses a commercially available starting compound in which a portion corresponding to the asymmetric carbon is already optically active (or, which can be produced according to a known method or in view of a known method) can be mentioned. In addition, there is a method by which the compounds of the present invention or a precursor thereof are resolved into optically active isomers by following a generally known method. Such method includes a high pressure liquid chromatography (HPLC) method using an optically active column, a traditional optical fractional crystallization in which a salt is formed with an optically active reagent, resolved by fractional crystallization, etc., and then degraded to give a free form, or a method in which a diastereomer is first formed by condensation with an optically active reagent, isolated and purified, and then degraded again. When a precursor is separated to give an optically active form, the preparation method as described above can be then carried out to produce the optically active compounds of the present invention.

When the compounds of the present invention comprise an acidic functional group such as a carboxy group, a phenolic hydroxy group, or a tetrazole ring and the like, it is possible to prepare them in a pharmaceutically acceptable salt form according to a known method (for example, an inorganic salt with sodium, ammonia and the like, or an organic salt with triethylamine). For example, for obtaining an inorganic salt, the compounds of the present invention are preferably dissolved in water containing at least one equivalent of hydroxides, carbonates, hydrocarbonates and the like which correspond to the desired inorganic salt. For the reaction, an organic solvent which is inert and can be mixed with water, such as methanol, ethanol, acetone, dioxane and the like, can be also incorporated. For example, when sodium hydroxide, sodium carbonate, or sodium hydrocarbonate is used, a solution of sodium salt can be obtained.

When the compounds of the present invention comprise an amino group or other basic functional group, or an aromatic ring having a basic property by itself (for example, a pyridine ring, etc.), it is possible to prepare them in a pharmaceutically acceptable salt form according to a known method (for example, an inorganic salt with hydrochloric acid, sulfuric acid and the like, or an organic salt with acetic acid, citric acid and the like). For example, for obtaining an inorganic salt, the compounds of the present invention are dissolved in water containing at least one equivalent of desired inorganic acid. For the reaction, an organic solvent which is inert and can be mixed with water, such as methanol, ethanol, acetone, dioxane and the like, can be also incorporated. For example, when hydrochloric acid is used, a solution of hydrochloric acid can be obtained.

Prodrugs of the compounds of the present invention are not specifically limited and examples thereof include a compound in which a group, which can form a prodrug, is introduced to at least one group selected from the hydroxyl group, the amino group and the carboxy group contained in the compounds of the present invention. As for a group which can form a prodrug with a hydroxy group or an amino group, an acyl group and an alkoxycarbonyl group can be exemplified. Preferred examples thereof include an acetyl group, a propionyl group, a methoxycarbonyl group, an ethoxycarbonyl group and the like. An ethoxycarbonyl group is more preferred. In addition, there is other embodiment in which an acetyl group is more preferred. In addition, there is other embodiment in which a propionyl group is more preferred. In addition, there is also other embodiment in which a methoxycarbonyl group is more preferred. As for a group which can form a prodrug with a carboxyl group, a methyl group, an ethyl group, n-propyl group, an isopropyl group, n-butyl group, an isobutyl group, s-butyl group, t-butyl group, amino group, methylamino group, ethylamino group, dimethylamino group, or diethylamino group and the like, can be exemplified. Preferred examples thereof include an ethyl group, n-propyl group, an isopropyl group and the like. An ethyl group is more preferred. In addition, there is other embodiment in which a n-propyl group is more preferred. In addition, there is also other embodiment in which an isopropyl group is more preferred.

With the S1P1 agonistic activity, the compounds of the present invention can be employed as an immunoregulatory agent which is useful for the prophylaxis or the treatment of an autoimmune disease or a chronic inflammatory disease. The compounds of the present invention are useful for inhibiting an immune system, for example, for a case in which an immunosuppressive activity remains normal, such as bone marrow or organ transplant or graft rejection, or for an autoimmune and chronic inflammatory diseases, including systemic lupus erythematosus, chronic rheumatoid arthritis, type I diabetes mellitus, inflammatory bowel disease, biliary cirrhosis, uveitis, multiple sclerosis and other disorders such as Crohn's disease, ulcerative colitis, bullous pemphigoid, sarcoidosis, psoriasis, autoimmune myositis, Wegener's granulomatosis, ichthyosis, Graves' opthalmopathy, atopic dermatitis and asthma.

With respect to the S1P1 which can be used for determination of S1P1 activity, examples include a publicly known human S1P1 (Accession No. NP_{—}001391) or the S1P1 variant which has the amino acid sequence of the human S1P1 with deletion, substitution or addition of one or more amino acids thereof and has S1P1 activity. According to one embodiment, S1P1 (Accession No. NP 001391) is more preferred.

More specifically, the compounds of the present invention are useful for the prophylaxis or the treatment of a condition or a disease selected from the group consisting of: transplantation of organs or tissue, graft-versus-host diseases caused by transplantation, autoimmune syndromes including rheumatoid arthritis, systemic lupus erythematosus, Hashimoto's thyroiditis, multiple sclerosis, myasthenia gravis, type I diabetes, uveitis, posterior uveitis, allergic encephalomyelitis, glomerulonephritis, post-infectious autoimmune diseases including rheumatic fever and post-infectious glomerulonephritis, inflammatory and hyperproliferative skin diseases, psoriasis, atopic dermatitis, contact dermatitis, eczematous dermatitis, seborrhoeic dermatitis, lichen planus, pemphigus, bullous pemphigoid, epidermolysis bullosa, urticaria, angioedemas, vasculitis, erythema, cutaneous eosinophilia, lupus erythematosus, acne, alopecia greata, keratoconjunctivitis, vernal conjunctivitis, uveitis associated with Behcet's disease, keratitis, herpetic keratitis, conical cornea, dystrophia epithelialis cornea, corneal leukoma, ocular pemphigus, Mooren's ulcer, scleritis, Graves' opthalmopathy, Vogt-Koyanagi-Harada syndrome, sarcoidosis, pollen allergies, reversible obstructive airway disease, bronchial asthma, allergic asthma, intrinsic asthma, extrinsic asthma, dust asthma, chronic or inveterate asthma, late asthma and airway hyper-responsiveness, bronchitis, gastric ulcers, vascular damage caused by ischemic diseases and thrombosis, ischemic bowel diseases, inflammatory bowel diseases, necrotizing enterocolitis, intestinal lesions associated with thermal burns, coeliac diseases, proctitis, eosinophilic gastroenteritis, mastocytosis, Crohn's disease, ulcerative colitis, migraine, rhinitis, eczema, interstitial nephritis, Goodpasture's syndrome, hemolytic-uremic syndrome, diabetic nephropathy, multiple myositis, Guillain-Barre syndrome, Meniere's disease, polyneuritis, multiple neuritis, mononeuritis, radiculopathy, hyperthyroidism, Basedow's disease, pure red cell aplasia, aplastic anemia, hypoplastic anemia, idiopathic thrombocytopenic purpura, autoimmune hemolytic anemia, agranulocytosis, pernicious anemia, megaloblastic anemia, anerythroplasia, osteoporosis, sarcoidosis, fibroid lung, idiopathic interstitial pneumonia, dermatomyositis, leukoderma vulgaris, ichthyosis vulgaris, photoallergic sensitivity, cutaneous T cell lymphoma, arteriosclerosis, atherosclerosis, aortitis syndrome, polyarteritis nodosa, myocardosis, scleroderma, Wegener's granuloma, Sjogren's syndrome, adiposis, eosinophilic fascitis, lesions of gingiva, periodontium, alveolar bone, substantia ossea dentis, glomerulonephritis, male pattern alopecia or alopecia senilis by preventing epilation or providing hair germination and/or promoting hair generation and hair growth, muscular dystrophy, pyoderma and Sezary's syndrome, Addison's disease, ischemia-reperfusion injury of organs which occurs upon preservation, transplantation or ischemic disease, endotoxin-shock, pseudomembranous colitis, colitis caused by drug or radiation, ischemic acute renal insufficiency, chronic renal insufficiency, toxinosis caused by lung-oxygen or drugs, lung cancer, pulmonary emphysema, cataracta, siderosis, retinitis pigmentosa, senile macular degeneration, vitreal scanning, corneal alkali burn, dermatitis erythema multiforme, linear IgA ballous dermatitis and cement dermatitis, gingivitis, periodontitis, sepsis, pancreatitis, diseases caused by environmental pollution, aging, carcinogenesis, metastasis of carcinoma and hypobaropathy, disease caused by histamine or leukotriene-C4 release, Behcet's disease, autoimmune hepatitis, primary biliary cirrhosis, sclerosing cholangitis, partial liver resection, acute liver necrosis, necrosis caused by toxin, viral hepatitis, shock, or anoxia, B-virus hepatitis, non-A/non-B hepatitis, cirrhosis, alcoholic cirrhosis, hepatic failure, fulminant hepatic failure, late-onset hepatic failure, “acute-on-chronic” liver failure, augmentation of chemotherapeutic effect, cytomegalovirus infection, HCMV infection, AIDS, cancer, senile dementia, trauma, and chronic bacterial infection.

The invention also encompasses a method for the prophylaxis or the treatment of transplantation rejection or resistance to transplanted organ or tissue in a mammalian patient in need of such treatment comprising administering to said patient the compounds of the present invention in a therapeutically effective amount.

Another embodiment of the invention encompasses a method of suppressing the immune system in a mammalian patient in need of immunosuppression comprising administering to said patient an immunosuppressively effective amount of the compounds of the present invention.

Most particularly, the method described herein encompasses a method of treating or preventing bone marrow or organ transplant rejection which is comprised of administering to a mammalian patient in need of such treatment or prevention the compounds of the present invention, or a pharmaceutically acceptable salt or hydrate thereof, in an amount that is effective for treating or preventing bone marrow or organ transplant rejection.

The compounds of the present invention are also useful for treating a respiratory disease or condition such as asthma, chronic bronchitis, chronic obstructive pulmonary disease, adult respiratory distress syndrome, infant respiratory distress syndrome, cough, eosinophilic granuloma, respiratory syncytial virus bronchiolitis, bronchiectasis, idiopathic pulmonary fibrosis, acute lung injury, and bronchiolitis obliterans organizing, pneumonia, and the like.

The compounds of the present invention, including salts and hydrates thereof, are useful in the treatment of autoimmune diseases, including the prevention of rejection of bone marrow transplant, foreign organ transplants and/or related afflictions, diseases and illnesses.

Furthermore, the compounds of the present invention are selective agonists of the S1P1 receptor having selectivity over S1P3 receptor. An S1P1 receptor selective agonist has advantages over current therapies and extends the therapeutic window of lymphocytes sequestration agents, allowing better tolerability with higher dosing and thus improving efficacy as monotherapy.

With respect to the S1P3 which can be used for determination of S1P3 activity, examples include a publicly known human S1P3 (Accession No. NP_{—}005217) or the S1P3 variant which has the amino acid sequence of the human S1P3 with deletion, substitution or addition of one or more amino acids thereof and has S1P3 activity. According to one embodiment, S1P3 (Accession No. NP_{—}005217) is more preferred.

Further, with respect to the S1P2 which can be used for determination of S1P2 activity as described in the following test example, examples include a publicly known human S1P2 (Accession No. NP_{—}004221) or the S1P2 variant which has the amino acid sequence of the human S1P2 with deletion, substitution or addition of one or more amino acids thereof and has S1P2 activity. According to one embodiment, S1P2 (Accession No. NP_{—}004221) is more preferred.

Further, with respect to the S1P4 which can be used for determination of S1P4 activity as described in the following test example, examples include a publicly known human S1P4 (Accession No. NP_{—}003766) or the S1P4 variant which has the amino acid sequence of the human S1P4 with deletion, substitution or addition of one or more amino acids thereof and has S1P4 activity. According to one embodiment, S1P4 (Accession No. NP 003766) is more preferred.

Further, with respect to the S1P5 which can be used for determination of S1P5 activity as described in the following test example, examples include a publicly known human S1P5 (Accession No. NP-110387) or the S1P5 variant which has the amino acid sequence of the human S1P5 with deletion, substitution or addition of one or more amino acids thereof and has S1P5 activity. According to one embodiment, S1P5 (Accession No. NP 110387) is more preferred.

By determining selectivity over S1P1 receptor and S1P3 receptor of the compounds of the present invention, salts or prodrugs thereof, any disparity between efficacy and undesirable bradycardia (i.e., reduced heart beat) can be shown.

Furthermore, as being useful for improving bradycardia, the compounds of the present invention have advantages over current therapies and extend the therapeutic window of lymphocytes sequestration agents, allowing better tolerability with higher dosing and thus improving efficacy as monotherapy.

Furthermore, a pharmaceutical agent which comprises the compounds of the present invention as an effective component can be used together with one or more other preventive or therapeutic agents or used in combination with them to treat the above described conditions or diseases of a mammal, preferably, human, pet including a dog, a cat, etc. or a companion animal, or livestock. Examples of a pharmaceutical agent which can be used together or in combination include the followings: immunosuppressive agents such as azathioprine, brequinar sodium, deoxyspergualin, mizaribine, mycophenolic acid morpholino ester, tacrolimus, cyclosporin, rapamycin and FTY720, or a preparation comprising the same; immunomodification anti-rheumatoid agents which are used as a therapeutic agent for treating chronic rheumatoid arthritis or metabolism antagonist, specifically, a gold agent, bucillamine, lobenzarit, salazosulfapyridine, methotrexate, azathiopurine, mizoribine, leflunomide, tacrolimus, cyclosporin, or a preparation comprising the same; an anti-cytokine antibody preparation against cytokines such as interluecin (IL)-1, IL-6 or tumor necrosis factor (TNF)-α and the like, or a soluble receptor preparation for such cytokines, specifically, infliximab, etanercept, and the like or a preparation comprising the same; a steroid preparation, specifically, dexamethasone, betamethasone, prednisolone, fluticasone, beclomethasone, and the like or a preparation comprising the same; bronchial dilating agents used for chronic bronchial asthma, specifically, salmeterol and salbutamol as an adrenaline β2 stimulating agent, ipratropium as an anti-cholinergic agent and the like, or a preparation comprising the same; a therapeutic agent for an allergic disorder, for example theophylline as a xanthine derivative, fexofenadine, epinastine, certirizine, ketotifen, sodium cromoglicatem, pemirolast and the like as an anti-allergic agent, or fexofenadine and certirizine as an anti-histamine agent, or a preparation comprising the same; irinotecn, 5-fluoro uracil and the like as an anti-tumor agent, or a preparation comprising the same. In addition, a pharmaceutical agent comprising the compounds of the present invention as an effective component can be used together with or in combination of radiotherapy.

Further, the compounds of the present invention, salts, or derivatives thereof useful as a prodrug have excellent safety (i.e., having favorable pharmacology regarding various toxicity and also safety) and pharmacokinetics of a drug, etc., and usefulness as an effective component for a pharmaceutical agent is confirmed.

Examples of safety test include the followings, but are not limited thereto. Cell toxicity test (test using HL60 cell or liver cell, etc.), Genetic Toxicity Test (Ames test, mouse lymphoma TK test, chromosome abnormality test, small nuclear test, etc.), skin sensitization test (Buehler method, GPMT method, APT method, LLNA test, etc.), skin photosensitization test (Adjuvant and Strip method, etc.), ocular irritation test (single application, continuous application for a short period of time, repeated application, etc.), safety pharmacology test regarding cardiovascular system (telemetry method, APD method, hERG inhibition evaluation test), safety pharmacology test regarding central nervous system (FOB method, modified Irwin method, etc.), safety pharmacology test regarding respiratory system (measurement using an instrument for measuring respiratory function, measurement using an instrument for determining blood gas analysis, etc.), general toxicity test, sexual reproduction toxicity test, etc. In addition, regarding a test for pharmacokinetics of a drug, the followings are included, but not limited thereto. Inhibition or induction test regarding cytochrome P450 enzyme, cell permeation test (i.e., a test using CaCO-2 cells or MDCK cells, etc), drug-transporter ATPase assay, oral absorption test, blood concentration time profile test, metabolism test (stability test, metabolic molecular species test, reactivity test, etc.), solubility test (i.e., solubility test based on turbidity, etc.) and the like.

Usefulness of the compounds of the present invention represented by the above Formula (1), salts or derivatives thereof useful as a prodrug as an effective component for a pharmaceutical agent can be determined based on a cell toxicity test, for example. Regarding a cell toxicity test, a method using various cultured cells like human pre-leukemia HL-60 cells, primarily-separated cultured liver cells, neutrophil fraction prepared from human peripheral blood, etc. can be mentioned. Test can be carried out according to the method described below, but it is not limited thereto. Cells are prepared in suspension comprising 10⁵ to 10⁷ cells/ml. 0.01 mL to 1 mL suspension is aliquoted to a micro tube or a micro plate, etc. Then, a solution comprising the compounds dissolved therein is added thereto in an amount of 1/100 to 1 times the cell suspension, followed by culturing in a cell culture medium having final concentration of the compounds at 0.001 μM to 1000 μM under the condition of 37° C., 5% CO₂ for 30 minutes to several days. Once the cell culture is completed, cell viability ratio is determined using MTT method or WST-1 method (Ishiyama, M., et. al., In Vitro Toxicology, 8, p. 187, 1995), etc. By measuring cell toxicity expressed by the compounds, their usefulness as an effective component of a pharmaceutical agent can be confirmed.

Usefulness of the compounds of the present invention represented by the above Formula (1), salts or derivatives thereof useful as a prodrug as an effective component for a pharmaceutical agent can be determined based on a Genetic Toxicity Test, for example. Examples of Genetic Toxicity Test include Ames test, mouse lymphoma TK test, chromosome abnormality test, small nuclear test, etc. The Ames test is a method for determining reversion mutation by culturing designated cells such as Salmonella or E. Coli on a culture dish comprising a compound (see, II-1. Genetic Toxicity Test under “Guidelines for Genetic Toxicity Test”, Pharmaceuticals Examination, Vol. 1604, 1999). Further, the mouse lymphoma TK test is a test for determining a mutational property of a gene in which thymidine kinase gene of mouse lymphoma cell L⁵¹⁷⁸Y cell is used as a target (see, II-3. Mouse Lymphoma TK Test under “Guidelines for Genetic Toxicity Test”, Pharmaceuticals Examination, Vol. 1604, 1999; Clive, D. et. al., Mutat. Res., 31, pp. 17-29, 1975; Cole, J., et. al., Mutat. Res., 111, pp. 371-386, 1983, etc.). Further, the chromosome abnormality test is a method in which mammalian cells are cultured in the presence of a compound and the cells are fixed, and the chromosome is stained and observed to determine any activity which may cause chromosomal abnormality (see, II-2. Chromosome Abnormality Test Using Cultured Mammalian Cells under “Guidelines for Genetic Toxicity Test”, Pharmaceuticals Examination, Vol. 1604, 1999). Further, the small nucleus test is a method of determining an ability to form a small nucleus which is caused by chromosomal abnormality, and it includes a method in which rodents are used (i.e., in vivo test, II-4. Small Nucleus Test Using Rodents, under “Guidelines for Genetic Toxicity Test”, Pharmaceuticals Examination, Vol. 1604, 1999; Hayashi, M. et. al., Mutat. Res., 312, pp. 293-304, 1994; Hayashi, M. et. al., Environ. Mol. Mutagen., 35, pp. 234-252, 2000) or cultured cells are used (i.e., in vitro test, Fenech, M. et. al., Mutat. Res., 147, pp. 29-36, 1985; Miller, B., et. al., Mutat. Res., 392, pp. 45-59, 1997), etc. By running one, two or more tests based on these methods, compounds' gene toxicity can be clearly identified so that their usefulness as an effective component of a pharmaceutical agent can be confirmed.

Usefulness of the compounds of the present invention represented by the above Formula (1), salts or derivatives thereof useful as a prodrug as an effective component for a pharmaceutical agent can be determined based on a skin sensitization test, for example. Examples of skin sensitization test include Buehler method (Buehler, E. V. Arch. Dermatol., 91, pp. 171-177, 1965), GPMT method (i.e., Maximization method, Magnusson, B. et. al., J. Invest. Dermatol., 52, pp. 268-276, 1969), APT method (i.e., Adjuvant and Patch method, Sato, Y. et. al., Contact Dermatitis, 7, pp. 225-237, 1981). Further, as a skin sensitization test, wherein a mouse is used, there is LLNA test (Local Lymph Node Assay method, OECD Guideline for the testing of chemicals 429, skin sensitization 2002; Takeyoshi, M. et. al., Toxicol. Lett., 119(3), pp. 203-8, 2001; Takeyoshi, M. et. al., J. Appl. Toxicol., 25(2), pp. 129-34, 2005) and the like. By running one, two or more tests based on these methods, compounds' skin sensitization property can be clearly identified so that their usefulness as an effective component of a pharmaceutical agent can be confirmed.

Usefulness of the compounds of the present invention represented by the above Formula (1), salts or derivatives thereof useful as a prodrug as an effective component for a pharmaceutical agent can be determined based on a skin photosensitization test, for example. Examples of skin photosensitization test include a test using a mormot (see, “Guidelines for Non-clinical test of pharmaceuticals—Explanation, 2002, YAKUJI NIPPO LIMITED 2002”, 1-9: Skin Photosensitization Test, etc.). Further, specific methods include adjuvant and strip method (Ichikawa, H. et. al., J. Invest. Dermatol., 76, pp. 498-501, 1981), Harber method (Harber, L. C., Arch. Dermatol., 96, pp. 646-653, 1967), Horio method (Horio, T., J. Invest. Dermatol., 67, pp. 591-593, 1976), Jordan method (Jordan, W. P., Contact Dermatitis, 8, pp. 109-116, 1982), Kochever method (Kochever, I. E. et. al., J. Invest. Dermatol., 73, pp. 144-146, 1979), Maurer method (Maurer, T. et. al., Br. J. Dermatol., 63, pp. 593-605, 1980), Morikawa method (Morikawa, F. et. al., “Sunlight and man”, Tokyo Univ. Press, Tokyo, pp. 529-557, 1974), Vinson method (Vinson, L. J., J. Soc. Cosm. Chem., 17, pp. 123-130, 1966) and the like. By running one, two or more tests based on these methods, compounds' skin photosensitization property can be clearly identified so that their usefulness as an effective component of a pharmaceutical agent can be confirmed.

Usefulness of the compounds of the present invention represented by the above Formula (1), salts or derivatives thereof useful as a prodrug as an effective component for a pharmaceutical agent can be determined based on an ocular irritation test, for example. Examples of ocular irritation test include a single application test (eye drop is applied only one time), a continuous application for a short period of time (eye drop is applied multiple times at regular intervals for a short period of time), a repeated application test (eye drop is applied intermittently for several days to several tens of days), etc. using a rabbit eye, a monkey eye, etc. In addition, there is a method by which eye irritation at certain time point after eye drop application is measured by Draize score, etc. (Fukui, N. et. al., Gendai no Rinsho, 4(7), pp. 277-289, 1970). By running one, two or more tests based on these methods, compounds' characteristics regarding eye irritation can be clearly identified so that their usefulness as an effective component of a pharmaceutical agent can be confirmed.

Usefulness of the compounds of the present invention represented by the above Formula (1), salts or derivatives thereof useful as a prodrug as an effective component for a pharmaceutical agent can be determined by carrying out a safety pharmacology test regarding cardiovascular system. Examples of safety pharmacology test regarding cardiovascular system include a telemetry method (i.e., a method by which compound's effect on an electrocardiogram, heart rate, blood pressure, blood flow amount, and the like is determined under non-anesthetized condition (Shigeru Kan-no, Hirokazu Tsubone, Yoshitaka Nakata eds., Electrocardiography, Echocardiography, Blood Pressure, and Pathology test of an Animal for Basic and Clinical Medicine, 2003, published by Maruzen)), APD method (i.e., a method for measuring action potential duration of a myocardial cell, (Muraki, K. et. al., AM. J. Physiol., 269, H524-532, 1995; Ducic, I. et. al., J. Cardiovasc. Pharmacol., 30(1), pp. 42-54, 1997)), measurement of hERG inhibition (patch clamp method (Chachin, M. et. al., Nippon Yakurigaku Zasshi, 119, pp. 345-351, 2002), Binding assay method (Gilbert, J. D. et. al., J. Pharm. Tox. Methods, 50, pp. 187-199, 2004), Rb⁺ efflux assay method (Cheng, C. S. et. al., Drug Develop. Indust. Pharm., 28, pp. 177-191, 2002), Membrane potential assay method (Dorn, A. et. al., J. Biomol. Screen., 10, pp. 339-347, 2005) etc.) etc. By running one, two or more tests based on these methods, compounds' effect on a cardiovascular system can be clearly identified so that their usefulness as an effective component of a pharmaceutical agent can be confirmed.

Usefulness of the compounds of the present invention represented by the above Formula (1), salts or derivatives thereof useful as a prodrug as an effective component for a pharmaceutical agent can be determined by carrying out a safety pharmacology test regarding a central nervous system. Examples of safety pharmacology test regarding a central nervous system include FOB method (i.e., a method for evaluating overall function, Mattson, J. L. et. al., J. American College of Technology 15 (3), pp. 239-254, 1996), modified Irwin method (i.e., a method for evaluating general symptoms and behavioral characteristics (Irwin, S. Comprehensive Observational Assessment (Berl.) 13, pp. 222-257, 1968)), etc. By running one, two or more tests based on these methods, compounds' effect on a central nervous system can be clearly identified so that their usefulness as an effective component of a pharmaceutical agent can be confirmed.

Usefulness of the compounds of the present invention represented by the above Formula (1), salts or derivatives thereof useful as a prodrug as an effective component for a pharmaceutical agent can be determined by carrying out a safety pharmacology test regarding a respiratory system, for example. Examples of safety pharmacology test regarding a respiratory system include a measurement using an instrument for measuring respiratory function (i.e., a method which measures breathing number, amount of air per single breathing, amount of breathing air per minute or hour, (Drorbaugh, J. E. et. al., Pediatrics, 16, pp. 81-87, 1955; Epstein, M. A. et. al., Respir. Physiol., 32, pp. 105-120, 1978), or a measurement using a blood gas analyzer (i.e., a method which measures blood gas, hemoglobin oxygen saturation, etc., Matsuo, S. Medicina, 40, pp. 188-, 2003), etc. By running one, two or more tests based on these methods, compounds' effect on a respiratory system can be clearly identified so that their usefulness as an effective component of a pharmaceutical agent can be confirmed.

Usefulness of the compounds of the present invention represented by the above Formula (1), salts or derivatives thereof useful as a prodrug as an effective component for a pharmaceutical agent can be determined by carrying out a general toxicity test. Specifically, according to a general toxicity test, a compound which is either dissolved or suspended in an appropriate solvent is orally administered or intravenously administered of a single time or multiple times to rodents such as rat, mouse, and the like or non-rodents such as monkey, dog and the like as a subject animal, and then animal's general state or any change in clinical chemistry or tissue in terms of pathology, etc. is determined. By identifying general toxicity of a compound based on this method, usefulness of a compound as an effective component for a pharmaceutical agent can be confirmed.

Usefulness of the compounds of the present invention represented by the above Formula (1), salts or derivatives thereof useful as a prodrug as an effective component for a pharmaceutical agent can be determined by carrying out a sexual reproduction toxicity test. The test is to determine any side effect caused by a compound on sexual reproduction process by using rodents such as rat, mouse, and the like or non-rodents such as monkey, dog and the like (“Guidelines for Non-clinical test of pharmaceuticals—Explanation, 2002”, YAKUJI NIPPO LIMITED 2002, 1-6: Sexual Reproduction Toxicity Test, etc.). With respect to a sexual reproduction toxicity test, a test relating to development of an early embryo from fertilization to implantation, a test relating to development before and after birth and an activity of a mother, a test relating to development of an embryo and a fetus (see, [3] Sexual Reproduction Toxicity Test under “Guidelines for Toxicity Test for Pharmaceuticals”, Pharmaceuticals Examination, Vol. 1834, 2000), etc. can be mentioned. By identifying sexual reproduction toxicity of a compound based on this method, usefulness of a compound as an effective component for a pharmaceutical agent can be confirmed.

Usefulness of the compounds of the present invention represented by the above Formula (1), salts or derivatives thereof useful as a prodrug as an effective component for a pharmaceutical agent can be determined by carrying out an inhibition or induction test of cytochrome P450 enzyme (Gomez-Lechon, M. J. et. al., Curr. Drug Metab. 5(5), pp. 443-462, 2004). Examples of the test include a method of determining in vitro an inhibitory effect of a compound on an enzyme activity by using cytochrome P450 enzyme of each molecular species that is either purified from a cell or prepared using a genetic recombinant, or a microsome as a human P450 expression system (Miller, V. P. et. al., Ann. N.Y. Acad. Sci., 919, pp. 26-32, 2000), a method of determining expression of cytochrome P450 enzyme for each molecular species or variation in enzyme activity by using a human liver microsome or cell homogenate (Hengstler, J. G. et. al., Drug Metab. Rev., 32, pp. 81-118, 2000), a method of examining compound's activity of inducing the enzyme by extracting the RNA from human liver cells that have been exposed to the compound and comparing the amount of mRNA expression with that of a control (Kato, M. et. al., Drug Metab. Pharmacokinet., 20(4), pp. 236-243, 2005), etc. By running one, two or more tests based on these methods, compounds' effect on induction or inhibition of cytochrome P450 enzyme can be clearly identified so that their usefulness as an effective component of a pharmaceutical agent can be confirmed.

Usefulness of the compounds of the present invention represented by the above Formula (1), salts or derivatives thereof useful as a prodrug as an effective component for a pharmaceutical agent can be determined by carrying out a cell permeation test, for example. Examples of the test include a method of determining compound's ability of penetrating cell membrane under in vitro cell culture system by using CaCO-2 cell, for example (Delie, F. et. al., Crit. Rev. Ther. Drug Carrier Syst., 14, pp. 221-286, 1997; Yamashita, S. et. al., Eur. J. Pham. Sci., 10, pp. 195-204, 2000; Ingels, F. M. et. al., J. Pham. Sci., 92, pp. 1545-1558, 2003), or a method of determining compound's ability of penetrating cell membrane under in vitro cell culture system by using MDCK cell (Irvine, J. D. et. al., J. Pham. Sci., 88, pp. 28-33, 1999) etc. By running one, two or more tests based on these methods, compounds' ability of penetrating cell membrane can be clearly identified so that their usefulness as an effective component of a pharmaceutical agent can be confirmed.

Usefulness of the compounds of the present invention represented by the above Formula (1), salts or derivatives thereof useful as a prodrug as an effective component for a pharmaceutical agent can be determined by carrying out a drug transporter ATPase assay using ATP-Binding Cassette (ABC) transporter, for example. Examples of the assay include a method of determining whether or not a compound is a substrate for P-gp by using P-glycoprotein (P-gp) baculovirus expression system (Germann, U. A., Methods Enzymol., 292, pp. 427-41, 1998), etc. Further, determination can be also carried out based on a transport assay using ooctyes obtained from Xenopus laevis, as a solute carrier (SLC) transporter. With respect to transport assay, oocytes which express OATP2 can be used to confirm whether or not the compound is a substrate for OATP2 (Tamai I. et. al., Pharm Res. 2001 September; 18(9): 1262-1269). By identifying compound's activity on ABC transporter or SLC transporter based on this method, usefulness of a compound as an effective component for a pharmaceutical agent can be confirmed.

Usefulness of the compounds of the present invention represented by the above Formula (1), salts or derivatives thereof useful as a prodrug as an effective component for a pharmaceutical agent can be determined by carrying out an oral absorptivity test, for example. Examples of the test include a method of determining blood transfer property of a compound after oral administration using LC-MS/MS method by preparing a certain amount of a compound dissolved or suspended in a solvent, orally administering it to a rodent, a monkey or a dog and measuring blood concentration of the compound over time (Harada Kenichi et. al., eds. “Newest aspects in mass spectrometry for biological sciences”, 2002, Kodansha Scientific, etc.). By identifying compound's oral absorptivity based on this method, usefulness of a compound as an effective component for a pharmaceutical agent can be confirmed.

Usefulness of the compounds of the present invention represented by the above Formula (1), salts or derivatives thereof useful as a prodrug as an effective component for a pharmaceutical agent can be determined by carrying out a blood concentration time profile test, for example. Examples of the test include a method of determining blood concentration profile of a compound using LC-MS/MS method by orally or parenterally (e.g., intravenous, intramuscular, intraperitoneal, subcutaneous, or trans-dermal administration, or administration into an eye or through nose, etc.) administering the compound to a rodent, a monkey or a dog and measuring blood concentration of the compound over time (Kenichi Harada et. al., eds. “Newest aspects in mass spectrometry for biological sciences”, 2002, Kodansha Scientific, etc.). By identifying compound's blood concentration time profile based on this method, usefulness of a compound as an effective component for a pharmaceutical agent can be confirmed.

Usefulness of the compounds of the present invention represented by the above Formula (1), salts or prodrugs thereof as an effective component for a pharmaceutical agent can be determined by carrying out a metabolism test, for example. Examples of the test include a method of determining stability in blood (i.e., a method by which in vivo metabolism clearance of a compound is calculated by measuring its metabolism rate in a liver microsome of a human or other animal; Shou, W. Z. et. al., J. Mass Spectrom., 40(10), pp. 1347-1356, 2005; L¹, C. et. al., Drug Metab. Dispos., 34(6), 901-905, 2006), a metabolite molecular species test, a reactive metabolite testing method, etc. By running one, two or more tests based on these methods, compounds' metabolic profile can be clearly identified so that their usefulness as an effective component of a pharmaceutical agent can be confirmed.

Usefulness of the compounds of the present invention represented by the above Formula (1), salts or derivatives thereof useful as a prodrug as an effective component for a pharmaceutical agent can be determined by carrying out a dissolution test, for example. Examples of the test include a method of determining solubility based on turbidity (Lipinski, C. A. et. al., Adv. Drug Deliv. Rev., 23, pp. 3-26, 1997; Bevan, C. D. et. al., Anal. Chem., 72, pp. 1781-1787, 2000), etc. By identifying compound's dissolution property based on this method, usefulness of a compound as an effective component for a pharmaceutical agent can be confirmed.

With respect to a dissolution test, methods described in the following can be mentioned, for example. In addition, by characterizing solubility of a compound using such methods, usefulness of the compound as an effective component for a pharmaceutical agent can be confirmed.

About 0.4 mg of each compound is weighed and dissolved in a dissolution solution to obtain the compound concentration of 1 mg/mL. Examples of a dissolution solution include the followings; (1) DMSO, (2) H₂O, (3) hydrochloric acid buffer solution having pH of 1.2, (4) phosphoric acid buffer solution having pH of 6.8, and (5) a solution comprising 20 mM bile acid in phosphoric acid buffer having pH of 6.8. These solutions are shaken for 1 hour at 37° C., and subjected to centrifugal filtration by using a filter (Ultrafree-MC PVDF 0.48 μm (Millipore)) for HPLC analysis of the filtrate. As an eluent, 0.1% acetic acid water and acetonitrile are used, and YMC-Pack C18 is used as a column. Detection is carried out at 254 nM and the column temperature is 40° C., and the flow rate is 1 mL/min. For the above case (1), the solubility is set to 100%. From the area obtained from the case (1) and the area obtained from each dissolution solution, solubility for the each dissolution solution is calculated.

Usefulness of the compounds of the present invention represented by the above Formula (1), salts or derivatives thereof useful as a prodrug as an effective component for a pharmaceutical agent can be determined by examining problems associated with an upper gastrointestinal tract or a kidney, etc., for example. With respect to a pharmacological test for an upper gastrointestinal tract, compound's effect on gastric mucosal membrane using a fasted rat having damaged gastric mucosal membrane can be mentioned. With respect to a pharmacological test for kidney function, a method of measuring renal blood flow amount and glomerular filtration rate [Physiology, 18^{th} ed. Bunkodo, 1986, Chapter 17] can be mentioned. By running one, two or more tests based on these methods, compounds' effect on an upper gastrointestinal tract or a kidney function can be clearly identified so that their usefulness as an effective component of a pharmaceutical agent can be confirmed.

With respect to the pharmaceutical agent of the present invention, the compounds of the present invention or pharmacologically acceptable salts thereof or a mixture comprising two or more kinds of them can be used by themselves. However, it is preferable that to the compounds of the present invention or pharmacologically acceptable salts thereof or to a mixture comprising two or more kinds of them, one, two or more kinds of pharmacologically acceptable carriers are added to prepare a pharmaceutical composition for administration. Types of the pharmacologically acceptable carriers are not specifically limited, but include an excipient, a binder, a disintegrating agent, a lubricating agent, or an additive, etc. Examples of an excipient include D-mannitol and the like. Examples of a binding agent include carboxymethylcellulose and the like. Examples of a disintegrating agent include corn starch and the like. Examples of a lubricating agent include glycerin and the like. Examples of an additive include para oxybenzoic acid ester, and the like. Further, examples of an additive include a surfactant like polyoxyethylene sorbitan monooleate (Tween 80) or HC60 and the like.

When the pharmaceutical agent of the present invention is administered to a human, it can be orally administered in a form a tablet, powder, a granule, a capsule, a sugar-coated tablet, a liquid or syrup, etc. Further, it can be also administered via parenteral route in a form including an injection solution, drops, a suppository, a trans-dermal or absorbing agent, etc. Still further, administration in a form of a spraying agent such as aerosol, dry powder, etc. can be also mentioned as a preferred administration form.

Administration period of the pharmaceutical agent of the present invention is not specifically limited. However, when it is administered under the purpose of treatment, a period during clinical signs of a disorder is found to be present can be taken as a time period for the administration in principle. In general, the administration is continued from several weeks to one year. However, depending on symptoms, it can be further administered, or can be continuously administered even after recovery from clinical symptoms. In addition, even when no clinical signs are observed based on clinician's judgment, it can be administered for a prophylactic purpose. Dosage of the pharmaceutical agent of the present invention is not specifically limited. For example, it can be generally in an effective amount of 0.01 to 2000 mg per day for an adult, a single or divided in several portions. Administration frequency can be from once a month to everyday. Preferably, it is once a week to three times a week, or five times a week, or can be administered everyday. Single time dosage, administration period, and administration frequency, etc. may be either increased or decreased according to age, body weight, overall health of a subject, or disorder to be treated and severeness of the disorder, etc.

It is evident that the pharmaceutical agent of the present invention can be administered with other curative or prophylactic agent that are used against various symptoms or disorders, aside from the curative and/or prophylactic purpose of the pharmaceutical agent of the present invention.

Herein below, the present invention will be explained in greater detail in view of the examples. However, scope of the present invention is not limited to them.

Regarding the examples described below, various analysis was carried out according to the followings.

### (1) Liquid Chromatography Mass Analysis Spectrum (LC-MS)

Mass spectrum was measured by the liquid chromatography mass analysis spectrum (LC-MS).

With regard to the analysis, the following conditions (A), (B) or (C) was applied.

In any case, “RT” is retention time of the liquid chromatography (unit: min) and the mass spectrum data of LC-MS is described as “MASS”

(A) As a mass spectrometer, Platform-LC type mass spectrometer (manufactured by Micromass, England) was used (ionization was carried out based on an electrospray method (ESI)). The liquid chromatography instrument manufactured by GILSON, France was used. As a separation column, Mightysil RP-18 GP 50-4.6 (product No. 25468-96, manufactured by Kanto Chemical Co., Inc., Japan) was used. Condition for elution was as follows.

flow rate: 2 mL/min
solvent: liquid A=water, contains 0.1% (v/v) acetic acid liquid B=acetonitrile, contains 0.1% (v/v) acetic acid from minute 0 to minute 2.8, liquid B with linear gradient
   of 5 to 98% (v/v).

(B) As a mass spectrometer, Platform-LC type mass spectrometer (manufactured by Micromass) was used and the measurement was carried out according to an electrospray method (ESI). The liquid chromatography instrument manufactured by GILSON was used. As a separation column, Develosil C30-UG-5 (50×4.6 mm, manufactured by Nomura Chemical Co., Ltd.) was used. In general, condition for elution was as follows—flow rate: 2 mL/min, solvent: liquid A=water containing 0.1% (v/v) acetic acid, liquid B=acetonitrile containing 0.1% (v/v) acetic acid, and from minute 0 to minute 5, liquid B with linear gradient of 5 to 98% (v/v) was applied and then to minute 6, 98% liquid B was applied as an eluent.

(C) As amass spectrometer, single quadrupole mass analysis apparatus: HPLC/SQD system [manufactured by WATERS] was used and the measurement was carried out according to an electrospray method (ESI). The liquid chromatography instrument manufactured by Acquity Ultra Performance LC [manufactured by WATERS] was used. As a separation column, ACQUITY HPLC BEH C18 (2.1×50 mm 1.7 μm, manufactured by WATERS) was used. In general, condition for elution was as follows—flow rate: 0.6 mL/min, liquid A=water containing 0.1% (v/v) acetic acid, liquid B=acetonitrile containing 0.1% (v/v) acetic acid, and from minute 0 to minute 2.0, liquid B with linear gradient of 5 to 90% (v/v) was applied and then from minute 2.0 to minute 2.5, linear gradient of 90 to 98% (v/v) liquid B was applied as an eluent.

Furthermore, unless specifically described otherwise, a condition for the measurement of the data described below is the same as condition (C) above.

### (2) Nuclear Magnetic Resonance Spectrum (NMR)

Gemini-300 (FT-NMR, manufactured by Varian, Inc.) was used for the measurement. As a solvent, deuterated chloroform (CDCl₃), deuterated methanol (CD₃OD) or deuterated dimethylsulfoxide (DMSO-d₆) was used, and unless specifically described otherwise, CDCl₃ was used for the measurement. For measurement of chemical shift, tetramethylsilane (TMS) was taken as an internal standard. The chemical shift value was expressed in δ (ppm). In addition, a coupling constant was expressed in J (Hz). Furthermore, symbols for a splitting pattern are as follow: s; singlet, d; doublet, t; triplet, q; quartet, qu; quintet, dd; doublet doublet, td; triplet doublet, m; multiplet, brs; broad singlet, brd; broad doublet, brdd; broad doublet doublet, brddd; broad doublet doublet doublet.

### (3) Thin Layer Chromatography (TLC)

TLC plate manufactured by Merck Co., Germany was used (Silica Gel 60 F₂₅₄, Product No. 1,05715). Detection of compounds was carried out according to a general method for detection, for example, illuminating a developed TLC plate with UV light having wavelength of 254 nm, etc.

### (4) Chromatography for Purification

In principle, one of the following four methods was used. (Purification method 1) Based on “Flash Column System” (manufactured by Biotage Co.), one or several cartridge columns selected from KP-Sil-12M, 40S, 40M, 12+M and 40+S, all manufactured by Biotage Co., are used depending on the amount of a sample.

(Purification method 2) Typical column chromatography was carried out by using Silica gel 60N (globular, neutral, 40 to 100 μm, manufactured by Kanto Chemical Co., Inc., Japan) depending on the amount of a sample.
(Purification method 3) “Yamazen Flash Column System” (manufactured by Yamazen Corp.) was used. In addition, one or multiple cartridge columns of “High Flash Column” S, M, L, 3L and 5L (manufactured by Yamazen Corp.) were used depending on the amount of a sample.
(Purification method 4) Regarding HPLC purification, Waters HPLC system (manufactured by WATERS) was used. As an eluent, solvent mixture water comprising 0.1% acetic acid-acetonitrile was used (if necessary, composition thereof will be described). As to a column, any one of the following four columns was used.
A: XBridge OBD (TM) (19 mmI.D.×50 mm) (manufactured by WATERS)
B: Mightysil R^{P}-18GP 25649-96 (20 mmI.D.×50 mm) (manufactured by Kanto Chemical Co., Inc.)
C: Develosil C30-UG-5 (20 mmI.D.×50 mm) (manufactured by Nomura Chemical Co., Ltd.)
D: Develosil ODS-HG-5 (20 mmI.D.×50 mm) (manufactured by Nomura Chemical Co., Ltd.)

When purification was carried out based on HPLC, to obtain the target compound, the solvent was removed by freeze drying, and by purging with nitrogen gas, unless specifically described otherwise.

For the examples described herein below, “LC-MS” means liquid chromatography mass analysis spectrum, “RT” means retention time in liquid chromatography (unit; min.), and mass spectrum data of LC-MS is indicated as “MASS.” In addition, meaning of the symbols included in each table is as follows. “Exp.”; Example compound No., “Ref.”; reference example No., “Syn.”; synthetic method, “SM”; starting material, “Supplier”: supplier of SM, “Structure”; structure of a target compound in each table. In addition, meaning of the symbols included in “Supplier” column is as follows. “TCI”; product of Tokyo Chemical Industry, Co., Ltd., “Wako”; product of Wako Pure Chemical Industries, Ltd., “Ald”; product of Aldrich Company, “Alfa”; product of Alfa Aesar Co., “Fro”; product of Frontier INC., “JWP”; product of J&W Pharmlab.

### Reference Example 1

### 1,3-Dibromoacetone dimethylacetal

To methanol solution (4 L) comprising acetone (575 g), bromine (3164 g) was added at 10° C. After stirring for 24 hours, the reaction solution was poured over water, and extracted with dichloromethane. The organic layer was washed with aqueous solution of sodium thiosulfate, and then dried over magnesium sulfate. Solids were removed, the filtrate was dried under reduced pressure, and recrystallized to obtain the title product (1850 g).

¹H-NMR (CDCl₃): 3.52 (4H, s), 3.24 (6H, s).

### Reference Example 2

### Diisopropyl 3,3-dimethoxycyclobutane-1,1-dicarboxylate

To DMF solution (1.8 L) comprising diisopropyl malonate (1437 g), sodium hydride (367 g) was added at 15° C. Subsequently, 1,3-dibromoacetone dimethylacetal obtained from Reference example 1 was added thereto, stirred at 130° C. for 24 hours, followed by further stirring for three days. Upon the completion of stirring, the reaction was terminated by adding aqueous solution of ammonium chloride, and extraction was carried out using hexane. The organic layer was washed with water, and then dried over magnesium sulfate. Solids were removed, and the filtrate was dried under reduced pressure. Thus obtained residues were subjected to flash column chromatography (using 10:1 (v/v) hexane/ethyl acetate as an eluent) to obtain the title compound (1.5 kg).

¹H-NMR (CDCl₃): 5.08 (2H, m), 3.15 (6H, s), 2.69 (4H, s), 1.24 (12H, m).

### Reference Example 3

### 3-Oxocyclobutane carboxylic acid

To diisopropyl 3,3-dimethoxycyclobutane-1,1-dicarboxylate (760 g) obtained from Reference example 2, 20% of hydrochloric acid solution (3.2 L) was added and the mixture was refluxed for four days. After that, the reaction solution was extracted with ethyl acetate, and then the organic layer was dried over magnesium sulfate. Solids were removed, the filtrate was dried under reduced pressure to obtain the title product (700 g).

¹H-NMR (CDCl₃): 11.2 (1H, s), 3.41 (5H, m).

### Reference Example 4

### Tert-butyl 3-oxocyclobutane carboxylate

To dichloromethane solution (1.6 L) comprising 3-oxocyclobutane carboxylic acid obtained from Reference example 3, tert-butanol (429 g), 4-dimethylaminopyridine (283 g), dichloromethane solution (700 mL) comprising DCC (656 g) was added and the mixture was stirred at room temperature for twenty hours. Upon the completion of stirring, the reaction solution was filtered using Celite, and washed with 1N hydrochloric acid solution. The organic layer was washed with saturated sodium bicarbonate solution, and then dried over magnesium sulfate. Solids were removed, and the filtrate was dried under reduced pressure to obtain the title compound (530 g).

¹H-NMR (CDCl₃): 3.28 (4H, m), 1.48 (9H, s).

### Reference Example 5

### Tert-butyl 3-azidecyclobutane carboxylate

### Step 1:

To ethanol solution (2.9 L) comprising tert-butyl 3-oxocyclobutane carboxylate (490 g) obtained from Reference example 4, ethanol suspension (1.8 L) comprising sodium borohydride (54.4 g) was added while maintaining the temperature at 5° C. After stirring for one hour, the reaction solution was added with ammonium chloride solution to terminate the reaction. The mixture was extracted with dichloromethane and the organic layer was dried over magnesium sulfate. Solids were removed, and the filtrate was dried under reduced pressure.

### Step 2:

To dichloromethane solution (2.4 L) comprising the residues (496 g) obtained from Step 1 above, triethylamine (583 g), 4-dimethylaminopyridine (176 g), and dichloromethane solution (1 L) comprising tosyl chloride (824 g) was added while maintaining the temperature at 5° C. After stirring for two hours, the reaction solution was poured over water, and then extracted with dichloromethane. The organic layer was dried over magnesium sulfate. Solids were removed, and the filtrate was dried under reduced pressure. The resulting residues were used for next reaction without purification.

### Step 3:

To ethanol solution (5 L) comprising the residues (940 g) obtained from Step 2 above, water (1.2 L) and sodium azide (280 g) were added. After refluxing for forty-two hours, the reaction solution was concentrated. The reaction solution was extracted with ethyl acetate. The organic layer was dried over magnesium sulfate. Solids were removed, and dried under reduced pressure. Thus obtained residues were subjected to flash column chromatography (using 10:1 (v/v) hexane/ethyl acetate as an eluent) to obtain the title compound (150 g).

¹H-NMR (CDCl₃): 2.98 (2H, m), 2.52 (2H, m), 1.45 (1H, m), 1.12 (1H, m).

### Reference Example 6

### Tert-butyl 1,3-trans-3-aminocyclobutane carboxylate

To methanol solution (1.2 L) comprising tert-butyl 3-azide cyclobutane carboxylate obtained from Reference example 5 in the above, 10% palladium-carbon (30 g) was added, followed by stirring for 24 hours at room temperature under hydrogen atmosphere. Upon the completion of stirring, the reaction solution was filtered using Celite and concentrated under reduced pressure.

Thus obtained residues were subjected to flash column chromatography (using ethyl acetate and methanol as an eluent) to obtain the title compound (112 g).

¹H-NMR (CDCl₃): 3.68 (1H, m), 2.89 (1H, m), 2.50 (2H, m), 1.92 (2H, m), 1.45 (9H, s).

### Reference Example 7

### Step 1. 4-Chloro-3,5-dimethylbenzoic acid

To THF/hexane solution (85 mL/17 mL) comprising 5-bromo-2-chloro-m-xylene (2.5 g, product of Fluorochem Co.), n-butyl lithium-hexane solution (1.58 M) (7.9 mL, product of Kanto Chemical Co., Inc.) was added dropwise at −78° C. for 10 minutes under nitrogen atmosphere. Then, at −78° C., dry ice was added thereto, and upon the completion of reaction, the reaction solution was added with water. The organic layer was concentrated, and added with 1N hydrochloric acid solution (product of Wako Pure Chemical Industries, Ltd.). The resultant was collected by filtration and dried under reduced pressure to obtain the title compound (1.76 g).

MASS: 183.0 (M−H), RT: 1.54 min.

### Step 2. Methyl 4-chloro-3,5-dimethylbenzoate

To methanol solution (40 mL) comprising 4-chloro-3,5-dimethylbenzoic acid obtained from Step 1 in the above, conc. hydrochloric acid (1.0 mL) was added followed by stirring for 16 hours under reflux. Upon the completion of stirring, the reaction solution was concentrated, poured over saturated sodium bicarbonate solution. Then the solution was extracted with ethyl acetate and the organic layer was again washed with saturated brine. After drying over sodium sulfate, the solids were removed and the filtrate was dried under reduced pressure to obtain the title compound (1.70 g).

¹H-NMR (CDCl₃): 7.75 (2H, s), 3.90 (3H, s), 2.42 (6H, s).

### Reference Example 8

### Step 1. 4-Chloro-2-fluoro-5-methylbenzoic acid

12% of sodium hypochloride solution (312 mL, product of Acros Co.) comprising 1-(4-chloro-2-fluoro-5-methylphenyl)-1-ethanone (25.0 g, product of Bionet Co.) was stirred for 19 hours at room temperature. Upon the completion of the reaction, the reaction solution was added with 5% of sodium acid sulfite solution (200 mL) at 0° C. Then pH of the system was adjusted to 1 with conc. hydrochloric acid and the product was collected by filtration, and dried under reduced pressure to obtain the title compound (16.8 g). MASS: 187.1 (M−H), RT: 1.38 min.

### Step 2. Methyl 4-chloro-2-fluoro-5-methylbenzoate

To methanol solution (350 mL) comprising 4-chloro-2-fluoro-5-methylbenzoic acid obtained from Step 1 in the above, conc. hydrochloric acid (10 mL) was added followed by stirring for 21 hours under reflux. Upon the completion of stirring, the reaction solution was concentrated, poured over 1N hydrochloric acid solution. Then the solution was extracted with ethyl acetate. After drying over sodium sulfate, the solids were removed and the filtrate was dried under reduced pressure to obtain the title compound (14.4 g).

¹H-NMR (CDCl₃): 7.81 (1H, d, J=7.7), 7.17 (1H, d, J=10.3), 3.92 (3H, s)

### Reference Example 9

### Step 1. Methyl 4-phenyl-3-methylbenzoate

To 1,4-dioxane solution (330 mL) comprising phenylboronic acid (12 g, product of Tokyo Chemical Industry, Co., Ltd.), methyl 4-bromo-3-methyl-benzoate (15 g, product of Tokyo Chemical Industry, Co., Ltd.), tris(benzylidene acetone) dipallaidum (6.0 g, product of Aldrich Company), tri-tert-butylphosphonium tetrafluoroborate (4.7 g, product of Aldrich Company), and cesium carbonate (32 g, product of Kanto Chemical Co., Inc.) were added, followed by stirring at temperature of 90° C. for 15 hours. Upon the completion of stirring, the reaction solution was filtered using Celite, and the filtrate was concentrated under reduced pressure. Thus obtained residues were subjected to flash column chromatography (using 20:1 (v/v) hexane/ethyl acetate as an eluent) to obtain the title compound.

¹H-NMR (CDCl₃): 7.93-7.97 (1H, m), 7.89 (1H, ddd, J=0.54, J=1.8, J=7.9), 7.47-7.28 (6H, m), 3.93 (3H, s).

### Step 2. 4-Phenyl-3-methylbenzoic acid

To methanol solution (360 mL) comprising methyl 4-phenyl-3-methylbenzoate obtained from step 1, 5N sodium hydroxide solution (40 mL, product of Wako Pure Chemical Industries, Ltd.) was added followed by stirring overnight. Upon the completion of stirring, the reaction solution was concentrated under reduced pressure, added with 1N hydrochloric acid solution. Then the resultant was collected by filtration and dried under reduced pressure to obtain the title compound (13 g).

MASS: 211.3 (M−H), RT: 1.55 min.

Except that any of the starting materials shown in Table 6 is used, the carboxylic acids described in Table 6 were synthesized in the same manner as Reference example 9. Meanwhile, the compounds described to have a supplier “syn” in Table 6 are the compounds which had been synthesized in Reference example 7 or 8.

TABLE 6

| | | | | | | LCMS (ESI−) | |
|---|---|---|---|---|---|---|---|
| ref. | SM. | Supplier | SM. | Supplier | Structure | MASS | RT |
| 10 | 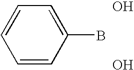 | TCI | 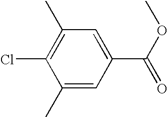 | syn | 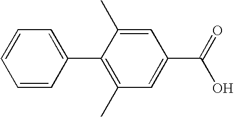 | 225.1 | 1.69 |
| 11 | 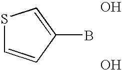 | Alfa Aesar? | 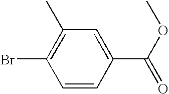 | TCI? | 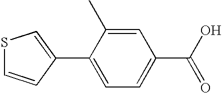 | 219.0 | 1.56 |
| 12 | 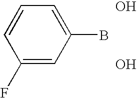 | WAKO | 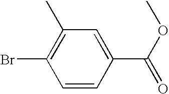 | TCI? | 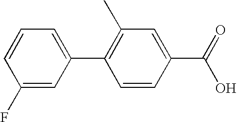 | 229.0 | 1.68 |
| 13 | 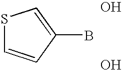 | Alfa Aesar? | 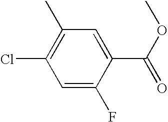 | syn | 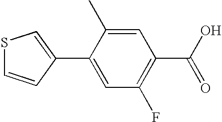 | 237.0 | 1.53 |
| 14 | 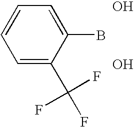 | WAKO | 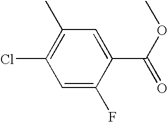 | syn | 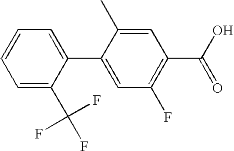 | 297.0 | 1.66 |
| 15 | 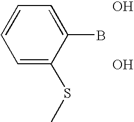 | Ald | 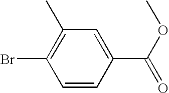 | TCI? | 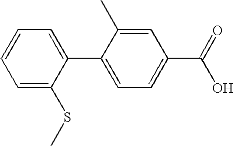 | 257.0 | 1.81 |

### Reference Example 16

### 1-Oxo-2,3-dihydro-1H-inden-5-carbonitrile

To N,N-dimethyl formamide solution (70 mL) comprising 5-bromo-1-indanone (2.5 g, product of Tokyo Chemical Industry, Co., Ltd.) and zinc cyanide (1.67 g, product of Aldrich Company) and tetrakistriphenylphosphine palladium (682 mg, product of Kanto Chemical Co., Inc.) was added at room temperature, followed by stirring at temperature of 105° C. for 4 hours. Upon the completion of stirring, the reaction solution was poured over saturated sodium bicarbonate solution and extracted with diethyl ether. The organic layer was washed with saturated brine and dried over magnesium sulfate. Thus solids were removed by filtration, the filtrate was dried under reduced pressure and obtained residues were subjected to flash column chromatography with Biotage 40S (using 8:1 (v/v) hexane/ethyl acetate as an eluent) to obtain the title compound (1.63 g).

¹H-NMR (CDCl₃): 7.85 (1H, d, J=9.0), 7.82 (1H, s), 7.67 (1H, d, J=9.0), 3.23 (2H, t, J=6.0), 2.78 (2H, t, J=6.0).

### Reference Example 17

### Tert-butyl 1,3-trans-3-(5-cyano-2,3-dihydro-1H-inden-1-ylamino)cyclobutanecarboxylate

To methanol solution (50 mL) comprising 1-oxo-2,3-dihydro-1H-inden-5-carbonitrile (1.11 g) and tert-butyl 1,3-trans-aminocyclobutanecarboxylate (1.21 g), acetic acid (537 μl, product of Wako Pure Chemical Industries, Ltd.) and sodium cyanoborohydride (666 mg, product of Tokyo Chemical Industry, Co., Ltd.) were added at room temperature, followed by stirring at the same temperature for two days. Upon the completion of stirring, the reaction solution was poured over saturated sodium bicarbonate solution and extracted with chloroform. The organic layer was washed with saturated brine and dried over sodium sulfate. Thus solids were removed by filtration, dried under reduced pressure and obtained residues were subjected to flash column chromatography with Biotage 40S (using 5:1 (v/v) to 1:1 (v/v) hexane/ethyl acetate as an eluent) to obtain the title compound (1.44 g).

MASS: 313.1 (M+H), RT: 1.15 min.

### Reference Example 18

### Tert-butyl 1,3-trans-3-(tert-butoxycarbonyl(5-cyano-2,3-dihydro-1H-inden-1-yl)amino)cyclobutane

To dichloromethane solution (30 mL) comprising tert-butyl 1,3-trans-3-(5-cyano-2,3-dihydro-1H-inden-1-ylamino)cyclobutanecarboxylic acid (1.44 g), triethylamine (1.91 mL, product of Wako Pure Chemical Industries, Ltd.) and di-tert-butyl carbonate (1.51 g, product of Wako Pure Chemical Industries, Ltd.) were added at room temperature, followed by stirring at the same temperature overnight. Upon the completion of stirring, the reaction solution was poured over saturated sodium bicarbonate solution and extracted with dichloromethane. The organic layer was washed with saturated brine and dried over magnesium sulfate. Thus solids were removed by filtration, the filtrate was dried under reduced pressure and obtained residues were subjected to flash column chromatography with Biotage 40S (using 6:1 (v/v) hexane/ethyl acetate as an eluent) to obtain the title compound (1.55 g).

MASS: 413.1 (M+H), RT: 2.18 min.

### Reference Example 19

### Tert-butyl 1,3-trans-3-(tert-butoxycarbonyl(5-(N′-hydroxycarbamimidoyl)-2,3-dihydro-1H-inden-1-yl)amino)cyclobutanecarboxylate

To methanol solution (30 mL) comprising tert-butyl 1,3-trans-3-(tert-butoxycarbonyl(5-cyano-2,3-dihydro-1H-inden-1-yl)amino)cyclobutane (979.4 mg), hydroxyammoniumchloride (329.4 mg, product of Kanto Chemical Co., Inc.), sodium hydrocarbonate (796.4 mg, product of Wako Pure Chemical Industries, Ltd.) were added at room temperature, followed by refluxing for 5 hours. Upon the completion of reflux, the reaction solution was poured over saturated brine and extracted with ethyl acetate. The organic layer was dried over magnesium sulfate. Thus solids were removed by filtration, the filtrate was dried under reduced pressure and obtained residues were subjected to flash column chromatography (using 1:1 (v/v) hexane/ethyl acetate as an eluent) to obtain the title compound (827 mg).

MASS: 446.2 (M+H), RT: 1.59 min.

### Reference Example 20

### Tert-butyl 1,3-trans-3-(tert-butoxycarbonyl(5-(5-(2-methylbiphenyl-4-yl)-1,2,4-oxadiazol-3-yl)-2,3-dihydro-1H-inden-1-ylamino)cyclobutanecarboxylate

To N,N-dimethylformamide solution (4 mL) comprising tert-butyl 1,3-trans-3-(tert-butoxycarbonyl(5-(N′-hydroxycarbamimidoyl)-2,3-dihydro-1H-inden-1-yl)amino)cyclobutanecarboxylate (50 mg) and 2-methylbiphenyl-4-carboxylic acid (25.5 mg), WSC HCl (26.8 mg, product of Tokyo Chemical Industry, Co., Ltd.) and HOBt (19.0 mg, product of Watanabe Chemical Company) were added at room temperature, followed by stirring at the same temperature for one hour. The mixture was further stirred overnight at 100° C. Upon the completion of stirring, the reaction solution was poured over saturated sodium bicarbonate solution and extracted with diethyl ether. The organic layer was washed with saturated brine and dried over magnesium sulfate. Thus solids were removed by filtration, the filtrate was dried under reduced pressure and obtained residues were subjected to flash chromatography (using 9:1 (v/v) hexane/ethyl acetate as an eluent) to obtain the title compound (18.1 mg).

¹H-NMR (CDCl₃): 8.01-8.15 (5H, m), 6.91-8.01 (6H, m), 2.87-3.13 (3H, m), 2.05-2.47 (4H, m), 0.86-1.28 (8H, m), 1.25 (18H, s).

### Example 1

### 1,3-Trans-3-(5-(5-(2-methylbiphenyl-4-yl)-1,2,4-oxadiazol-3-yl)-2,3-dihydro-1H-inden-1-ylamino)cyclobutanecarboxylic acid hydrochloride salt

To tert-butyl 1,3-trans-3-(tert-butoxycarbonyl-(5-(5-(2-methylbiphenyl-4-yl)-1,2,4-oxadiazol-3-yl)-2,3-dihydro-1H-inden-1-yl)amino)cyclobutane carboxylate (18.1 mg), 4N hydrochloric acid dioxane solution (5 mL, product of KOKUSAN CHEMICAL Co., Ltd.) was added at room temperature. After stirring overnight at the same temperature, the solvent was removed by purging with nitrogen gas to obtain the title compound.

MASS: 466.0 (M+H), RT: 1.51 min.

Except that any of the starting materials shown in Table 7 is used, the compounds described in Table 7 were synthesized in the same manner as Reference example 20 and Example 1.

TABLE 7

| | | | LCMS (ESI+) | |
|---|---|---|---|---|
| Exp. | SM | Structure | MASS | RT |
| 2 | 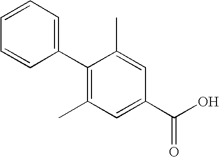 | 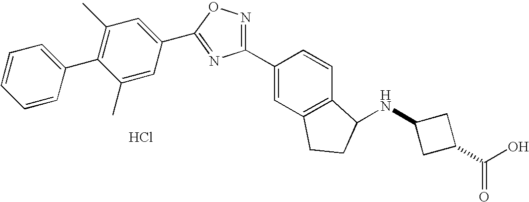 | 480 | 1.63 |
| 3 | 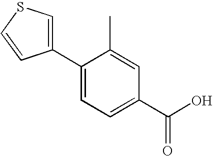 | 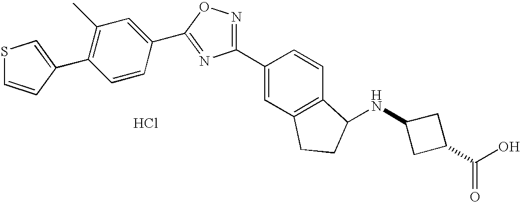 | 472 | 1.51 |
| 4 | 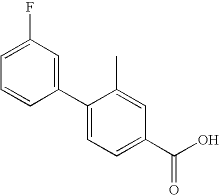 | 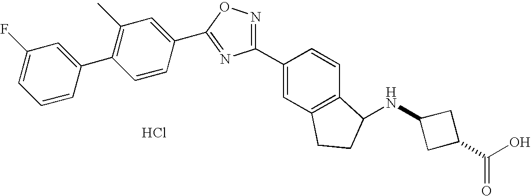 | 484 | 1.51 |
| 5 | 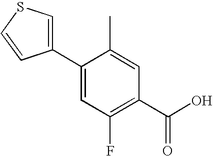 | 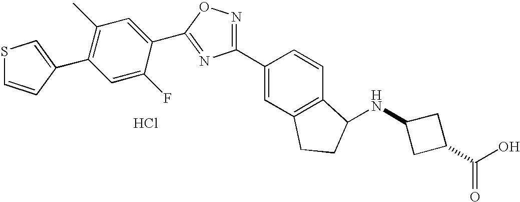 | 490 | 1.52 |
| 6 | 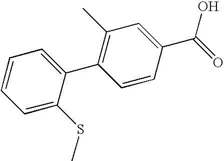 | 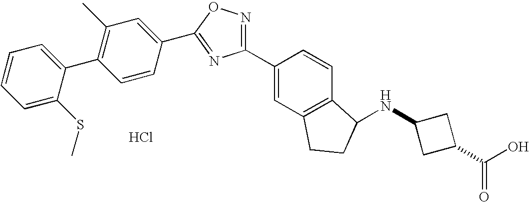 | 512 | 1.56 |

### Reference Example 21

### 6-Cyano-1-tetralone

Step 1: To dichloromethane solution (60 mL) comprising 6-hydroxy-1-tetralone (1.0 g, product of Aldrich Company), N-phenylbis(trifluoromethanesulfoneimide) (2.2 g, product of Tokyo Chemical Industry, Co., Ltd.) and diisopropylethylamine (1.57 mL, product of Tokyo Chemical Industry, Co., Ltd.) were added at room temperature, followed by stirring overnight. Upon the completion of stirring, the reaction solution was poured over saturated sodium bicarbonate solution and extracted with dichloromethane. The organic layer was washed with saturated brine and dried over magnesium sulfate. Thus solids were removed by filtration, the filtrate was dried under reduced pressure and obtained residues were subjected to flash column chromatography (using 10/1 (v:v) hexane/ethyl acetate as an eluent).

Step 2: Except that the product of Step 1 is used instead of 5-bromo-1-indanone, the title compound was synthesized in the same manner as Reference example 16.

¹H-NMR (CDCl₃): 8.10-8.12 (1H, m), 7.57-7.60 (2H, m), 2.99-3.03 (2H, m), 2.69-2.74 (2H, m), 2.14-2.23 (2H, m).

### Example 7

### 1,3-Trans-3-(6-(5-(3-methyl-4-(thiophen-3-yl)phenyl)-1,2,4-oxadiazol-3-yl)-1,2,3,4-tetrahydronaphthalen-1-ylamino)cyclobutanecarboxylic acid hydrochloride salt

Except that 6-cyano-1-tetralone is used instead of 1-oxo-2,3-dihydro-1H-inden-5-carbonitrile, the processes same as Reference example 17 to 19 were carried out. In addition, except that tert-butyl 1,3-trans-3-(tert-butoxycarbonyl(6-(N′-hydroxycarbamimidoyl)-1,2,3,4-tetrahydronaphthalen-1-yl)amino)cyclobutanecarboxylate is used instead of tert-butyl 1,3-trans-3-(tert-butoxycarbonyl(5-(N′-hydroxycarbamimidoyl)-2,3-dihydro-1H-inden-1-yl)amino)cyclobutanecarboxylate and 3-methyl-4-(thiophen-3-yl)benzoic acid is used instead of 2-methylbiphenyl-4-carboxylic acid, the title compound was synthesized in the same manner as Reference example 20 and Example 1.

MASS: 486.0 (M+H), RT: 1.62 min.

### Reference Example 22

### 1,2-Bis(hydroxymethyl)-4-bromobenzene

To THF solution (40 mL) comprising 4-bromophtahalic acid (1.0 g, product of Tokyo Chemical Industry, Co., Ltd.), borane-dimethylsulfide (2.0 M THF solution, product of Aldrich Company, 5.1 mL), was added at room temperature, followed by refluxing under heating for 16 hours. Methanol was added to the reaction solution, and liquid separation was carried out by using saturated sodium bicarbonate solution and saturated brine in order. Then, the aqueous layer was extracted with ethyl acetate and the collected organic layer was dried over sodium sulfate. Thus solids were removed by filtration, the filtrate was dried under reduced pressure and obtained residues were subjected to flash column chromatography (using 4/1 to 0/1 (v:v) hexane/ethyl acetate as an eluent) to obtain the title compound (784 mg).

MASS: 214.8 (M−H), RT 1.07 min.

### Reference Example 23

### 1,2-Bis((tert-butyldimethylsilyloxy)methyl)-4-bromobenzene

To DMF solution (6.0 mL) comprising 1,2-bis(hydroxymethyl)-4-bromobenzene (684 mg), imidazole (1.07 g, product of Tokyo Chemical Industry, Co., Ltd.), tert-butyldimethylsilyl chloride (1.42 g, product of Tokyo Chemical Industry, Co., Ltd.) were added at room temperature, followed by stirring overnight. Upon the completion of stirring, saturated brine was poured over the reaction solution and liquid separation was carried out. Then, the aqueous layer was extracted with diethyl ether and the collected organic layer was dried over sodium sulfate. Thus solids were removed by filtration, the filtrate was dried under reduced pressure and obtained residues were subjected to column chromatography (using 50/1 (v:v) hexane/ethyl acetate as an eluent) to obtain the product, which was then used for a next step without further purification.

¹H-NMR (CDCl₃): 7.49 (1H, d, J=2.2), 7.29 (1H, d, J=2.2, J=8.4), 7.19 (1H, d, J=8.4), 4.59 (4H, d, J=13.2), 0.85 (9H, s), 0.84 (9H, s), 0.02 (6H, d), 0.00 (6H, s).

### Reference Example 24

### 3,4-Bis((tert-butyldimethylsilyloxy)methyl)benzonitrile

Except that 1,2-bis((tert-butyldimethylsilyloxy)methyl)-4-bromobenzene is used as a starting material, the title compound was obtained in the same manner as Reference example 16.

¹H-NMR (CDCl₃): 7.61 (1H, s), 7.46 (2H, s), 4.60 (4H, d, J=11.3), 0.83 (18H, s), 0.01 (6H, d), 0.01 (6H, s).

### Reference Example 25

### 3,4-Bis((tert-butyldimethylsilyloxy)methyl)-N′-hydroxybenzimidamide

Except that 3,4-bis((tert-butyldimethylsilyloxy)methyl)benzonitrile is used as a starting material, the title compound was obtained in the same manner as Reference example 19.

MASS: 425.1 (M+H), RT: 2.41 min.

### Reference Example 26

### 1,2-Bis(hydroxymethyl)-4-(5-(2-methylbiphenyl-4-yl-(1,2,4-oxadiazol-3-yl)benzene

### Step 1:

To THF solution (2.0 mL) comprising 3,4-bis((tert-butyldimethylsilyloxy)methyl)-N′-hydroxybenzimidamide (80 mg), THF solution comprising tetrabutyl ammonium fluoride (1.0 M THF, product of Tokyo Chemical Industry, Co., Ltd., 564 μl) was added. The mixture was stirred at room temperature for 2.5 hours, followed by drying under reduced pressure to obtain the residues, which were then used for a next step without further purification.

### Step 2:

A mixture comprising 2-methylbiphenyl-4-carboxylic acid (42.4 mg), WSC.HCl (38.3 mg, product of Tokyo Chemical Industry, Co., Ltd.), HOBt (27 mg, product of nacalai tesque Ltd.), and DMF (2.0 mL) were stirred at room temperature for 4.5 hours. The residues (26.1 mg) obtained from above Step 1 were added thereto, and stirred for 3.5 hours at room temperature. The mixture was again stirred at 120° C. for six hours, followed by concentration under reduced pressure. By carrying out column chromatography (using 5/1 to 1/1 (v:v) hexane/ethyl acetate as an eluent), the title compound was obtained (60.5 mg).

MASS: 373.0 (M+H), RT: 1.86 min.

Except that any of the starting materials shown in Table 8 is used, the compounds described in Table 8 were synthesized in the same manner as Step 2 of Reference example 26.

TABLE 8

| | | | | LCMS (ESI+) | |
|---|---|---|---|---|---|
| ref. | SM | SM | Structure | MASS | RT |
| 27 | 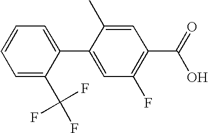 | 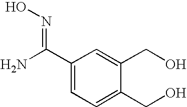 | 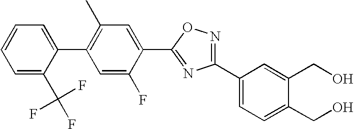 | 459.0 | 1.88 |
| 28 | 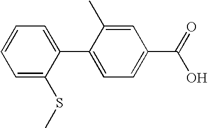 | 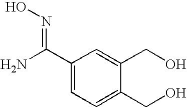 | 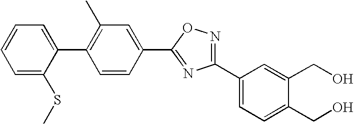 | 419.0 | 1.88 |

### Reference Example 29

### 3-(3,4-Bis(bromomethyl)phenyl)-5-(2-methylbiphenyl-4-yl)-1,2,4-oxadiazole

To dichloromethane solution comprising 1,2-bis(hydroxymethyl)-4-(5-(2-methylbiphenyl-4-yl)-1,2,4-oxadiazol-3-yl)benzene (60.5 mg), carbon tetrabromide (118 mg, product of Tokyo Chemical Industry, Co., Ltd.), triphenyl phosphine (93.4 mg, product of Tokyo Chemical Industry, Co., Ltd.) were added at 0° C., followed by stirring. After 3.5 hours, triphenyl phosphine (21.2 mg) and carbon tetrabromide (26.8 mg) were further added at 0° C. and the mixture was stirred for 1.5 hours. By adding saturated sodium hydrocarbonate solution, liquid separation was carried out. Saturated brine was added to the organic layer for liquid separation, and the aqueous layer was extracted with chloroform. The organic layer was collected and dried over sodium sulfate. The solids were removed by filtration, the filtrate was dried under reduced pressure and obtained residues were subjected to column chromatography (using 100/1 (v:v) hexane/ethyl acetate as an eluent) to obtain the title compound (53.3 mg).

¹H-NMR (CDCl₃): 8.25-8.20 (1H, m), 8.16-8.03 (3H, m), 7.56-7.32 (7H, m), 4.73 (2H, s), 4.71 (2H, s), 2.38 (3H, s).

### Reference Example 30

### Tert-butyl 1,3-trans-3-(5-(5-(2-methylbiphenyl-4-yl)-1,2,4-oxadiazol-3-yl)isoindolin-2-yl)cyclobutanecarboxylate

The DMF (2.0 mL) solution comprising 3-(3,4-bis(bromomethyl)phenyl)-5-(2-methylbiphenyl-4-yl)-1,2,4-oxadiazole (53.3 mg), tert-butyl 1,3-trans-aminocyclobutanecarboxylate (21.9 mg), and potassium carbonate (37 mg, product of Wako Pure Chemical Industries, Ltd.) was stirred overnight at 90° C. Upon the completion of the reaction, 1N sodium hydroxide solution was added thereto for liquid separation. Saturated brine was added to the organic layer for liquid separation, and the aqueous layer was extracted with diethyl ether. The organic layer was collected and dried over sodium sulfate. The solids were removed by filtration, the filtrate was dried under reduced pressure and obtained residues were subjected to column chromatography (using 10/1 to 5/1 (v:v) hexane/ethyl acetate as an eluent) to obtain the title compound (22 mg).

MASS: 508.1 (M+H), RT: 1.81 min.

### Example 8

### 1,3-Trans-3-(5-(5-(2-methylbiphenyl-4-yl)-1,2,4-oxadiazol-3-yl)isoindolin-2-yl)cyclobutanecarboxylic acid hydrochloride salt

Except that tert-butyl 1,3-trans-3-(5-(5-(2-methylbiphenyl-4-yl)-1,2,4-oxadiazol-3-yl)isoindolin-2-yl)cyclobutanecarboxylate is used as a starting material, the title compounds was synthesized in the same manner as Example 1.

MASS: 452.0 (M+H), RT: 1.47 min.

Except that any of the starting materials shown in Table 9 is used, the compounds described in Table 9 were synthesized in the same manner as Reference examples 29 and 30 and Example 8.

TABLE 9

| | | | LCMS (ESI+) | |
|---|---|---|---|---|
| Exp. | SM | Structure | MASS | RT |
| 9 | 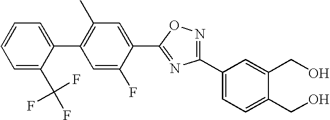 | 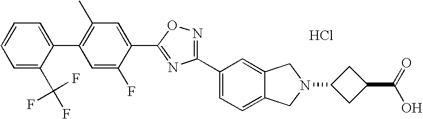 | 538.0 | 1.50 |
| 10 | 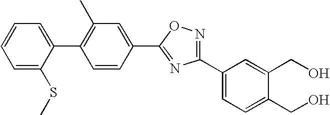 | 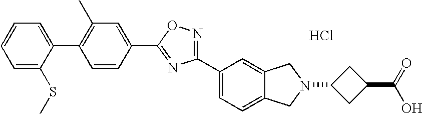 | 498.0 | 1.52 |

### Example 11

### Optically active 1,3-trans-3-(5-(5-(2-methyl-2′-methylthiobiphenyl-4-yl)-1,2,4-oxadiazol-3-yl)-2,3-dihydro-1H-inden-1-ylamino)cyclobutane carboxylic acid

1,3-Trans-3-(5-(5-(2-methyl-2′-methylthiobiphenyl-4-yl)-1,2,4-oxadiazol-3-yl)-2,3-dihydro-1H-inden-1-ylamino)cyclobutane carboxylic acid hydrochloric acid salt obtained from Example 6 was fractionated by using a chiral column under the following condition; column: CHIRALPAK AD-H 4.6 mm×250 mm (manufactured by Daicel Chemical Industries, Ltd.), column temperature: 40° C., detection: UV-254 nm; flow rate: 0.5 mL/min, mobile phase: n-Hex/EtOH/TFA/diethylamine=80/20/0.1/0.1). One optically active form was eluted at 17.6 min (Example 11-1), while the other optically active form was eluted at 22.9 min (Example 11-2). Next, each of the fractionates was dissolved in water and then applied to Sep-Pak C18 cartridge (1 cc) for solid phase extraction. After washing the cartridge with water, acetonitrile was added for elution. With concentration of an eluted solution, the title compound was obtained.

### Reference Example 31

### 3-Fluoro-2-trifluoromethylphenylboronic acid pinacol ester

To dioxane solution (20 mL) comprising 2-bromo-6-fluorobenzotrifluoride (500 mg, manufactured by Apollo Chemical Corp.), bispinacolate diborone (574 mg, manufactured by Aldrich Company), 1,1′-bis(diphenylphosphino)ferrocene]dichloropalladium-dichloromethane complex (1:1) (167 mg, manufactured by Aldrich Company), and potassium acetate (404 mg, manufactured by Kanto Chemical Co., Inc.) were added, followed by stirring at 95° C. for three hours. Upon the completion of stirring, the reaction solution was filtered using Celite, and the filtrate was concentrated under reduced pressure. Thus obtained residues were subjected to flash column chromatography (using 100:1 (v/v) to 50:1 (v/v) hexane/ethyl acetate as an eluent) to obtain the title compound (661 mg).

¹H-NMR (CDCl₃): 7.50 (1H, ddd, J=7.32, J=4.74), 7.31 (1H, d, J=7.32), 7.17 (1H, dd, J=8.40, J=11.0), 1.38 (12H, s).

### Reference Example 32

### 1-Bromo-2,3-bis(trifluoromethyl)benzene

### Step 1. 2,3-Bis(trifluoromethyl)aniline

To methanol solution (80 mL) comprising 2,3-bis(trifluoromethyl)nitrobenzene (2.0 g, manufactured by Apollo Chemical Corp.), 10 wt % Pd/C (100 mg, manufactured by Aldrich Company) was added, followed by stirring under hydrogen atmosphere for four hours. Upon the completion of stirring, the reaction solution was filtered using Celite, and the filtrate was concentrated under reduced pressure to obtain the title compound (1.61 g).

¹H-NMR (DMSO-d₆): 7.41 (1H, brt, J=8.04), 7.15 (1H, d, J=8.04), 7.03 (1H, d, J=8.04), 6.11 (2H, s).

MASS: 228.2 (M−H), RT: 1.70 min.

### Step 2. 1-Bromo-2,3-bis(trifluoromethyl)benzene

The acetonitrile solution (8.0 mL, manufactured by Kanto Chemical Co., Inc.) comprising 2,3-bis(trifluoromethyl) aniline (192 mg) obtained from the Step 1 was cooled down to 0° C., and then copper (II) bromide (223 mg, manufactured by Wako Pure Chemical Industries, Ltd.) and tert-butyl nitrite (120 μl, manufactured by Tokyo Chemical Industry, Co.) were added thereto. After stirring the mixture at 0° C. for two hours, the reaction was further carried out at room temperature for two hours. In addition, copper (II) bromide (187 mg) and tert-butyl nitrite (100 μl) were further added followed by stirring for two hours. Upon the completion of stirring, the reaction solution was poured over saturated brine, extracted with ethyl acetate, and dried over sodium sulfate. The solids were removed by filtration. After drying under reduced pressure, thus obtained residues were subjected to flash column chromatography (using 10/0 to 10/1 (v/v) hexane/ethyl acetate as an eluent) to obtain the title compound (89.4 mg).

¹H-NMR (CDCl₃): 7.95 (1H, d, J=8.04), 7.82 (1H, d, J=8.07), 7.46 (1H, brt, J=8.04).

### Reference Example 33

### 1-Bromo-3-nitro-2-trifluoromethylbenzene

### Step 1. 4-Chloro-3-nitro-2-trifluoromethyl aniline

To 4N ethyl acetate hydrochloric acid solution (110 mL, manufactured by KOKUSAN CHEMICAL Co., Ltd.) comprising 1-chloro-2,4-dinitro-3-trifluoromethylbenzene (3.0 g, manufactured by Marshallton company), iron powder (619 mg, manufactured by Wako Pure Chemical Industries, Ltd.) was added and the mixture was stirred for five hours. Iron powder was added again (620 mg) and the mixture was stirred for 2.5 hours. Iron powder was added again (300 mg) and the mixture was stirred for 1.5 hours. After concentrating the reaction solution to half or so, it was poured over saturated sodium bicarbonate solution and extracted with ethyl acetate. The organic layer was washed with saturated brine and dried over sodium sulfate. The solids were removed by filtration, and the filtrate was dried under reduced pressure to obtain the title compound (2.78 g)

¹H-NMR (CDCl₃): 7.38 (1H, d, J=8.79), 6.79 (1H, d, J=8.76), 4.55 (2H, s).

MASS: 239.1 (M−H), RT: 1.64 min.

### Step 2. 3-Nitro-2-trifluoromethyl aniline

To isopropanol solution (120 mL, manufactured by Kanto Chemical Co., Inc.) comprising 4-chloro-3-nitro-2-trifluoromethyl aniline (2.78 g) obtained from the Step 1, bis(dibenzylideneacetone)palladium (0) (1.33 g, manufactured by Tokyo Chemical Industry, Co.), (2-biphenyl)dicyclohexylphosphine (2.43 g, manufactured by Aldrich Company), and potassium phosphate (3.20 g, manufactured by Wako Pure Chemical Industries, Ltd.) were added, followed by stirring at 90° C. for fifty-four hours. Upon the completion of reaction, the reaction solution was filtered using Celite, and the filtrate was concentrated under reduced pressure. Thus obtained residues were subjected to flash column chromatography (using 20/1 to 4/1 (v:v) hexane/ethyl acetate as an eluent) to obtain the title compound (2.42 g).

¹H-NMR (CDCl₃): 7.32 (1H, d, J=8.04), 6.91-6.83 (2H, m), 4.65 (2H, s).

MASS: 205.1 (M−H), RT: 1.44 min.

### Step 3. 1-Bromo-3-nitro-2-trifluoromethylbenzene

The acetonitrile solution (100 mL, manufactured by Kanto Chemical Co., Inc.) comprising 3-nitro-2-trifluoromethyl aniline (2.20 g) obtained from the Step 2 was cooled down to 0° C., and then copper (II) bromide (2.86 g, manufactured by Wako Pure Chemical Industries, Ltd.) and tert-butyl nitrite (1.53 mL, manufactured by Tokyo Chemical Industry, Co.) were added thereto. After stirring the mixture at 0° C. for forty-five minutes, the reaction was further carried out at room temperature for two hours. In addition, copper (II) bromide (950 mg) and tert-butyl nitrite (500 μl) were further added followed by stirring for forty-five minutes at room temperature. Upon the completion of reaction, the reaction solution was poured over saturated brine, extracted with diethyl ether, and dried over sodium sulfate. The solids were removed by filtration, and the filtrate was dried under reduced pressure to obtain the title compound (2.89 g).

MASS: N.D., RT: 1.76 min. (N.D. means that molecular weight was impossible to detect)

Except that any of the starting materials shown in Table 10 is used, the compounds described in Table 10 were synthesized in the same manner as Reference example 31. Meanwhile, the compounds described to have a supplier “syn” are the compounds which had been synthesized in any of Reference example 32 or 33.

TABLE 10

| ref. | SM. | Supplier | Structure | ¹H-NMR (CDCl₃) |
|---|---|---|---|---|
| 34 | 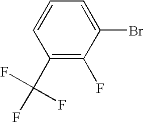 | Ald | 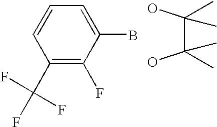 | 7.93 (1H, ddd, J = 7.32, J = 5.49, J = 1.82), 7.74-7.65 (1H, m), 7.28-7.19 (1H, m), 1.37 (12H, s). |
| 35 | 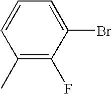 | Apollo | 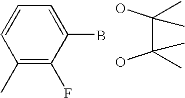 | 7.54 (1H, ddd, J = 7.32, J = 5.49, J = 1.83), 7.31-7.23 (1H, m), 7.03 (1H, t, J = 7.32), 2.26 (3H, d, J = 2.19), 1.36 (12H, s). |
| 36 | 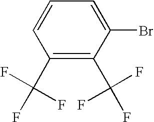 | syn. | 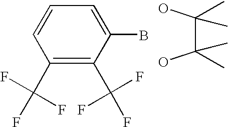 | 7.85 (1H, d, J = 8.04), 7.74 (1H, d, J = 7.32), 7.63 (1H, t, J = 7.68), 1.38 (12H, s). |
| 37 | 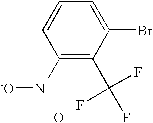 | syn. | 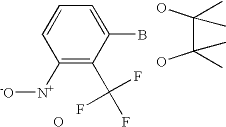 | 7.80 (1H, d, J = 7.32), 7.74 (1H, dd, J = 7.32, J = 7.68), 7.67 (1H, d, J = 7.68), 1.39 (12H, s). |

### Reference Example 38

### Step 1. Methyl 3′-fluoro-2-methyl-2′-(trifluoromethyl)biphenyl-4-carboxylate

To a mixture solution of 2.0 mL toluene and 0.2 mL water comprising methyl 4-iodo-3-methylbenzoate (317 mg, manufactured by Wako Pure Chemical Industries, Ltd.), 3-fluoro-2-trifluoromethylphenylboronic acid pinacol ester obtained from the Reference example 31 (500 mg), palladium acetate (51.4 mg, manufactured by Kanto Chemical Co., Inc.), 2-dicyclohexylphosphino-2′-6′-dimethoxybiphenyl (188 mg, manufactured by Aldrich Company) and potassium phosphate (488 mg, manufactured by Wako Pure Chemical Industries, Ltd.) were added, followed by stirring at 100° C. for twelve hours. Upon the completion of stirring, the reaction solution was filtered using Celite, and the filtrate was concentrated under reduced pressure. Thus obtained residues were subjected to flash column chromatography (using 200:1 (v/v) to 100:1 (v/v) hexane/ethyl acetate as an eluent) to obtain the title compound (333 mg). 0.55 (1H, ddd, J=7.68, J=5.13), 7.28-7.20 (1H, m), 7.18 (1H, d, J=7.68), 6.97 (1H, d, J=7.68), 3.94 (3H, s), 2.11 (3H, s).

### Step 2.

### 3′-Fluoro-2-methyl-2′-(trifluoromethyl)biphenyl-4-carboxylic acid

To methanol solution (3.0 mL) comprising methyl 3′-fluoro-2-methyl-2′-(trifluoromethyl)biphenyl-4-carboxylate obtained from the Step 1, 5N sodium hydroxide solution (1.0 mL, manufactured by Wako Pure Chemical Industries, Ltd.) was added, followed by stirring for twelve hours. Upon the completion of stirring, the reaction solution was concentrated under reduced pressure. 1N hydrochloric acid solution was added thereto and the resulting product was collected by filtration, dried under reduced pressure to obtain the title compound (209 mg).

MASS: 297.2 (M−H), RT: 1.74 min.

### Reference Example 39

### 3′-Chloro-2′-fluoro-2-methylbiphenyl-4-carboxylic acid

To DMF solution (38 mL) comprising 4-bromo-3-methylbenzoic acid (821 mg, manufactured by Wako Pure Chemical Industries, Ltd.), 3-chloro-2-fluorophenylboronic acid (1.0 g, manufactured by Aldrich Company), [1,1′-bis(diphenylphosphino)ferrocene]dichloropalladium-dichloromethane complex (1:1) (619 mg, manufactured by Aldrich Company), and cesium carbonate (1.87 g, manufactured by Kanto Chemical Co., Inc.) were added, followed by stirring at 120° C. for fifteen hours. Upon the completion of stirring, the reaction solution was filtered using Celite, and the filtrate was concentrated under reduced pressure. To thus obtained residues 1N hydrochloric acid solution (manufactured by Wako Pure Chemical Industries, Ltd.) was added, and extracted with ethyl acetate. The organic layer was dried over magnesium sulfate and the solids were removed. Thus obtained residues were subjected to flash column chromatography (using 10:1 (v/v) to 3:1 (v/v) hexane/ethyl acetate as an eluent) to obtain the title compound (717 mg)

MASS: 263.2 (M−H), RT: 1.77 min.

### Reference Example 40

### Methyl 3,5-dimethyl-4-iodobenzoate

### Step 1. 4-Amino-3,5-dimethylbenzoic acid

To methanol solution (50 mL) comprising 3,5-dimethyl-4-nitrobenzoic acid (2.43 g, manufactured by Lancaster Chemicals), a big spoonful of 10% palladium-carbon (manufactured by Merck Company) was added and the mixture was stirred under hydrogen atmosphere at room temperature for 14 hours. After the filtration, the filtrate was concentrated to obtain the title compound.

MASS: 166.1 (M−H), RT: 2.50 min. (Condition (B) described above)

### Step 2. Methyl 3,5-dimethyl-4-iodobenzoate

To conc. Hydrochloric acid (5 mL) and H₂O (15 mL) solution comprising 4-amino-3,5-dimethylbenzoic acid (2.71 g) obtained from the Step 1, sodium nitrite (748 mg, manufactured by Wako Pure Chemical Industries, Ltd.) was added followed by dropwise addition of H₂O (10 mL) solution comprising potassium iodide (3.58 g, manufactured by Merck Company). After stirring the mixture for 15 hours, the mixture was extracted with ethyl acetate, and the organic layer was washed with saturated sodium bicarbonate solution and saturated brine, and then dried over magnesium sulfate. The solids were removed by filtration, and the filtrate was dried under reduced pressure. To methanol solution (60 mL) of the obtained residues, thionyl chloride (3 mL, manufactured by Wako Pure Chemical Industries, Ltd.) was added dropwise at 0° C. After stirring at room temperature for about 17 hours, the reaction was quenched by using H₂O. The reaction solution was extracted with dichloromethane, and the organic layer was washed with saturated brine, and then dried over magnesium sulfate. Thus obtained residues were subjected to flash column chromatography (using 15:1 (v/v) hexane/ethyl acetate as an eluent) to obtain the title compound (1.52 g).

¹H-NMR (CDCl₃): 7.74 (2H, s), 3.90 (3H, s), 2.51 (6H, s).

Except that any of the starting materials shown in Table is used, the carboxylic acids described in Table 11 were synthesized in the same manner as Reference example 9, 38 and 39. The compounds described to have “ref. 9” for “condition” in Table 11 were synthesized according to the method described in Reference example 9. Similarly, the compounds described to have “ref. 38” or “ref. 39” for “condition” were synthesized according to the method described in Reference example 38 or 39 above. Further, the compounds described to have a supplier “syn” for benzoic acid methyl ester in Table 11 were synthesized according to the method described in any of Reference example 7, 8 or 40 above.

TABLE 11

| | condi- | | | |
|---|---|---|---|---|
| ref. | tion | SM. | Supplier | SM. |
| 41 | ref.9 | 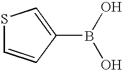 | TCI | 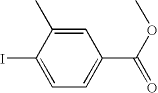 |
| 42 | ref.9 | 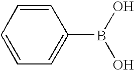 | TCI | 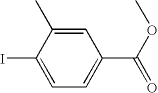 |
| 43 | ref.9 | 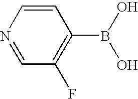 | Frontier | 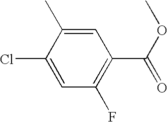 |
| 44 | ref.9 | 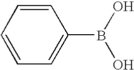 | TCI | 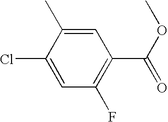 |
| 45 | ref.9 | 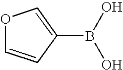 | Ald | 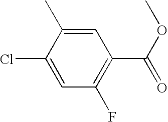 |
| 46 | ref.9 | 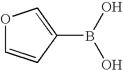 | Ald | 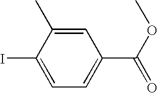 |
| 47 | ref.9 | 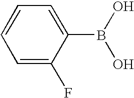 | WAKO | 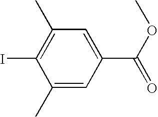 |
| 48 | ref.9 | 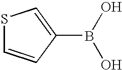 | TCI | 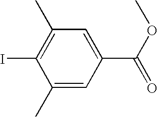 |
| 49 | ref.9 | 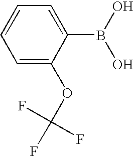 | WAKO | 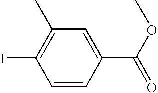 |
| 50 | ref.9 | 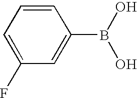 | WAKO | 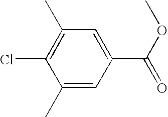 |
| 51 | ref.9 | 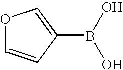 | Ald | 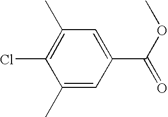 |
| 52 | ref.39 | 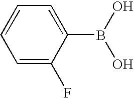 | WAKO | 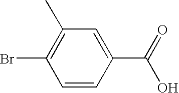 |
| 53 | ref.39 | 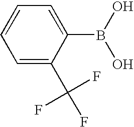 | WAKO | 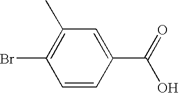 |
| 54 | ref.9 | 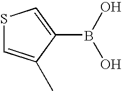 | Ald | 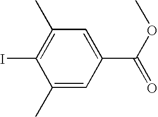 |
| 55 | ref.9 | 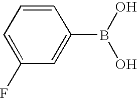 | WAKO | 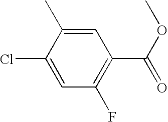 |
| 56 | ref.9 | 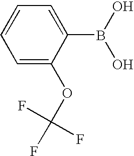 | WAKO | 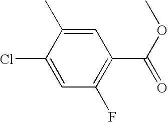 |
| 57 | ref.9 | 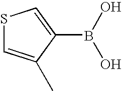 | Ald | 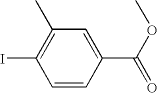 |
| 58 | ref.9 | 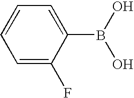 | WAKO | 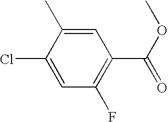 |
| 59 | ref.9 | 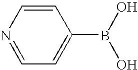 | WAKO | 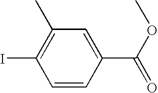 |
| 60 | ref.9 | 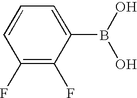 | Ald | 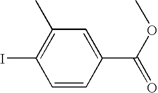 |
| 61 | ref.9 | 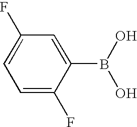 | Ald | 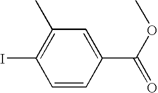 |
| 62 | ref.9 | 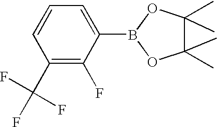 | syn. | 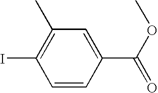 |
| 63 | ref.9 | 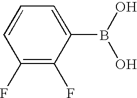 | Ald | 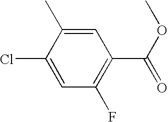 |
| 64 | ref.9 | 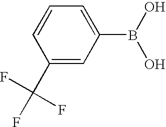 | WAKO | 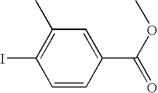 |
| 65 | ref.39 | 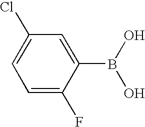 | Ald | 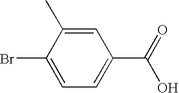 |
| 66 | ref.38 | 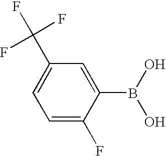 | Ald | 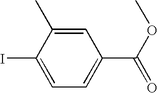 |
| 67 | ref.38 | 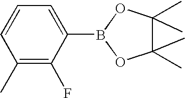 | syn. | 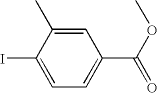 |
| 68 | ref.38 | 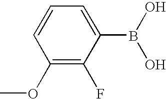 | Ald | 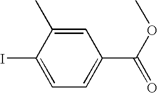 |
| 69 | ref.38 | 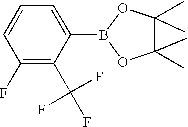 | syn. | 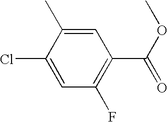 |
| 70 | ref.38 | 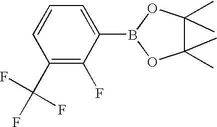 | syn. | 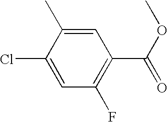 |
| 71 | ref.38 | 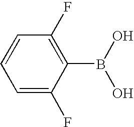 | Ald | 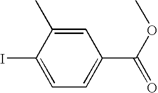 |
| 72 | ref.38 | 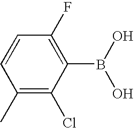 | Ald | 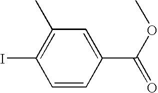 |
| 73 | ref.38 | 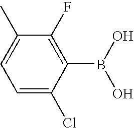 | Ald | 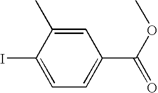 |
| 74 | ref.38 | 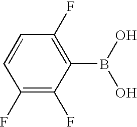 | Ald | 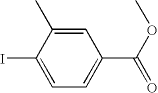 |
| 75 | ref.38 | 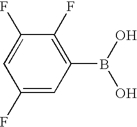 | Ald | 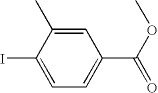 |
| 76 | ref.38 | 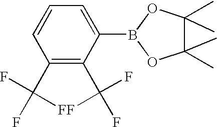 | syn. | 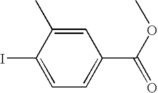 |
| 77 | ref.38 | 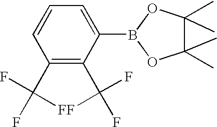 | syn. | 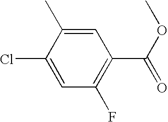 |
| 78 | ref.38 | 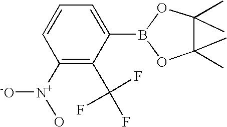 | syn. | 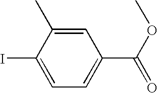 |

| | | | LCMS | |
|---|---|---|---|---|
| | | | (ESI−) | |
| ref. | Supplier | Structure | MASS | RT |
| 41 | WAKO | 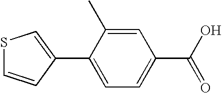 | 217.1 | 1.61 |
| 42 | WAKO | 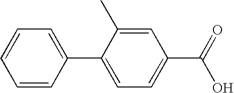 | 211.2 | 1.66 |
| 43 | syn. | 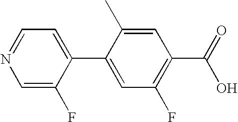 | 247.9 | 1.13 |
| 44 | syn. | 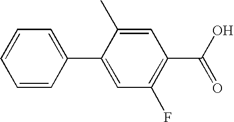 | 229.0 | 1.60 |
| 45 | syn. | 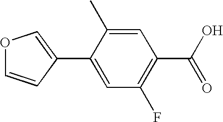 | 219.1 | 1.47 |
| 46 | WAKO | 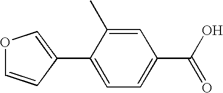 | 201.1 | 1.48 |
| 47 | syn. | 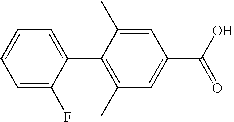 | 243.2 | 1.72 |
| 48 | syn. | 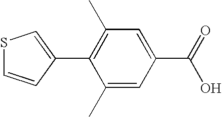 | 231.2 | 1.71 |
| 49 | WAKO | 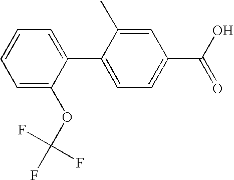 | 295.2 | 1.79 |
| 50 | syn. | 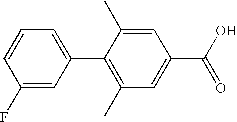 | 243.3 | 1.73 |
| 51 | syn. | 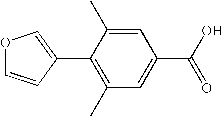 | 215.2 | 1.59 |
| 52 | WAKO | 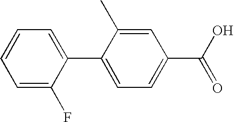 | 229.0 | 1.60 |
| 53 | WAKO | 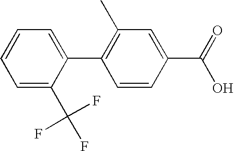 | 278.9 | 1.71 |
| 54 | syn. | 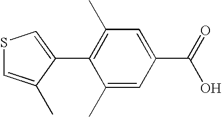 | 245.2 | 1.81 |
| 55 | syn. | 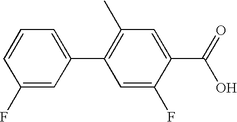 | 247.2 | 1.67 |
| 56 | syn. | 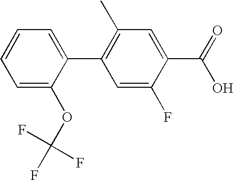 | 313.0 | 1.73 |
| 57 | WAKO | 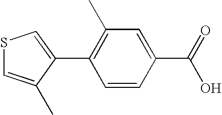 | 231.0 | 1.66 |
| 58 | syn. | 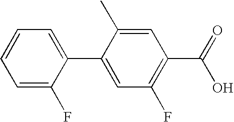 | 247.2 | 1.64 |
| 59 | WAKO | 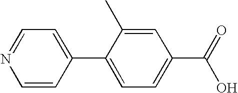 | 211.9 | 0.72 |
| 60 | WAKO | 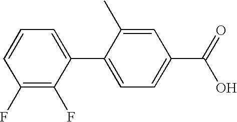 | 247.0 | 1.61 |
| 61 | WAKO | 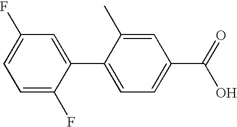 | 247.0 | 1.61 |
| 62 | WAKO | 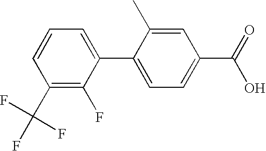 | 297.0 | 1.78 |
| 63 | syn. | 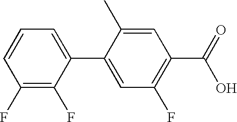 | 265.2 | 1.66 |
| 64 | WAKO | 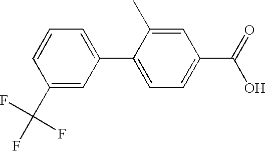 | 279.0 | 1.81 |
| 65 | WAKO | 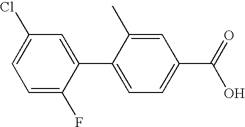 | 263.2 | 1.78 |
| 66 | WAKO | 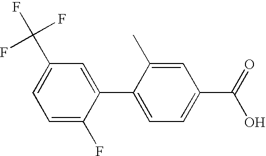 | 297.2 | 1.79 |
| 67 | WAKO | 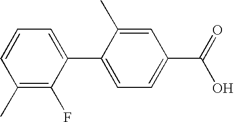 | 243.2 | 1.78 |
| 68 | WAKO | 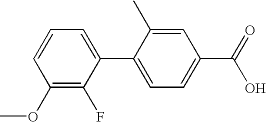 | 259.3 | 1.58 |
| 69 | syn. | 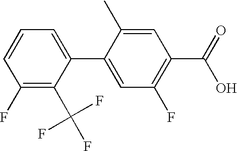 | 315.2 | 1.72 |
| 70 | syn. | 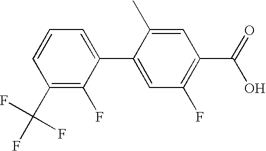 | 315.1 | 1.77 |
| 71 | WAKO | 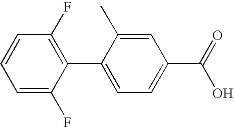 | 247.2 | 1.61 |
| 72 | WAKO | 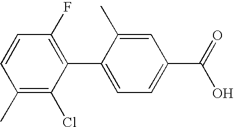 | 277.0 | 1.79 |
| 73 | WAKO | 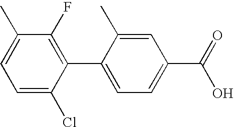 | 277.0 | 1.79 |
| 74 | WAKO | 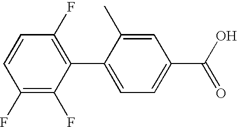 | 265.1 | 1.60 |
| 75 | WAKO | 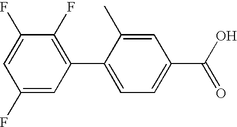 | 265.1 | 1.68 |
| 76 | WAKO | 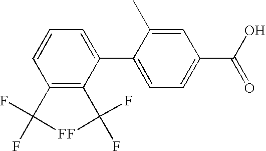 | 324.1 | 1.83 |
| 77 | syn. | 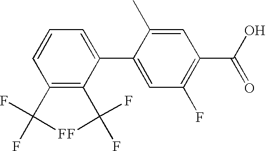 | 365.1 | 1.78 |
| 78 | WAKO | 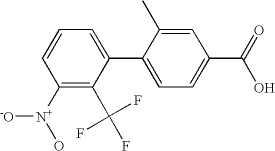 | 324.1 | 1.60 |

In addition, for Reference example 72 to 75, purification was carried out according to the method C described above (i.e., purification method 4).

### Reference Example 79

### Methyl 3-chloro-4-trifluoromethanesulfonyloxybenzoate

Step 1: To methanol solution (150 mL) comprising 3-chloro-4-hydroxybenzoic acid (7.31 g, manufactured by Tokyo Chemical Industry, Co.), thionyl chloride (15 mL, manufactured by Wako Pure Chemical Industries, Ltd.) was added and the mixture was stirred overnight at room temperature. Upon the completion of the reaction, the mixture was added with water, extracted with ethyl acetate and the organic layer was washed with water. After drying over magnesium sulfate, solids were removed by filtration, and the filtrate was dried under reduced pressure to obtain the residues (7.47 g).

Step 2. To dichloromethane solution (30 mL) comprising the residues (2.0 g) of the Step 1 above, triethylamine (1 mL, manufactured by Wako Pure Chemical Industries, Ltd.), and anhydrous trifluoromethane sulfonic acid (2.8 mL, manufactured by Tokyo Chemical Industry, Co.) were added, followed by stirring overnight at room temperature. The mixture was added with water, extracted with dichloromethane. The organic layer was washed with water and dried over magnesium sulfate, and the solids were removed by filtration. The filtrate was dried under reduced pressure and subjected to purification by using Yamazen Flash Column System (using 3 L High Flash column, and 10:1 (v/v) hexane/ethyl acetate as an eluent) to obtain the title compound (2.94 g).

¹H-NMR (CDCl₃): 8.20 (1H, brs), 8.01 (1H, d, J=9.0), 7.43 (1H, d, J=9.0), 3.93 (3H, s)

Except that any of the starting materials shown in Table 12 is used, the carboxylic acids described in Table 12 were synthesized in the same manner as Reference example 9, 38 and 39. The compounds described to have “ref. 9” for “condition” in Table 12 were synthesized according to the method described in Reference example 9. Similarly, the compounds described to have “ref. 38” or “ref. 39” for “condition” were synthesized according to the method described in Reference example 38 or 39 above. Further, the compounds described to have a supplier “syn” for the benzoic acid methyl ester in Table 12 were synthesized according to the method described in Reference example 9 or 79 above.

TABLE 12

| | condi- | | | |
|---|---|---|---|---|
| ref. | tion | SM. | Supplier | SM. |
| 80 | ref.9 | 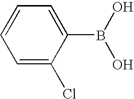 | Ald | 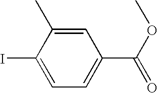 |
| 81 | ref.9 | 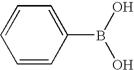 | Ald | 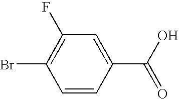 |
| 82 | ref.9 | 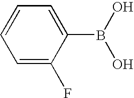 | WAKO | 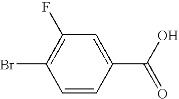 |
| 83 | ref.9 | 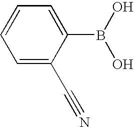 | Ald | 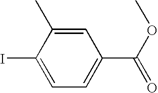 |
| 84 | ref. 39 | 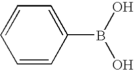 | Ald | 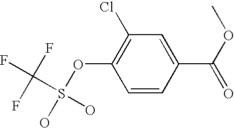 |
| 85 | ref. 39 | 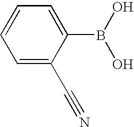 | Ald | 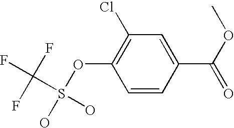 |
| 86 | ref. 38 | 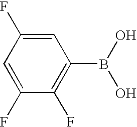 | Ald | 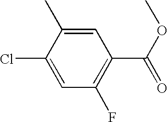 |
| 87 | ref. 38 | 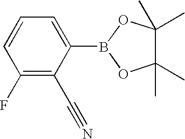 | Combi- Blocks | 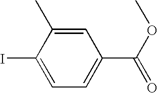 |
| 88 | ref. 38 | 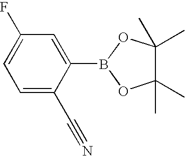 | Frontier | 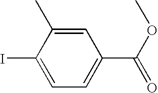 |

| | | | LCMS | |
|---|---|---|---|---|
| | | | (ESI−) | |
| ref. | Supplier | Structure | MASS | RT |
| 80 | WAKO | 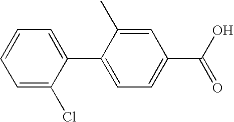 | 245.2 | 4.38 |
| 81 | WAKO | 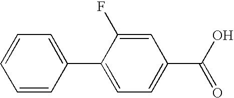 | 251.0 | 1.54 |
| 82 | WAKO | 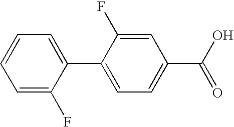 | 233.1 | 1.51 |
| 83 | WAKO | 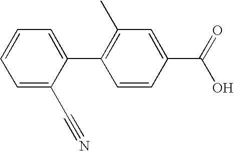 | 236.1 | 1.39 |
| 84 | syn. | 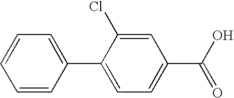 | 231.0 | 4.40 |
| 85 | syn. | 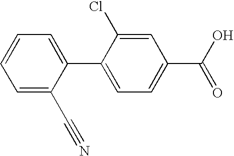 | 256.0 | 1.40 |
| 86 | syn. | 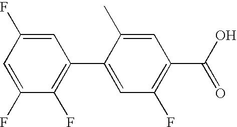 | 283.0 | 1.66 |
| 87 | WAKO | 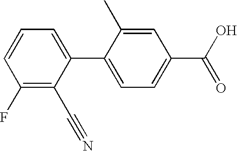 | 254.1 | 1.42 |
| 88 | WAKO | 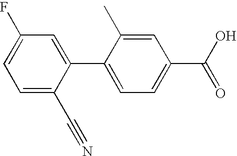 | 254.1 | 1.42 |

In addition, for Reference example 80 to 84, “LCMS” was carried out according to the Condition B described above.

### Reference Example 89

### Step 1. Methyl 3′-amino-2-methyl-2′-trifluoromethylbiphenyl-4-carboxylate

To methanol solution (5 mL, manufactured by Wako Pure Chemical Industries, Ltd.) comprising methyl 2-methyl-3′-nitro-2′-trifluoromethylbiphenyl-4-carboxylate mg) obtained from the synthetic process of the Reference example 78, 10 wt % Pd/C (30 mg, manufactured by Aldrich Company) was added, followed by stirring under hydrogen atmosphere for forty-two hours. Upon the completion of reaction, the reaction solution was filtered using Celite, and the filtrate was concentrated under reduced pressure to obtain the title compound mg).

¹H-NMR (CDCl₃): 7.90 (1H, brs), 7.85 (1H, dd, J=8.04, J=1.47), 7.29-7.23 (1H, m), 7.15 (1H, d, J=8.04), 6.76 (1H, d, J=8.43), 6.44 (1H, d, J=7.68), 4.33 (2H, s), 3.93 (3H, s), 2.12 (3H, s).

MASS: 310.2 (M+H), RT: 1.80 min.

### Step 2. Methyl 3′-chloro-2-methyl-2′-trifluoromethylbiphenyl-4-carboxylate

The acetonitrile solution (4.5 mL, manufactured by Kanto Chemical Co., Inc.) comprising methyl 3′-amino-2-methyl-2′-trifluoromethylbiphenyl-4-carboxylate mg) obtained from the Step 1 was cooled down to 0° C., and then copper (II) bromide (76.6 mg, manufactured by Wako Pure Chemical Industries, Ltd.) and tert-butyl nitrite (68.3 μl, manufactured by Tokyo Chemical Industry, Co.) were added thereto. After stirring the mixture at 0° C. for thirty minutes, the reaction was further carried out at room temperature for eighty minutes. Upon the completion of stirring, the reaction solution was poured over saturated brine, extracted with ethyl acetate, and dried over sodium sulfate. The solids were removed by filtration, and the filtrate was dried under reduced pressure and subjected to flash column chromatography (using 100/1 (v:v) hexane/ethyl acetate as an eluent) to obtain the title compound (93.4 mg)

¹H-NMR (CDCl₃): 7.93 (1H, s), 7.88 (1H, brd, J=7.68), 7.57 (1H, d, J=8.04), 7.47 (1H, t, J=8.04), 7.14 (1H, d, J=8.04), 7.06 (1H, d, J=7.50), 3.93 (3H, s), 2.10 (3H, s).

### Step 3.

### 3′-Chloro-2-methyl-2′-(trifluoromethyl)biphenyl-4-carboxylic acid

To methanol solution (3.0 mL, manufactured by Wako Pure Chemical Industries, Ltd.) comprising methyl 3′-chloro-2-methyl-2′-(trifluoromethyl)biphenyl-4-carboxylate (93.4 mg) obtained from the Step 2, 5N sodium hydroxide solution (1.0 mL, manufactured by Wako Pure Chemical Industries, Ltd.) was added, followed by stirring for 12 hours. Upon the completion of the reaction, the reaction system was concentrated and added with 1N hydrochloric acid solution. The resulting solids were collected by filtration, washed with hexane and dried under reduced pressure to obtain the title compound (61.9 mg).

MASS: 313.0 (M−H), RT: 1.82 min.

Except that any of the starting materials shown in Table 13 is used, the compounds described in Table 13 were synthesized in the same manner as Step 2 and Step 3 of Reference example 89. Meanwhile, for Reference example 91 in Table 13, potassium iodide was used instead of copper (II) bromide for the step which corresponds to the Step 2 of Reference example 89 and the reaction temperature was 70° C. instead of the room temperature.

TABLE 13

| | | | LCMS | |
|---|---|---|---|---|
| | | | (ESI−) | |
| ref. | SM. | Structure | MASS | RT |
| 90 | 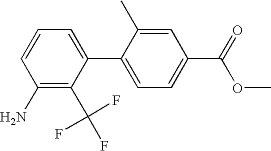 | 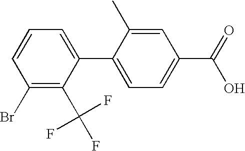 | 357.0 | 1.83 |
| 91 | 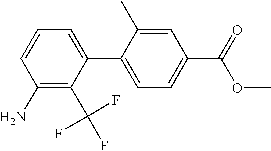 | 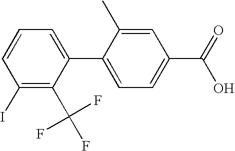 | 405.0 | 1.88 |

Except that any of the starting materials shown in Table is used, the compounds described in Table 14 were synthesized in the same manner as Reference example 20 and Example 1.

TABLE 14

| | | | LCMS (ESI+) | |
|---|---|---|---|---|
| Exp. | SM | Structure | MASS | RT |
| 12 | 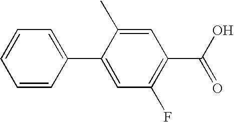 | 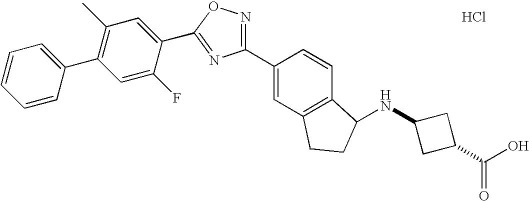 | 484.4 | 1.56 |
| 13 | 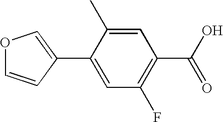 | 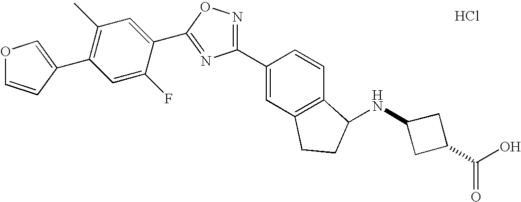 | 474.4 | 1.45 |
| 14 | 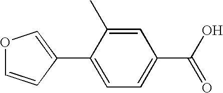 | 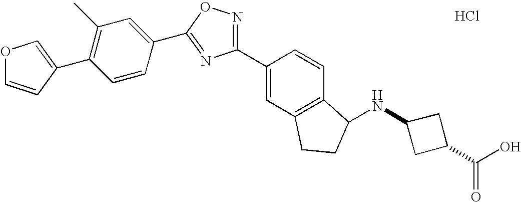 | 456.4 | 1.39 |
| 15 | 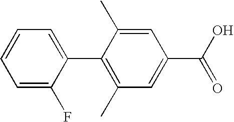 | 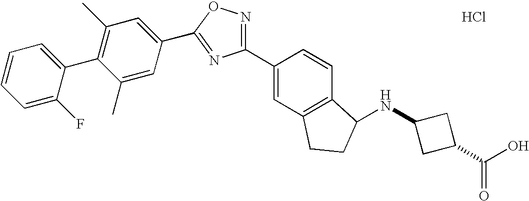 | 498.4 | 1.55 |
| 16 | 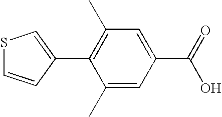 | 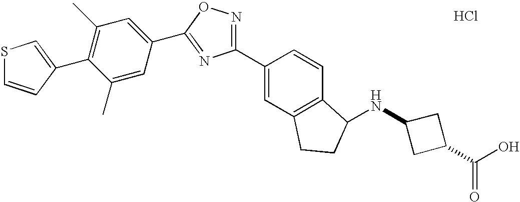 | 486.4 | 1.56 |
| 17 | 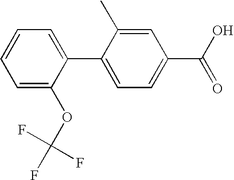 | 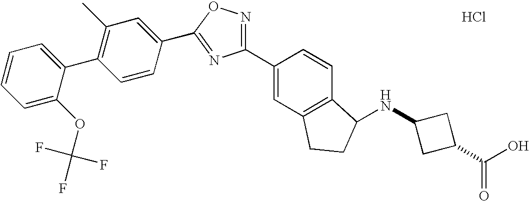 | 550.4 | 1.52 |
| 18 | 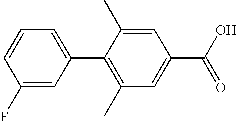 | 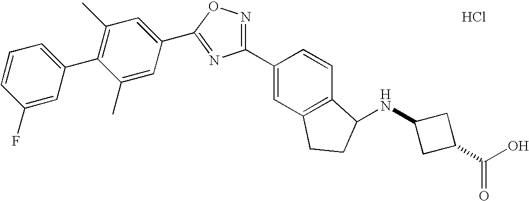 | 498.4 | 1.61 |
| 19 | 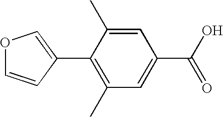 | 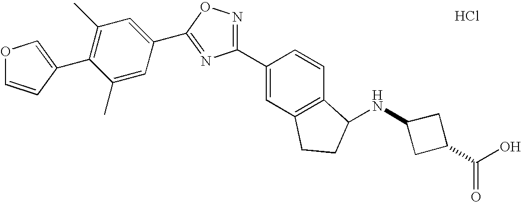 | 438.4 | 1.52 |
| 20 | 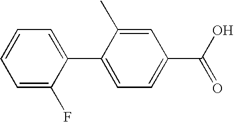 | 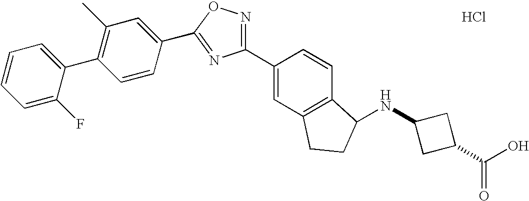 | 484.4 | 1.61 |
| 21 | 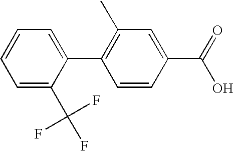 | 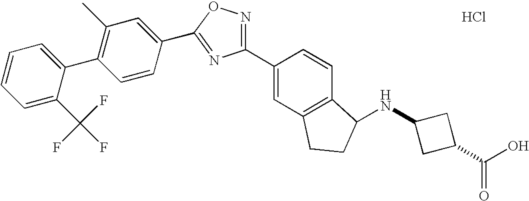 | 534.4 | 1.65 |
| 22 | 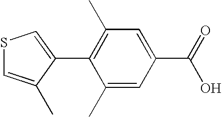 | 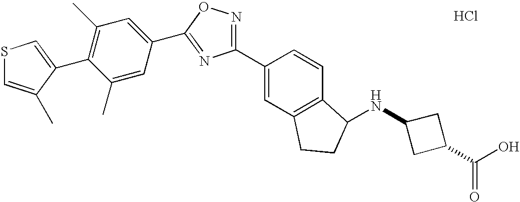 | 500.4 | 1.73 |
| 23 | 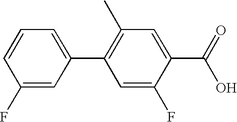 | 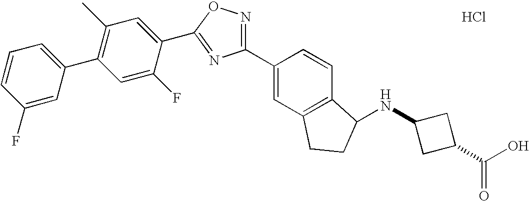 | 502.4 | 1.60 |
| 24 | 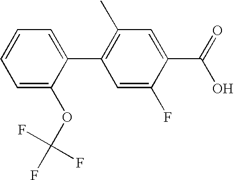 | 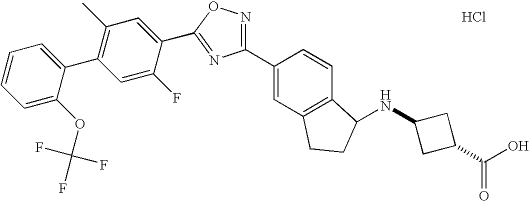 | 568.4 | 1.68 |
| 25 | 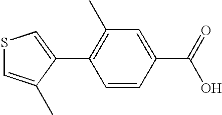 | 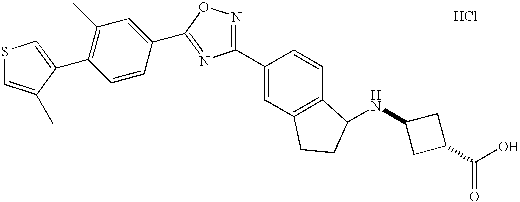 | 486.4 | 1.67 |
| 26 | 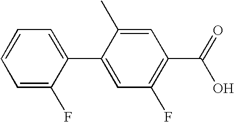 | 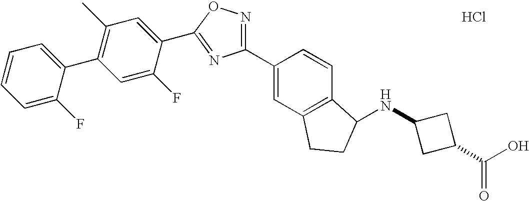 | 502.4 | 1.61 |
| 27 | 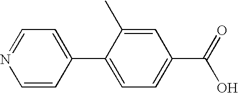 | 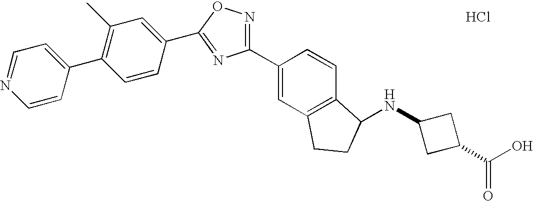 | 467.2 | 1.04 |
| 28 | 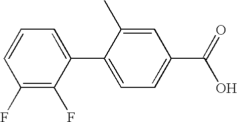 | 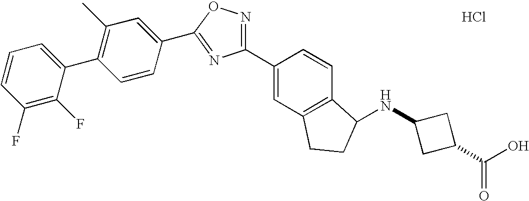 | 502.2 | 1.52 |
| 29 | 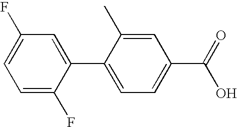 | 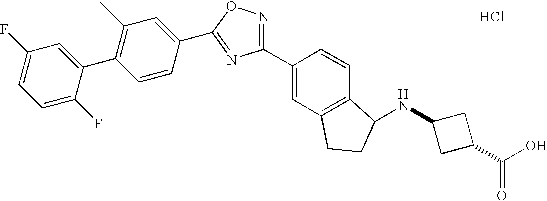 | 502.2 | 1.50 |
| 30 | 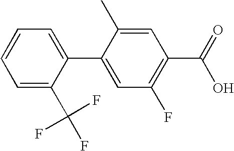 | 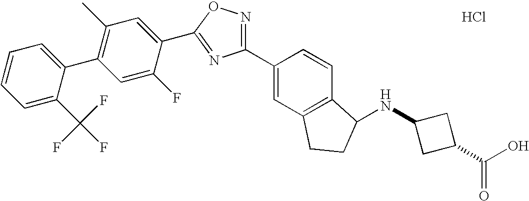 | 552.2 | 1.58 |
| 31 | 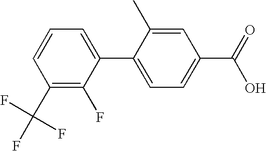 | 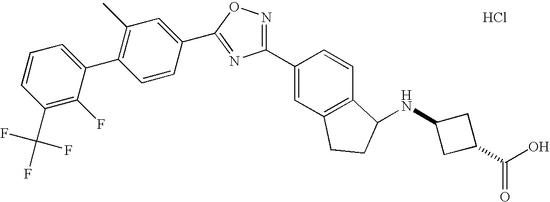 | 552.3 | 1.65 |
| 32 | 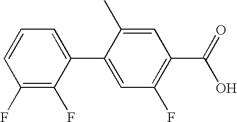 | 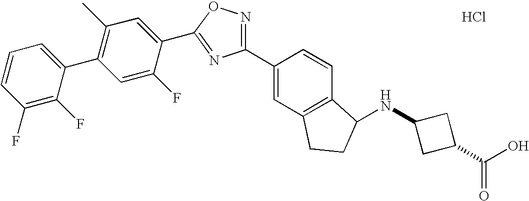 | 520.3 | 1.55 |
| 33 | 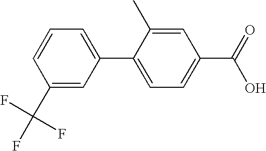 | 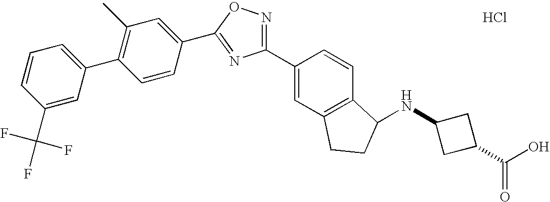 | 534.3 | 1.65 |
| 34 | 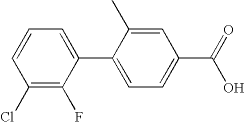 | 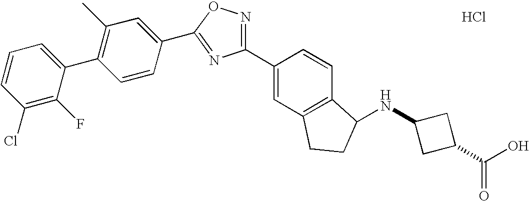 | 518.4 | 1.59 |
| 35 | 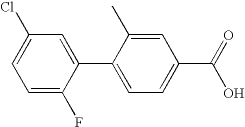 | 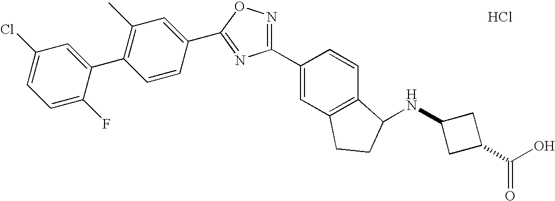 | 518.4 | 1.62 |
| 36 | 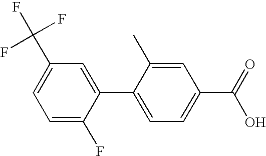 | 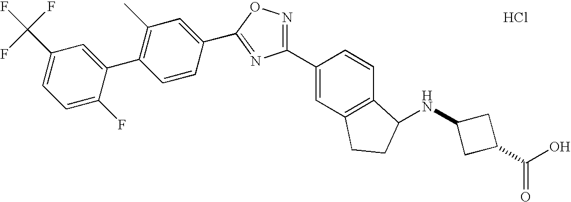 | 552.4 | 1.64 |
| 37 | 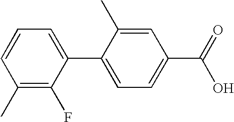 | 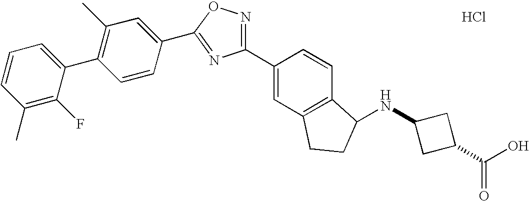 | 498.5 | 1.65 |
| 38 | 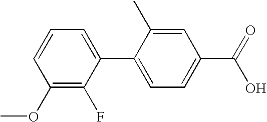 | 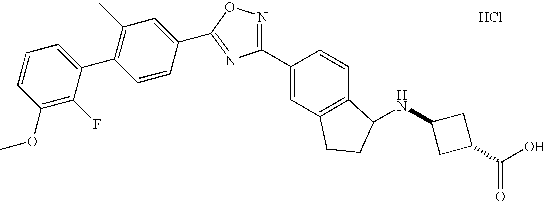 | 514.5 | 1.54 |
| 39 | 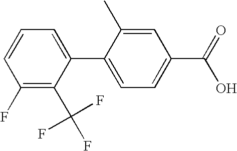 | 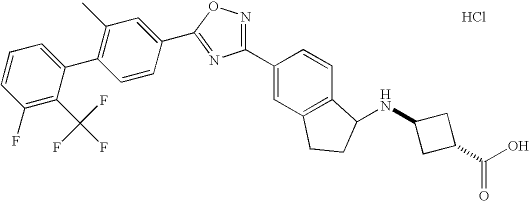 | 552.4 | 1.62 |
| 40 | 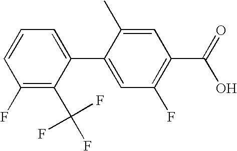 | 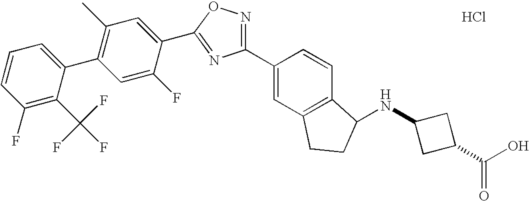 | 570.4 | 1.61 |
| 41 | 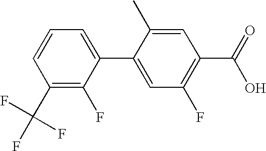 | 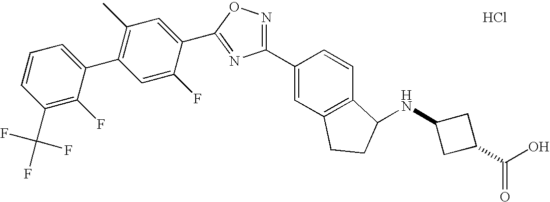 | 570.4 | 1.61 |
| 42 | 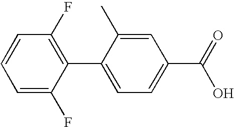 | 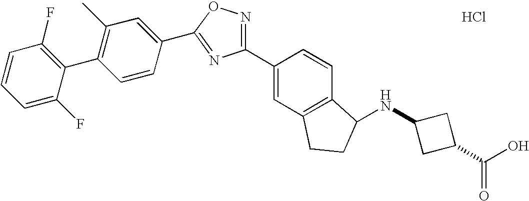 | 502.4 | 1.53 |

### Reference Example 92

### Optically active 1-hydroxy-2,3-dihydro-1H-inden-5-carbonitrile

To ethyl acetate solution (50 mL) comprising 1-oxo-2,3-dihydro-1H-inden-5-carbonitrile (2.56 g) obtained from the Reference example 16, RuCl [(S,S)-Tsdpen] (mesitylene) (51.4 mg, manufactured by Kanto Chemical Co., Inc.), formic acid (3.2 mL, manufactured by Wako Pure Chemical Industries, Ltd.) and triethylamine (3.88 mL, manufactured by Wako Pure Chemical Industries, Ltd.) were added. After stirring the mixture at room temperature overnight, RuCl [(S,S)-Tsdpen] (mesitylene) (50.8 mg) was added thereto and the mixture was stirred for 3.75 hours. After adding sodium bicarbonate solution, the reaction solution was extracted with ethyl acetate. The organic layer was washed with water and dried over magnesium sulfate. The solids were removed by filtration, and the filtrate was dried under reduced pressure to obtain the title compound (2.58 g)

¹H-NMR (CDCl₃): 7.56-7.49 (3H, m), 5.32-5.25 (1H, dd), 3.13-3.03 (1H, m), 2.91-2.80 (1H, m), 2.62-2.52 (1H, m), 2.04-1.92 (1H, m).

### Reference Example 93

### Tert-butyl 1,3-trans-3-(2-nitrophenylsulfonamide)cyclobutane carboxylate

To dry THF (100 mL) solution comprising tert-butyl 1,3-trans-3-aminocyclobutane carboxylate (4.39 g) obtained from the Reference example 6, triethylamine (7.4 mL, manufactured by Wako Pure Chemical Industries, Ltd.) and 2-nitrophenylsulfonyl chloride (7.02 g, manufactured by Aldrich Company) were added at room temperature, followed by stirring for 1.5 hours. The reaction solution was added with water and extracted with ethyl acetate. The organic layer was washed with water and brine, and dried over magnesium sulfate. Thereafter, solids were removed and the filtrate was dried under reduced pressure, and subjected to purification by using Yamazen Flash Column System (using 3 L High Flash column, and 80:20 to 70:30 (v/v) hexane/ethyl acetate as an eluent) to obtain the title compound (6.77 g).

¹H-NMR (CDCl₃): 8.14-8.11 (1H, m), 7.84-7.81 (1H, m), 7.76-7.69 (2H, m), 5.48 (1H, d, J=6.0), 4.14-4.06 (1H, m), 2.89-2.81 (1H, m), 2.45-2.37 (2H, m), 2.19-2.09 (2H, m), 1.41 (9H, s).

### Reference Example 94

### Optically active tert-butyl 1,3-trans-3-(N-(5-cyano-2,3-dihydro-1H-inden-1-yl)-2-nitrophenylsulfonamide)cyclobutane carboxylate

To dry THF (60 mL) solution comprising 1-hydroxy-2,3-dihydro-1H-inden-5-carbonitrile (1.56 g) obtained from the Reference example 92, tert-butyl 1,3-trans-3-(2-nitrophenylsulfonamide)cyclobutane carboxylate (3.19 g) obtained from the Reference example 93 and di-tert-butylazodicarboxylate (3.27 g, manufactured by Aldrich Company), tributylphosphine (1.6 mL, manufactured by Tokyo Chemical Industry, Co.) was added at room temperature, followed by stirring for 2 hours. The reaction solution was concentrated, and subjected to purification by using Yamazen Flash Column System (using 3 L High Flash column, and 8:1 to 6:4 (v/v) hexane/ethyl acetate as an eluent) to obtain the title compound (3.53 g).

MASS: 498.4 (M+H), RT: 5.33 min. (Condition (B) described above)

### Reference Example 95

### Optically active tert-butyl 1,3-trans-3-(5-cyano-2,3-dihydro-1H-inden-1-yl)aminocyclobutane carboxylate

To acetonitrile (30 mL) solution comprising tert-butyl 1,3-trans-3-(N-(5-cyano-2,3-dihydro-1H-inden-1-yl)-2-nitrophenylsulfonamide)cyclobutane carboxylate obtained from the Reference example 94 (1.39 g) and cesium carbonate (2.87 g, manufactured by Kanto Chemical Co., Inc.), thiophenol (518 μl, manufactured by Tokyo Chemical Industry, Co.) was added at room temperature, followed by stirring for 3.5 hours. The reaction solution was concentrated, added with water and extracted with ethyl acetate. The organic layer was washed with water, and dried over magnesium sulfate. Thereafter, solids were removed and the filtrate was dried under reduced pressure, and subjected to purification by using Yamazen Flash Column System (using 3 L High Flash column, and 8:2 to 5:5 (v/v) hexane/ethyl acetate as an eluent) to obtain the title compound (0.89 g).

MASS: 313.2 (M+H), RT: 2.54 min. (Condition (B) described above)

### Reference Example 96

### Optically active tert-butyl 1,3-trans-3-(tert-butoxycarbonyl(5-(N′-hydroxycarbamimidoyl)-2,3-dihydro-1H-inden-1-yl)amino)cyclobutane carboxylate

Except that tert-butyl 1,3-trans-3-(5-cyano-2,3-dihydro-1H-inden-1-yl)aminocyclobutane carboxylate obtained from the Reference example 95 is used, the title compound was obtained in the same manner as Reference examples 18 and 19.

Except that Any of the starting materials shown in Table and the compound obtained from the Reference example 96 are used, the compounds described in Table 15 were synthesized in the same manner as Reference example 20 and Example 1.

TABLE 15

| | | | LCMS (ESI+) | |
|---|---|---|---|---|
| Exp. | SM | Structure | MASS | RT |
| 43 | 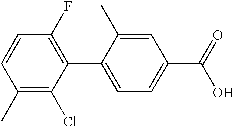 | 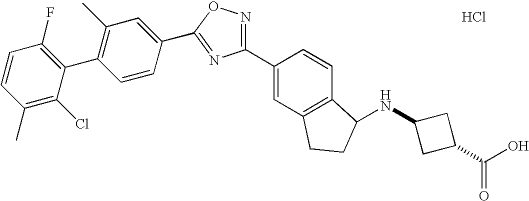 | 532.2 | 1.72 |
| 44 | 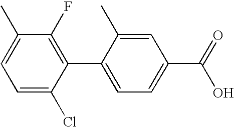 | 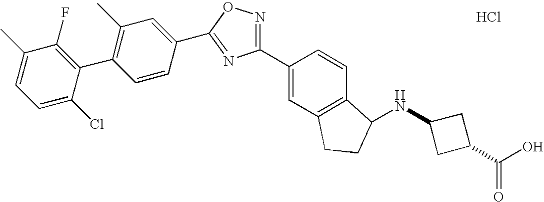 | 532.2 | 1.76 |
| 45 | 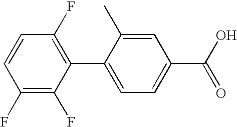 | 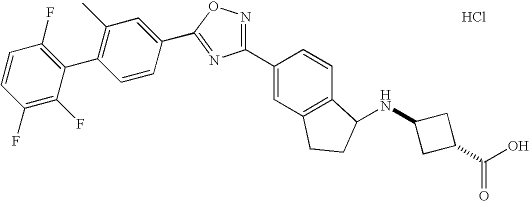 | 520.2 | 1.50 |
| 46 | 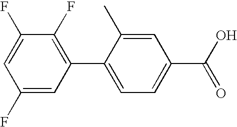 | 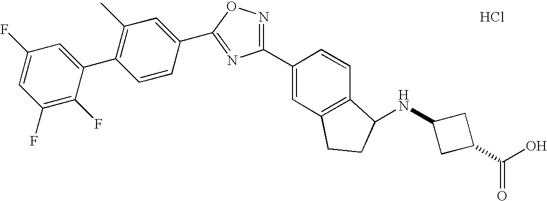 | 520.2 | 1.57 |
| 47 | 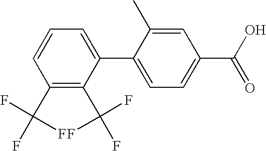 | 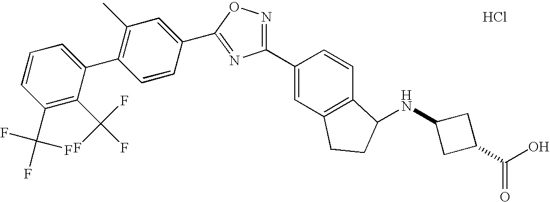 | 602.2 | 1.78 |
| 48 | 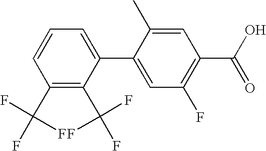 | 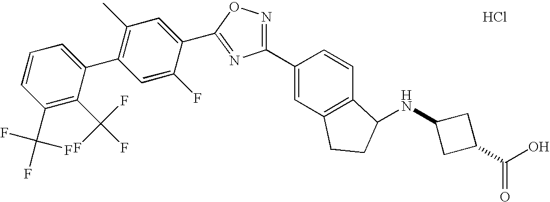 | 620.2 | 1.66 |
| 49 | 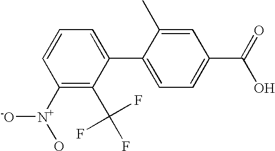 | 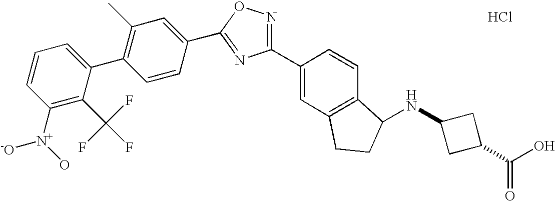 | 579.2 | 1.55 |
| 50 | 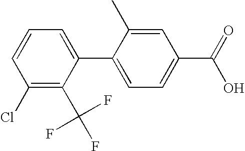 | 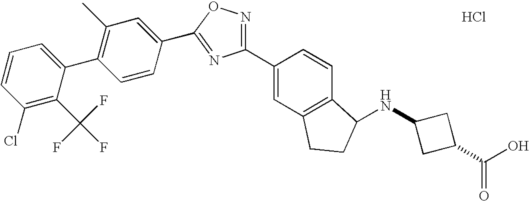 | 568.2 | 1.73 |
| 51 | 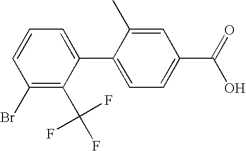 | 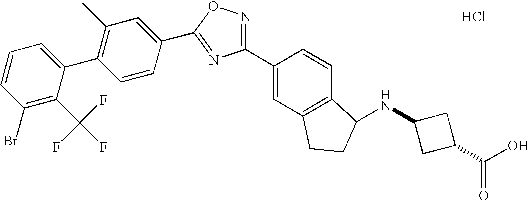 | 612.1 | 1.70 |
| 52 | 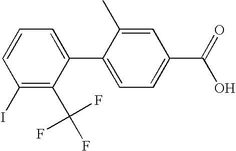 | 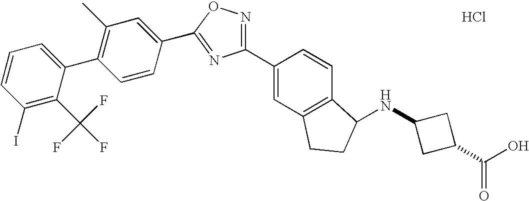 | 660.1 | 1.66 |

### Reference Example 97

### 4-Benzyloxy-6-methyl-2H-pyran-2-one

To acetonitrile (20 mL) solution comprising 4-hydroxy-6-methyl-2H-pyran-2-one (3.0 g, manufactured by Aldrich Company), benzyl bromide (5.7 g, manufactured by Wako Pure Chemical Industries, Ltd.) and DBU (5.34 mL, manufactured by Aldrich Company) were added and the mixture was stirred overnight while refluxing. After cooling, the mixture was concentrated under reduced pressure and subjected to flash column chromatography by using Biotage 40M (using 5:1 to 1:1 (v/v) hexane/ethyl acetate as an eluent) to obtain the title compound (2.48 g).

¹H-NMR (CDCl₃): 7.44-7.32 (5H, m), 5.84-5.83 (1H, m), 5.49 (1H, d, J=2.22), 5.00 (2H, s), 2.20 (3H, s).

### Reference Example 98

### Dimethyl 5-benzyloxy-3-methylphthalate

To 4-benzyloxy-6-methyl-2H-pyran-2-one (2.48 g) obtained from the Reference Example 97, dimethyl acetylene dicarboxylate (2.44 g, manufactured by Tokyo Chemical Industry, Co.) was added and the mixture was stirred overnight at 170° C. After cooling, the mixture was subjected to flash column chromatography by using Biotage 40M (using 6:1 to 4:1 (v/v) hexane/ethyl acetate as an eluent) to obtain the title compound (2.51 g).

¹H-NMR (CDCl₃): 7.43-7.27 (6H, m), 6.98 (1H, d, J=2.19), 5.09 (2H, s), 3.90 (3H, s), 3.87 (3H, s), 2.33 (3H, s).

### Reference Example 99

### 1,2-Bis(hydroxymethyl)-5-benzyloxy-3-methylbenzene

To THF (80 mL) solution comprising dimethyl 5-benzyloxy-3-methylphthalate (2.51 g) obtained from the Reference Example 98, lithium aluminum hydride (909 mg, manufactured by Wako Pure Chemical Industries, Ltd.) was added under ice cooling. After stirring for 7 hours, the mixture was added with saturated brine and extracted with ethyl acetate. Then, the mixture was dried over sodium sulfate, and the solids were removed and the filtrate was dried under reduced pressure to obtain the title compound (2.02 g).

¹H-NMR (CDCl₃): 7.43-7.25 (5H, m), 6.82-6.78 (2H, m), 5.04 (2H, s), 4.68 (2H, s)-4.65 (2H, s), 2.40 (3H, s).

### Reference Example 100

### 1,2-Bis(acetoxymethyl)-5-benzyloxy-3-methylbenzene

To 1,2-bis(hydroxymethyl)-5-benzyloxy-3-methylbenzene (2.02 g) obtained from the Reference Example 99, acetic anhydride (7.4 mL, manufactured by Kanto Chemical Co., Inc.), pyridine (6.3 mL, manufactured by Wako Pure Chemical Industries, Ltd.) and 4-dimethylaminopyridine (95.5 mg, manufactured by Wako Pure Chemical Industries, Ltd.) were added and the mixture was stirred overnight at room temperature. After concentration under reduced pressure, the reaction solution was subjected to flash column chromatography by using Biotage 40M (using 15:1 to 4:1 (v/v) hexane/ethyl acetate as an eluent) to obtain the title compound (2.56 g).

¹H-NMR (CDCl₃): 7.45-7.28 (5H, m), 6.88 (1H, d, J=2.75), 6.83 (1H, d, J=2.75), 5.174 (2H, s), 5.167 (2H, s), 5.06 (2H, s), 2.38 (3H, s), 2.07 (3H, s), 2.05 (3H, s).

### Reference Example 101

### 1,2-Bis(acetoxymethyl)-5-hydroxy-3-methylbenzene

To ethyl acetate solution (100 mL) comprising 1,2-bis(acetoxymethyl)-5-benzyloxy-3-methylbenzene (1.71 g) obtained from the Reference example 100, 10 wt % palladium-carbon (342 mg, manufactured by Aldrich Company) was added and the mixture was stirred under hydrogen atmosphere at room temperature for 3.5 hours. After the filtration, the filtrate was concentrated under reduced pressure, and subjected to flash column chromatography by using Biotage 40M (using 5:1 to 1:1 (v/v) hexane/ethyl acetate as an eluent) to obtain the title compound (590 mg).

¹H-NMR (CDCl₃): 6.76 (1H, d, J=2.58), 6.71 (1H, d, J=2.58), 6.57 (1H, s), 5.17 (4H, s), 2.35 (3H, s), 2.08 (3H, s), 2.06 (3H, s).

### Reference Example 102

### 1,2-Bis(acetoxymethyl)-3-methyl-5-trifluoromethanesulfonyloxybenzene

To chloroform (25 mL) solution comprising 1,2-bis(acetoxymethyl)-5-hydroxy-3-methylbenzene (635 mg) obtained from the Reference example 101, N,N-diisopropylethylamine (652 μl, manufactured by Wako Pure Chemical Industries, Ltd.), and N-phenylbis(trifluoromethanesulfonimide) (manufactured by Tokyo Chemical Industry, Co., 1.17 g) were added at 0° C., followed by stirring overnight at room temperature. Then, N,N-diisopropylethylamine (217 μl), and N-phenylbis(trifluoromethanesulfonimide) (0.45 g) were added, followed by stirring for 6.5 hours at room temperature. Saturated brine was added thereto and the reaction solution was extracted with chloroform. Next, the organic layer was dried over magnesium sulfate. Thereafter, solids were removed and the filtrate was dried under reduced pressure, and the resulting residues were subjected to flash column chromatography by using Yamazen High Flash Column L (using 5:1 (v/v) hexane/ethyl acetate as an eluent) to obtain the title compound (309 mg).

¹H-NMR (CDCl₃): 7.20 (1H, d, J=2.55), 7.12 (1H, d, J=2.55), 5.24 (2H, s), 5.21 (2H, s), 2.47 (3H, s), 2.11 (3H, s), 2.07 (3H, s).

### Reference Example 103

### 3,4-Bis(acetoxymethyl)-5-methylbenzonitrile

To DMF (8.0 mL) solution comprising 1,2-bis(acetoxymethyl)-3-methyl-5-trifluoromethanesulfonyloxybenzene (309 mg) obtained from the Reference example 102, tetrakis(triphenylphosphine)palladium (186 mg, manufactured by Aldrich Company) and zinc cyanide (189 mg, manufactured by Aldrich Company) were added, followed by stirring overnight at 120° C. Then, zinc cyanide (94.4 mg), and tetrakis(triphenylphosphine)palladium (929 mg) were further added, followed by stirring for 3 hours at 120° C. Saturated brine was added thereto and the mixture was extracted with diethyl ether. Next, the organic layer was dried over sodium sulfate. Thereafter, solids were removed and the filtrate was dried under reduced pressure, and the resulting residues were subjected to flash column chromatography by using Yamazen High Flash Column L (using 7:1 to 5:1 (v/v) hexane/ethyl acetate as an eluent) to obtain the title compound (107 mg).

¹H-NMR (CDCl₃): 7.57 (1H, s), 7.49 (1H, s), 5.26 (2H, s), 5.24 (2H, s), 2.47 (3H, s), 2.12 (3H, s), 2.08 (3H, s).

### Reference Example 104

### 3,4-Bis(hydroxymethyl)-5-methylbenzonitrile

To methanol (4.5 mL) solution comprising 3,4-bis(acetoxymethyl)-5-methylbenzonitrile (107 mg) obtained from the Reference example 103, potassium carbonate (141 mg, manufactured by Wako Pure Chemical Industries, Ltd.) was added, followed by stirring for 3 hours at room temperature. Saturated brine was added thereto and the mixture was extracted with ethyl acetate. Next, the organic layer was dried over sodium sulfate. Thereafter, solids were removed and the filtrate was dried under reduced pressure. To the methanol (4.5 mL) solution comprising the resulting residues, potassium carbonate (141 mg) was added and the mixture was stirred for 2.5 hours at room temperature. To the reaction solution, 1N hydrochloric acid and saturated brine were added, and extraction was carried out by using ethyl acetate. Then, the organic layer was dried over sodium sulfate. Thereafter, solids were removed and the filtrate was dried under reduced pressure to obtain the title compound (68.7 mg).

¹H-NMR (CDCl₃): 7.58 (1H, s), 7.43 (1H, s), 4.79 (2H, s), 4.74 (2H, s) 4.67 (2H, s), 2.42 (3H, s).

MASS: 176.0 (M−H), RT: 0.85 min.

### Reference Example 105

### 1,2-Bis(hydroxymethyl)-3-methyl-5-(5-(2-methylbiphenyl-4-yl)-1,2,4-oxadiazol-3-yl)benzene

Step 1. To 3,4-bis(hydroxymethyl)-5-methylbenzonitrile (68.7 mg) obtained from the Reference example 104, hydroxylamine hydrochloric acid salt (58.9 mg, manufactured by Kanto Chemical Co., Inc.), DMF (8.0 mL) and triethylamine (119 μl, manufactured by Wako Pure Chemical Industries, Ltd.) were added. After the filtration, the mixture was stirred overnight at 100° C. Then the reaction solution was dried under reduced pressure.

MASS: 211.1 (M+H), RT: 0.37 min.

Step 2. The resulting compound of the Step 1 is used as a reacting material, the title compound was obtained in the same manner as Step 2 of the Reference example 26.

MASS: 387.2 (M+H), RT: 1.98 min.

### Reference Example 106

### 1,2-Bis(bromomethyl)-3-methyl-5-(5-(2-methylbiphenyl-4-yl)-1,2,4-oxadiazol-3-yl)benzene

1,2-bis(hydroxymethyl)-3-methyl-5-(5-(2-methylbiphenyl-4-yl)-1,2,4-oxadiazol-3-yl)benzene is used as a reacting material, the title compound was obtained in the same manner as the Reference example 29.

¹H-NMR (CDCl₃): 8.14 (1H, s), 8.10-8.065 (2H, m), 8.01 (1H, s), 7.50-7.34 (6H, m), 4.74 (2H, s), 7.41 (2H, s), 2.54 (3H, s), 2.39 (3H, s).

### Example 53

### 1,3-Trans-3-(4-methyl-6-(5-(2-methylbiphenyl-4-yl)-1,2,4-oxadiazol-3-yl)isoindolin-2-yl)cyclobutane carboxylic acid hydrochloric acid salt

### Step 1.

1,2-bis(bromomethyl)-3-methyl-5-(5-(2-methylbiphenyl-4-yl)-1,2,4-oxadiazol-3-yl)benzene obtained from the Reference example 106 is used, synthesis was carried out in the same manner as the Reference example 30, and the resulting compound is used for a further reaction.

Step 2. The resulting compound of the Step 1 is used as a starting material, the title compound was obtained in the same manner as the Example 1.

MASS: 466.2 (M+H), RT: 1.71 min.

Except that the starting material shown in Table 16 is used, the compound described in Table 16 was synthesized in the same manner as Step 2 of the Reference example 26, the Reference Example 29 and the Example 53.

TABLE 16

| | | | LCMS (ESI+) | |
|---|---|---|---|---|
| Exp. | SM | Structure | MASS | RT |
| 54 | 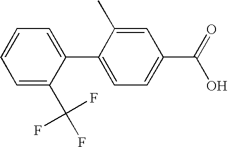 | 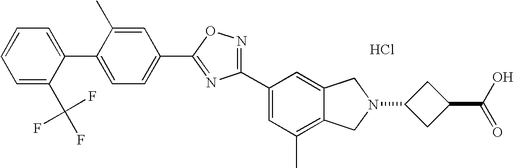 | 534.2 | 1.76 |

### Example 55

### 1,3-Trans-3-(5-(5-(2-chloro-2′-fluorobiphenyl-4-yl)-1,2,4-oxadiazol-3-yl)-2,3-dihydro-1H-inden-1-ylamino)cyclobutane carboxylic acid hydrochloric acid salt

Step 1. To 2-chloro-2′-fluorobiphenyl-4-carboxylic acid (52.6 mg), thionyl chloride (500 μl, manufactured by Wako Pure Chemical Industries, Ltd.) was added, stirred for 2 hours at 120° C. followed by concentration under reduced pressure. Next, tert-butyl 1,3-trans-3-(tert-butoxycarbonyl(5-(N′-hydroxycarbamimidoyl)-2,3-dihydro-1H-inden-1-yl)amino)cyclobutane carboxylate (83.7 mg) obtained from the Reference example 96, dichloromethane (3 mL) and triethylamine (250 μl, manufactured by Wako Pure Chemical Industries, Ltd.) were added thereto, and the mixture was stirred for 15 minutes. The resulting mixture was concentrated under reduced pressure, added with DMF (3 mL) and acetic acid (1 mL, manufactured by Aldrich Company), followed by stirring overnight at 120° C. The reaction solution was extracted with ethyl acetate. Next, the organic layer was washed with water and dried over magnesium sulfate. Thereafter, solids were removed and the filtrate was dried under reduced pressure, and subjected to purification by using Yamazen Flash Column (High Flash Column L, using 94:6 (v/v) hexane/ethyl acetate as an eluent) to obtain the residues (82.1 mg), which were used for the next reaction.

Step 2. By using the resulting product of the Step 1, the title compound was obtained in the same manner as the Example 1.

MASS: 504.5 (M+H), RT: 3.47 min. (Condition (B) described above).

Any of the starting materials shown in Table 17 and the resulting compound obtained from the Reference example 96 are used, the compounds described in Table 17 were synthesized in the same manner as Example 55.

TABLE 17

| | | | LCMS (ESI+) | |
|---|---|---|---|---|
| Exp. | SM | Structure | MASS | RT |
| 56 | 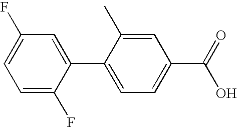 | 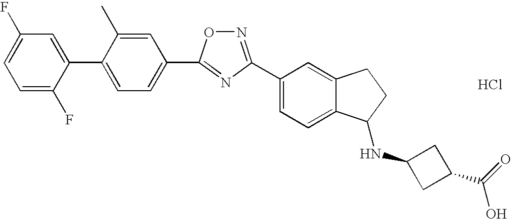 | 502.2 | 1.50 |
| 57 | 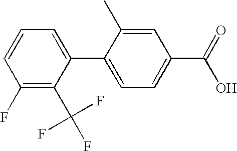 | 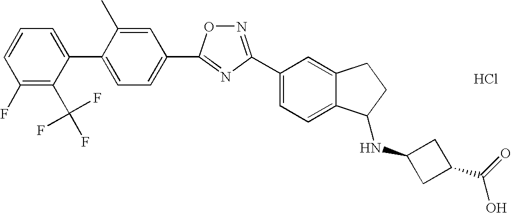 | 552.4 | 1.62 |
| 58 | 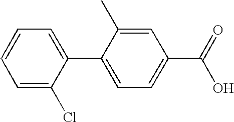 | 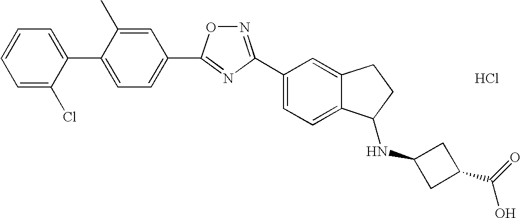 | 500.2 | 1.73 |
| 59 | 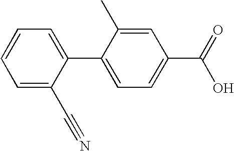 | 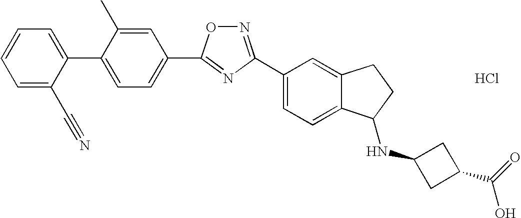 | 491.2 | 1.51 |
| 60 | 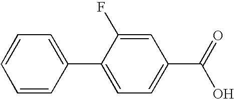 | 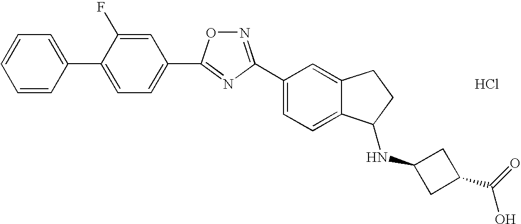 | 470.2 | 1.65 |
| 61 | 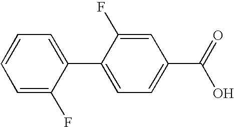 | 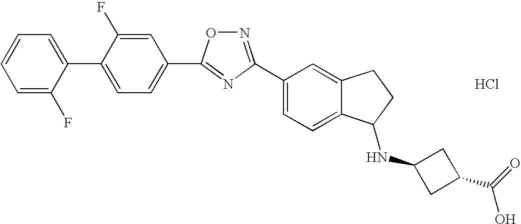 | 488.2 | 1.68 |
| 62 | 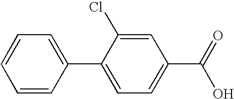 | 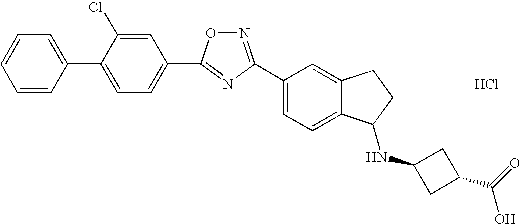 | 486.2 | 1.83 |
| 63 | 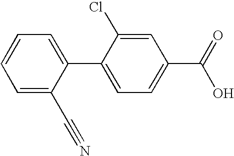 | 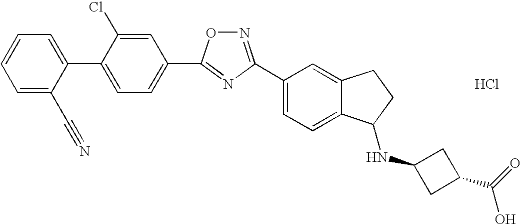 | 511.2 | 1.54 |
| 64 | 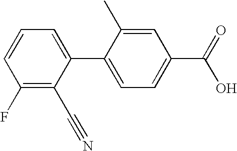 | 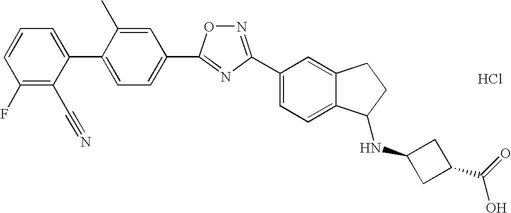 | 509.2 | 1.36 |
| 65 | 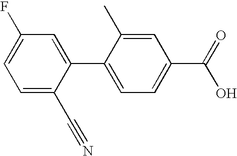 | 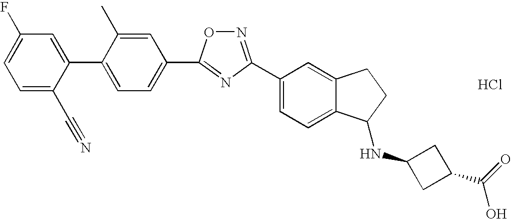 | 509.2 | 1.38 |

### Reference Example 107

### 1,2-Bis(bromomethyl)-4-bromobenzene

By using 1,2-bis(hydroxymethyl)-4-bromobenzene obtained from the Reference example 22, the title compound was obtained in the same manner as the Reference example 29.

¹H-NMR (CDCl₃): 7.56-7.51 (1H, m), 7.49-7.41 (1H, m), 7.27-7.21 (1H, m), 4.59 (2H, s), 4.57 (2H, s).

### Reference Example 108

### Tert-butyl 1,3-trans-3-(5-bromoisoindolin-2-yl)cyclobutane carboxylate

By using 1,2-bis(bromomethyl)-4-bromobenzene obtained from the Reference example 107, the title compound was obtained in the same manner as the Reference example 30.

MASS: 352.0 (M+H), RT: 1.18 min.

### Reference Example 109

### Tert-butyl 1,3-trans-3-(5-cyanoisoindolin-2-yl)cyclobutane carboxylate

By using tert-butyl 1,3-trans-3-(5-bromoisoindolin-2-yl)cyclobutane carboxylate obtained from the Reference example 108, the title compound was obtained in the same manner as the Reference example 16.

MASS: 299.1 (M+H), RT: 0.98 min.

### Reference Example 110

### Tert-butyl 1,3-trans-3-(5-(N′-hydroxycarbamimidoyl)isoindolin-2-yl)cyclobutane carboxylate

By using tert-butyl 1,3-trans-3-(5-cyanoisoindolin-2-yl)cyclobutane carboxylate obtained from the Reference example 109, the title compound was synthesized in the same manner as Step 1 of the Reference example 105.

MASS: 332.1 (M+H), RT: 0.72 min.

Any of the starting materials shown in Table 18 and the resulting compound obtained from the Reference example 110 are used, the compounds described in Table 18 were synthesized in the same manner as Example 55.

TABLE 18

| | | | LCMS (ESI+) | |
|---|---|---|---|---|
| Exp. | SM | Structure | MASS | RT |
| 66 | 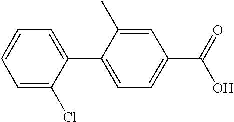 | 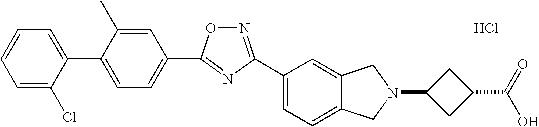 | 486.2 | 1.74 |
| 67 | 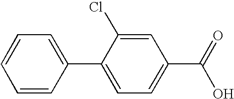 | 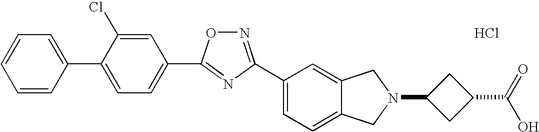 | 472.2 | 1.75 |
| 68 | 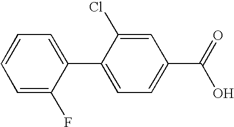 | 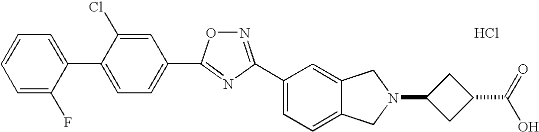 | 490.2 | 1.66 |
| 69 | 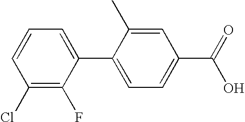 | 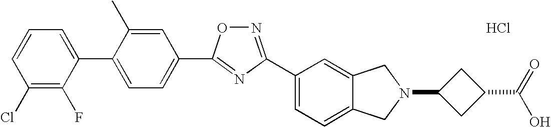 | 504.2 | 1.56 |
| 70 | 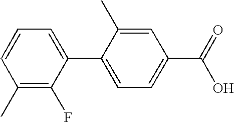 | 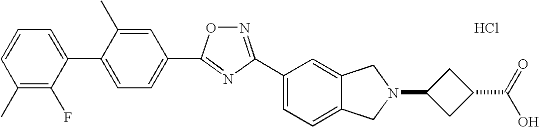 | 484.3 | 1.56 |
| 71 | 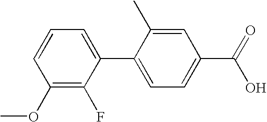 | 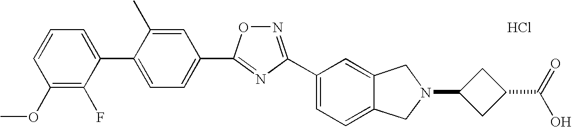 | 500.3 | 1.41 |
| 72 | 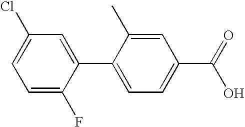 | 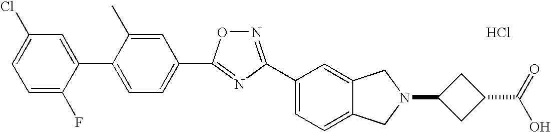 | 504.2 | 1.60 |
| 73 | 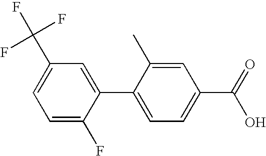 | 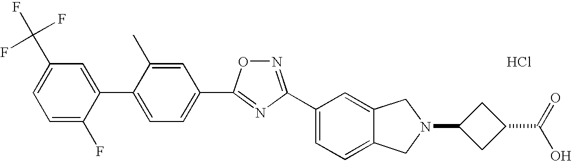 | 538.3 | 1.58 |
| 74 | 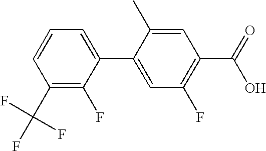 | 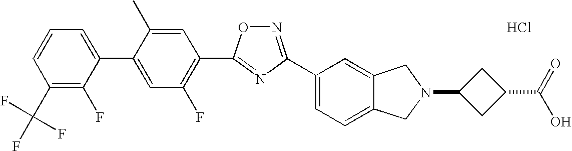 | 556.3 | 1.56 |
| 75 | 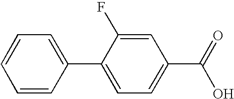 | 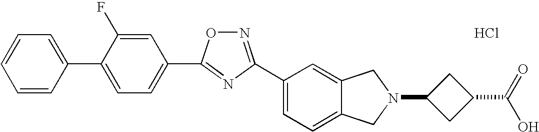 | 456.4 | 1.41 |
| 76 | 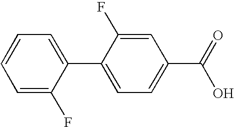 | 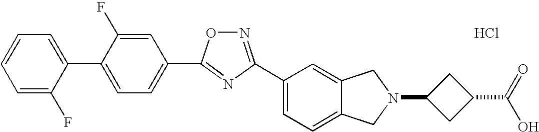 | 474.2 | 1.41 |

### Reference Example 111

### 3-Chloro-5-nitrophthalonitrile

To acetonitrile solution (150 mL) comprising 2-amino-3-chloro-5-nitrobenzonitrile (3.0 g, manufactured by Aldrich Company), copper (I) cyanide (2.04 g, manufactured by Aldrich Company) and tert-butyl nitrite (2.73 mL, manufactured by Kanto Chemical Co., Inc.) were added at 0° C. After stirring the mixture for one hour, the temperature was raised to room temperature and the mixture was stirred for 2 hours, followed by raising the temperature to 65° C. and stirring at the same temperature for 15 hours. Then, copper (I) cyanide (2.04 g) and tert-butyl nitrite (2.73 mL) were added again thereto and the mixture was stirred for 9 hours. Upon the completion of stirring, the reaction solution was poured over saturated brine, extracted with ethyl acetate, and the organic layer was dried over sodium sulfate. The solids were removed by filtration, and the filtrate was dried under reduced pressure and subjected to flash column chromatography (using 10/1 (v:v) hexane/ethyl acetate as an eluent) to obtain the title compound (299 mg).

¹H-NMR (CDCl₃): 8.62 (1H, dd, J=1.83), 8.55 (1H, dd, J=1.83).

### Reference Example 112

### 5-Amino-3-chlorophthalonitrile

To 4N hydrochloric acid ethyl acetate solution (10 mL, manufactured by KOKUSAN CHEMICAL Co., Ltd.) comprising 3-chloro-5-nitrophthalonitrile (199 mg) obtained from the Reference example 111, iron powder (267 mg, manufactured by Wako Pure Chemical Industries, Ltd.) was added and the mixture was stirred for 4.5 hours. After concentrating the reaction solution to half or so, it was poured over saturated sodium bicarbonate solution and extracted with ethyl acetate. The organic layer was dried over sodium sulfate. The solids were removed by filtration, and the filtrate was dried under reduced pressure to obtain the title compound (151 mg).

MASS: 176.0 (M−H), RT: 1.24 min.

### Reference Example 113

### 5-Bromo-3-chlorophthalonitrile

To acetonitrile solution (8.5 mL, manufactured by Kanto Chemical Co., Inc.) comprising 5-amino-3-chlorophthalonitrile (151 mg) obtained from the Reference example 112, copper (II) bromide (228 mg, manufactured by Wako Pure Chemical Industries, Ltd.) and tert-butyl nitrite (122 μl, manufactured by Tokyo Chemical Industry, Co.) were added at 0° C. After stirring the mixture at 0° C. for three hours, copper (II) bromide (190 mg) and tert-butyl nitrite (102 μl) were further added followed by stirring for one hour at 0° C. Upon the completion of stirring, the reaction solution was poured over saturated brine, extracted with ethyl acetate, and dried over sodium sulfate. The solids were removed by filtration, the filtrate was dried under reduced pressure, and then subjected to flash column chromatography (using 10/1 (v:v) hexane/ethyl acetate as an eluent) to obtain the title compound (145 mg).

¹H-NMR (CDCl₃): 7.97 (1H, dd, J=1.83), 7.88 (1H, dd, J=1.83).

### Reference Example 114

### 5-Bromo-3-chlorophthalic acid

To 2-methoxy ethanol solution (2.5 mL) comprising 5-bromo-3-chlorophthalonitrile (145 mg) obtained from the Reference example 113, 2.5N sodium hydroxide solution (2.5 mL, prepared from 5N sodium hydroxide solution, manufactured by Wako Pure Chemical Industries, Ltd.) was added. The mixture was stirred at 95° C. for 13.5 hours. Upon the completion of reaction, the reaction solution was concentrated, poured over 1N hydrochloric acid solution, and extracted with ethyl acetate. The organic layer was dried over sodium sulfate. The solids were removed by filtration, the filtrate was dried under reduced pressure to obtain the title compound (191 mg).

MASS: 276.8 (M−H), RT: 0.93 min.

### Reference Example 115

### 5-Bromo-3-chloro-1,2-bis(hydroxymethyl)benzene

To THF solution (7.0 mL) comprising 5-bromo-3-chlorophthalic acid (191 mg) obtained from the Reference example 114, borane-dimethylsulfide (2.0M THF solution, 855 μl, manufactured by Aldrich Company) was added thereto. The mixture was refluxed under heating for 12.5 hours. Upon the completion of reaction, the reaction solution was added with methanol, poured over saturated brine, and extracted with ethyl acetate. The organic layer was dried over sodium sulfate. The solids were removed by filtration, the filtrate was dried under reduced pressure and then the obtained residue was subjected to flash column chromatography (using 3/1 to 1/1 (v:v) hexane/ethyl acetate as an eluent) to obtain the title compound (67.0 mg).

¹H-NMR (CDCl₃): 7.54 (1H, dd, J=1.83), 7.44 (1H, dd, J=1.83), 4.86 (2H, s), 4.72 (2H, s).

MASS: 249.0 (M−H), RT: 1.17 min.

### Reference Example 116

### 3-Chloro-4,5-bis(hydroxymethyl)benzonitrile

To DMF (3.0 mL) solution comprising 5-bromo-3-chloro-1,2-bis(hydroxymethyl)benzene (67.0 mg) obtained from the Reference example 115, tetrakis(triphenylphosphine)palladium (61.6 mg, manufactured by Aldrich Company) and zinc cyanide (62.6 mg, manufactured by Aldrich Company) were added, followed by stirring for 12 hours at 120° C. Upon the completion of reaction, the reaction solution was filtered using Celite and concentrated. The resulting residuals were subjected to flash column chromatography (using 3:1 to 3:2 (v/v) hexane/ethyl acetate as an eluent) to obtain a crude product of the title compound (42.0 mg).

MASS: 196.0 (M−H), RT: 0.88 min.

### Reference Example 117

### 3-Chloro-N′-hydroxy-4,5-bis(hydroxymethyl)benzimidamide

To 3-chloro-4,5-bis(hydroxymethyl)benzonitrile obtained from the Reference example 116, hydroxylamine hydrochloric acid salt (29.5 mg, manufactured by Kanto Chemical Co., Inc.), DMF (2.5 mL) and triethylamine (65.3 μl, manufactured by Wako Pure Chemical Industries, Ltd.) were added. After filtration, the mixture was stirred at 100° C. for 12 hours. The reaction solution was dried under reduced pressure and used for the next reaction.

MASS: 231.1 (M−H), RT: 0.47 min.

Except that the starting materials shown in Table 19 and the resulting compound obtained from the Reference example 117 were used, the compound described in Table 19 was synthesized in the same manner as Step 2 of the Reference example 26.

TABLE 19

| | | | LCMS (ESI+) | |
|---|---|---|---|---|
| ref. | SM | Structure | MASS | RT |
| 118 | 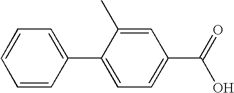 | 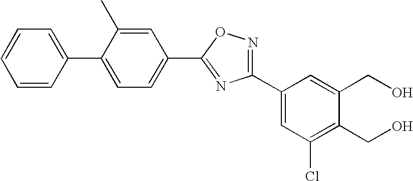 | 407.2 | 2.05 |

The starting materials shown in Table 20 were used, the compound described in Table 20 was synthesized in the same manner as the Reference examples 29 and 30, and the Example 8.

TABLE 20

| | | | LCMS (ESI+) | |
|---|---|---|---|---|
| Exp. | SM | Structure | MASS | RT |
| 77 | 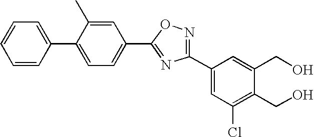 | 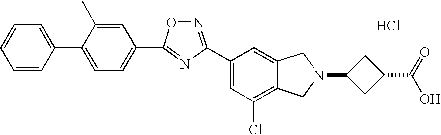 | 486.2 | 1.67 |

### Reference Example 119

### 1,2-Bis(hydroxymethyl)-4-(5-(3-methyl-4-(thiophen-3-yl)phenyl)-1,2,4-oxadiazol-3-yl)benzene

By using 1,2-bis(hydroxymethyl)-4-bromobenzene obtained from the Reference example 22, the title compound was obtained in the same manner as Reference example 16 and 19 and Step 2 of the Reference example 26 in order.

MASS: 379.0 (M+), RT: 1.79 min.

Except that the any of starting materials shown in Table 21 was used, the compounds described in Table 21 were synthesized in the same manner as the Reference example 119. However, for Reference examples of Table 21 excluding Reference example 121, 122, 123, 125, 126, 127, and 129, THF was used as a reaction solvent for the step which corresponds to Step 2 of the Reference example 26.

TABLE 21

| | | | LCMS (ESI+) | |
|---|---|---|---|---|
| ref. | SM | Structure | MASS | RT |
| 120 | 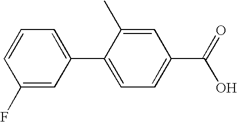 | 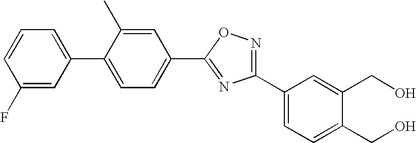 | 391.1 | 1.86 |
| 121 | 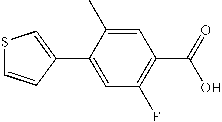 | 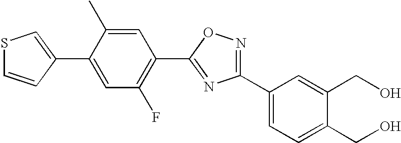 | 397.0 | 1.79 |
| 122 | 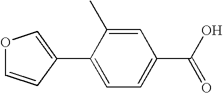 | 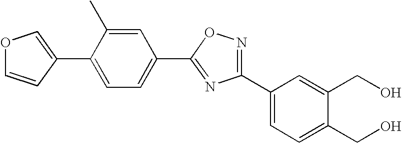 | 363.1 | 1.66 |
| 123 | 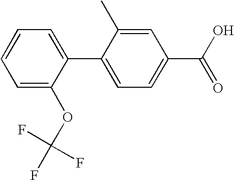 | 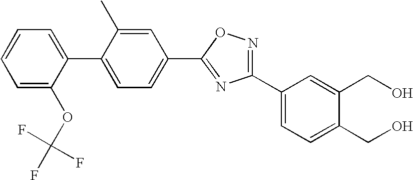 | 457.1 | 1.94 |
| 124 | 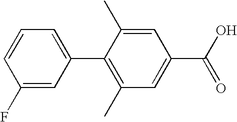 | 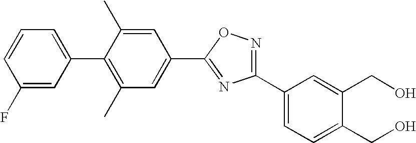 | 405.1 | 1.94 |
| 125 | 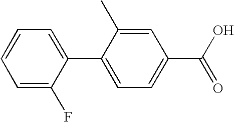 | 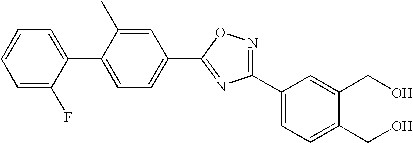 | 391.1 | 1.81 |
| 126 | 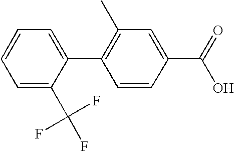 | 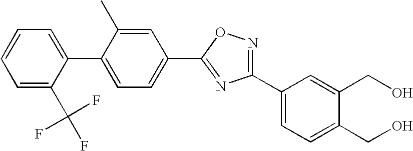 | 441.1 | 1.89 |
| 127 | 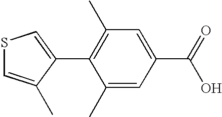 | 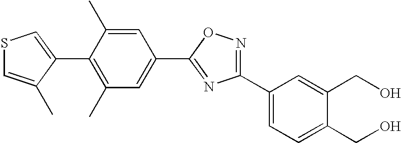 | 407.1 | 1.99 |
| 128 | 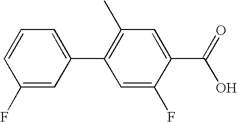 | 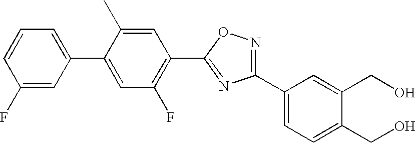 | 409.1 | 1.84 |
| 129 | 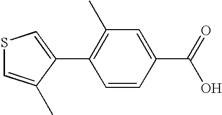 | 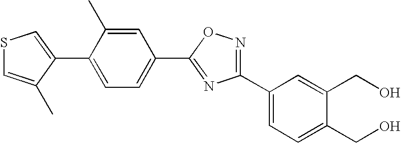 | 393.1 | 1.89 |
| 130 | 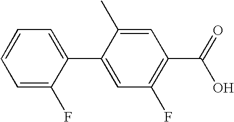 | 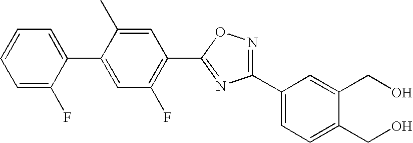 | 409.2 | 1.81 |
| 131 | 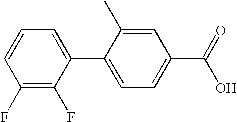 | 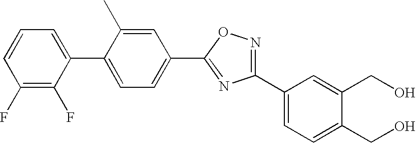 | 409.1 | 1.83 |
| 132 | 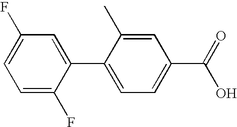 | 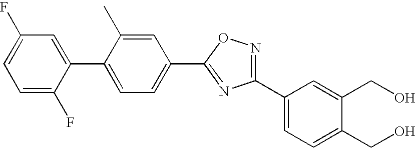 | 409.2 | 1.82 |
| 133 | 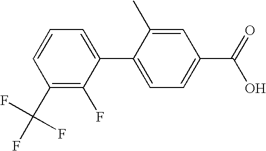 | 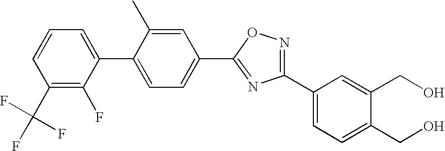 | 459.2 | 1.93 |
| 134 | 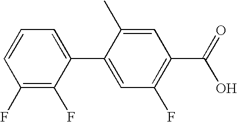 | 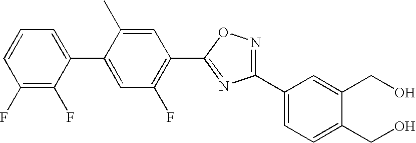 | 427.3 | 1.84 |
| 135 | 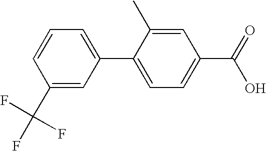 | 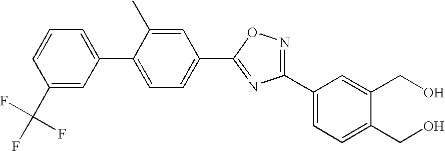 | 441.2 | 1.97 |
| 136 | 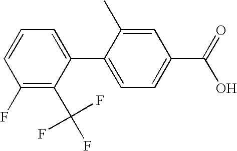 | 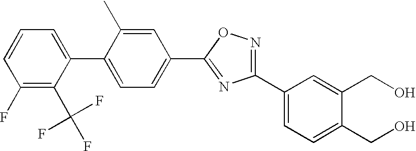 | 459.1 | 1.89 |
| 137 | 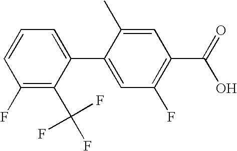 | 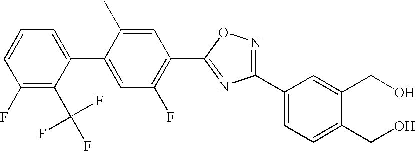 | 477.3 | 1.89 |
| 138 | 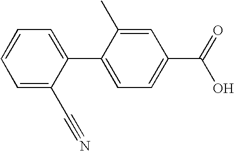 | 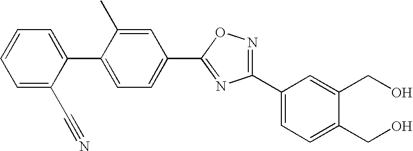 | 398.2 | 1.61 |
| 139 | 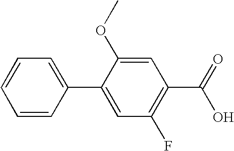 | 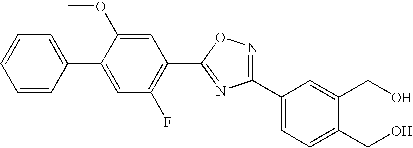 | 407.1 | 1.77 |
| 140 | 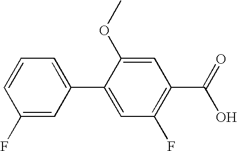 | 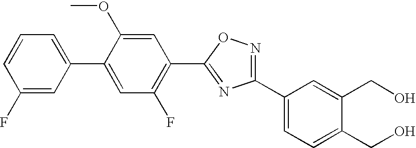 | 425.1 | 1.79 |
| 141 | 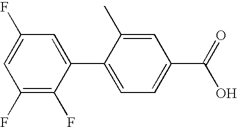 | 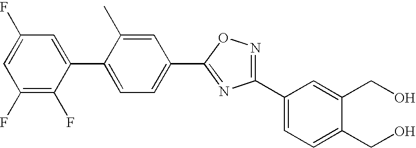 | 427.1 | 1.87 |
| 142 | 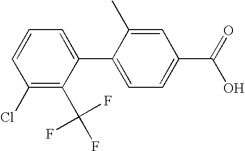 | 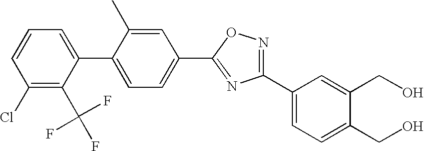 | 475.1 | 2.01 |
| 143 | 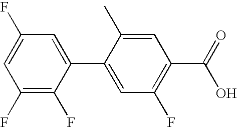 | 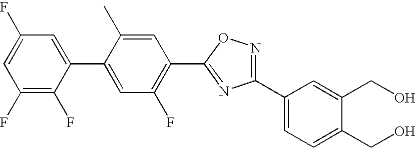 | 445.1 | 1.86 |
| 144 | 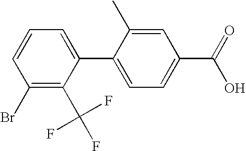 | 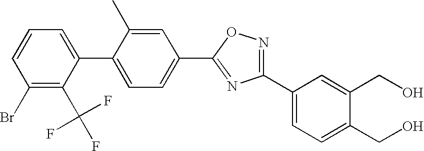 | 519.1 | 2.03 |

Except that the any of starting materials shown in Table is used, the compounds described in Table 22 were synthesized in the same manner as the Reference example 29 and 30 and the Example 8.

TABLE 22

| | | | LCMS (ESI+) | |
|---|---|---|---|---|
| Exp. | SM | Structure | MASS | RT |
| 78 | 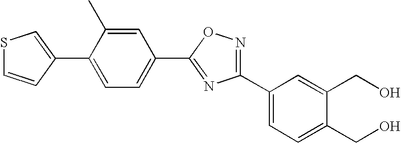 | 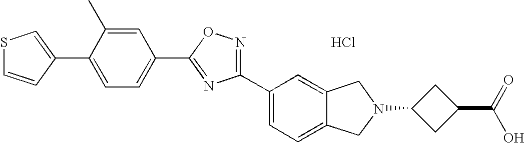 | 379.0 | 1.79 |
| 79 | 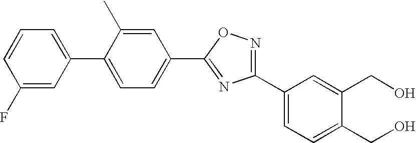 | 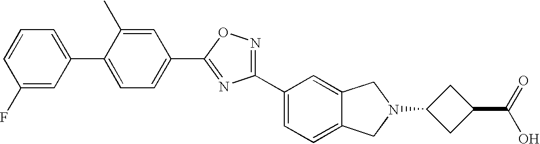 | 470.1 | 1.48 |
| 80 | 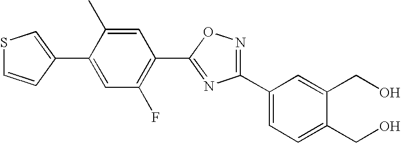 | 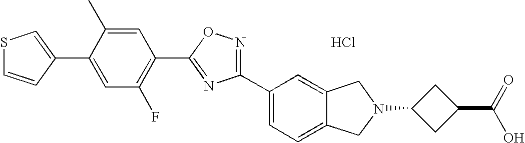 | 476.1 | 1.43 |
| 81 | 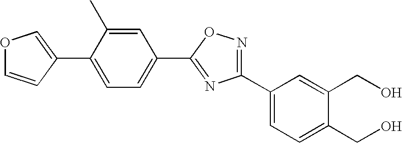 | 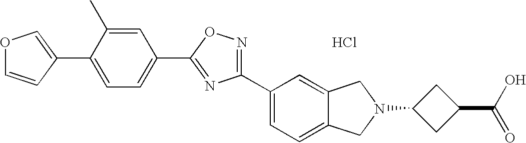 | 442.1 | 1.32 |
| 82 | 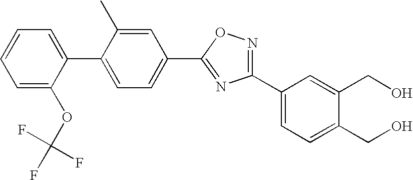 | 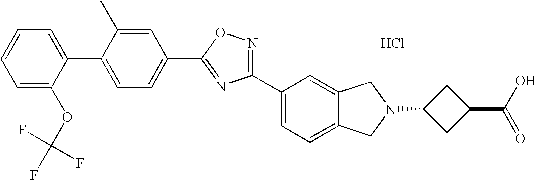 | 536.1 | 1.50 |
| 83 | 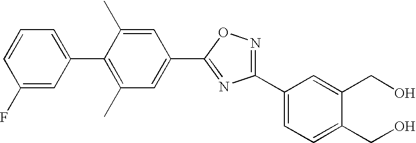 | 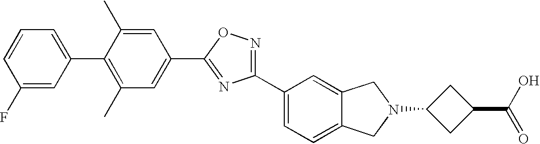 | 484.2 | 1.48 |
| 84 | 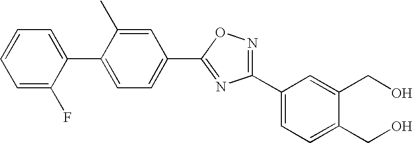 | 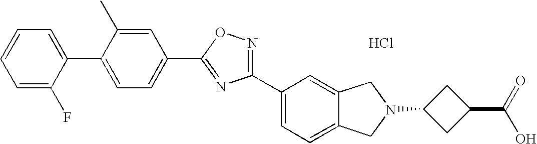 | 470.1 | 1.40 |
| 85 | 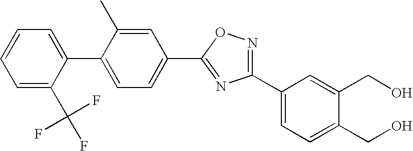 | 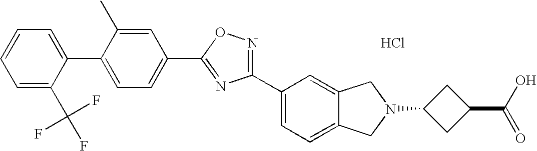 | 520.1 | 1.53 |
| 86 | 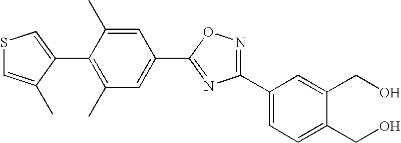 | 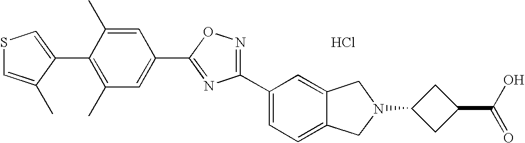 | 486.1 | 1.49 |
| 87 | 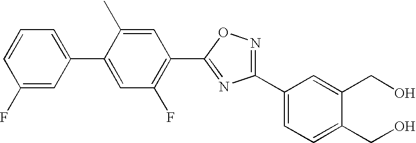 | 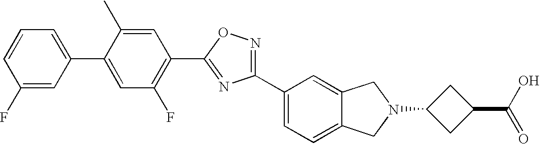 | 488.1 | 1.49 |
| 88 | 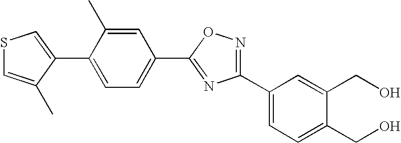 | 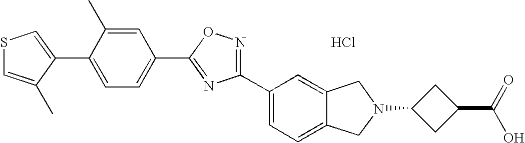 | 472.1 | 1.45 |
| 89 | 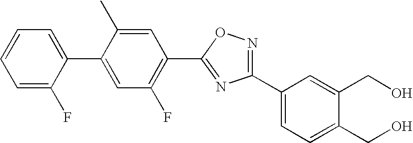 | 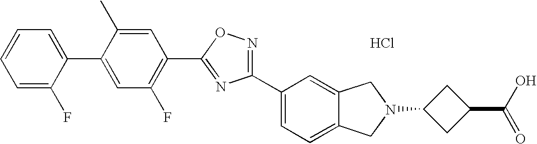 | 488.2 | 1.40 |
| 90 | 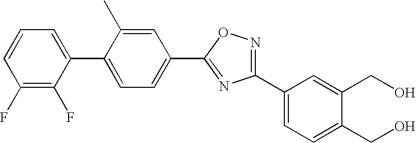 | 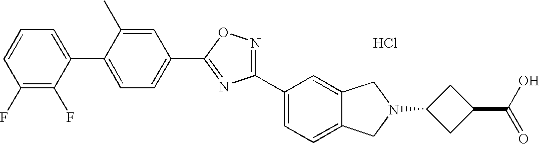 | 488.2 | 1.44 |
| 91 | 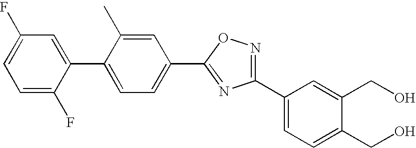 | 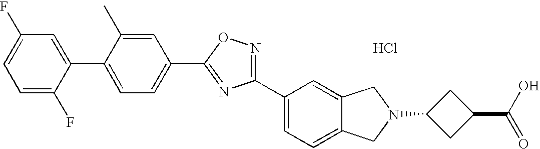 | 488.2 | 1.37 |
| 92 | 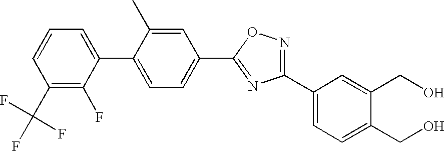 | 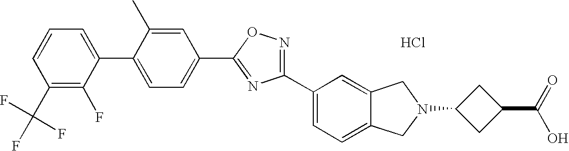 | 538.2 | 1.54 |
| 93 | 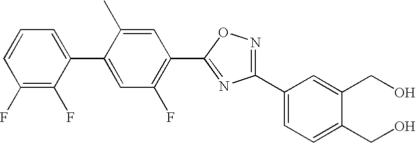 | 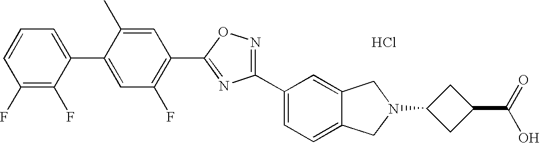 | 506.4 | 1.44 |
| 94 | 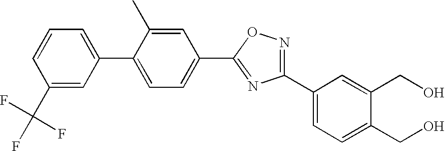 | 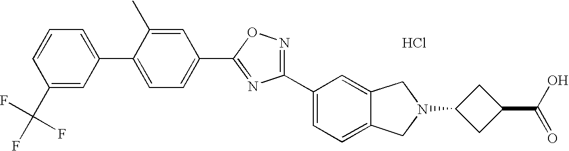 | 520.2 | 1.51 |
| 95 | 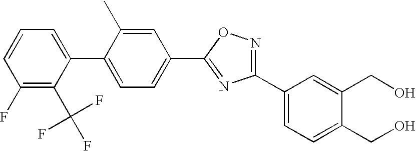 | 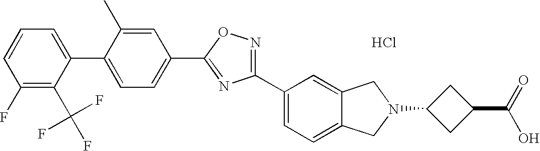 | 538.4 | 1.50 |
| 96 | 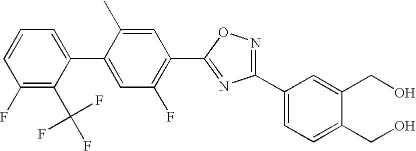 | 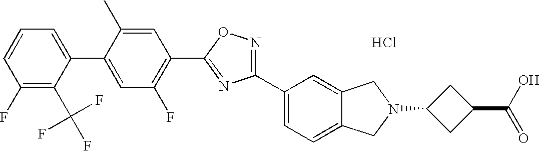 | 556.4 | 1.51 |
| 97 | 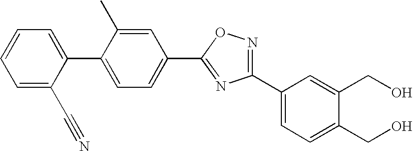 | 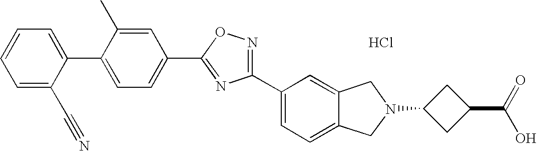 | 477.4 | 1.29 |
| 98 | 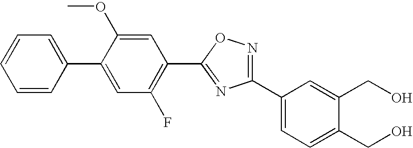 | 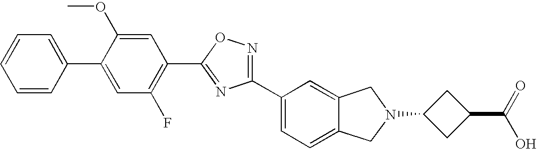 | 486.1 | 1.42 |
| 99 | 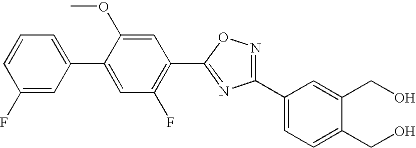 | 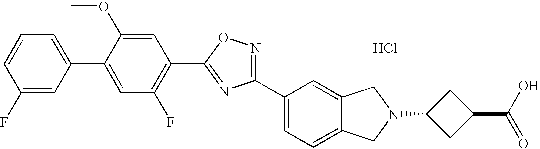 | 504.1 | 1.42 |
| 100 | 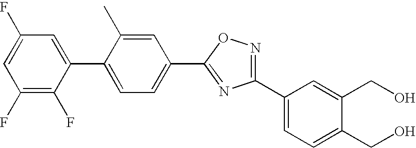 | 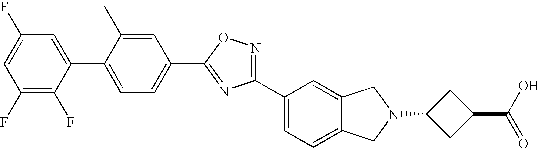 | 506.5 | 1.44 |
| 101 | 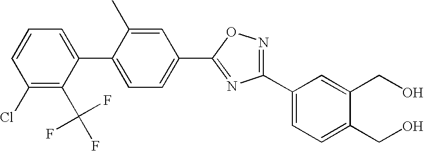 | 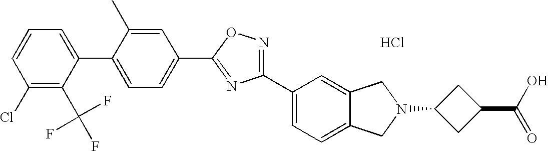 | 554.2 | 1.67 |
| 102 | 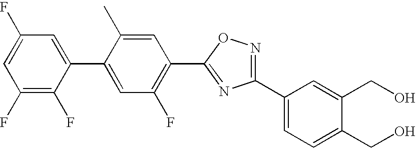 | 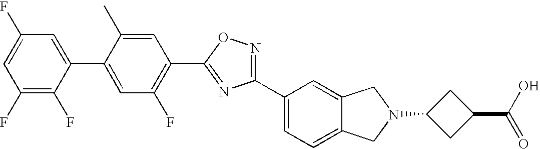 | 524.2 | 1.62 |
| 103 | 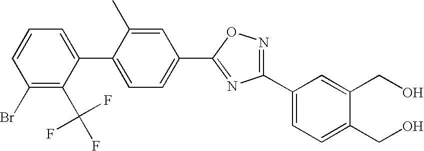 | 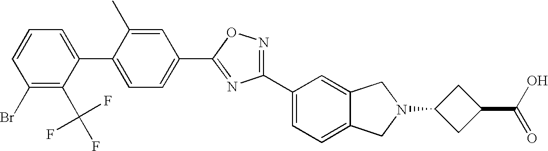 | 598.2 | 1.71 |

In addition, for Example 79, 83, 87, 98, 100, 102, and 103, purification was carried out according to Condition D described above (i.e., purification method 4).

### Reference Example 145

### Tert-butyl 1,3-cis-3-(tert-butoxycarbonylamino)cyclobutane carboxylate

To THF solution (5.0 mL) comprising 1,3-cis-3-(tert-butoxycarbonylamino)cyclobutane carboxylic acid (100 mg, manufactured by Albany Molecular Research, Inc.), DMAP (79.4 mg, manufactured by Wako Pure Chemical Industries, Ltd.) and di-t-butyl bicarbonate (128 μl, manufactured by Wako Pure Chemical Industries, Ltd.) were added at 0° C., and the mixture was stirred for 13 hours. Upon the completion of stirring, the reaction solution was poured over saturated brine, and extracted with ethyl acetate. The organic layer was dried over sodium sulfate. The solids were removed by filtration, the filtrate was dried under reduced pressure and then subjected to flash column chromatography (using 10/1 (v:v) hexane/ethyl acetate as an eluent) to obtain the title compound (89.7 mg).

¹H-NMR (CDCl₃): 4.87 (1H, brs), 4.17-3.97 (1H, m), 2.73-2.47 (3H, m), 2.03 (2H, ddd, J=2.37, J=9.15, J=18.1).

### Reference Example 146

### Tert-butyl 1,3-cis-3-aminocyclobutane carboxylate hydrochloric acid salt

To tert-butyl 1,3-cis-3-(tert-butoxycarbonylamino)cyclobutane carboxylate (89.7 mg) obtained from the Reference example 145, 1N hydrochloric acid ethyl acetate solution (1.65 mL, prepared from 4N e hydrochloric acid ethyl acetate solution that is manufactured by KOKUSAN CHEMICAL Co., Ltd.) was added and the mixture was stirred for 36 hours. Upon the completion of the stirring, diethyl ether was added and the solids were removed by filtration to obtain the title compound (78.9 mg).

¹H-NMR (DMSO-d₆): 3.56 (1H, tt, J=8.79, J=7.68), 2.85 (1H, tt, J=9.54, J=8.24), 2.37 (2H, ddd, J=2.55, J=7.70, J=17.2), 2.21 (2H, ddd, J=2.55, J=9.54, J=19.0).

Except that the starting materials shown in Table 23 and tert-butyl 1,3-cis-3-aminocyclobutane carboxylate hydrochloric acid salt obtained from the Reference example 146 are used, the compounds described in Table 23 were synthesized in the same manner as the Reference example 29 and 30 and the Example 8.

TABLE 23

| | | | LCMS (ESI+) | |
|---|---|---|---|---|
| Exp. | SM | Structure | MASS | RT |
| 104 | 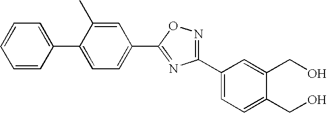 | 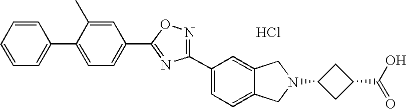 | 452.4 | 1.42 |
| 105 | 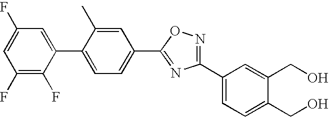 | 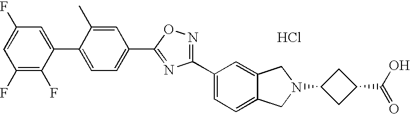 | 506.4 | 1.52 |
| 106 | 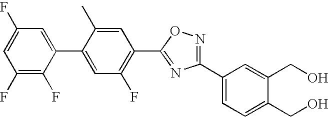 | 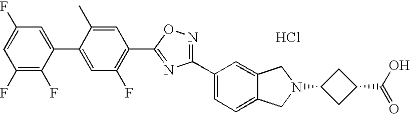 | 524.4 | 1.49 |

Further, for Example 105, purification was carried out according to the above described Condition D (i.e., purification method 4).

### Example 107

### 1,3-Trans-3-(5-(5-(3′-amino-2-methyl-2′-trifluoromethylbiphenyl-4-yl)-1,2,4-oxadiazol-3-yl)-2,3-dihydro-1H-inden-1-ylamino)cyclobutane carboxylic acid

To the 5N hydrochloric acid solution (1 mL, manufactured by Wako Pure Chemical Industries, Ltd.) comprising the compound of Example 49 (5 mg), iron powder (4.5 mg, manufactured by Wako Pure Chemical Industries, Ltd.) was added and stirred. After stirring for 15 hours, excess amount of iron powder and 5N hydrochloric acid solution (2.0 mL, manufactured by Wako Pure Chemical Industries, Ltd.) were added followed by stirring for six hours.

Separately, to the 5N hydrochloric acid solution (5 mL, manufactured by Wako Pure Chemical Industries, Ltd.) comprising the compound of the Example 49 (10 mg), iron powder (9.1 mg, manufactured by Wako Pure Chemical Industries, Ltd.) was added and stirred. After stirring for 5 hours, excess amount of iron powder was added followed by stirring for 15 hours.

Both reaction solutions were mixed with each other, and the solvent was distilled off. HPLC purification was carried out (XBridge OBD (TM), 19 mmI.D.×50 mm, manufactured by WATERS), by using water, contains 0.1% acetic acid-acetonitrile solvent as an eluent. As a result, the title compound was obtained (2.5 mg).

MASS: 549.2 (M+H), RT: 1.64 min.

### Test Example 1

### ³⁵S-GTPγS Binding Assay Using a Membrane Prepared from CHO Cells Which Stably Express S1P1 Receptor

Functional activation of human S1P1 by the compounds was determined based on quantification of ³⁵S-GTPγS binding to G protein due to the receptor activation, by using a cell membrane fraction prepared from CHO cells in which human S1P1 are stably expressed. As for human S1P1, the above described human S1P1 was used. Membrane proteins as prepared and various concentrations of sphingosine-1-phosphate or the compounds diluted in a solvent such as DMSO were incubated in a solution comprising 20 mM Tris-Cl (pH 7.5), 100 mM NaCl, 10 mM MgCl₂, 5 μm GDP (Upstate), 0.1% BSA (fatty acid free, Sigma) and 25 pM ³⁵S-GTPγS (specific activity 1250 Ci/mmol) in 96 well microtiter plates. Binding was performed by incubating them for 90 minutes at room temperature, and then terminated by harvesting the membrane proteins onto multiscreeen harvest FB plates (Millipore) using Millipore multiscreen separation system. After drying the harvest plates at least for 12 hours, 25 μl of MicroScint-O (Perkin Elmer) was added to each well and radioactivity was measured using a Top Counter.

Having the value measured for the well to which a solvent has been added as a control value, the agonist activity of the compounds was determined by comparing the increase in the well to which a test compound has been added to the control value, and therefore an increase ratio for each concentration of the compounds was obtained. EC₅₀ values were culcurated as defined to be the concentration of agonist required to give 50% of its own maximum increase ratio.

All of the compounds of the above Examples have EC₅₀ values less than 100 nM. Further, the compounds of the Example Nos. 1 to 6, 8, 10 to 17, 20 to 26, 28 to 32, 34 to 47, 49 to 52, 54 to 68, 77 to 78, 84 to 85, 88 to 90, 92 to 93, 95, 100, 102, 104 and 106 have EC₅₀ values less than 10 nM.

Meanwhile, the ratio compared to EC₅₀ for human S1P3 receptor as described in Test example 2 can be also calculated (i.e., S1P1/S1P3). In addition, the ratio compared to EC₅₀ for human S1P2 receptor as described in Test example 3 (i.e., S1P1/S1P2), the ratio compared to EC₅₀ for human S1P4 receptor as described in Test example 4 (i.e., S1P1/S1P4), and the ratio compared to EC₅₀ for human S1P5 receptor as described in Test example 5 (i.e., S1P1/S1P5) can be also obtained. According to this, the usefulness of the compounds of the present invention as an effective component of a pharmaceutical agent can be confirmed.

### Test Example 2

### ³⁵S-GTPγS Binding Assay Using a Membrane Preparated from CHO Cells Which Stably Express Human S1P3

³⁵S-GTPγS binding via human S1P3 was measured in the same manner as the ³⁵S-GTPγS binding via human S1P1. According to this test, a membrane protein of CHO cells in which human S1P3 has been stably expressed was prepared and used. In addition, as for human S1P3, the above described human S1P3 was used.

Having the value measured for the well to which a solvent has been added as a control value, the agonist activity of the compounds was determined by comparing the increase in the well to which a test compound has been added to the control value, and therefore an increase ratio for each concentration of the compounds was obtained. EC₅₀ values were culculated as defined to be the concentration of agonist required to give 50% of its own maximum increase ratio.

All of the evaluated compounds of the above Examples have EC₅₀ values the same or greater than 500 nM. Further, the compounds of the Example Nos. 1 to 4, 6 to 12, 14 to 68, 77 to 82, 84 to 87, 89 to 103, and 106 to 107 have EC₅₀ values the same or greater than 1000 nM.

Furthermore, from the results of the Examples 1 and 2, it was found that the ratio between EC₅₀ value for human S1P1 and EC₅₀ value for human S1P3 is at least 200 for all the compounds described in the Examples, except the Example Nos. 13, 19, 27, 53, 67, 79, 81 to 83, 86 to 87, 91, 94, 96, 98 to 99, 101, and 103.

### Test Example 3

### ³⁵S-GTPγS Binding Assay Using a Membrane Preparated from CHO Cells Which Stably Express Human S1P2

³⁵S-GTPγS binding via human S1P2 was measured in the same manner as the test relating to ³⁵S-GTPγS binding via human S1P1. According to this test, a membrane protein of CHO cells in which human S1P2 has been stably expressed was prepared and used. In addition, as for human S1P2, the above described human S1P2 can be used.

Having the value measured for the well to which a solvent has been added as a control value, the agonist activity of the compounds was determined by comparing the increase in the well to which a test compound has been added to the control value, and therefore an increase ratio for each concentration of the compounds was obtained. EC₅₀ values were culculated as defined to be the concentration of agonist required to give 50% of its own maximum increase ratio.

### Test Example 4

### ³⁵S-GTPγS Binding Assay Using a Membrane Preparated from CHO cells Which Stably Express Human S1P4

³⁵S-GTPγS binding via human S1P4 was measured in the same manner as the test relating to ³⁵S-GTPγS binding via human S1P1 receptor. According to this test, a membrane protein of CHO cells in which human S1P4 has been stably expressed was prepared and used. In addition, as for human S1P4, the above described human S1P4 can be used.

Having the value measured for the well to which a solvent has been added as a control value, the agonist activity of the compounds was determined by comparing the increase in the well to which a test compound has been added to the control value, and therefore an increase ratio for each concentration of the compounds was obtained. EC₅₀ values were culculated as defined to be the concentration of agonist required to give 50% of its own maximum increase ratio.

### Test Example 5

### ³⁵S-GTPγS Binding Assay Using a Membrane Preparated from CHO Cells Which Stably Express Human S1P5

³⁵S-GTPγS binding via human S1P5 was measured in the same manner as the test relating to ³⁵S-GTPγS binding via human S1P1. According to this test, a membrane protein of CHO cells in which human S1P5 has been stably expressed was prepared and used. In addition, as for human S1P5, the above described human S1P5 can be used.

Having the value measured for the well to which a solvent has been added as a control value, the agonist activity of the compounds was determined by comparing the increase in the well to which a test compound has been added to the control value, and therefore an increase ratio for each concentration of the compounds was obtained. EC₅₀ values were culculated as defined to be the concentration of agonist required to give 50% of its own maximum increase ratio.

### Test Example 6

### Assay for Ligand Binding to Human S1P1 Receptor

Binding activity of the compound to human S1P1 can be evaluated based on a binding assay by using ³³P-sphingosine-1-phosphate and a cell membrane fraction prepared from CHO cells in which human S1P1 are stably expressed. Furthermore, as for human S1P1, the above described human S1P1 was used.

Similar to the Test example 1, membrane proteins prepared from CHO cells in which human S1P1 are stably expressed and various concentrations of ³³P-sphingosine-1-phosphate (20 pM; specific activity 3000 Ci/mmol, American Radiolabeled Chemicals) and the compounds diluted in a solvent such as DMSO were incubated in a solution comprising 20 mM Tris-Cl (pH7.5), 100 mM NaCl, 15 mM NaF, 2 mM deoxypyridoxine (Sigma), 4 mg/mL BSA (fatty acid free, Sigma) in 96 well microtiter plates. Binding was performed for 60 minutes at 30° C., and terminated by harvesting the membrane proteins onto GF/C unifilter plates (Perkin Elmer) using Millipore multiscreen separation system. After drying the filter plates for more than 12 hours, 25 μl of Microscint-0 (Perkin Elmer) was added to each well and radioactivity was measured using a Top Counter. Non-specific binding was defined as the amount of residual radioactivity in the presence non-radioactive sphingosine-1-phosphate having concentration of at least 1 NM. Regarding the binding activity of the compounds to the receptor, the value for the well to which a solvent has been added was taken as a maximum value, and according to comparison with non-specific binding value, an inhibition ratio for binding of ³³P-sphingosine-1-phosphate was determined for each concentration of the compounds. IC₅₀ values were defined to be the concentration of the compound required to give 50% inhibition of binding and culculated.

Meanwhile, the ratio compared to IC₅₀ for human S1P3 receptor as described in Test example 7 can be also calculated (i.e., S1P1/S1P3). In addition, the ratio compared to IC₅₀ for human S1P2 receptor as described in Test example 8 (i.e., S1P1/S1P2), the ratio compared to IC₅₀ for human S1P4 receptor as described in Test example 9 (i.e., S1P1/S1P4), and the ratio compared to IC₅₀ for human S1P5 receptor as described in Test example 10 (i.e., S1P1/S1P5) can be also obtained.

In addition, based on comparison with the results of ³⁵S-GTPγS binding assay shown in the Test example 1, the agonist or the antagonist activity of a compound for the human S1P1 receptor can be determined.

### Test Example 7

### Assay for Ligand Binding to Human S1P3 Receptor

Activity of the compounds to human S1P3 can be also determined according to a ligand binding assay. Ligand binding assay regarding human S1P3 receptor can be carried out in the same manner as the ligand binding assay regarding human S1P1 receptor. Similar to the Test example 2, the membrane proteins which prepared from CHO cells in which human S1P3 has been stably expressed, are used. As for human S1P3 receptor, the above described human S1P3 can be used.

In addition, based on comparison with the results of ³⁵S-GTPγS binding assay shown in the Test example 2, the agonist or the antagonist activity of a compound for the human S1P3 receptor can be determined.

### Test Example 8

### Assay for Ligand Binding to Human S1P2 Receptor

Activity of the compounds to human S1P2 can be also determined according to a ligand binding assay. Ligand binding assay regarding human S1P2 receptor can be carried out in the same manner as the ligand binding assay regarding human S1P1 receptor. Similar to the Test example 3, the membrane proteins which prepared from CHO cells in which human S1P2 has been stably expressed, are used. As for human S1P2 receptor, the above described human S1P2 can be used.

In addition, based on comparison with the results of ³⁵S-GTPγS binding assay shown in the Test example 3, the agonist or the antagonist activity of a compound for the human S1P2 receptor can be determined.

### Test Example 9

### Assay for Ligand Binding to Human S1P4 Receptor

Activity of the compounds to human S1P4 can be also determined according to a ligand binding assay. Ligand binding assay regarding human S1P4 receptor can be carried out in the same manner as the ligand binding assay regarding human S1P1 receptor. Similar to the Test example 4, membrane proteins are prepared from CHO cells in which human S1P4 are stably expressed and used. In addition, as for human S1P4, the above described human S1P4 can be used.

In addition, based on comparison with the results of ³⁵S-GTPγS binding assay shown in the Test example 4, the agonist or the antagonist activity of a compound for the human S1P4 receptor can be determined.

### Test Example 10

### Assay for Ligand Binding to Human S1P5 Receptor

Activity of the compounds to human S1P5 can be also determined according to a ligand binding assay. Ligand binding assay regarding human S1P5 receptor can be carried out in the same manner as the ligand binding assay regarding S1P1 receptor. Similar to the Test example 5, membrane proteins are prepared from CHO cells in which human S1P5 are stably expressed and used. In addition, as for human S1P5, the above described human S1P5 can be used.

In addition, based on comparison with the results of ³⁵S-GTPγS binding assay shown in the Test example 5, the agonist or the antagonist activity of a compound for the human S1P5 receptor can be determined.

### Test Example 11

### Evaluation of Peripheral Blood Lymphocyte Reduction

The compound or a solvent is orally administered to a rat. 3, 6, 24, 48 or 72 hours after the administration of the compounds, blood is drawn from the rat tail. Hematological testis carried out for the whole blood sample. Using an automated analyzer (Sysmex 2000Xi), total number of peripheral lymphocytes is obtained. By having at least three animals per group, activity of the compound on the total number of peripheral lymphocytes was evaluated. Lymphocyte reduction caused by the compound administration was evaluated via comparison with an animal group which had received the solvent only. Specifically, average number of the lymphocytes for the solvent administered group was taken as 100%, and from average number of the lymphocytes for the compound administered group, control % value was calculated. Further, in view of the dosage of the compound and the dosage of the compound that is required to reduce number of lymphocytes by 50% six hours after the administration compared to the control % value was calculated as ED₅₀.

The compounds of the Example 1, 4, 28, 29, 32 and 48 have ED₅₀ value of less than 1 mg/kg.

### Test Example 12

### Evaluation of an Effect on Heart

Activity of the compounds on a cardiac function is monitored using an apparatus for recording electrocardiogram (Power Lab 4/25T). For an anesthetized rat, mouse, or guinea pig, an electrocardiogram is recorded before and after the administration of the compounds. Heart rate is also measured.

A solution comprising the compounds of the present invention is administered intravenously to the animal and a change in heart rate over thirty minutes or more after the administration is measured. By having at least three animals per group, activity of the compound on the animal's heart rate is evaluated. Change in heart rate caused by the compound administration is evaluated by comparing the heart rate with that of solvent administered group or that before the administration.

Meanwhile, by comparing the evaluation results of the Test example 12 with the evaluation results of the Test example 11 and 13 to 15, usefulness of the compound of the present invention as an effective component for a pharmaceutical agent can be confirmed.

Furthermore, after obtaining maximum blood concentration (Cmax) for ED₅₀ dosage in a test for measuring peripheral lymphocyte reduction (Test example 11) and drug concentration in serum (C₀) right after the application (t=0) of the compound having dosage which does not cause any missing QRS waves in an electrocardiogram measurement, the ratio between two concentrations is calculated. From the ratio, usefulness of the compound of the present invention as an effective component for a pharmaceutical agent can be confirmed, specifically in view of disparity between an activity of causing peripheral lymphocyte reduction and an effect on heart.

### Test Example 13

### Rat DTH model

Abdomen of female Lewis rat is shaved with a shaver, and by continuously applying for two days a solution comprising 1% dinitrofluorobenzene (DNFB, 100 μl), sensitization is carried out. Five days after the start of the sensitization, 0.5% DNFB solution (20 μl) is applied to the right auricle (i.e., external portion of the right ear). The compounds to be tested are suspended in 1% methyl cellulose solution, and force the animal to be orally administered with the suspension into the stomach by using a sonde, once a day for six days from the start of the sensitization. Twenty-four or forty-eight hours after the DNFB application, thickness of the auricle is measured by using a thickness gauge (Mitsutoyo Co., Ltd.) to determine the auricle swelling.

Furthermore, after obtaining maximum blood concentration (Cmax) for dosage with which the efficacy is shown in the present test and drug concentration in serum (C₀) right after the application (t=0) of the compound having dosage which does not cause any missing QRS waves in an electrocardiogram measurement for evaluation of the effect on heart (Test example 12) (i.e., dosage which does not cause bradycardia), the ratio between two concentrations is calculated. From the ratio, usefulness of the compound of the present invention as an effective component for a pharmaceutical agent can be confirmed, specifically in view of disparity between an activity according to the present test and an effect on heart.

### Test Example 14

### Animal Model Having Arthritis Caused by an Adjuvant

Seven-week old female Lewis rat is used for the evaluation. After measuring the volume of the hind leg of the rat, M. tuberculosis H37 RA (manufactured by Difco, 500 μg/100 μl) which has been suspended in fluid paraffin as an adjuvant, is subcutaneously injected in the sole of rear left foot of the rat to prepare a rat having arthritis caused by an adjuvant. The compounds to be tested are suspended in 1% methyl cellulose solution, and force the animal to be orally administered with the suspension into the stomach by using a sonde, once a day for twenty-one days from the start of the adjuvant injection. Evaluation of arthritis is carried out by measuring volume of the foot of each animal by using a plethysmometer (manufactured by UGO BASILE). By comparing the value for the group administered with the compounds of the present invention with the solvent administered group, effect of the compounds is determined. Specifically, the swelling of the sole of the rear left foot of the solvent administered group is taken as 100%, and in view of the swelling of the animals of the compound administered group, control % values are calculated. Further, in view of the dosage of the compound and the dosage of the compound that is required to reduce the swelling of sole of the rear left foot by 50% compared to the control % value twenty-one days after the administration is obtained as ED₅₀ value.

The compounds of Example 1 have ED₅₀ value of less than 1 mg/kg.

Furthermore, after obtaining maximum blood concentration (Cmax) for ED₅₀ dosage in the present test and drug concentration in serum (C₀) right after the application (t=0) of the compound having dosage which does not cause any missing QRS waves in an electrocardiogram measurement for evaluation of the effect on heart (Test example 12) (i.e., dosage which does not cause bradycardia), the ratio between two concentrations is calculated. From the ratio, usefulness of the compound of the present invention as an effective component for a pharmaceutical agent can be confirmed, specifically in view of disparity between an activity according to the present test and an effect on heart.

### Test Example 15

### Animal Model Having Arthritis Caused by Collagen

A seven-week old female DBA1J mouse is used. Type II collagen solution prepared with chicken cartilage (1% solution, Nippon Ham, 300-31601) and complete Freund's adjuvant (231131, DIFCO) are mixed with each other to provide an emulsion. Thus-prepared emulsion (100 μl, comprising 100 μg collagen) is intradermally administered to the root region of the rat tail. In addition, three weeks later, 100 μl of the emulsion which has been prepared in the same manner as described above is again intradermally administered to the root region of the rat tail as post-sensitization to induce arthritis. The compounds to be tested are suspended in 1% methyl cellulose solution, and force the animal to be orally administered with the suspension into the stomach by using a sonde, at least once a day from the first day of collagen injection or after the post-sensitization. Until the final evaluation day of arthritis, the administration is repeated.

For the evaluation of arthritis, degree of arthritis found in each of the four limbs is given with a specific score (full score; 5). The effect of the compounds is determined by comparing the score for the group administered with the compounds and the group administered with a solvent only.

Furthermore, after obtaining maximum blood concentration (Cmax) for dosage with which the efficacy is shown in the present test and drug concentration in serum (C₀) right after the application (t=0) of the compound having dosage which does not cause any missing QRS waves in an electrocardiogram measurement for evaluation of the effect on heart (Test example 12) (i.e., dosage which does not cause bradycardia), the ratio between two concentrations is calculated. From the ratio, usefulness of the compound of the present invention as an effective component for a pharmaceutical agent can be confirmed, specifically in view of disparity between an activity according to the present test and an effect on heart.

### INDUSTRIAL APPLICABILITY

Compounds of the present invention, a possible stereoisomer, a racemate, a pharmaceutically acceptable salt, a hydrate, a solvate or a prodrug thereof have an agonist activity for S1P1/Edg1 receptor, and as a result, they are useful as an effective component for a pharmaceutical agent having an immunosuppressive activity and can be favorably used for an industrial field relating to the corresponding pharmaceutical agents.

1. Compounds represented by Formula (1), a possible stereoisomer, a racemate, a pharmaceutically acceptable salt, a hydrate, a solvate or a prodrug thereof: 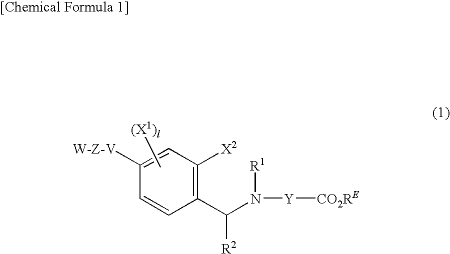
[in the Formula (1), W represents a monovalent group derived from a compound selected from benzene, thiophene, furan and pyridine by the removal of one hydrogen atom and the W may be substituted with one or two X^{W}, wherein X^{W} indicates a C1-C4 alkyl group which may be substituted with 1 to 9 fluorine atoms, a C1-C4 alkoxy group which may be substituted with 1 to 9 fluorine atoms, a halogen atom, a cyano group, a C1-C4 alkylthio group which may be substituted with 1 to 9 fluorine atoms, a C1-C4 alkylsulfinyl group which may be substituted with 1 to 9 fluorine atoms, a C1-C4 alkylsulfonyl group which may be substituted with 1 to 9 fluorine atoms, a C1-C4 acylamide group which may be substituted with 1 to 7 fluorine atoms, a C1-C4 alkylcarbamoyl group which may be substituted with 1 to 9 fluorine atoms, a C1-C4 alkylsulfonamide group which may be substituted with 1 to 9 fluorine atoms, a C1-C4 alkylsulfamoyl group which may be substituted with 1 to 9 fluorine atoms, a C1-C4 acyl group which may be substituted with 1 to 7 fluorine atoms, or a C1-C4 alkyl group which is substituted with one C1-C4 alkoxy group which may be substituted with 1 to 9 fluorine atoms or with one —OH, and when it is substituted with two X^{W}, they can be the same or different from each other;
Z represents a divalent group derived from benzene by the removal of two hydrogen atoms, which binds to W— and —V— at para position and may be substituted with 1 to 4 X^{Z} wherein X^{Z} indicates a C1-C4 alkyl group which may be substituted with 1 to 9 fluorine atoms, a C1-C4 alkoxy group which may be substituted with 1 to 9 fluorine atoms, a halogen atom or a cyano group, and when it is substituted with two or more X^{Z}, they can be the same or different from each other;
V represents a divalent group derived from [1,2,4]-oxadiazole by the removal of two hydrogen atoms or —(CR^{V1}R^{V2})ₙ—(CR^{V3}R^{V4})ₖ—O—;
R^{V1}, R^{V2}, R^{V3}, and R^{V4} can be the same or different from each other, and each independently represent a hydrogen atom, a halogen atom, or a C1-C4 alkyl group which may be substituted with 1 to 5 halogen atoms;
n indicates an integer of 0 to 2, and when n is O, —(CR^{V1}R^{V2})ₙ— means a single bond;
k indicates an integer of 0 or 1 and when k is O, —(CR^{V3}R^{V4})ₖ— means a single bond;
X¹ indicates a C1-C4 alkyl group which may be substituted with 1 to 9 fluorine atoms, a C1-C4 alkoxy group which may be substituted with 1 to 9 fluorine atoms, or a halogen atom,
l indicates an integer of 0 to 3;
when l is 2 or 3, X¹ can be the same or different from each other;
R¹ indicates a hydrogen atom or a C1-C4 alkyl group which may be substituted with 1 to 5 halogen atoms, or is linked to X² via a C1 alkylene to form a 5-membered ring, wherein the C1 alkylene may be substituted with one or two C1-C4 alkyl groups (they may be also substituted with 1 to 5 halogen atoms);
R² indicates a hydrogen atom or a C1-C4 alkyl group which may be substituted with 1 to 5 halogen atoms, or is linked to X² via a C2 alkylene to form a 5-membered ring, wherein the C2 alkylene may be substituted with one or two C1-C4 alkyl groups (they may be also substituted with 1 to 5 halogen atoms), or is linked to X² via a C3 alkylene to form a 6-membered ring,
wherein the C3 alkylene may be substituted with one or two C1-C4 alkyl groups (they may be also substituted with 1 to 5 halogen atoms);
any one of R¹ and R² is linked to X² to form a ring;
X² indicates a single bond;
Y indicates a cyclobutylene group and may be substituted with 1 to 4 X^{Y}, and it binds to —CO₂R^{E} and —NR¹— at position 1 and position 3 of the cyclobutylene group, respectively;
X^{Y} indicates —OH, a halogen atom, or a C1-C4 alkyl group which may be substituted with 1 to 5 halogen atoms;
R^{E} indicates a hydrogen atom, a C1-C4 alkyl group, (CH₂)ₘN(R^{E1})(R^{E2}) or —C(R^{E3})₂OC(O)A^{E}R^{E4};
m indicates an integer of 2 or 3;
R^{E1} and R^{E2} can be the same or different from each other and each independently represent a methyl group, an ethyl group, or a propyl group, or a nitrogen-containing saturated cycloalkyl group in which R^{E1} and R^{E2} are linked to each other to form a 3- to 6-membered ring together with a nitrogen atom, or form a morpholino group together with a nitrogen atom;
R^{E3} indicates a hydrogen atom, a methyl group, an ethyl group, or a propyl group;
R^{E4} indicates a C1-C4 alkyl group, C3-C6 cycloalkyl group, or a phenyl group, and;
A^{E} indicates a single bond or an oxygen atom.].

2. The compounds according to claim 1, a possible stereoisomer, a racemate, a pharmaceutically acceptable salt, a hydrate, a solvate or a prodrug thereof, wherein R¹ is linked to X² via a C1 alkylene which may be substituted with one or two C1-C4 alkyl groups, to form a 5-membered ring.

3. The compounds according to claim 1, a possible stereoisomer, a racemate, a pharmaceutically acceptable salt, a hydrate, a solvate or a prodrug thereof, wherein R² is linked to X² via a C2 alkylene which may be substituted with one or two C1-C4 alkyl groups, to form a 5-membered ring.

4. The compounds according to claim 1, a possible stereoisomer, a racemate, a pharmaceutically acceptable salt, a hydrate, a solvate or a prodrug thereof, wherein R² is linked to X² via a C3 alkylene which may be substituted with one or two C1-C4 alkyl groups, to form a 6-membered ring.

5. The compounds according to any one of claims 1 to 4, a possible stereoisomer, a racemate, a pharmaceutically acceptable salt, a hydrate, a solvate or a prodrug thereof, wherein Y is an unsubstituted cyclobutylene group.

6. The compounds according to any one of claims 1 to 5, a possible stereoisomer, a racemate, a pharmaceutically acceptable salt, a hydrate, a solvate or a prodrug thereof, wherein -Z-V— is represented by Formula (2) (in the Formula (2), Z is as defined above).

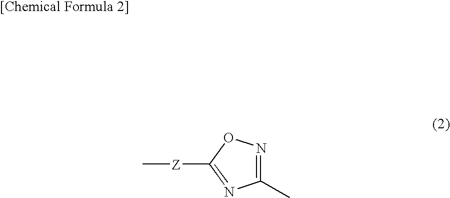

7. The compounds according to any one of claims 1 to 5, a possible stereoisomer, a racemate, a pharmaceutically acceptable salt, a hydrate, a solvate or a prodrug thereof, wherein -Z-V— is -Z-CR^{V1}R^{V2}—O— (Z, R^{V1}, and R^{V2} areas defined above).

8. The compounds according to any one of claims 1 to 5, a possible stereoisomer, a racemate, a pharmaceutically acceptable salt, a hydrate, a solvate or a prodrug thereof, wherein -Z-V— is -Z-(CR^{V1}R^{V2})—(CR^{V3}R^{V4})—O— (Z, R^{V1}, R^{V2}, R^{V3}, and R^{V4} are as defined above).

9. The compounds according to any one of claims 1 to 8, a possible stereoisomer, a racemate, a pharmaceutically acceptable salt, a hydrate, a solvate or a prodrug thereof, wherein bonding between Y and —NR¹— and bonding between Y and —CO₂R^{E} are in trans configuration.

10. The compounds according to any one of claims 1 to 9, a possible stereoisomer, a racemate, a pharmaceutically acceptable salt, a hydrate, a solvate or a prodrug thereof, wherein X^{W} is a C1-C4 alkyl group which may be substituted with 1 to 9-fluorine atoms, a C1-C4 alkoxy group which may be substituted with 1 to 9 fluorine atoms, a halogen atom, or a C1-C4 alkylthio group which may be substituted with 1 to 9 fluorine atoms.

11. The compounds according to any one of claims 1 to 10, a possible stereoisomer, a racemate, a pharmaceutically acceptable salt, a hydrate, a solvate or a prodrug thereof, wherein W is substituted with one or two X^{W}, and at least one X^{W} is a C1-C4 alkylthio group which may be substituted with 1 to 9 fluorine atoms, a C1-C4 acyl group which may be substituted with 1 to 7 fluorine atoms, or a C1-C4 alkyl group which is substituted with one C1-C4 alkoxy group which may be substituted with 1 to 9 fluorine atoms or with one —OH, and when W is substituted with two X^{W}, they can be the same or different from each other.

12. The compounds according to any one of claims 1 to 11, a possible stereoisomer, a racemate, a pharmaceutically acceptable salt, a hydrate, a solvate or a prodrug thereof, wherein Z may be substituted with one to three X^{Z}, and X^{Z} is a C1-C4 alkyl group which may be substituted with 1 to 9 fluorine atoms, a C1-C4 alkoxy group which may be substituted with 1 to 9 fluorine atoms, or a fluorine atom, and when Z is substituted with two or more X^{Z}, they can be the same or different from each other.

13. The compounds according to any one of claims 1 to 12, a possible stereoisomer, a racemate, a pharmaceutically acceptable salt, a hydrate, a solvate or a prodrug thereof, wherein Z is substituted with one to three X^{Z}, and X^{Z} is a methyl group or a fluorine atom, and when Z is substituted with two or more X^{Z}, they can be the same or different from each other.

14. The compounds according to any one of claims 1 to 13, a possible stereoisomer, a racemate, a pharmaceutically acceptable salt, a hydrate, a solvate or a prodrug thereof, wherein Z is substituted with two X^{Z}, and X^{Z} is a methyl group or a fluorine atom, and two X^{Z} can be the same or different from each other.

15. The compounds according to any one of claims 1 to 14, a possible stereoisomer, a racemate, a pharmaceutically acceptable salt, a hydrate, a solvate or a prodrug thereof, wherein W-Z-V— is represented by Formula (3) (in the Formula (3), W and V are as defined above).

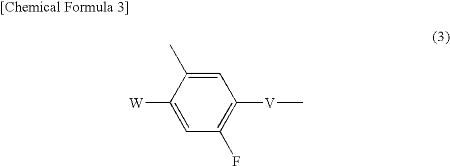

16. The compounds according to any one of claims 1 to 14, a possible stereoisomer, a racemate, a pharmaceutically acceptable salt, a hydrate, a solvate or a prodrug thereof, wherein W-Z-V— is represented by Formula (4) (in the Formula (4), W and V are as defined above).

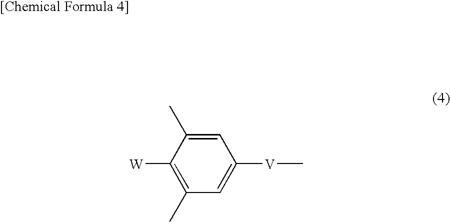

17. The compounds according to any one of claims 1 to 13, a possible stereoisomer, a racemate, a pharmaceutically acceptable salt, a hydrate, a solvate or a prodrug thereof, wherein W-Z-V— is represented by Formula (5) (in the Formula (5), W and V are as defined above).

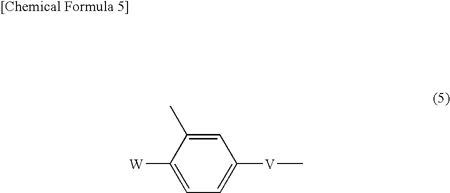

18. The compounds according to any one of claims 1 to 17, a possible stereoisomer, a racemate, a pharmaceutically acceptable salt, a hydrate, a solvate or a prodrug thereof, wherein X¹ is a trifluoromethyl group, a methyl group, an ethyl group, a fluorine atom, or a chlorine atom, and when two or more X¹ are present, they can be the same or different from each other.

19. The compounds according to any one of claims 1 to 18, a possible stereoisomer, a racemate, a pharmaceutically acceptable salt, a hydrate, a solvate or a prodrug thereof, wherein 1 is 1 and X¹ is a methyl group, a fluorine atom, or a chlorine atom.

20. The compounds according to any one of claims 1 to 19, a possible stereoisomer, a racemate, a pharmaceutically acceptable salt, a hydrate, a solvate or a prodrug thereof, wherein W is a monovalent group derived from benzene by the removal of one hydrogen atom.

21. The compounds according to any one of claims 1 to 19, a possible stereoisomer, a racemate, a pharmaceutically acceptable salt, a hydrate, a solvate or a prodrug thereof, wherein W is a monovalent group derived from thiophene by the removal of one hydrogen atom.

22. The compounds according to any one of claims 1 to 19, a possible stereoisomer, a racemate, a pharmaceutically acceptable salt, a hydrate, a solvate or a prodrug thereof, wherein W is a monovalent group derived from pyridine by the removal of one hydrogen atom.

23. A pharmaceutical agent which comprises as an effective component the compounds according to any one of claims 1 to 22, a possible stereoisomer, a racemate, a pharmaceutically acceptable salt, a hydrate, a solvate or a prodrug thereof.

24. A S1P1/Edg1 receptor agonist which comprises as an effective component the compounds according to any one of claims 1 to 22, a possible stereoisomer, a racemate, a pharmaceutically acceptable salt, a hydrate, a solvate or a prodrug thereof.

25. The pharmaceutical agent according to claim 24 which is used for prophylaxis and/or treatment of an autoimmune disease of mammals.

26. A method for the prophylaxis and/or treatment of an autoimmune disease of a mammal comprising administering to the mammal including human an effective amount of the compounds according to any one of claims 1 to 22, a possible stereoisomer, a racemate, a pharmaceutically acceptable salt, a hydrate, a solvate or a prodrug thereof.
